(19) United States
(12) Patent Application Publication
(10) Pub. No.: US 2022/0218878 A1
(43) Pub. Date: Jul. 14, 2022

(54) CONTROLLED HEAT DELIVERY COMPOSITIONS

(71) Applicant: BAMBU VAULT LLC, Lowell, MA (US)

(72) Inventors: Prakash RAI, Lowell, MA (US); Satish C. AGRAWAL, Sudbury, MA (US); Michael G. HORNER, West Roxbury, MA (US)

(73) Assignee: BAMBU VAULT LLC, Lowell, MA (US)

(21) Appl. No.: 17/613,423

(22) PCT Filed: May 25, 2020
(86) PCT No.: PCT/US2020/034460

§ 371 Date: Nov. 22, 2021

Related U.S. Application Data
(60) Provisional application No. 62/852,653, filed on May 24, 2019.
(60) Provisional application No. 62/852,679, filed on May 24, 2019.
(60) Provisional application No. 62/852,684, filed on May 24, 2019.
(60) Provisional application No. 62/852,702, filed on May 24, 2019.
(60) Provisional application No. 62/852,712, filed on May 24, 2019.

(51) Int. Cl. A61L 27/54; A61L 27/18; A61L 27/52; A61N 5/06; A61L 24/00; A61B 18/24

(52) U.S. Cl. CPC A61L 27/54; A61L 27/18; A61L 27/52; A61B 2018/00404; A61L 24/0015; A61B 18/24; A61N 5/062

(57) ABSTRACT

The disclosure describes a heat delivery medium and composition for biomedical applications with controlled conversion of energy from an exogenous source to heat.

The present application claims priority to U.S. Provisional Application No. 62/852,653, filed on May 24, 2019, U.S. Provisional Application No. 62/852,684, filed on May 24, 2019, U.S. Provisional Application No. 62/852,679, filed on May 24, 2019, U.S. Provisional Application No. 62/852,702, filed on May 24, 2019, and U.S. Provisional Application No. 62/852,712, filed on May 24, 2019, each of which is incorporated herein by reference in its entirety for all purposes.

### FIELD OF THE INVENTION

This invention is in the field of novel heat delivery compositions, particle heaters, and methods for their biomedical applications with controlled conversion of energy from an exogenous source into heat for tunable local temperature control.

### BACKGROUND OF THE INVENTION

Stimuli-responsive materials are a new class of soft materials that through their responsivity, layering, gradient, micro- and meso-patterns can have dramatic and surprising changes in shape and function in response to exogenous stimuli, or their physiological and environmental cues. These stimuli-responsive materials may include electrically responsive material, photothermal-responsive material, magneto-responsive material, or biomolecule-responsive material. These materials have various biomedical applications, especially in medical therapies, drug delivery, surgical devices, medical devices and tissue engineering. Stimuli-responsive materials offer significant prospects for a new class of dynamic, “smart”, and multifunctional materials, as well as adaptive structures, with a wide-range of applications. Current methods for heat delivery in the medical field suffer from high collateral damage with limited control on targeting the delivery of the heat to the target site.

Millions of surgeries and traumatic wound closures are performed worldwide every year. Most of these wounds are closed using mechanical methods such as sutures and staples. These methods suffer from several disadvantages such as patient discomfort, a risk of infection, and possible damage to the surrounding tissue. In recent years, surgical adhesives have been used for sealing wounds, but most of these adhesives do not work well. In addition, it is challenging to make medical adhesives that are nontoxic and biocompatible. Medical adhesives are increasingly being used after surgery and in emergencies for wound closure. Most tissue adhesives involve the use of cyanoacrylate, which can polymerize in the presence of moisture to form a seal. Other medical adhesives involve the use of poly(methyl methacrylate) (PMMA) or polyurethanes. Biocompatible tissue adhesives as well as novel resorbable bone glues are also being developed for bone fusion in case of bone injuries or fractures. However, these medical adhesives require over a minute of persistent pressure be applied in the right direction to cure the glue and seal the desired tissues. Gaps can form in between the tissues if the tissues to be sealed are not held in the correct position with the right pressure for the proper duration of time to allow the adhesive to cure. These gaps can act as open wounds for microbial infections, which can complicate healing. These openings can also result in excessive scar formation, which can further complicate the process. The idea to glue broken bones with a suitable biocompatible and bioabsorbable adhesive remains extremely attractive to orthopedic surgeons. Typically, the surgeon uses a liquid solution containing the monomer, which self-polymerizes in the presence of an initiator to form the pliable polymer in situ. Some residual monomers left behind after the surgery have acute and chronic toxicity which can cause complications.

Therefore, there exists a need for an on-demand medical adhesive or glue that can address the problems mentioned above. The present invention provides such on-demand medical adhesive or glue that can be cured by an exogenous source, resulting in complete wound closure and faster healing. The present invention also accelerates the polymerization reaction and reduces the amount of residual monomers and toxicity thereof without reducing the mechanical strength and flexibility of the adhesives.

Composites consisting of polymerizable resins and fillers have been widely used as dental compositions, for example, dental restoration composition, cements etc.

For almost 60 years, poly(methyl methacrylate) (PMMA)-based bone cement, commonly known as acrylic bone cement, has been used for fixation of total joint replacement prosthetic devices to periprosthetic bone. Today, most acrylic bone cements on the market consist of two components: a liquid and a powder, which are mixed in the operating room until they become dough-like and are then applied to the bone prior to insertion of the component of the joint replacement prosthesis. The primary function of cements is to fix the joint replacement prosthesis to the periprosthetic bone tissue. In the fixation of the joint replacement, the self-curing cement fills the free space between the prosthesis, and the bone. The cement grout serves to immobilize the implant and to transfer service loadings from it to the bone. Bone cement also provides a mechanical buffer between the bone and the prosthetic components, reducing stress and absorbing mechanical shocks.

PMMA based bone cements must be pre-mixed to form a dough like material prior to applying them at the bone because the polymerization reaction can be very exothermic. Unreacted monomer (methyl methacrylate) and initiators that may be part of the “dough” applied at the bone site can cause acute and chronic toxicities. Radical polymerization of the MMA in bone cement generally does not proceed to completion, because the mobility of remaining monomer molecules is inhibited at high conversion rates. There will therefore remain some residual methyl methacrylate monomer. Directly after curing, the content of residual monomer is approximately 2%-6%. The rates of curing of the bone cements and dental fillers can be very sensitive to environmental factors. For example, low ambient temperatures during storing and mixing, and high humidity can both prolong setting time. As time goes by, the cured acrylic bone cements can also shrink in volume thereby creating open spaces or gaps around the joints that they are supposed to fill. PMMA has several recognized shortcomings as a structural material. Aseptic loosening remains the major long-term problem with total joint replacement. In a bone cement system, there are three different materials (bone, cement, and implant) and two interfaces (bone and bone cement, bone cement and implant). The properties at the interfaces are mismatched because the cement is much weaker than the bone and the implant. Fatigue and fracture of cement have been implicated in the failure of these devices.

Alternative approaches that can reduce variabilities in curing rates, improve strength and flexibility at the interfaces as well as reduce the acute and chronic toxicities associated with acrylic bone cements are therefore needed.

The conventional approach to joining tissue segments following surgery, injury or the like, has been to employ mechanical sutures or staples. Most conventional suturing devices only achieve partial wound closure due to loose stitches, leaving open spaces or gaps in the wound area, which can result in infection and slow healing response. These in turn can increase hospital discharge times and add to the healthcare costs. However, over-tightening of stitches can add extra pressure to the wounded area, delay healing, increase the risk of local wound complications and caused unwanted scars. Over-tightening of the sutures may also cause the stitches to rip which would cause wound opening.

There exists a need for better devices and methods for accurately controlling the formation of anastomotic bonds, which avoid thermal damage and achieve optimal results. This disclosure provides suture devices and methods, which can tighten sutures to the desired level, accelerating the wound healing.

Damage to a blood vessel can lead to rapid blood loss, hypothermia and even death. Blood vessel damage can occur during surgery or due to injury through accidents or during war. Severe traumatic injuries can often lead to hypothermic bleeding. The process of stopping the loss of blood is called hemostasis that involves the formation of a temporary block by creating blood clots to reduce bleeding. Applying pressure (or compression) to the injury/wound site can usually reduce the flow of blood and allow the clot to form without a lot of blood loss.

Hemostasis in cases where bleeding results in body hypothermia is difficult as the clot formation can take a long time. Hemostats are agents that accelerate blood clotting. Hemostats are used if the bleeding is heavy or the subject is suffering from certain conditions or genetic diseases that prevent blood clotting. The use of hemostats must be swift, localized to the wound/injury site and carefully controlled. Reducing clotting time by even a few seconds to under 2 minutes can be valuable to save lives in the surgery room.

Current hemostatic agents on the market can make blood clot in approximately 200 seconds in standard in vitro tests. Reducing this time to clot formation by even 20-30% can dramatically reduce loss of lives and improve wound healing and recovery times. Hemostasis failure rates can be high (as high as 50%) with some current hemostats. Therefore, more efforts are needed in further research in finding better hemostatic agents and reducing the time to hemostasis. This is especially critical for severe bleeding leading to body hypothermia.

Thermal coagulation of serum has been known for at least 70 years. Photothermal coagulation is a technique that consists of irradiating the tissue with light energy that is subsequently converted into thermal energy, thereby inducing coagulation of the blood vessel. Photothermal coagulation is typically achieved using a laser device with a wavelength in the range of 700 nm to 10,000 nm wherein the light is absorbed mainly by the water present in the tissues. Such a procedure generally does not demonstrate selective action on the hematic components but induces coagulation of all the tissue and often has an excessive thermal effect that consequently causes collateral damage to the surrounding tissues.

There exists a need for selective thermal heating of blood components to minimize collateral damage to the surrounding tissues.

This disclosure provides novel controlled heat delivery compositions and particles that are responsive to an exogenous source with minimal collateral damage. This application also provides methods and applications for use of the inventive heat delivery compositions and particles.

### SUMMARY OF THE INVENTION

In some embodiments, this disclosure provides a heat delivery medium comprising of a carrier and a material that interacts with an exogenous source, wherein the material absorbs energy from the exogenous source and converts the absorbed energy to heat, wherein the heat travels outside the medium in a controlled temperature range to initiate or accelerate a physical, chemical or biological activity, and wherein the medium passes the Extractable Cytotoxicity Test. In some embodiments, the heat delivery medium further passes the Thermal Cytotoxicity Test.

In some embodiments, the heat delivery medium further passes the Efficacy Determination Protocol. In some embodiments, the heat delivery medium further passes the Thermal Cytotoxicity Test.

In some embodiments, the material exhibits at least 20% energy-to-heat conversion efficiency. In some embodiments, the material exhibits at least 20% efficiency of conversion of the energy from the exogenous source to heat.

In some embodiments, the carrier is selected from the group consisting of oil carriers, including fatty ester oils, squalene, squalene, hydrocarbon oil, light mineral oil, isoparaffin, paraffin oil, water, alcohol solution in water (C1-C4 alcohols), aqueous solution of polyhydric alcohol (e.g. glycerol, ethylene glycol, 1,3-propanediol, 1,4-butanediol), emulsion, saline, PBS buffer, and combinations thereof. In some embodiments, the carrier is selected from the group consisting of lipid, film forming polymer, thermoresponsive polymer, pressure sensitive adhesive, shape memory polymer, hydrogel, and combinations thereof. In some embodiments, the carrier is a coating composed of film forming polymer. In some embodiments, the film forming polymer is selected from the group consisting of poly(methyl methacrylate), poly(lactide-co-glycolide) (PLGA), block copolymer of PLGA, polyethylene glycol (PLGA-PEG), and combinations thereof.

In some embodiments, the material has significant absorption of photonic energy in the near infrared spectrum region having a wavelength range from 750 nm to 1100 nm.

In some embodiments, the material interacting with the exogenous source has significant absorption of photonic energy in the visible range. In some embodiments, the material absorbs light at a wavelength ranging from 400 nm to 750 nm. In some embodiments, the material absorbs light at a wavelength selected from the group consisting of 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, and 750 nm. In some embodiments, the material is selected from the group consisting of a tetrakis aminium dye, a cyanine dye, a squaraine dye, a squarylium dye, iron oxide, a plasmonic absorber, a zinc iron phosphate pigment, and combinations thereof.

In an embodiment, this disclosure provides a heat delivery composition comprising the heat delivery medium as disclosed herein and a structural element selected from a group consisting of a fiber, a film, a sheet, an implant scaffold, a tape, a stent, a hydrogel, a patch, an adhesive, a woven fabric, a nonwoven fabric, a biocompatible crosslinked polymer, and combinations thereof.

In some embodiments, the heat delivery medium is embedded within or layered on the surface of the structural element as a coating.

In some embodiments, the structural element comprises a biocompatible cross-linked polymer. In some embodiments, the biocompatible cross-linked polymer comprises a thermoresponsive polymer. In some embodiments, the biocompatible cross-linked polymer comprises a thermoresponsive shape memory polymer.

In some embodiments, the structural element further comprises an inorganic agent. In some embodiments, the inorganic agent is selected from the group consisting of apatite, hydroxyapatite, hydroxycarbonate apatite, calcium carbonate, calcium phosphate including monocalcium phosphate, dicalcium phosphate, tricalcium phosphate, and tetracalcium phosphate, and combinations thereof.

In an embodiment, this disclosure provides a particle having the carrier admixed with the material that interacts with an exogenous source. In some embodiments, the particle is a nanoparticle or a microparticle. In some embodiments, the particle maintains integrity after interacting with the exogenous source. In some embodiments, the particle structure is altered after interacting with the exogenous source.

In some embodiments, the particle may further comprise a shell to form a core-shell particle. In some embodiments, the shell comprises an agent selected from the group consisting of Au, Ag, Cu, iron oxide, and combinations thereof. In some embodiments, the shell comprises a plasmonic absorber. In some embodiments, the plasmonic absorber comprises plasmonic nanomaterials of noble metal gold (Au), silver (Ag) and copper (Cu) nanoparticles doped with sulfur (S), selenium (Se) or tellurium (Te) having a plasmonic resonance at a NIR wavelength.

In some embodiments, the carrier comprises a lipid, an inorganic agent, an organic polymer, or combinations thereof.

In some embodiments, the carrier comprises a biocompatible material selected from the group consisting of mesoporous silica, poly(methyl methacrylate), polyester including poly(lactic acid-co-glycolic acid) (PLGA), polyglycolic acid (PGA), polylactic acid (PLA), polycaprolactone (PCL), poly(trimethylene carbonate), poly (alpha-esters), polyurethanes, poly(allylamine hydrochloride), poly(ester amides), poly (ortho esters), polyanyhydrides, poly (anhydride-co-imide), crosslinked polyanhydrides, pseudo poly(amino acids), poly (alkylcyanoacrylates), polyphosphoesters, polyphosphazenes, chitosan, collagen, gelatin, natural or synthetic poly(amino acids), elastin, elastin-linked polypeptides, albumin, fibrin, polysiloxanes, polycarbosiloxanes, polysilazanes, polyalkoxysiloxanes, polysaccharides (e.g. chitosan), cross-linkable polymers, block co-polymers comprising polyethylene glycol, block co-polymers comprising polyoxyalkylene, and combinations thereof.

In some embodiments, the carrier is selected from the group consisting of poly (lactic acid) (PLA); poly(glycolic acid) (PGA); poly(lactide-co-glycolide) (PLGA); block copolymer of polyethylene glycol-b-poly lactic acid-co-glycolic acid (PEG-PLGA); polycaprolactone (PCL); poly-L-lysine (PLL); random graft co-polymer with a poly(L-lysine) backbone and poly(ethylene glycol) (PLL-g-PEG); dendritic polymer including polyethyleneimine (PEI) and derivatives thereof, dendritic polyglycerol and derivatives thereof, dendritic polylysine; and combinations thereof. In some embodiments, the carrier comprises polyester selected from the group consisting of poly(lactic acid) (PLA), poly(glycolic acid) (PGA), PLGA, and combinations thereof. In some embodiments, copolymers of PEG or derivatives thereof with any of the polymers described above may be used as carrier to make the polymeric particles. In some embodiments, the carrier comprises a polymer blend containing PLGA 75:25 and PLGA-PEG 75:25 with lactide:glycolide monomer ratio of 75:25.

In some embodiments, the carrier is a lipid. In some embodiments, the lipid is selected from the group consisting of 1,2-dipalmitoyl-sn-glycero-3-phospho-(1′-rac-glycerol) (DPPG), 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-distearoyl-sn-glycero-3-phosphoethanolamine (DSPE, 18:0), 1,2-diarachidyl-sn-glycero-3-phosphoethanolamine (20:0 PE), 1-stearoyl-2-linoleoyl-sn-glycero-3-phosphoethanolamine, 1,2-dipalmitoyl-sn-glycero-3-phosphocholine (DPPC, 16:0 PC), 1,2-diheptadecanoyl-sn-glycero-3-phosphocholine (17:0 PC), 1,2-distearoyl-sn-glycero-3-phosphocholine (DSPC, 18:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof. In some embodiments, the lipid comprises a thermoresponsive lipid/polymer hybrid. In some embodiments, the thermoresponsive lipid/polymer hybrid is selected from the group consisting of triblock copolymer of [poly(2-isopropyl-2-oxazoline)-b-poly(dimethylsiloxane)-b-poly(2-isopropyl-2-oxazoline] and 1,2-dimyristoyl-sn-glycero-3-phosphocholine (DMPC) composite, block copolymers poly(cholesteryl acrylate)-b-poly(N-isopropylacrylamide) (PNIPAAm) and lipid composite, and combinations thereof.

In some embodiments, this disclosure provides a method for controlled heat generation comprising contact of the heat delivery medium, or heat delivery composition, or particle heater as disclosed herein with an exogenous source.

In some embodiments, the exogenous source is selected from the group consisting of an electromagnetic radiation, an electrical field, a microwave, a radio wave, ultrasonic radiation, a magnetic field, and combinations thereof. In some embodiments, the exogenous source comprises a laser light. In some embodiments, the exogenous source comprises a LED light. In some embodiments, the laser light is a pulsed laser light. In some embodiments, the laser pulse duration is in a range from milliseconds to nanoseconds, and the laser has an oscillation wavelength at 1064 nm. In some embodiments the laser emits light at 808 nm. In some embodiments the laser emits light at 805 nm

In some embodiments, the heat delivery medium absorbs the laser light having a wavelength ranging from 750 nm to 1400 nm. In some embodiments, the material is a tetrakis aminium dye. In some embodiments, the material is a cyanine dye. In some embodiments, the material is a squaraine dye. In some embodiments, the material is a squarylium dye. In some embodiments, the material is iron oxide. In some embodiments, the material is a plasmonic absorber. In some embodiments, the material is a zinc iron phosphate pigment.

In some embodiments, the method further comprises heating the surrounding area in the proximity of the heat delivery medium or the particle heater by transferring heat from the medium to the surrounding area to induce localized hyperthermia. In some embodiments, the induced hyperthermia is a mild hyperthermia at a temperature ranging from about 38.0° C. to about 41.0° C. In some embodiments, the induced hyperthermia is a moderate hyperthermia at a temperature ranging from about 41.1° C. to about 45.0° C. In some embodiments, the induced hyperthermia is a profound hyperthermia at a temperature ranging from about 45.1° C. to about 52.0° C.

In an embodiment, this disclosure provides a hemostatic composition useful for the enhancement of the clotting of blood in a subject. The hemostatic composition comprises (i) a particle heater having a carrier admixed with a material interacting with an exogenous source, and (ii) a physiologically acceptable medium, wherein the material absorbs the energy from the exogenous source and converts the absorbed energy in to heat, wherein the heat travels outside the hemostatic composition to an area surrounding the hemostatic composition, wherein the heat causes a controlled temperature rise to initiate or accelerate the formation of a blood clot, and wherein the hemostatic composition passes the Extractable Cytotoxicity Test. In some embodiments, the particle heater is a microparticle or nanoparticle. In an embodiment, the subject is a warm-blooded animal. In an embodiment, the subject is a human.

In some embodiments, the hemostatic composition further comprises a hemostatic or coagulative agent selected from the group consisting of chitosan, calcium-loaded zeolite, silicate including kaolin, microfibrillar collagen, oxidized regenerated cellulose, anhydrous aluminum sulfate, silver nitrate, potassium alum, titanium oxide, fibrinogen, epinephrine, calcium alginate, poly-N-acetyl glucosamine, thrombin, coagulation factor(s) including Factor VII (FVII), Factor IX, Factor X, FVIIa, Von Willebrand factor, procoagulants including propyl gallate, antifibrinolytics including—ε-aminocaproic acid, coagulation proteins that generate Factor VII or FVIIa including Factor XII, Factor XIIa, Factor X, Factor Xa, protein C, protein S, and prothrombin, and combinations thereof.

### BRIEF DESCRIPTION OF THE DRAWINGS

FIG. 1 is a flowchart of the feedback loop for identifying optimal particle structure.

FIG. 2 illustrates the particle size distribution measured by Horiba LA-950 particle size analyzer in de-ionized water with pH 7.4.

FIG. 3 illustrates the degradation of Epolight™ 1117 measured at 1064 nm wavelength after exposure to 80° C.

FIG. 4 illustrates the controlled heat generation from laser excited Epolight™ 1117 IR dye-loaded particles dispersed in gelatin. A red 50° C. thermochromic dye was suspended in gelatin as an indicator of heat generation by the color change from red color to colorless. Spots 1, 4, 5, 6, 7 of FIG. 4 were exposed to laser irradiation from a Lutronic laser with a pulse width of 10 ns operated under Q-switched mode. Spots 2 and 3 were exposed with the Lutronic laser with a pulse width of 350 μs. Spots 8-16 were exposed with a semiconductor laser using various pulse widths from 10-250 ms.

FIG. 5 illustrates the suspension of red thermochromic dye prior to laser exposure.

FIG. 6 illustrates the color change at spot 9 after two exposures with a semiconductor laser operated at a wavelength of 980 nm with a pulse width of 250 ms to produce a total fluence of 70.7 J/cm².

FIG. 7A illustrate the melting of gelatin and decolorization of red dye without any clearing of the IR dye at the spots 15 and 16 after laser irradiation at 980 nm and a total fluence of 14.7 J/cm² (FIG. 7B, Spot 15) and 14.1 J/cm² (FIG. 7C, Spot 16).

FIG. 7B illustrates the color state at spot 15 after irradiating Spot 15 with seven exposures of 30 ms at 980 nm and a total fluence of 14.7 J/cm².

FIG. 7C illustrates the color state at spot 16 after irradiating Spot 16 with a single exposure of 200 ms at 980 nm and a total fluence of 14.1 J/cm².

FIG. 8A and FIG. 8B illustrate the laser-triggered blood clotting by particle heaters.

FIG. 9 illustrates the laser-triggered blood clotting by particle heaters for blood samples under different temperatures.

### DETAILED DESCRIPTION OF THE INVENTION

Molecules and materials that can absorb energy of an exogenous source to generate heat for controlled and localized temperature increments are potentially valuable for numerous applications in the biomedical field.

One of the challenges associated with the biomedical applications of the energy-to-heat materials is non-uniform and inefficient heating during and after the irradiation of the composition containing photo-absorbing chromophores such like indocyanine green, vital blue, and carbon black with an exogenous light source supplied in situ due to the poor penetration of the radiation through the tissue. Additionally, production of sufficient and uniform heat using this technique remains a challenge. Some of these chromophores may cause toxicity to the body. Furthermore, the chromophores may be degraded by the body into unwanted chemicals that are toxic to the body. Degradation of the chromophores by the body may also lead to insufficient heating at the site of action and thereby increase the dose required for effective heating which can compound toxicity to the body. Thermal cytotoxicity due to the heat generated following the irradiation of the energy-to-heat materials can also be a problem that has not been adequately addressed in the prior art on energy-to-heat materials.

Therefore, there exists a need for an easier, less toxic, more efficient and less invasive way to heat stimuli-responsive thermal materials in vivo, to a desired temperature above the body temperature without causing collateral tissue damage. Furthermore, such techniques should also be able to uniformly heat the area surrounding the stimuli-responsive thermal material.

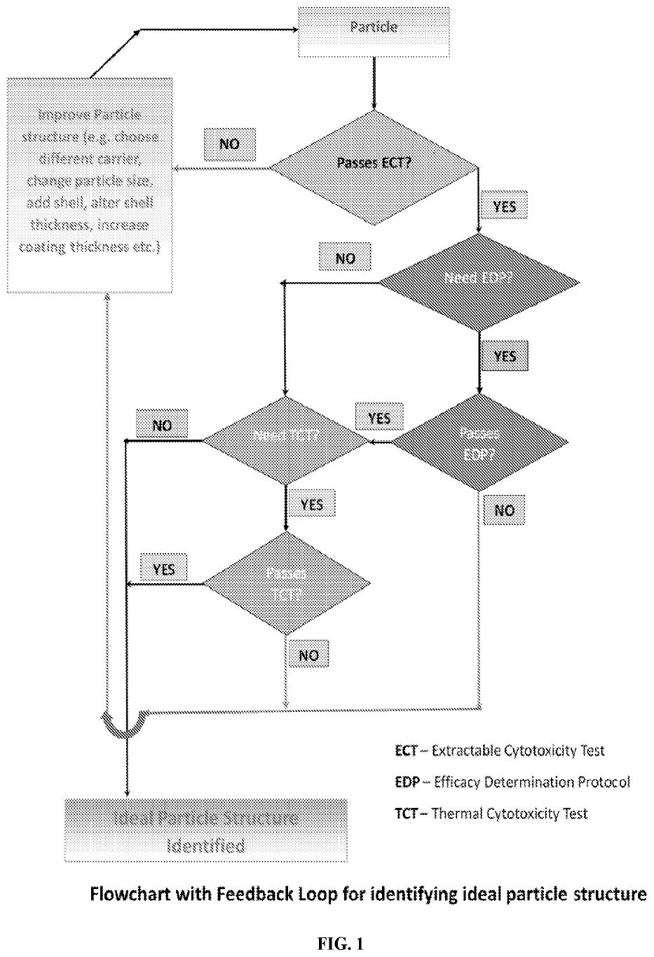

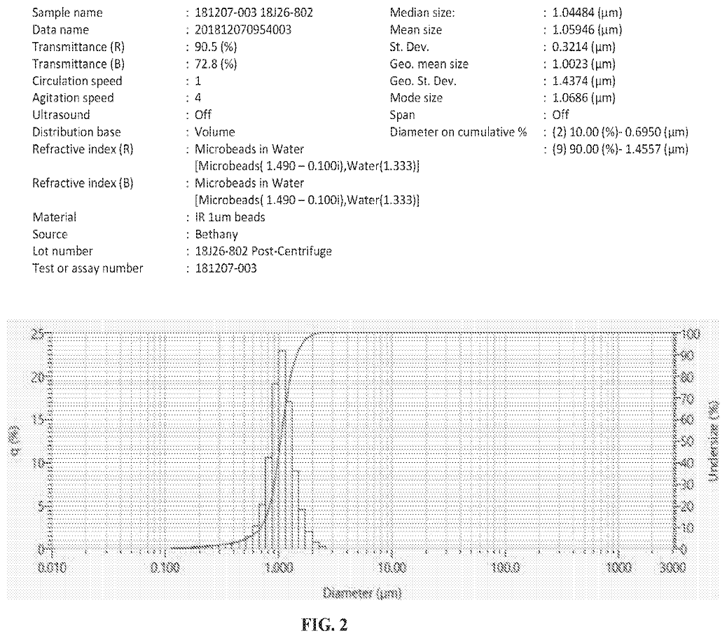

In some embodiments, the energy-to-heat conversion efficiency is measured by the stability of an IR absorbing dye encapsulated within the particle after exposure to optical irradiation such as from a pulsed laser. For example, the particle is considered passing the Efficacy Determination Protocol if the material exhibits at least 20% efficiency of conversion of the energy from the exogenous source to heat and/or the material exhibits at least 20% energy-to-heat conversion efficiency. In some embodiments, the degree of degradation for the material encapsulated within the particle can be determined using the IR dye loading determination protocol set forth in Example 2 below. The degradation of non-encapsulated IR absorbing dye can also be compared to that of encapsulated IR absorbing dye to evaluate the effect of encapsulation in particles. Depending on the application, different biological agents can be added to the cell culture media to simulate conditions that occur in vivo. This protocol in conjunction with the Extractable Cytotoxicity Test and/or Thermal Cytotoxicity Test will provide feedback (feedback loop protocol) to optimize the particle structure such that the material can be protected from the degradation by body chemicals. The Extractable Cytotoxicity Test is conducted according to the protocols described elsewhere herein (See FIG. 1). The particle structure characteristics (e.g. carrier material selection, particle size, morphology, particle surface modification etc.) and the laser irradiation method characteristics (e.g. laser wavelength, pulse duration and energy efficiency) are optimized sequentially based on the structure-property relationship feedbacks provides from the tests in the flow chart of FIG. 1 including Extractable Cytotoxicity Test, Efficacy Determination Test and/or Thermal Cytotoxicity Test. The ideal particle heaters possess the characteristics of high energy-to-heat conversion efficiency, stability (including thermal stability), and low collateral damage.

### Definitions

As used in the preceding sections and throughout the rest of this specification, unless defined otherwise, all technical and scientific terms used herein have the same meaning as is commonly understood by one skilled in the art to which this invention belongs. All patents and publications referred to herein are incorporated by reference in their entireties.

The term “a”, “an”, or “the” as used herein, generally is construed to cover both the singular and the plural forms.

The term “about” as used herein, generally refers to a particular numeric value that is within an acceptable error range as determined by one of ordinary skill in the art, which will depend in part on how the numeric value is measured or determined, i.e., the limitations of the measurement system. For example, “about” can mean a range of ±20%, ±10%, or ±5% of a given numeric value.

The term “absorption” as used herein, generally refers to the process of matter taking up exogenous source energy to transform the state of that matter to a higher electronic state when interacting with an exogenous source described herein. The process of absorption leads to an attenuation in the intensity of the exogenous source energy.

The term “energy-to-heat conversion efficiency” describes the percentage of absorbed exogenous energy that is converted into heat, as determined by a rise in temperature.

The term “photothermal conversion efficiency” describes the percentage of absorbed radiant energy that is converted into heat, as determined by a rise in temperature.

The term “amphiphilic block copolymer” as used herein refers to block copolymer having an average molecular weight 5 KDa to 500 KDa comprising at least one hydrophilic block and at least one hydrophobic block. Amphiphilic block copolymers undergo two basic processes in solvent media: micellization and gelation.

The term “biocompatibility” as used herein, refers to the capability of a substance implanted in the body to perform with an appropriate host response in a specific application without causing deleterious changes.

The term “biocompatible polymer” as used herein, generally refers to polymers that are intended to interface with biological systems to evaluate, treat, augment or replace any tissue, organ or function of the body. Some of the characteristic properties of the biocompatible polymers include not having toxic or injurious effects on biological systems, the ability of a polymer to perform with an appropriate host response in a specific application, and ability of a bio polymer to perform its desired function with respect to a medical therapy, without eliciting any undesirable local or systemic effects in the recipient or beneficiary of that therapy, but generating the most appropriate beneficial cellular or tissue response in that specific situation, and optimizing the clinically relevant performance of that therapy.

The term “body chemicals” as used herein, generally refers to the existing chemicals in any one of the bodily fluids, neutrophil media, macrophage media, or any complete cell growth media.

The term “bodily fluid” as used herein, generally refers to the natural fluid found in one of the fluid compartments of the human body. The principal fluid compartments are intracellular and extracellular. A much smaller segment, the transcellular compartment, includes fluid in the tracheobronchial tree, the gastrointestinal tract, and the bladder; cerebrospinal fluid; and the aqueous humor of the eye. The bodily fluid includes blood plasma, serum, cerebrospinal fluid, or saliva. In an embodiment, the bodily fluid contains neutrophils and macrophages.

“Chitosan” refers to a cationic polysaccharide derived from chitin, a biopolymer found in the shells of crustaceans. Generally, chitosan is obtained by removing about 50% or more of acetyl groups of acetamide from chitin, and chitosan generally has a degree of acetylation of less than 50%. Chitosan comprises (1,4)-linked N-acetyl-D-glucosamine and D-glucosamine units. Chitosan exhibits relatively poor water solubility.

As used herein, “curing,” “polymerization,” and “cross-linking” are used interchangeably.

As used herein, the term “cross-linkable” refers to a chemical agent that is capable of forming covalent bonds between molecules, and in particular, polymer chains. Such inter-molecular cross-linking may also be accompanied by intra-molecular cross-linking, e.g. formation of covalent bonds between functional groups having complementary reactivity, such as reaction between —COOH and —NH₂. The cross-linkable material will generally be polymeric or macromolecular in form, the effect of the cross-linking being to form covalent bonds between such molecules, and so to establish a three-dimensional network or matrix.

“Degree of acetylation” refers to the ratio or percentage of amine groups along the backbone of a chitosan or chitosan derivative molecule (such as glycol chitosan or glycol chitin) that are acetylated.

The term “Efficacy Determination Protocol” as used herein, generally refers to the method used for determining the degree of the degradation of the material inside a particle after the material being treated with body chemicals for a period of time that simulates the use environment. Various analytical tools, like UV-VIS-NIR, NMR, HPLC, LCMS, etc., would be used to quantify the concentration of the material in the extracts and control. The details of the Efficacy Determination Protocol are described in Examples section of the disclosure. In some instances, if the degradation of the material is less than 90% after being subjected to the body chemicals, then the particle is considered passing the Efficacy Determination Protocol. In some instances, depending on the energy absorbance efficiency of the material and the physicochemical property of the material, if the degradation of the material is less than 85%, 80%, 75%, 70%, 65%, 60%, 55%, 50%, 45%, 40%, 35%, 30%, 25%, 20%, 15%, 10%, or 5%, then the particle is considered passing the Efficacy Determination Protocol.

The term “Extractable Cytotoxicity Test” as used herein, generally refers to an in vitro leaching protocol (using physiologically relevant media that contains serum proteins at physiological temperature) that can be used to extract the material from the particles. The extract can then be used as is (“neat” or 1×) or in serial dilutions (up to 10,000× dilutions) with the media in a cytotoxicity test against healthy cells (different cells will be chosen depending upon the application) as a surrogate measurement for the porosity of the particles. The neat or dilution of the extract that kills 30% of the cells can be measured and is referred to as an IC₃₀. Likewise, the neat or dilution of the extract that kills 10% of the cells can be measured and is referred to as an IC₁₀. The neat or dilution of the extract that kills 20% of the cells or below can be measured and is referred to as an IC₂₀. The neat or dilution of the extract that kills 40% or below of the cells can be measured and is referred to as an IC₄₀. The neat or dilution of the extract that kills 50% or below of the cells can be measured and is referred to as an IC₅₀. The neat or dilution of the extract that kills 60% or below of the cells can be measured and is referred to as an IC₆₀. The neat or dilution of the extract that kills 70% or below of the cells can be measured and is referred to as an IC₇₀. The neat or dilution of the extract that kills 80% or below of the cells can be measured and is referred to as an IC₈₀. The neat or dilution of the extract that kills 90% or below of the cells can be measured and is referred to as an IC₉₀. Details of the Extractable Cytotoxicity Test are described in Examples section of the disclosure. The Extractable Cytotoxicity Test is compliant with the international standards: ISO-10993-5 “Tests for cytotoxicity—in vitro methods”. In some instances, if the neat or dilution concentration of the material in the leachate is independently less than IC₁₀, IC₃₀, IC₄₀, IC₅₀, IC₆₀, IC₇₀, IC₈₀, or IC₉₀, the particle passes the Extractable Cytotoxicity Test.

The term “electromagnetic radiation” (EMR) as used herein, generally refers to a complex system of exogenous source energy composed of waves and energy bundles that are organized according to the length of the propagating wave. It includes radio waves, microwaves, infrared (IR), visible light, ultraviolet, X-rays, and gamma rays.

The term “energy fluence” as used herein, generally refers to the areal density of the energy of the light and expressed in joules/area, for example, joules/m² or joules/cm².

The term “gelation” as used herein refers to a process involving continuous increase in viscosity accompanied by gradual enhancement of elastic properties. The main cause of gelation in polymer systems is the enhancement of interactions between the dissolved polymer or their aggregates. In contrast to micellization, gelation occurs from the semi-dilute to the high concentration of block copolymer solution and results from an arrangement of ordered micelles.

The term “gelation temperature” as used herein, generally refers to the temperature at which the thermoresponsive hydrogel undergoes a sol-gel transition under a given set of conditions (e.g. pH, hydrogel pre-polymer concentration).

“Glycol chitosan” is a chitosan derivative that exhibits improved water solubility compared to chitosan due to the introduction of hydrophilic ethylene glycol groups. Glycol chitosan generally has a degree of acetylation of less than 50%.

“Glycol chitin” refers to an N-acetylated derivative of glycol chitosan having a degree of acetylation of at least 50%.

The term “hydrogel” as used herein refers to three dimensional networks made of cross-linked hydrophilic or amphiphilic polymers that are swollen in liquid without dissolving in them. Hydrogel has the capability to absorb a large amount of water. Hydrogels are low-volume-fraction 3D networks of molecules, fibers or particles with intermediate voids, filled with water or aqueous media. Hydrogels can be classified into two classes: one class is physical gel resulted from physical association of polymer chains, and the other class is chemical gels (or irreversible gel) of which the network linked by covalent bonds. The inclusion of functional groups as pendant groups or on the backbone of the 3D network allows the synthesis of hydrogels that swell in response to a variety of stimuli including temperature, electromagnetic fields, chemicals and biomolecules.

The term “hydrophilic,” as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water. Generally, materials with a water contact angle of less than 90° are considered to be hydrophilic.

The term “hydrophobic,” as used herein, refers to the property of lacking affinity for, or even repelling water. For example, the more hydrophobic a polymer (or polymer segment), the more that polymer (or polymer segment) tends to not dissolve in, not mix with, or not be wetted by water. Generally, materials with a water contact angle of greater than 90° are considered to be hydrophobic.

The term “infrared radiation” or “infrared” (IR) as used herein, generally refers to electromagnetic radiation (EMR) with longer wavelengths than those of visible light. IR wavelengths extend from the nominal red edge of the visible spectrum at 750 nm (frequency 400 THz), to 1 mm (300 GHz). IR is absorbed by a wide range of substances, causing them to increase in temperature as the vibrations dissipate as heat.

The term “the material” as used herein, refers to the material that interacts with an exogenous source described in the disclosure.

The term “localized surface plasmon resonance” (LSPRs, localized SPRs) as used herein refers to collective electron charge oscillations in metallic nanoparticles that are excited by light. In contrast with the case of bulk metal, when an agent existing on a local surface such as metal nanoparticles is irradiated with light having various wavelengths, polarization occurs on the surface of the metal nanoparticles and exhibits a unique characteristic of increasing the intensity of the electric field. Electrons excited by such polarized light form a group (plasmon) and locally vibrate on the surface of the metal nanoparticles. This phenomenon is called localized surface plasmon resonance (LSPR). They exhibit enhanced near-field amplitude at the resonance wavelength.

The term “Material Process Stability” as used herein refers to the preservation of the optical and physical characteristics of the material under conditions of use such that it can deliver heat as intended upon stimulation by the exogenous source.

The term “micellization” as used herein refers to the process of micelle formation in a block copolymer solution, in which the solvent is thermodynamically favorable for one block and unfavorable for the other. When the micellization takes place in diluted solutions of block copolymer at a certain temperature above a concentration, the concentration is called the critical micelle concentration (CMC). The thermoresponsive copolymers in the solution evolve to form micelles at a certain temperature which is called the critical micelle temperature (CMT).

The term “polymer molecular weight” as used herein might mean any one of three different things. The term might refer (1) to “average molecular weight” (Mi) that is the molecular weight as calculated by the weight of the molecule that is most prevalent in the mix that makes up copolymer. The term might refer (2) to “number average molecular weight” (Mn) that is the molecular weight as calculated by taking all the different-sized molecules in the mix that makes up polymer and calculating the average weight, i.e., adding up the weight of each molecule and dividing by the number of molecules. Or, the term might refer (3) to “weight average molecular weight” (Mw) that is the molecular weight as calculated by taking all the different-sized molecules in the mix that makes up copolymer and calculating their average weight while giving heavier molecules a weight-related bonus when doing so. The unit for the molecular weight is Dalton (Da), kilodalton (KDa, plural kilodaltons).

The term “near infrared radiation” (NIR) as used herein, generally refers to commonly used subdivision scheme for Infrared EMR with wavelengths extending from 750 nm (400 THz) to 1400 nm (214 THz).

The term “Nd:YAG” as used herein, generally refers to Neodymium-doped Yttrium Aluminum Garnet (YAG), a widely used solid-state crystal composed of yttrium and aluminum oxides and a small amount of the rare earth neodymium.

The term “photothermal therapy” (PTT) as used herein refers to a minimally invasive therapy in which photon energy is converted into heat in order to kill unwanted cells such as microbes, viruses, and bacteria.

The term “Polydispersity Index” (PdI) is defined as the square of the ratio of standard deviation (σ) of the particle diameter distribution divided by the mean particle diameter (2a), as illustrated by the formula: PdI=(σ/2a)². PdI is used to estimate the degree of non-uniformity of a size distribution of particles, and larger PdI values correspond to a larger size distribution in the particle sample. PdI can also indicate particle aggregation along with the consistency and efficiency of particle surface modifications. A sample is considered monodisperse when the PdI value is less than 0.1.

The term “power” as used herein, generally refers to the rate at which energy is emitted from a laser.

The term “power density (irradiance)” as used herein, generally refers to the quotient of incident laser power on a unit surface area, expressed as watts/cm² (W/cm²).

The term “pulse” as used herein, generally refers to the brief span of time for which, the focused and scanned laser beam interacts with a given point on the skin (usually ranging from picoseconds to milliseconds).

The term “Q-Switch” as used herein, generally refers to an optical device (Pockels cell) that controls the storage or release of laser energy from a laser optical cavity. Q-switching is a means of creating very short pulses (5-100 ns) with extremely high peak powers. Q stands for quality.

The term “stimuli-sensitive block copolymer hydrogel” refers to the reversible polymer networks formed by physical interactions and exhibit a sol-gel phase transition. Hydrogels can change gel structure in response to environmental stimuli such as temperature.

The term “solid solution” as used herein, refers to the material molecularly dissolved in the solid excipient matrix such as hydrophobic polymers, wherein the material is miscible with the polymer matrix excipient.

The term “solid dispersion” as used herein, refers to the material dispersed as crystalline or amorphous particles, wherein the material is dispersed in an amorphous polymer and is distributed randomly within the polymer matrix excipient.

The term “thermogelation” as used herein refers to a temperature triggered reversible solution-to-gel phase transition phenomenon. Amphiphilic block copolymers are one class of polymers which display thermogelation behavior. Factors that control thermogelation in amphiphilic block copolymers include molecular weight of block segments, chemical composition of blocks, polymer concentration in solution and end group functionality.

The term “Thermal Cytotoxicity Test” as used herein refers to an in vitro test specifically designed to test the compositions and the specific exogenous source(s) for their ability to spare healthy cells during use. The thermal cytotoxicity test is a trans-well assay wherein healthy cells are grown and exposed to different doses of the composition and the exogenous source. Viability of the healthy cells are assessed using standard colorimetric assays 24 hours after exposure of the cells to the compositions and exogenous source. Different types of healthy cells can be selected for this test for different applications. The composition and light dose(s) that do not kill any more than 30% of the healthy cells are considered passing the Thermal Cytotoxicity Test.

The term “thermal relaxation time (TRT)” as used herein, generally refers to a simplified mathematical model to estimate the time taken for the target to dissipate about 50% of the incident thermal energy. It is related to the size of the targeted particle, e.g., 10 picoseconds (4 nm particle), 400 picoseconds (50 nm particle), a few nanoseconds (particles ranging in size from 40-300 nm), 200-1000 nanoseconds (melanosomes, 0.5 μm), to hundreds of milliseconds (leg venules). Longer TRT means the target takes longer time to cool to 50% of the temperature achieved. For spherical targets with radius R the TRT may be determined using Eqn. (I). TRT=R²/6.75 k, Eqn. (I) where k is thermal diffusivity. For R=10 nanometers, 50 nanometers, and 5 picometers, TRT is about 160 picoseconds, 4 nanoseconds, and 40 picoseconds, respectively. Even if the epidermis is a strong competing absorber, it can be spared as long as the TRT of the target is longer than that of epidermis (3-5 milliseconds).

### 1. Heat Delivery Medium and Heat Delivery Composition

In some embodiments, this disclosure provides a heat delivery medium comprising a carrier and a material interacting with an exogenous source. The material absorbs energy from the exogenous source and converts the absorbed energy to heat. The heat travels outside the medium in a controlled temperature range to initiate or accelerate a physical, chemical, or biological activity. The medium passes an Extractable Cytotoxicity Test. In some embodiments, the material is embedded within, dispersed in, or forms a solid solution in the carrier. In some embodiments, the heat delivery medium may be prepared by molding, extrusion, electrospinning, spray drying, lyophilization, crosslinking, in situ crosslinking, and any method that is known in the art.

In some embodiments, the heat delivery medium may have a physical form selected from the group consisting of solutions, dispersions, suspensions, coating formulations, dry coating layers, lotions, granules, powders, microspheres, flakes, films, gel ointments, sponges, foams, pastes, adhesives, semisolid, hydrogel, and combinations thereof.

In some embodiments, the heat delivery medium has the physical form of a coating layer. In some embodiments, the heat delivery medium has the physical form of a hydrogel.

In some embodiments, the carrier and the material form a particle. In some embodiments the particles are microparticles and/or nanoparticles. In some embodiments, the heat delivery medium has the physical form of a solution, dispersion, or suspension.

In an embodiment, this disclosure provides a heat delivery composition comprising the heat delivery medium and a structural element selected from the group consisting of a fiber, a film, a sheet, an implant scaffold, a stent, a hydrogel, a patch, an adhesive, a woven fabric, a nonwoven fabric, a biocompatible cross-linked polymer, and combinations thereof. In some embodiments, the biocompatible cross-linked polymer comprises reactive functional groups selected from the group consisting of vinyl dimethyl sulfone group, hydroxyl group (—OH), thiol group (—SH), amine group (—NH₂), aldehyde group (—CHO), carboxylic acid group (—COOH), epoxy group, and combinations thereof.

In some embodiments, the heat delivery medium is embedded within or disposed on the surface of the structural element as a coating.

In some embodiments, the heat delivery composition comprises a biocompatible cross-linked polymer. In some embodiments, the biocompatible cross-linked polymer comprises a thermoresponsive hydrogel.

In some embodiments, the heat delivery composition comprises a liquid formulation, a fiber, a coating, an implant scaffold, a hydrogel, an adhesive, a tape, a patch, a woven fabric, a nonwoven fabric, a film, a sheet, a multilayered structure, or a biocompatible cross-linked polymer.

### (i) Carrier

In some embodiments, the carrier is selected from a group consisting of lipid, film forming polymer, thermoresponsive polymer, pressure sensitive adhesive, shape memory polymer, hydrogel, and combinations thereof. In some embodiments, the carrier comprises a liquid composition selected from the group consisting of oil carrier such as fatty ester oils, squalene, squalene, hydrocarbon oils such as light mineral oil, isoparaffin, paraffin oils, water, alcohol solution in water (C1-C4 alcohols), aqueous solution of polyhydric alcohol (e.g. glycerol, ethylene glycol, 1,3-propanediol, 1,4-butanediol), emulsion, saline, and PBS buffer.

In some embodiments, the carrier may include a lipid selected from the group consisting of lipid, polymer-lipid conjugate, carbohydrate-lipid conjugate, peptide-lipid conjugate, protein-lipid conjugate, and combinations thereof. In some embodiments, the lipid has a phase transition temperature (Tₘ) ranging from about 35° C. to about 120° C. In some embodiments, the lipid has a melting temperature Tₘ ranging from about 55° C. to about 60° C.

In some embodiments, the carrier may comprise a lipid selected from the group consisting of lipid, polymer-lipid conjugate, carbohydrate-lipid conjugate, peptide-lipid conjugate, protein-lipid conjugate, and combinations thereof. In some embodiments, the lipid may include one or more of the following: phospholipids such as phosphatidylcholines, phosphatidylserines, phosphatidylinositides, phosphatidylethanolamines, phosphatidylglycerols, phosphatidic acids; sphingolipids such as sphingomyelins, ceramides, phytoceramides, cerebrosides; sterols such as cholesterol, desmosterol, lanthosterol, stigmasterol, zymosterol, diosgenin, and combinations thereof.

In some embodiments, the carrier comprises a polymer-lipid conjugate, wherein the polymers conjugated to polar head groups of the lipid may include polyethylene glycol, polyoxazolines, polyglutamines, polyasparagines, polyaspartamides, polyacrylamides, polyacrylates, polyvinylpyrrolidone, or polyvinylmethyether.

In some embodiments, the carrier comprises a carbohydrate-lipid conjugate, wherein the carbohydrate is conjugated to the lipid and may include monosaccharides (glucose, fructose, glyceraldehydes etc.), disaccharides, oligosaccharides or polysaccharides such as glycosaminoglycan (hyaluronic acid, keratan sulfates, heparin sulfate or chondroitin sulfate), carrageenan, microbial exopolysaccharides, alginate, chitosan, pectins, chitin, cellulose, or starch.

In one embodiment, the phospholipid is selected from the group consisting of dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-dimyristoyl-sn-glycero-3-phosphorylglycerol (DMPG); 1,2-distearoyl-sn-glycero-3-phosphoethanolamine (DSPE); 1,2-dioleoyl-sn-glycero-3-phosphocholine (DOPC); 1,2-dioleoyl-sn-glycero-3-phosphoethanolamine (DOPE); 1,2-dipalmitoyl-sn-glycero-3-phospho-(1′-rac-glycerol) (DPPG); 1,2-distearoyl-sn-glycero-3-phosphocholine (DSPC), distearoylphosphoethanolamine conjugated with polyethylene glycol (DSPE-PEG); phosphatidylserine (PS), phosphatidylethanolamine (PE), phosphatidylglycerol (PG), phosphatidylcholine (PC), and combinations thereof. In an embodiment, the particle comprise the lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, PG, and combinations thereof.

In some embodiments, the lipid is selected from the group consisting of 1,2-dipalmitoyl-sn-glycero-3-phospho-(1′-rac-glycerol) (DPPG), 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-diarachidyl-sn-glycero-3-phosphoethanolamine (20:0 PE), 1-stearoyl-2-linoleoyl-sn-glycero-3-phosphoethanolamine, 1,2-diheptadecanoyl-sn-glycero-3-phosphocholine (17:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

In some embodiments, the lipid comprises a thermoresponsive lipid/polymer hybrid. In some embodiments, the thermoresponsive lipid/polymer hybrid contains a triblock copolymer of [poly(2-isopropyl-2-oxazoline)-b-poly(dimethylsiloxane)-b-poly(2-isopropyl-2-oxazoline] and 1,2-dimyristoyl-sn-glycero-3-phosphocholine (DMPC), and a lipid. In some embodiments, the thermoresponsive lipid/polymer hybrid contains a block copolymers poly(cholesteryl acrylate)-b-poly(N-isopropylacrylamide) (PNIPAAm) and a lipid.

In an embodiment, the carrier may include a lipid selected from the group consisting of lipid, polymer-lipid conjugate, carbohydrate-lipid conjugate, peptide-lipid conjugate, protein-lipid conjugate, and mixtures thereof. In some embodiments, the lipid may include one or more of the following: phospholipids such as phosphatidylcholines, phosphatidylserines, phosphatidylinositides, phosphatidylethanolamines, phosphatidylglycerols, phosphatidic acids; sphingolipids such as sphingomyelins, ceramides, phytoceramides, cerebrosides; sterols such as cholesterol, desmosterol, lanthosterol, stigmasterol, zymosterol, or diosgenin. In some embodiments, the carrier comprises a polymer-lipid conjugate, wherein the polymers conjugated to polar head groups of the lipid may include polyethylene glycol, polyoxazolines, polyglutamines, polyasparagines, polyaspartamides, polyacrylamides, polyacrylates, polyvinylpyrrolidone, or polyvinylmethyether. In some embodiments, the carrier comprises a carbohydrate-lipid conjugate, wherein the carbohydrates conjugated to the lipid may include monosaccharides (glucose, fructose), disaccharides, oligosaccharides or polysaccharides such as glycosaminoglycan (hyaluronic acid, keratan sulfates, heparin sulfate or chondroitin sulfate), carrageenan, microbial exopolysaccharides, alginate, chitosan, pectin, chitin, cellulose, or starch.

In some embodiments, the carrier comprises an inorganic agent. In some embodiments, the inorganic agent is selected from the group consisting of apatite, hydroxyapatite, hydroxycarbonate apatite, calcium carbonate, calcium phosphate including monocalcium phosphate, dicalcium phosphate, tricalcium phosphate, and tetracalcium phosphate, and combinations thereof.

In an embodiment, the carrier comprises a polymer. In some embodiments, the polymer is a biocompatible polymer. In some embodiments, the polymer is a biodegradable polymer. In some embodiments, the carrier is a polymeric fiber. In some embodiments, the polymeric fiber comprises biocompatible polymers. In some embodiments, the polymeric fiber comprises biodegradable polymers.

In some embodiments, the polymers may include, but are not limited to, a polyester, a polyurea, a polyanhydride, a polysaccharide, a polyphosphoester, a poly(ortho ester), a poly(amino acid), a protein, and combinations thereof.

In one embodiment, the carrier is a polyester. Polyesters are a class of polymers characterized by ester linkages in the backbone, such as poly (lactic acid) (PLA), poly(glycolic acid) (PGA), PLGA, etc. PLGA is one of the commonly used polymers in developing particulate drug delivery systems. PLGA degrades via hydrolysis of its ester linkages in the presence of water.

In some embodiments, the polymer selected from the group consisting of poly(lactic acid) (PLA), poly(glycolic acid) (PGA), PLGA, poly(lactic acid)-polyethylene glycol-poly(lactic acid) (PLA-PEG-PLA), poly (L-co-D, L lactic acid) 70:30 (PLDLA); poly-L-lactic acid-co-glycolic acid, poly-D,L-lactic acid-co-glycolic acid; poly-valerolactone, poly-hydroxyl butyrate and poly-hydroxyl valerate, polycaprolactone (PCL), γ-polyglutamic acid graft with poly (L-phenylalanine) (γ-PGA-g-L-PAE), poly(cyanoacrylate) (PCA), polydioxanone, polyvinylpyrrolidone (povidone, PVP), poly(butylene succinate), polyalkyleneoxalate, polyalkylene succinate, poly(maleic acid), poly(trimethylene carbonate), poly(p-dioxanone), poly(butylene terephthalate), poly(P-hydroxyalkanoate)s, poly(hydroxybutyrate), and poly(hydroxybutyrate-co-hydroxyvalerate), poly (ε-lysine), poly-L-lysine (PLL), poly(valeric acid), and poly-L-glutamic acid, poly(ester amide), poly(ester ether) diblock copolymer of poly(sebacic acid) and polyethylene glycol (PSA-PEG), trimethylene carbonate, poly(β-hydroxybutyrate), poly(g-ethyl-L-glutamate), poly(iminocarbonate), poly(bisphenol A iminocarbonate), polyphosphazene, collagen, albumin, gluten, chitosan, hyaluronate, hyaluronic acid, cellulose, alginate, starch, gelatin, pectin, crosslinked dextran as reaction product of dextran with epihalogenohydrins, dihalogenohydrins, 1:2,3:4-diepoxybutane, diepoxy-propylether, and combinations thereof.

In some embodiments, the carrier is selected from the group consisting of poly (lactic acid) (PLA); poly(glycolic acid) (PGA); poly(lactide-co-glycolide) (PLGA); block copolymer of polyethylene glycol-b-poly lactic acid-co-glycolic acid (PEG-PLGA); polycaprolactone (PCL); poly-L-lysine (PLL); random graft co-polymer with a poly(L-lysine) backbone and poly(ethylene glycol) (PLL-g-PEG); dendritic polymer including polyethyleneimine (PEI) and derivatives thereof, dendritic polyglycerol and derivatives thereof, dendritic polylysine; and combinations thereof.

In some embodiments, copolymers of PEG or derivatives thereof with any of the polymers described above may be used as carrier to make the polymeric particles. In some embodiments, the carrier comprises a polymer blend containing PLGA 75:25 and PLGA-PEG 75:25 with lactide:glycolide monomer ratio of 75:25. In some embodiments, the carrier comprises PEG5000-PCL10000 (PEG-PCL, 5000 and 10000 are molecular weight of the block), Maleimide-PEG5000-PCL10000 (Mal-PEG-PCL).

In some embodiments, the carrier comprises PEG grafted dendritic polymer PEI, polyglycerol, and polylysine. In some embodiments, the carrier comprises PEG grafted dendritic polymer PEI, polyglycerol, and polylysine, wherein the PEG is terminated with a reactive functional group selected from the group consisting of vinyl group (—CH═CH₂), ethynyl group (—C≡C—), vinyl methylsulfone group, hydroxyl group (—OH), thiol group (—SH), amine group (—NH₂), aldehyde group (—CHO), carboxylic acid group (—COOH), and combinations thereof. In some embodiments, the carrier comprises PEG grafted dendritic polymer PEI, polyglycerol, and polylysine, wherein the PEG is terminated with an amine group (—NH₂), wherein the amine group becomes cationically charged under mildly acidic condition (e.g. pH=4-6). In some embodiments, the carrier comprises PEG grafted dendritic polymer PEI, polyglycerol, and polylysine, wherein the PEG is terminated with a thiol group (—SH).

In some embodiments, the PEG or derivatives may be located in the interior positions of the triblock copolymer (e.g. PLA-PEG-PLA). Alternatively, the PEG or derivatives may be located near or at the terminal positions of the block copolymer. In some embodiments, the nanoparticles are formed under conditions that allow regions of PEG to phase separate or otherwise to reside on the surface of the particles.

In some embodiments, the carrier comprises PLGA. PLGA denotes a copolymer (or co-condensate) of lactic acid and glycolic acid. The PLGA copolymers for use in the present invention are preferably biodegradable, i.e. they degrade in an organism over time by enzymatic or hydrolytic action or by similar mechanisms, thereby producing pharmaceutically acceptable degradation products, and biocompatible, i.e. that do not cause toxic or irritating effects or immunological rejection when brought into contact with a body fluid. The lactic acid units may be L-lactic acid, D-lactic acid or a mixture of both.

In some embodiments, the polymethacrylate copolymer is MMA/BMA copolymer and the weight ratio of MMA to BMA is 96:4 (e.g. NeoCryl® 805 by DSM, acid value less than 1). In one embodiment, the carrier is poly (methyl methacrylate) (PMMA). In some embodiments, the carrier is a polyacrylate blend comprising 96% methyl methacrylate and 4% butyl methacrylate. In some embodiments, the carrier is a methyl methacrylate/butyl methacrylate copolymer comprising 96% methyl methacrylate repeating units and 4% butyl methacrylate repeating units. In some embodiments, the polymethyl methacrylate is a copolymer of methyl methacrylate/butyl methacrylate (NeoCryl® B-805, T_{g} 99° C., average molecular weight 85,000 Da).

In some embodiments, the carrier comprises cross-linkable reactive groups selected from vinyl group (—CH═CH₂), ethynyl group (—C≡C—), vinyl dimethyl sulfone group, hydroxyl group (—OH), thiol group (—SH), amine group (—NH₂), aldehyde group (—CHO), carboxylic acid group (—COOH), and combinations thereof. In some embodiments, the carrier comprises cross-linkable polysaccharides.

In some embodiments, the carrier is a protein selected from the group consisting of albumin, fibrin, lipoproteins, apoproteins, chylomicrons, silk fibroin, keratin, collagen, gelatin, ovalbumin, serum albumin, corn zein, soy protein, gluten, milk protein, and combinations thereof.

In some embodiments, the carrier comprises one or more polysaccharides selected from the group consisting of carrageenan, microbial exopolysaccharides, alginate, chitosan, pectins, chitin, cellulose, starch, and combinations thereof. In some embodiments, the carrier comprises cationically charged chitosan.

In some embodiments, the carrier comprises an organic polymer susceptible to proteolytic degradation by a protease. In some embodiments, the organic polymer is a protein selected from the group consisting of silk fibroin, lipoproteins, apoproteins, chylomicrons, keratin, collagen, gelatin, ovalbumin, serum albumin, elastin, corn zein, soy protein, gluten, milk protein, and combinations thereof. In some embodiments, the protein is silk fibroin. In some embodiments, the protein is milk protein. In some embodiments, the protein is collagen or gelatin. In some embodiments, the protein is ovalbumin or serum albumin. In some embodiments, the protein is a lipoprotein including low density, very low density, intermediate density, high-density lipoproteins, apoproteins, or chylomicrons.

In some embodiments, the protein is crosslinked. In some embodiments, the crosslinker reagent is selected from the group consisting of glutaraldehyde, tannin, dopamine, reducing sugar (Maillard reaction), genipin, and combinations thereof.

In some embodiments, the protein is selected from the group consisting of silk fibroin, keratin, collagen, lipoprotein, apoproteins, chylomicrons, gelatin, ovalbumin, serum albumin, elastin, corn zein, soy protein, gluten, milk protein, and combinations thereof. In some embodiments, the second protein is milk protein or collagen. In some embodiments, the milk protein is selected from the group consisting of casein (CAS), whey proteins (WP), β-lactoglobulin (β-LG), lactoferrin (Lf), and combinations thereof. In some embodiments, the second protein is silk fibroin. In some embodiments, the second protein is milk protein. In some embodiments, the second protein is collagen or gelatin.

In some embodiments, the second protein is casein. The term “casein” as used here refers to a group of casein proteins (αs1, β, αs2 and κ) found in milk as the major components. The dominant feature of milk is the casein micelle; a supramolecular aggregate imparts the white characteristic of milk. Because αs1, 2-caseins and β-caseins are highly phosphorylated, they are believed to bind with calcium to form the aggregates. κ-casein is thought to predominate on the micellar surface. Casein may be purified from milk. Casein exists in milk as the calcium salt, calcium caseinate. Calcium caseinate has its isoelectric point at a pH lower than the pH of milk; therefore, the casein micelle is solubilized. If acid is added to milk, the casein precipitates. Further extraction with ethanol allows for further purification In certain embodiments, the disclosure contemplates that casein is derived from other animals such as humans, buffaloes, goats, camels and sheep. In certain embodiments, the disclosure contemplates that the casein proteins may be produced by recombinant methods.

In some embodiments, the carrier is present in the particle at a weight percentage of the total weight of the particle selected from the group consisting of about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, or about 20.0 wt. %, about 25.0 wt. %, about 30.0 wt. %, about 35.0 wt. %, about 40.0 wt. %, about 45.0 wt. %, about 50.0 wt. %, about 55.0 wt. %, about 60.0 wt. %, about 65.0 wt. %, about 70.0 wt. %, about 75.0 wt. %, about 80.0 wt. %, about 85.0 wt. %, about 90.0 wt. %, about 95.0 wt. %, and about 99.0 wt. %. In some embodiments, the carrier is present in the particle at a weight percentage of the total weight of the particle ranging from about 1 wt. % to about 99 wt. %. In some embodiments, the carrier is present in the particle at a weight percentage of the total weight of the particle ranging from about 10.0 wt. % to about 90.0 wt. %. In some embodiments, the carrier is present in the particle at a weight percentage of the total weight of the particle ranging from about 50.0 wt. % to about 90.0 wt. %. In some embodiments, the carrier is present in the particle at a weight percentage of the total weight of the particle ranging from about 25.0 wt. % to about 50.0 wt. %. In some embodiments, the carrier is present in the particle at a weight percentage of the total weight of the particle ranging from about 75.0 wt. % to about 90.0 wt. %.

In some embodiments, the particle comprises NeoCryl® B-805 (copolymer of 96.0 wt. % methylmethacrylate/4.0 wt. % butyl methacrylate) in an amount ranging from about 60.0 wt. % to about 80 wt. % by the total weight of the particle. In some embodiments, the particle comprises NeoCryl® B-805 in an amount selected from the group consisting of 62.0 wt. %, 70.0 wt. %, 75.0 wt. %, and 78.3 wt. % by the total weight of the particle. In some embodiments, the particle comprises NeoCryl® B-805 in an amount selected from the group consisting of about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, and about 80 wt. % by the total weight of the particle.

In some embodiments, the polymer has a glass transition temperature (T_{g}) of at least 35° C. In some embodiments, the polymer has a glass transition temperature ranging from 35° C. to 120° C. In some embodiments, the polymer has a glass transition temperature ranging from 35° C. to 50° C. In some embodiments, the polymer has a glass transition temperature ranging from 45° C. to 100° C. In some embodiments, the polymer has a glass transition temperature ranging from 55° C. to 100° C. In some embodiments, the polymer has a glass transition temperature ranging from 75° C. to 100° C. In some embodiments, the polymer has a glass transition temperature ranging from 95° C. to 100° C. In some embodiments, the polymer has a glass transition temperature selected from the group consisting of 35° C., 36° C., 37° C., 38° C., 39° C., 40° C., 41° C., 42° C., 43° C., 44° C., 45° C., 46° C., 47° C., 48° C., 49° C., 50° C., 55° C., 60° C., 65° C., 70° C., 75° C., 80° C., 85° C., 90° C., 95° C., 100° C., 110° C., and 120° C. In some embodiments, the polymer has a glass transition temperature is selected from the group consisting of 95° C., 96° C., 97° C., 98° C., 99° C., and 100° C. In some embodiments, the polymer has a glass transition temperature at 99° C. It is preferred that the T_{g} of the polymer is greater than about 37° C.

In some embodiments, the carrier comprises a biocompatible material selected from the group consisting of mesoporous silica, poly(methyl methacrylate), polyester including poly(lactic acid-co-glycolic acid) (PLGA), polyglycolic acid (PGA), polylactic acid (PLA), polycaprolactone (PCL), poly(trimethylene carbonate), poly (alpha-esters), polyurethanes, poly(allylamine hydrochloride), poly(ester amides), poly (ortho esters), polyanyhydrides, poly (anhydride-co-imide), crosslinked polyanhydrides, pseudo poly(amino acids), poly (alkylcyanoacrylates), polyphosphoesters, polyphosphazenes, chitosan, collagen, gelatin, natural or synthetic poly(amino acids), elastin, elastin-linked polypeptides, albumin, fibrin, polysiloxanes, polycarbosiloxanes, polysilazanes, polyalkoxysiloxanes, polysaccharides (e.g. chitosan), cross-linkable polymers, block co-polymers comprising polyethylene glycol, block co-polymers comprising polyoxyalkylene, and combinations thereof. In some embodiments, the carrier comprises a crosslinked biocompatible and biodegradable polymer. In some embodiments, the crosslinked biocompatible polymer comprises a crosslinked polysaccharide. In some embodiments, the polysaccharide is selected from the group consisting of hyaluronic acid, alginate, alginic acid, starch, carrageenan, and combinations thereof. In some embodiments, the carrier comprises a methyl methacrylate/butyl methacrylate copolymer comprising 96% methyl methacrylate repeating units and 4% butyl methacrylate repeating units.

In some embodiments, the polymer is selected from the group consisting of PDMS (poly (dimethyl siloxane) (PDMS)), polydioxanone, poliglecaprone polypropylene, polyvinylidene fluoride, polyethylene terephthalate, polyethylene including ultra-high-molecular-weight polyethylene (UHMWPE), cross-linked UHMWPE, low density polyethylene (LDPE), high density polyethylene (HDPE), polyketones, polystyrene, polyvinyl chloride, poly (meth) acrylamides, polyetheretherketone (PEEK), poly(methyl methacrylate), polyester including poly(lactic acid-co-glycolic acid) (PLGA), polyglycolic acid (PGA), polylactic acid (PLA), polycaprolactone (PCL), poly(trimethylene carbonate), poly (alpha-esters), polyurethanes, poly(allylamine hydrochloride), poly(ester amides), poly (ortho esters), polyanyhydrides, poly (anhydride-co-imide), cross-linked polyanhydrides, pseudo poly(amino acids), poly (alkylcyanoacrylates), polyphosphoesters, polyphosphazenes, chitosan, collagen, gelatin, natural or synthetic poly(amino acids), elastin, elastin-linked polypeptides, albumin, fibrin, polysiloxanes, polycarbosiloxanes, polysilazanes, polyalkoxysiloxanes, polysaccharides, cross-linkable polymers, thermoresponsive polymers, thermo-thinning polymers, thermo-thickening polymers, block co-polymers comprising polyethylene glycol, and combinations thereof.

In some embodiments, the carrier is selected from the group consisting of PGA, PLA, PLGA, polydioxanone, polycaprolactone, and combinations thereof.

In some embodiments, the carrier is a film forming polymer selected from the group consisting of PLGA, PCL, PLGA-PEG, PMMA, and combinations thereof. In some embodiments, the carrier and the material forms a coating composition. In some embodiments, the carrier and the material forms a particle.

In some embodiments, the carrier comprises hydrogel having dendritic polymer. In some embodiments, the dendritic polymer comprises polyglycerol and dendritic polylysine.

In some embodiments, the carrier comprises a biocompatible cross-linked polymer. In some embodiments, the biocompatible cross-linked polymer comprises a hydrogel. In some embodiments, the hydrogel is a thermoresponsive hydrogel. In some embodiments, the thermoresponsive hydrogel is formed from hydrogel precursors.

Amphiphilic block copolymers are one class of polymers which display thermogelation behavior. Amphiphilic block copolymers comprising PLGA and poly(ethylene glycol) (PEG) are thermogelling polymers with biodegradable segments. The hydrophilic PEG block introduces the biocompatibility to the block copolymers. The PLGA block introduces biodegradability due to ester bonds with LA and GA repeating units.

Factors that control thermogelation in amphiphilic block copolymers include the molecular weight of block segments, the chemical composition of blocks, the polymer concentration in solution and end group functionality. The most important factors influencing the properties and the applications of the PLGA/PEG block copolymers are the chemical nature and the size of the hydrophobic segment, e.g. the molecular weight ratio between PEG and the PLGA blocks may be 0.56 or less in order to obtain the thermoresponsive hydrogels. In some embodiments, the thermoresponsive hydrogel comprises PLGA-PEG-PLGA with respective Mn of 1000-1000-1000 Da, wherein PLGA comprises 1:1 mole ratio of LA:GA. In some embodiments, the thermoresponsive hydrogel comprises PLGA-PEG-PLGA with respective Mn of 800-1000-800 Da, wherein PLGA comprises 1:1 mole ratio of LA:GA. In some embodiments, the thermoresponsive hydrogel comprises PLGA-PEG-PLGA with respective Mn of 1500-1200-1500 Da, wherein PLGA comprises 1:1 mole ratio of LA:GA. In some embodiments, the thermoresponsive hydrogel comprises PLGA-PEG-PLGA with respective Mn of 1500-1500-1500 Da, wherein PLGA comprises 1:1 mole ratio of LA:GA.

In one embodiments, the thermoresponsive hydrogel comprises poly(lactic acid-co-glycolic acid)-block-poly(ethylene glycol)-block-poly(lactic acid-co-glycolic acid) (PLGA-PEG-PLGA) copolymers. The use of block copolymers containing poly(ethylene glycol) and poly(lactide-co-glycolide) designed with low overall molecular weight and an appropriate balance between hydrophilic and hydrophobic blocks allows for the formation of biodegradable polymeric materials which can form aqueous solutions that undergo thermogelation at physiological temperatures. This is a useful property for a wide array of biomedical applications.

The thermogelation onset temperature of the copolymers can be tailored by the molecular weight of the degradable block segment and the ratio of lactide to glycolide. In some embodiments, the thermoresponsive hydrogel comprises PLGA-PEG-PLGA with respective Mn of 1500-1000-1500 Da, wherein PLGA comprises 1:1 mole ratio of LA:GA; thermogelation temperature for a 20 wt. % aqueous solution thereof is 17-23° C. In some embodiments, the thermoresponsive hydrogel comprises PLGA-PEG-PLGA with respective Mn of 1500-1000-1500 Da, wherein PLGA comprises 3:1 mole ratio of LA:GA; thermogelation temperature for a 20 wt. % aqueous solution thereof is 20-25° C.

In some embodiments, the thermoresponsive hydrogel exhibits a gelation temperature ranging from about 30° C. to about 36° C. In some embodiments, the thermoresponsive hydrogel may exhibit a gelation temperature of about 30° C., about 31° C., about 32° C., about 33° C., about 34° C., about 35° C., or about 36° C. at physiological pH. In some embodiments, the thermoresponsive hydrogel may exhibit a gelation temperature of 35° C. at pH 3.8. In some embodiments, the thermoresponsive hydrogel may exhibit a gelation temperature of 34° C. at pH 4.2, In some embodiments, the thermoresponsive hydrogel may exhibit a gelation temperature of 33° C. at pH 4.8.

In some embodiments, the hydrogel precursor forming the thermoresponsive hydrogel is selected from the group consisting of poly(propylene oxide), poly(ethylene oxide), poloxamers (pluronics), chitosan, gelatin, cellulose derivatives, glycol chitin, poly(N-isopropylacrylamide (PNIPAAm), PEG-PLGA-PEG, [poly(D, L-lactide)-poly(ethyleneglycol)-poly(D,L-lactide) (PDLLA-PEG-PDLLA), and combinations thereof. In some embodiments, the hydrogel precursor comprises a sprayable liquid formulation. In some embodiments, the hydrogel precursor comprises an injectable liquid formulation. Under NIR irradiation, the hydrogel precursor rapidly forms a hydrogel locally induced by the energy-to-heat conversion effects of the material in the heat delivery medium or particle heaters. In some embodiments, the hydrogel precursor comprises chitosan and glycol chitosan. In some embodiments, the hydrogel precursor comprises glycol chitin. In some embodiments, the hydrogel precursor is an amphiphilic block copolymer comprising at least on hydrophobic polymer block and at least one hydrophilic polymer block. In some embodiments, the amphiphilic block copolymer is PEG-PLGA-PEG or PDLLA-PEG-PDLLA. In some embodiments, the thermoresponsive hydrogel comprises PEG-PLGA-PEG with respective Mn of 800-1000-800 Da, wherein PLGA comprises 1:1 mole ratio of LA:GA. In some embodiments, the thermoresponsive hydrogel comprises PEG-PLGA-PEG with respective Mn of 800-1500-800 Da, wherein PLGA comprises 1:1 mole ratio of LA:GA.

In some embodiments, the thermoresponsive hydrogel is formed of polysaccharide. In some embodiments, the polysaccharide is selected from hyaluronic acid, glycosamine, carrageenan, alginate, and combinations thereof.

In some embodiments, the hydrogel is formed from reacting two hydrogel precursors having cross-linkable reactive groups with complementary reactivity (e.g. one pre-polymer having reactive —COOH group and the other pre-polymer having —NH₂ group).

In some embodiments, the carrier is formed of polymer or co-polymers; examples include but may not limited to polycarbonate polyacrylates, polymethacrylates and copolymers thereof, polyurethanes, polyureas, cellulosic materials, polymaleic acid and its derivatives, and polyvinyl acetate. In some embodiments, the carrier comprises polymethacrylates and copolymers thereof.

In some embodiments, the carrier comprises a hydrogel precursor for in situ hydrogel formation. In some embodiments, the hydrogel precursor has reactive functional groups. In some embodiments, the reactive functional groups are selected from the group consisting of vinyl dimethylsulfone group, hydroxyl group (—OH), thiol group (—SH), amine group (—NH₂), aldehyde group (—CHO), carboxylic acid group (—COOH), epoxy group, and combinations thereof.

In some embodiments, the cross-linkable reactive groups are selected from the group consisting of vinyl group (—CH═CH₂), ethynyl group (—C≡C—), vinyl methylsulfone group, hydroxyl group (—OH), thiol group (—SH), amine group (—NH₂), aldehyde group (—CHO), carboxylic acid group (—COOH), and combinations thereof. In some embodiments, the carrier comprises the cross-linkable polysaccharides. In some embodiments, the cross-linkable polysaccharides may include alginic acid, sodium alginate, or carrageenan.

In some embodiments, the hydrogel precursor comprises cross-linkable polysaccharides. In some embodiments, cross-linkable polysaccharides may include alginate and/or carrageenan. In some embodiments, the crosslinking agent for the cross-linkable polysaccharides comprises a metal salt having a divalent metal ion, for example calcium ion. In some embodiments, the metal salt is calcium chloride.

In some embodiments, the carrier comprises adhesives. In some embodiments, the adhesive is a pressure sensitive adhesive (PSA). In some embodiments, the pressure sensitive adhesive cross-linked polymers. In some embodiments, the pressure sensitive adhesive comprises silicone polymer, or polyacrylates. In some embodiments, the silicone polymer or polyacrylates of the PSA is cross-linked.

In some embodiments, the carrier comprises cross-linked polymers formed from reacting the cross-linkable reactive groups attached to the carrier with a cross-linking reagent. In some embodiments, the degree of cross-linking can be tuned by controlling the weight ratio of the cross-linker reagent to the carrier having cross-linkable reactive groups in the cross-linking reaction.

In some embodiments, the crosslinking reagent is selected from the group consisting of ethylene glycol dimethacrylate (EGDMA), 1,3-butylene glycol dimethacrylate (BGDMA), 1,4-butane diol diacrylate (BDDA), 1,6-hexane diol diacrylate (HDDA), hexanediol dimethacrylate (HDDMA), 1-carboxy-N-methyl-N-di(2-methacryloyloxy-ethyl) methanaminium inner salt (CBMX), bis(meth)acrylamides, neopentylglycol diacrylate (NPGDA), trimethylolpropane triacrylate (TMPTA), and combinations thereof.

In some embodiments, the cross-linking reagent for cross-linking hydroxyl groups (—OH), thiol groups (—SH), or amine groups (—NH₂) attached to the carrier may include dithiobis(succinimidyl) propionate (Lomant's reagent), cystamine bisacrylamide, bisacryloyloxyethyl disulfide, N,N′-(ethane-1,2-diyl)diacrylamide, N,N′-(2-hydroxypropane-1,3-diyl)diacrylamide, polyisocyanate, polyisothiocyanate, dimethyl adipimidate, dimethyl pimelimidate, dimethyl suberimidate, dimethyl 3,3′-dithiobispropionimidate, glutaraldehyde, glyoxal, glyoxal-trimer dihydrate, dimethyl suberimidate, dimethyl 3,3′-dithiobispropionimidate glutaraldehyde, epoxides, bis-oxiranes, p-azidobenzoyl hydrazide, N-α-maleimidoacetoxy succinimide ester, p-azidophenyl glyoxal monohydrate, bis-((beta)-(4-azidosalicylamido)ethyl)disulfide, succinimidyl iodoacetate, succinimidyl 3-(bromoacetamido)propionate, 4-(iodoacetyl)aminobenzoate, N-α-maleimidoacetoxysuccinimide ester, N-β-maleimidopropyloxysuccinimide ester, N-γ-maleimidobutyryloxysuccinimide ester, m-maleimidobenzoyl-N-hydroxysuccinimide ester, succinimidyl 4-(N-maleimidomethyl)cyclohexane-1-carboxylate, N-ε-malemidocaproyl oxysuccinimide ester, succinimidyl 4-(p-maleimidophenyl)butyrate, succinimidyl 6-β-maleimidopropionamido)hexanoate, succinimidyl 3-(2-pyridyldithio)propionate (SPDP), PEG4-SPDP, PEG12-SPDP, disuccinimidyl tartrate, 4-succinimidyloxycarbonyl-α-methyl-α-(2-pyridyldithio)toluene, disuccinimidyl glutarate, ethylene glycol bis(succinimidylsuccinate), bis-(sulfosuccinimidyl) (ethylene glycol) bis(succinimidylsuccinate), bis-sulfosuccinimidyl suberate, disuccinimidyl-suberate, tris-succinimidyl aminotriacetate, diacylchlorides, or polyphenolic compounds (e.g. tannic acid or tannin as cross-linker for cross-linking protein such as collagen, gelatin etc., dopamine and its derivatives).

In some embodiments, the cross-linking reagent for cross-linking hydroxyl groups (—OH), thiol groups (—SH), or amine groups (—NH₂) attached to the carrier may include carboxyl group terminated polyethylene glycol having 2-8 branching arms (used with carboxylic acid activation agent N-hydroxysuccinimide esters (NHS) and/or (1-ethyl-3-(3-dimethylaminopropyl)-carbodiimide (EDC)), for example, 4-arm PEG carboxyl (pentaerythritol core), 6-arm PEG carboxyl (hexaglycerin core), or 8-arm PEG carboxyl (tripentaerythritol core). In some embodiments, the cross-linker reagent for cross-linking hydroxyl groups (—OH), thiol groups (—SH), or amine groups (—NH₂) attached to the carrier may include bis-succinimide ester terminated polyethylene glycol or star shaped succinimide ester terminated polyethylene glycol having 3-8 branching arms, for example, 4-arm PEG succinimidyl (pentaerythritol core) or 6-arm PEG succinimidyl (hexaglycerin core). In some embodiments, the succinimide ester, or carboxyl group terminated polyethylene glycol type cross-linker reagent may have a number average molecular weight ranging from about 150 Daltons (Da) to about 10 KDa. In some embodiments, the succinimide ester, or carboxyl group terminated polyethylene glycol type cross-linker reagent may have a number average molecular weight ranging from about 1 KDa to about 10 KDa. In some embodiments, the succinimide ester, or carboxyl group terminated polyethylene glycol type cross-linker reagent may have a number average molecular weight ranging from about 1 KDa to about 5 KDa. In some embodiments, the succinimide ester, or carboxyl group terminated polyethylene glycol type cross-linker reagent may have a number average molecular weight ranging from about 150 Da to about 1 KDa. In some embodiments, the succinimide ester, or carboxyl group terminated polyethylene glycol type cross-linker reagent may have a number average molecular weight ranging from about 150 Da to about 750 Da.

In some embodiments, the cross-linking reagent for cross-linking reactive aldehyde groups, vinyl methylsulfone groups, or carboxylic acid groups (activation with N-hydroxysuccinimide esters (NHS) or (1-Ethyl-3-(3-dimethylaminopropyl)-carbodiimide (EDC)) attached to the carrier may include polyamine compounds such as spermine, polyspermine, low molecular weight polyethylenimine (PEI), dilysine, linear or branched trilysine, tetralysine, pentalysine, hexylysine, heptalysine, octalysine, nonalysine, decalysine, undecalysine, dodecalysine, tridecalysine, tetradecalysine, pentadecalysine, or hyperbranched polylysines, polyols such as pentaerythritol, ethylene glycol, polyethylene glycol, glycerol, polyglycerol, sucrose, sorbitol etc.

In some embodiments, the cross-linking reagent for cross-linking aldehyde groups, vinyl methylsulfone groups, or carboxylic acid groups (activation with N-hydroxysuccinimide esters (NHS) or (1-ethyl-3-(3-dimethylaminopropyl)-carbodiimide (EDC)) attached to the carrier may include amine terminated polyethylene glycols having 2-8 branching arms, for example, 4-arm PEG amine (pentaerythritol core), 6-arm PEG amine (hexaglycerin core), or 8-arm PEG amine (tripentaerythritol core). In some embodiments, the amine terminated polyethylene glycol type cross-linker reagents may have a number average molecular weight ranging from 150 Da to 10 KDa. In some embodiments, the amine terminated polyethylene glycol type cross-linker reagents may have a number average molecular weight ranging from 1 KDa to 10 KDa. In some embodiments, the amine terminated polyethylene glycol type cross-linker reagents may have a number average molecular weight ranging from 1 KDa to 5 KDa. In some embodiments, the amine terminated polyethylene glycol type cross-linker reagents may have a number average molecular weight ranging from 150 Da to 1 KDa. In some embodiments, the amine terminated polyethylene glycol type cross-linker reagent may have a number average molecular weight ranging from 150 Da to 750 Da.

In some embodiments, the carrier comprises a shape memory polymer (SMP). In some embodiments, the carrier comprises thermoresponsive SMPs which regain their original shape from a temporary fixed shape to a permanent shape in response to a stimulus (i.e. heat). In some embodiments, the SMPs comprises crosslinked network structure. The temporary shape for the SMP is obtained by processing the SMP at a temperature (T) higher than the glass transition temperature of SMP (T_{g}) (T>T_{g}) while preventing polymer chain relaxation through a crosslinked network structure, followed by cooling to T<T_{g} for chain freezing. Subsequently, if the SMP is heated to a temperature higher than T_{g}, chain relaxation occurs and the SMP recovers its permanent shape.

In some embodiments, the carrier comprises SMPs, wherein the carrier admixed with the material forms a coating layer, wherein the material is homogeneously distributed within the SMPs.

In some embodiments, the heat delivery medium comprises a thermosensitive shape memory polymer and NIR absorbing dye composite, wherein the NIR absorbing dye is loaded in a crosslinked SMP network. In some embodiments, the heat delivery medium comprises a thermosensitive shape memory polymer and tetrakis aminium dye composite, wherein the tetrakis aminium dye is loaded in a crosslinked SMP network. In some embodiments, the heat delivery medium comprises a laser triggered and spatially controllable thermosensitive shape memory polymer and indocyanine green dye (ICG) composite, wherein the indocyanine dye is loaded in a crosslinked SMP network. In some embodiments, the thermosensitive shape memory polymer and NIR absorbing dye composite forms a coating layer. In some embodiments, the thermosensitive shape memory polymer and tetrakis aminium dye composite forms a coating layer. In some embodiments, the thermosensitive shape memory polymer and ICG dye composite forms a coating layer.

In some embodiments, the laser causes the NIR absorbing dye to generate heat that, in turn, heats the SMP to a temperature higher than the polymer glass transition temperature (T_{g}) and triggers the shape recovery.

In some embodiments, the shape memory polymer comprises thermoplastic shape memory polymers (e.g. thermoplastic polyurethanes), or thermoset shape memory polymers (e.g. thermoset polyurethane). In some embodiments, the thermoset polyurethane shape memory polymer having a glass transition temperature (T_{g}) of between 25° C. and 120° C., characterized in that said polymer has a transformation temperature of at least 130° C.

In some embodiments, the shape memory polymer comprises an aromatic diepoxy/diamine system with a T_{g} of about 90° C. In some embodiments, the aromatic diepoxy component is replaced systematically with an aliphatic diepoxy to yield a series of epoxy shape memory polymers with T_{g} ranging from 25° C. to 90° C. In some embodiments, the shape memory polymer comprises poly(lactic acid) (PLA) combined with hydroxyapatite.

In some embodiments, the shape memory polymer is selected from the group consisting of α-olefin/vinyl or vinylidene aromatic and/or hindered aliphatic vinyl or vinylidene interpolymers, crosslinked polyurethanes based on poly(ε-caprolactone) (PCL), poly(ethylene adipate) and polyisocyanate, PEG-α-cyclodextrin, poly(l-lactide) (PLLA)-PCL diblock copolymer, poly(3-hydroxy butyrate)-co-(3-hydroxy valerate) (PHBV), poly(t-butyl acrylate) crosslinked with poly(ethylene glycol dimethacrylate), poly(t-butyl acrylate) crosslinked with poly(β-aminoester), branched oligo(3-caprolactone) cross-linked with hexamethylene diisocyanate (HMDI), polyethylene glycol and polydimethacrylate (DMA) copolymer crosslinked with methyl methacrylate (MMA), crosslinked low density polyethylene (LDPE), furan-terminated telechelic polyesters of poly(1,4-butylenesuccinate-co-1,3-propylene succinate), polytetramethylene oxide/poly(acrylic acid-co-acrylonitrile) (PTMO-[P(AA-co-AN)]), PEG dimethacrylate and methacrylate copolymer (PEGDMA), PLGA triol, tetraols crosslinked with aliphatic diisocyanates, polyacrylamide and polyethylene oxide diblock copolymer, and combinations thereof.

In some embodiments, the carrier is present at a weight percentage by the total weight of the heat delivery medium or particle heater selected from the group consisting of about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, or about 20.0 wt. %, about 25.0 wt. %, about 30.0 wt. %, about 35.0 wt. %, about 40.0 wt. %, about 45.0 wt. %, about 50.0 wt. %, about 55.0 wt. %, about 60.0 wt. %, about 65.0 wt. %, about 70.0 wt. %, about 75.0 wt. %, about 80.0 wt. %, about 85.0 wt. %, about 90.0 wt. %, about 95.0 wt. %, and about 99.0 wt. %. In some embodiments, the carrier is present at a weight percentage by the total weight of the heat delivery medium or particle heater ranges from about 1 wt. % to about 99 wt. %. In some embodiments, the carrier is present at a weight percentage by the total weight of the heat delivery medium or particle heater ranges from about 10.0 wt. % to about 90.0 wt. %. In some embodiments, the carrier=is present at a weight percentage by the total weight of the heat delivery medium or particle heater ranges from about 50.0 wt. % to about 90.0 wt. %. In some embodiments, the carrier is present at a weight percentage by the total weight of the heat delivery medium or particle heater ranges from about 25.0 wt. % to about 50.0 wt. %. In some embodiments, the carrier=is present at a weight percentage by the total weight of the heat delivery medium or particle heater ranges from about 75.0 wt. % to about 90.0 wt. %.

In some embodiments, the crosslinker is present at a weight percentage range from about 3.0 wt. % to about 30.0 wt. % by the weight of the heat delivery medium. In some embodiments, the crosslinker is present at a weight percentage range from about 5.0 wt. % to about 20.0 wt. % by the weight of the heat delivery medium. In some embodiments, the crosslinker is present at a weight percentage range from about 5.0 wt. % to about 15.0 wt. % by the weight of the heat delivery medium. In some embodiments, the crosslinker is present at a weight percentage range from about 5.0 wt. % to about 10.0 wt. % by the weight of the heat delivery medium. In some embodiments, the crosslinker is present at a weight percentage selected from the group consisting of about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, or about 20.0 wt. %, about 25.0 wt. %, and about 30.0 wt. % by the weight of the heat delivery medium. In some embodiments, the crosslinker is present at a weight percentage of about 5.0 wt. % by the weight of the heat delivery medium. In some embodiments, the crosslinker is present at a weight percentage of about 6.0 wt. % by the weight of the heat delivery medium. In some embodiments, the crosslinker is present at a weight percentage of about 6.5 wt. % by the weight of the heat delivery medium. In some embodiments, the crosslinker is present at a weight percentage of about 7.0 wt. % by the weight of the heat delivery medium. In some embodiments, the crosslinker is present at a weight percentage of about 8.0 wt. % by the weight of the heat delivery medium. In some embodiments, the crosslinker is present at a weight percentage of about 9.0 wt. % by the weight of the heat delivery medium. In some embodiments, the crosslinker is present at a weight percentage of about 10.0 wt. % by the weight of the heat delivery medium. In some embodiments, the crosslinker is present at a weight percentage of about 15.0 wt. % by the weight of the heat delivery medium. In some embodiments, the crosslinker is present at a weight percentage of about 20.0 wt. % by the weight of the heat delivery medium.

(ii) Material Interacting with the Exogenous Sources

In some embodiments, the material interacts with the exogenous source to produce heat that performs a function, like accelerating a physical, chemical or biological activity by raising the temperature to above normal body temperature. In some embodiments, the exogenous source is electromagnetic radiation, microwaves, radio waves, sound waves, electrical or magnetic field.

In some embodiments, the exogenous source is selected from the group consisting of an electromagnetic radiation, an electrical field, a microwave, a radio wave, ultrasonic, a magnetic field, and combinations thereof. In some embodiments, the exogenous source comprises a laser light. In some embodiments, the exogenous source comprises an LED light. In some embodiments, the laser light is a pulsed laser light. In some embodiments, the laser pulse duration is in a range from milliseconds to nanoseconds, and the laser has an oscillation wavelength at 1064 nm. In some embodiments the laser emits light at 808 nm. In some embodiments the laser emits light at 805 nm.

In some embodiments the exogenous source is an ultrasound (US) producing machine. In some embodiments the therapeutic ultrasound is either pulsed or continuous.

In some embodiments, the exogenous source may have a cold tip to cool the target tissue area before, during and after application of the exogenous energy. In some embodiments the cold tip may be at a temperature from about 2° C. to about 8° C.

The frequency of ultrasound dictates the depth of penetration and impacts the efficiency of particle heaters. To reach deeper tissues (up to 5 cm or more), a frequency of 1 MHz should be selected. When the target tissue is within 2.5 cm from the surface of the skin, a frequency of 3 MHz should be selected. It is important to note that 3 MHz will produce heat from particle heaters approximately 3-times faster than 1 MHz, creating a higher efficiency in heating when compared to 1 MHz ultrasound for the same particle heater. For continuous US, frequencies within the range of 1-3 MHz at intensities of 0.5-10 W/cm² for a duration of 1-15 minutes at 100% duty cycle should be useful for in vivo applications. In some embodiments the US frequencies of 1-2 MHz at intensity ranges from 0.5-5 W/cm² are applied for 1-5 minutes at 100% duty cycle. 3 MHz ultrasound is absorbed more rapidly in the tissues, and therefore is considered to be most appropriate for superficial lesions, whilst the 1 MHz energy is absorbed less rapidly with deeper progression through the tissues and can therefore be more effective at greater depth. The boundary between superficial and deep tissues is in some ways arbitrary, but somewhere around the 2 cm depth is often taken as a useful boundary. Hence, if the target tissue is within 2 cm (or just under an inch) of the skin surface, 3 MHz treatments will be effective whilst treatments to deeper tissues will be more effectively achieved with 1 MHz ultrasound. One important factor is that some of the ultrasonic energy (US) delivered to the tissue surface will/may be lost before the target tissue (i.e. in the normal or uninjured tissues which lie between the skin surface and the target). In order to account for this, it may be necessary to deliver more US energy at the surface than is required, therefore allowing for some absorption before the target tissue, and allowing sufficient remaining US energy to achieve the desired effect. To identify the appropriate dose from the machine, one has to determine (a) the estimated depth of the lesion to be treated and (b) the intensity of US energy required at that depth to achieve the desired effect. For example, to achieve a 0.5 W/cm² intensity at 1 cm tissue depth, one would select 3 MHz treatment option and set machine to 0.7 W/cm² which will result in 0.5 W/cm² intensity at a 1 cm tissue depth. The rate at which US energy is absorbed in the tissues can be approximately determined by the half-value depth. The half-value depth is the tissue depth at which 50% of the US energy delivered at the surface has been absorbed. The average half-value depth of 3 MHz ultrasound is taken at 2.5 cm and that of 1 MHz ultrasound as 4.0 cm, although there are numerous debates that continue with regards the most appropriate half-value depth for different frequencies.

In some embodiments pulsed ultrasound is used. The pulse ratio determines the concentration of the sound energy on a time basis. The pulse ratio determines the proportion of time that the ultrasound machine is “ON” compared with the “OFF” time. A pulse ratio of 1:1 for example means that the machine delivers one ‘unit’ of US energy followed by an equal duration during which no energy is delivered. The machine duty cycle is therefore 50%. A machine pulsed at a ratio of 1:4 will deliver one unit of US energy followed by 4 units of rest, therefore the machine is on for 20% of the time (some machines use ratios, and some percentages). The selection of the most appropriate pulse ratio essentially depends on the state of the target tissue(s). The less dense the target tissue state, the more energy sensitive it is, and appears to respond more favorably to energy delivered with a larger pulse ratio (lower duty cycle). As the tissue becomes denser, it appears to respond preferentially to a more ‘concentrated’ energy delivery, thus reducing the pulse ratio (or increasing the duty cycle). It has been suggested that pulse ratios of 1:4 would be best suited to the treatment of low density tissues, reducing this as the tissue increases in density, moving through 1:3 and 1:2 to end up with 1:1 or continuous modes. As a general rule, pulsing at a 1:4 or 1:3 ratio will be used for the less dense tissues, 1:2 and 1:1 ratio for the medium density tissues and 1:1 or Continuous for the dense tissues. It is of note that it is the state of the tissue that determines the most appropriate pulse ratio rather than simply the duration since the onset of the lesion. In a similar way to the clinical decision-making process in other therapies, tissue reactivity is the key. The final compilation of the treatment dose which is most likely to be effective is based on the principle that about 1-minute worth of US energy (at an appropriate frequency and intensity) should be delivered for every US head that needs to be covered. The size of the treatment area will influence the treatment time, as will the pulse ratio being used. The larger the treatment area, the longer the treatment will take. The more pulsed the energy output from the machine, the longer it will take to deliver about a 1-minute worth of US energy. Sound dose will obviously also depend on particle the heater concentration at the target tissue

In some embodiments, the exogenous source may be electromagnetic radiation (EMR). In some embodiments, the material interacting with the exogenous source comprises a dye capable of absorbing EMR and converting the energy to heat (photothermal conversion).

In some embodiments, the material interacting with the exogenous source comprises a dye capable of absorbing electromagnetic radiation and converting the energy to heat (photothermal conversion). In some embodiments, the material interacting with the exogenous source has significant absorption in the near infrared spectrum region (NIR). In some embodiments, the material interacting with the exogenous source has significant absorption at NIR wavelengths in the range from 750 nm to 1500 nm. In some embodiments, the material interacting with the exogenous source has significant absorption at NIR wavelengths in the range from 750 nm to 1400 nm. In some embodiments, the material interacting with the exogenous source has significant absorption at NIR wavelengths in the range from 750 nm to 1300 nm. In some embodiments, the material interacting with the exogenous source has significant absorption at a NIR wavelength in the range from 750 nm to 900 nm. In some embodiments, the material interacting with the exogenous source has significant absorption at NIR wavelengths in the range from 750 nm to 950 nm. In some embodiments, the material interacting with the exogenous source has significant absorption at NIR wavelengths in the range from 800 nm to 1100 nm. In some embodiments, the material interacting with the exogenous source has significant absorption at NIR wavelengths in the range from 1000 nm to 1400 nm. In some embodiments, the material interacting with the exogenous source has significant absorption at NIR wavelengths in the range from 1000 nm to 1300 nm. In some embodiments, the material interacting with the exogenous source has significant absorption at NIR wavelengths in the range from 1000 nm to 1100 nm. In some embodiments, the material interacting with the exogenous source has significant absorption at a wavelength selected from the group consisting of 750 nm, 751 nm, 752 nm, 753 nm, 754 nm, 755 nm, 756 nm, 757 nm, 756 nm, 756 nm, 758 nm, 759 nm, 760 nm, 761 nm, 762 nm, 763 nm, 764 nm, 765 nm, 766 nm, 767 nm, 768 nm, 769 nm, 770 nm, 771 nm, 772 nm, 773 nm, 774 nm, 775 nm, 776 nm, 777 nm, 778 nm, 779 nm, 780 nm, 781 nm, 782 nm, 783 nm, 784 nm, 785 nm, 786 nm, 787 nm, 789 nm, 790 nm, 791 nm, 792 nm, 793 nm, 794 nm, 795 nm, 796 nm, 797 nm, 798 nm, 799 nm, 800 nm, 801 nm, 802 nm, 803 nm, 804 nm, 805 nm, 806 nm, 807 nm, 808 nm, 809 nm, 810 nm, 811 nm, 812 nm, 813 nm, 814 nm, 815 nm, 816 nm, 817 nm, 818 nm, 819 nm, 820 nm, 821 nm, 822 nm, 823 nm, 824 nm, 825 nm, 826 nm, 827 nm, 828 nm, 829 nm, 830 nm, 831 nm, 832 nm, 833 nm, 834 nm, 835 nm, 836 nm, 837 nm, 838 nm, 839 nm, 840 nm, 841 nm, 842 nm, 843 nm, 844 nm, 845 nm, 846 nm, 847 nm, 848 nm, 849 nm, 850 nm, 851 nm, 852 nm, 853 nm, 854 nm, 855 nm, 856 nm, 857 nm, 858 nm, 859 nm, 860 nm, 861 nm, 862 nm, 863 nm, 864 nm, 865 nm, 866 nm, 867 nm, 868 nm, 869 nm, 870 nm, 871 nm, 872 nm, 873 nm, 874 nm, 875 nm, 876 nm, 877 nm, 878 nm, 879 nm, 880 nm, 881 nm, 882 nm, 883 nm, 884 nm, 885 nm, 886 nm, 887 nm, 888 nm, 889 nm, 890 nm, 891 nm, 892 nm, 893 nm, 894 nm, 895 nm, 896 nm, 897 nm, 898 nm, 899 nm, 900 nm, 901 nm, 902 nm, 903 nm, 904 nm, 905 nm, 906 nm, 907 nm, 908 nm, 909 nm, 910 nm, 911 nm, 912 nm, 913 nm, 914 nm, 915 nm, 916 nm, 917 nm, 918 nm, 919 nm, 920 nm, 921 nm, 922 nm, 923 nm, 924 nm, 925 nm, 926 nm, 927 nm, 928 nm, 929 nm, 930 nm, 931 nm, 932 nm, 933 nm, 934 nm, 935 nm, 936 nm, 937 nm, 938 nm, 939 nm, 940 nm, 941 nm, 942 nm, 943 nm, 944 nm, 945 nm, 946 nm, 947 nm, 948 nm, 949 nm, 950 nm, 951 nm, 952 nm, 953 nm, 954 nm, 955 nm, 956 nm, 957 nm, 958 nm, 959 nm, 960 nm, 961 nm, 962 nm, 963 nm, 964 nm, 965 nm, 966 nm, 967 nm, 968 nm, 969 nm, 970 nm, 971 nm, 972 nm, 973 nm, 974 nm, 975 nm, 976 nm, 977 nm, 978 nm, 979 nm, 980 nm, 981 nm, 982 n, 983 nm, 984 nm, 985 nm, 986 nm, 987 nm, 988 nm, 989 nm, 990 nm, 991 nm, 992 nm, 993 nm, 994 nm, 995 nm, 996 nm, 997 nm, 998 nm, 999 nm, 1000 nm, 1001 nm, 1002 nm, 1003 nm, 1004 nm, 1005 nm, 1006 nm, 1007 nm, 1008 nm, 1009 nm, 1010 nm, 1011 nm, 1012 nm, 1013 nm, 1014 nm, 1015 nm, 1016 nm, 1017 nm, 1018 nm, 1019 nm, 1020 nm, 1021 nm, 1022 nm, 1023 nm, 1024 nm, 1025 nm, 1026 nm, 1027 nm, 1028 nm, 1029 nm, 1030 nm, 1031 nm, 1032 nm, 1033 nm, 1034 nm, 1035 nm, 1036 nm, 1037 nm, 1038 nm, 1039 nm, 1040 nm, 1041 nm, 1042 nm, 1043 nm, 1044 nm, 1045 nm, 1046 nm, 1047 nm, 1048 nm, 1049 nm, 1050 nm, 1051 nm, 1052 nm, 1053 nm, 1054 nm, 1055 nm, 1056 nm, 1057 nm, 1058 nm, 1059 nm, 1060 nm, 1061 nm, 1062 nm, 1063 nm, 1064 nm, 1065 nm, 1066 nm, 1067 nm, 1068 nm, 1069 nm, 1070 nm, 1071 nm, 1072 nm, 1073 nm, 1074 nm, 1075 nm, 1076 nm, 1077 nm, 1078 nm, 1079 nm, 1080 nm, 1081 nm, 1082 nm, 1083 nm, 1084 nm, 1085 nm, 1086 nm, 1087 nm, 1088 nm, 1089 nm, 1090 nm, 1091 nm, 1092 nm, 1093 nm, 1094 nm, 1095 nm, 1096 nm, 1097 nm, 1098 nm, 1099 nm, and 1100 nm. In some embodiments, the material interacting with the exogenous source has significant absorption at a wavelength selected from the group consisting of 700 nm, 766 nm, 777 nm, 780 nm, 783 nm, 785 nm, 800 nm, 808 nm, 810 nm, 820 nm, 825 nm, 900 nm, 948 nm, 950 nm, 960 nm, 980 nm, 1000 nm, 1064 nm, 1065 nm, 1070 nm, 1071 nm, 1073 nm, 1098 nm, and 1100 nm. In some embodiments, the material interacting with the exogenous source has significant absorption at 1064 nm wavelength.

In some embodiments, the material interacting with the exogenous source has significant absorption of photonic energy in the visible range. In some embodiments, the material absorbs light at a wavelength ranging from 400 nm to 750 nm. In some embodiments, the material absorbs light at a wavelength selected from the group consisting of 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, and 750 nm.

In some embodiments, the material interacting with exogenous source is an IR absorbing material. In some embodiments, the IR absorbing material comprises organic dyes or inorganic pigments. In some embodiments, the IR absorbing material is an IR dye. In some embodiments, the IR dye is an aminium and/or di-imonium dye having hexafluoroantimonate, tetrafluoroborate, or hexafluorophosphate as counterion. In some embodiments, an IR absorbing material, N,N,N,N-tetrakis(4-dibutylaminophenyl)-p-benzoquinone bis(iminium hexafluoroantimonate), commercially available as ADS1065 from American Dye Source, Inc., may be utilized. The absorption spectrum of ADS1065 dye has a maximum absorption at about 1065 nm, with low absorption in the visible region of the spectrum.

In some embodiments, the material is an IR absorbing organic dye such as those Epolight™ aminium dyes made by Epolin Inc. of Newark, N.J. In some embodiments, the IR absorbing dye is an di-imonium dye (also aminium dye) having formula (I)

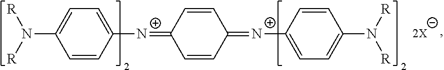

wherein R is a substituted or unsubstituted aryl, heteroaryl, C1-C8 alkyl, C1-C8 alkenyl, or C1-C8 alkynyl group, wherein the C1-C8 alkyl, C1-C8 alkenyl, or C1-C8 alkynyl group may be linear or branched, wherein X⁻ is a counterion selected from the group consisting of hexafluoroarsenate (AsF₆⁻), hexafluoroantimonate (SbF₆⁻), hexafluorophosphate (PF₆⁻), tetrakis(perfluorophenyl)borate (C₆F₅)₄B⁻, tetrafluoroborate (BF₄⁻), and combinations thereof. In some embodiments, the di-imonium dye of formula (I) has hexafluorophosphate as counterion. In some embodiments, the di-imonium dye of formula (I) has hexafluoroantimonate as counterion. In some embodiments, the di-imonium dye of formula (I) has tetrakis(perfluorophenyl)borate as counterion. In some embodiments, the IR absorbing dye is a tetrakis aminium dye, with a counterion containing metal element such as boron or antimony. In some embodiments, the tetrakis aminium dye compounds have formula (II)

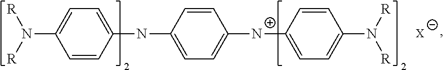

wherein R is a substituted or unsubstituted aryl, heteroaryl, C1-C8 alkyl, C1-C8 alkenyl, or C1-C8 alkynyl group, wherein the C1-C8 alkyl, C1-C8 alkenyl, or C1-C8 alkynyl group may be linear or branched, wherein X⁻ is a counterion selected from the group consisting of hexafluoroarsenate (AsF₆⁻), hexafluoroantimonate (SbF₆⁻), hexafluorophosphate (PF₆⁻), (C₆F₅)₄B⁻, tetrafluoroborate (BF₄⁻), and combinations thereof. In some embodiments, the tetrakis aminium dyes are narrow band absorbers including commercially available dyes sold under the trademark names Epolight™ 1117 (tetrakis aminium dye having hexafluorophosphate counterion, peak absorption, 1071 nm), Epolight™ 1151 (tetrakis aminium dye, peak absorption, 1070 nm), or Epolight™ 1178 (tetrakis aminium dye, peak absorption, 1073 nm). Epolight™ 1151 (tetrakis aminium dye, peak absorption, 1070 nm), or Epolight™ 1178 (tetrakis aminium dye, peak absorption, 1073 nm). In some embodiments, the tetrakis aminium dyes are broad band absorbers including commercially available dyes sold under the trademark names Epolight™ 1175 (tetrakis aminium dye, peak absorption, 948 nm), Epolight™ 1125 (tetrakis aminium dye, peak absorption, 950 nm), and Epolight™ 1130 (tetrakis aminium dye, peak absorption, 960 nm).

In some embodiments, the tetrakis aminium dye is Epolight™ 1178 made by Epolin. In some embodiments, the IR absorbing material is a tetrakis aminium dye has minimal visible color. In some embodiments, the tetrakis aminium dye is Epolight™ 1117 (molecular weight, 1211 Da, peak absorption 1098 nm).

Other suitable aminium and/or di-imonium dyes suitable for the invention in this disclosure may be found in U.S. Pat. Nos. 3,440,257, 3,484,467, 3,400,156, 5,686,639, all of which are hereby fully incorporated by reference herein in their entirety. Additional counterions for the aminium and/or di-imonium dyes may be found in U.S. Pat. No. 7,498,123, which is hereby fully incorporated by reference herein in its entirety.

In some embodiments, the material is an IR absorbing material selected from the group consisting of 1-butyl-2-(2-[3-[2-(1-butyl-1H-benzo[cd]indol-2-ylidene)-ethylidene]-2-chloro-cyclohex-1-enyl]-vinyl)-benzo[cd]indolium tetrafluoroborate, 1-butyl-2-(2-[3-[2-(1-butyl-1H-benzo[cd]indol-2-ylidene)-ethylidene]-2-phenyl-cyclopent-1-enyl]-vinyl)-benzo[cd]indolium tetrafluoroborate, 1-butyl-2-(2-[3-[2-(1-butyl-1H-benzo[cd]indol-2-ylidene)-ethylidene]-2-phenyl-cyclohex-1-enyl]-vinyl)-benzo[cd]indolium tetrafluoroborate, 1-butyl-2-(2-[3-[2-(1-butyl-1H-benzo[cd]indol-2-ylidene)-ethylidene]-2-diphenylamino-cyclopent-1-enyl]vinyl)-benzo[cd]indolium tetrafluoroborate, 1-Butyl-2-[2-[3-[(1-butyl-6-chlorobenz[cd]indol-2(1H)-ylidene)ethylidene]-2-chloro-1-cyclohexen-1-yl]ethenyl]-6-chlorobenz[cd]indolium tetrafluoroborate (IR 1048), 1-butyl-2-[2-[3-[(1-butyl-6-chlorobenz[cd]indol-2(1H)-ylidene)ethylidene]-2-chloro-5-methyl-1-cyclohexen-1-yl]ethenyl]-6-chlorobenz[cd]indolium tetrafluoroborate (Lumogen™ IR 1050 by BASF), 4-[2-[2-chloro-3-[(2,6-diphenyl-4H-thiopyran-4-ylidene)ethylidene]-1-cyclohexen-1-yl]ethenyl]-2,6-diphenylthiopyrylium tetrafluoroborate (IR 1061), dimethyl {4-[1,7,7-tris(4-dimethylaminophenyl)-2,4,6-heptatrienylidene]-2,5-cyclohexadien-1-ylidene}ammonium perchlorate (IR 895), 2-[2-[2-chloro-3-[[1,3-dihydro-1,1-dimethyl-3-(4-sulfobutyl)-2H-benzo[e]indol-2-ylidene]-ethylidene]-1-cyclohexen-1-yl]-ethenyl]-1,1-dimethyl-3-(4-sulfobutyl)-1H-benzo[e]indolium hydroxide inner salt, sodium salt (IR820, new ICG dye), heptamethine cyanine (IR825), heptamethine cyanine (IR780), 4-hydroxybenzoic acid appended heptamethine cyanine, amine functionalized heptamethine cyanine, hemicyanine rhodamine, cryptocyanine, diketopyrrolopyrole, diketopyrrolopyrole-croconaine, 1,3-bis(5-(ethyl(2-(prop-2-yn-1-yloxy)ethyl)amino)thiophen-2-yl)-4,5-dioxocyclopent-2-en-1-ylium-2-olate (diaminothiophene-croconaine dye), potassium 1,1′-((2-oxido-4,5-dioxocyclopent-2-en-1-ylium-1,3-diyl)bis(thiophene-5,2-diyl))bis(piperidine-4-carboxylate) (dipiperidylthiophene-croconaine dye), indocyanine green (ICG), Cyanine 7 (Cy7®), and combinations thereof. In some embodiments, the material is an IR-absorbing agent selected from the group consisting of phthalocyanines. naphthalocyanines, and combinations thereof. In some embodiments, the IR absorbing material is selected from the group consisting of a tris-aminium dye, a tetrakis aminium dye, a squarylium dye, a cyanine dye, zinc copper phosphate pigment, palladate compounds, platinate compounds, and combinations thereof. In some embodiments, the IR absorbing material comprises cyanine dyes selected from the group consisting of indocyanine dye (ICG), 2-[2-[2-chloro-3-[[1,3-dihydro-1,1-dimethyl-3-(4-sulfobutyl)-2H-benzo[e]indol-2-ylidene]-ethylidene]-1-cyclohexen-1-yl]-ethenyl]-1,1-dimethyl-3-(4-sulfobutyl)-1H-benzo[e]indolium hydroxide inner salt, sodium salt (IR820, new ICG dye), heptamethine cyanine (IR825), heptamethine cyanine (IR780), and combinations thereof.

In some embodiments, the IR absorbing material is indocyanine green (ICG).

In some embodiments, the squarylium dye is a benzopyrylium squarylium dyes having formula (III)

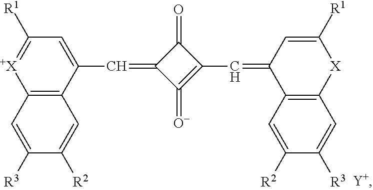

wherein each X is independently O, S, Se; Y⁺ is a counterion selected from the group consisting of hexafluoroarsenate (AsF₆⁻), hexafluoroantimonate (SbF₆⁻), hexafluorophosphate (PF₆⁻), (C₆F₅)₄B⁻, tetrafluoroborate (BF₄⁻), and combinations thereof; each R¹ is a non-aromatic organic substituent, each R²═H or OR³, R³=cycloalkyl, alkenyl, acyl, silyl; each R³═—NR⁴R⁵, each R⁴, R⁵ is independently H, C1-8 alkyl. In some embodiments, the squarylium dye of formula (III) is a compound when R¹═—CMe₃, R²═OCHMeEt, X═O with a strong absorption at 788 nm. In some embodiments, the squarylium dye of formula (III) is a compound when R¹═—CMe₃, R²═H, R³═—NEt₂, X═O with a strong absorption at 808 nm (IR 193 dye).

In some embodiments, the IR absorbing material may include a squarylium dye. In some embodiments, the IR absorbing material may include squaraine dye. In some embodiments, the IR absorbing material may include a squarylium dye selected from the group consisting of IR 193 dye, 1,3-bis[[2-(1,1-dimethylethyl)-4H-1-benzopyran-4-ylidene]methyl]-2,4-dihydroxy−cyclobutenediylium salt, 1,3-dihydroxy-2,4-bis[(2-phenyl-4H-1-benzopyran-4-ylidene)methyl]-cyclobutenediylium salt, 1,3-bis[[2-(1,1-dimethylethyl)-6-methyl-4H-1-benzopyran-4-ylidene]methyl]-2,4-dihydroxy-cyclobutenediylium salt, 1,3-bis[[2-(1,1-dimethylethyl)-7-hydroxy-4H-1-benzopyran-4-ylidene]methyl]-2,4-dihydroxy-cyclobutenediylium salt, 1,3-bis[[2-(1,1-dimethylethyl)-6-(1-methylethyl)-4H-1-benzopyran-4-ylidene]methyl]-2,4-dihydroxy-cyclobutenediylium salt, 1,3-dihydroxy-2,4-bis[1-(2-phenyl-4H-1-benzopyran-4-ylidene)ethyl]-cyclobutenediylium salt, 1,3-dihydroxy-2,4-bis[(2-phenyl-4H-naphtho[1,2-b]pyran-4-ylidene)methyl]-cyclobutenediylium salt, 1,3-dihydroxy-2,4-bis[[6-(1-methylethyl)-2-phenyl-4H-1-benzopyran-4-ylidene]methyl]-cyclobutenediylium salt, 1,3-bis[[6-(1,1-dimethylethyl)-2-phenyl-4H-1-benzopyran-4-ylidene]methyl]-2,4-dihydroxy-cyclobutenediylium salt, 1,3-bis[(2-cyclohexyl-7-methoxy-4H-1-benzopyran-4-ylidene)methyl]-2,4-dihydroxy-cyclobutenediylium salt, 1,3-bis[[2-(1,1-dimethylethyl)-6-(1-methylpropoxy)-4H-1-benzopyran-4-ylidene]methyl]-2,4-dihydroxy-cyclobutenediylium salt, 1,3-bis[[8-chloro-2-(1,1-dimethylethyl)-6-(1-methylethyl)-4H-1-benzopyran-4-ylidene]methyl]-2,4-dihydroxy-cyclobutenediylium salt, 1,3-bis[[7-(dimethylamino)-2-(1,1-dimethylethyl)-4H-1-benzopyran-4-ylidene]methyl]-2,4-dihydroxy-cyclobutenediylium salt, 1-[[7-(diethylamino)-2-(1,1-dimethylethyl)-4H-1-benzopyran-4-ylidene]methyl]-3-[[7-(dimethylamino)-2-(1,1-dimethylethyl)-4H-1-benzopyran-4-ylidene]methyl]-2,4-dihydroxy-cyclobutenediylium salt, 1,3-bis[[7-(diethylamino)-2-(1,1-dimethylethyl)-4H-1-benzopyran-4-ylidene]methyl]-2,4-dihydroxy-cyclobutenediylium salt, 1,3-bis[1-[7-(diethylamino)-2-(1,1-dimethylethyl)-4H-1-benzopyran-4-ylidene]ethyl]-2,4-dihydroxy-cyclobutenediylium salt, 1-[[7-(diethylamino)-2-(1,1-dimethylethyl)-4H-1-benzopyran-4-ylidene]methyl]-3-[[2-(1,1-dimethylethyl)-7-(2-ethylbutoxy)-4H-1-benzopyran-4-ylidene]methyl]-2,4-dihydroxy-cyclobutenediylium salt, 1,3-bis[[2-cyclohexyl-7-(diethylamino)-4H-1-benzopyran-4-ylidene]methyl]-2,4-dihydroxy-cyclobutenediylium salt, 1,3-bis[[2-(1,1-dimethylethyl)-7-(1-piperidinyl)-4H-1-benzopyran-4-ylidene]methyl]-2,4-dihydroxy-cyclobutenediylium salt, 1,3-bis[[2-(1,1-dimethylethyl)-7-(hexahydro-1H-azepin-1-yl)-4H-1-benzopyran-4-ylidene]methyl]-2,4-dihydroxy-cyclobutenediylium salt, 1,3-bis[[2-(1,1-dimethylethyl)-7-(4-morpholinyl)-4H-1-benzopyran-4-ylidene]methyl]-2,4-dihydroxy-cyclobutenediylium salt, 1,3-bis[[11-(1,1-dimethylethyl)-2,3,6,7-tetrahydro-1H,5H,9H-[1]benzopyrano[6,7,8-ij]quinolizin-9-ylidene]methyl]-2,4-dihydroxy-cyclobutenediylium salt, 1,3-bis[[2-(1,1-dimethylethyl)-6-(4-morpholinyl)-4H-1-benzopyran-4-ylidene]methyl]-2,4-dihydroxy-cyclobutenediylium salt, 1,3-bis[[2-bicyclo[2.2.1]hept-5-en-2-yl-7-(diethylamino)-4H-1-benzopyran-4-ylidene]methyl]-2,4-dihydroxy-cyclobutenediylium salt, 1,3-bis[[7-(2,3-dihydro-1Hindol-1-yl)-2-(1,1-dimethylethyl)-4H-1-benzopyran-4-ylidene]methyl]-2,4-dihydroxy-cyclobutenediylium salt, 1,3-bis[[7-(diethylamino)-2-[(1R,5S)-6,6-dimethylbicyclo[3.1.1]hept-2-en-2-yl]-4H-1-benzopyran-4-ylidene]methyl]-2,4-dihydroxy-cyclobutenediylium salt, 1,3-bis[[7-(diethylamino)-2-(6,6-dimethylbicyclo[3.1.1]hept-2-en-3-yl)-4H-1-benzopyran-4-ylidene]methyl]-2,4-dihydroxy-cyclobutenediylium salt, 1,3-dihydroxy-2,4-bis[[7-(4-morpholinyl)-2-tricyclo[3.3.1.13,7]dec-1-yl-4H-1-benzopyran-4-ylidene]methyl]-cyclobutenediylium salt, 2,4-bis[[7-(diethylamino)-2-(1,1-dimethylethyl)-4H-1-benzopyran-4-ylidene]methyl]-1,3-cyclobutanedione, and combinations thereof.

In some embodiments, the infrared-absorbing materials are inorganic substances that contain specific chemical elements having an incomplete electronic d-shell (i.e. atoms or ions of transition elements), and whose infrared absorption is a consequence of electronic transitions within the d-shell of the atom or ion. In some embodiments, the inorganic IR absorbing materials comprise one or more transition metal elements in the form of an ion such as a palladium(II), a platinum(II), a titanium(III), a vanadium(IV), a chromium(V), an iron(II), a nickel(II), a cobalt(II) or a copper(II) ion (corresponding to the chemical formulas Ti³⁺, VO²⁺, Cr⁵⁺, Fe²⁺, Ni²⁺, Co²⁺, and Cu²⁺). In some embodiments, the materials are inorganic IR absorbing materials with near-infrared absorbing properties selected from the group consisting of zinc copper phosphate pigment ((Zn,Cu)₂P₂O₇), zinc iron phosphate pigment ((Zn,Fe)₃(PO₄)₂), magnesium copper silicate ((Mg,Cu)₂Si₂O₆ solid solutions), and combinations thereof. In some embodiments, the inorganic IR absorbing material is a zinc iron phosphate pigment. In some embodiments, the inorganic IR absorbing material may include palladate (e.g. barium tetrakis(cyano-C)palladate tetrahydrate, BaPd(CN)₄.4H₂O, [Pd(dimit)₂]²⁻, bis(1,3-dithiole-2-thione-4,5-dithiolate)palladate(II). In some embodiments, the inorganic IR absorbing material may include platinate, e.g. platinum-based polypyridyl complexes with dithiolate ligands, Pt(II)(diamine)(dithiolate) with 3,3′-, 4,4′-, 5,5′-bipyridyl substituents.

In some embodiments, the material is selected from the group consisting of indocyanine green dye (ICG), new ICG dye (IR820), IR 193 dye, squaraine dye, iron oxide nanoparticle, a plasmonic absorber, a tetrakis aminium dye, and combinations thereof. In some embodiments, the plasmonic absorbers comprise gold nanostructures. In some embodiments, the plasmonic absorbers comprise gold nanostructures such as nanoporous gold thin films, or gold nanospheres, gold nanorods, gold nanoshells, gold nanocages, silver nanoparticles, and Cu₉S₅ nanoparticle.

In some embodiments, the IR absorbing material is admixed within the carrier to form a homogeneous dispersion or a solid solution. In some embodiments, the IR absorbing material and the carrier may have oppositely charged functional group(s) (e.g. IR absorbing material is positively charged tetrakis aminium dye, and the carrier has negatively charged functional group such as carboxylate anion of polymethacrylate polymers) such that the IR absorbing dye attaches to the carrier via hydrogen bond or via ionic electrostatic interactions.

The preferred concentration of the material responsive to the exogenous source is dependent on the amount required to obtain the desired response at the site of action. For example, in the case of an IR dye needed to absorb incident IR radiation, too little dye can limit the temperature rise that would be obtained. Likewise, too high a concentration can lead to dye aggregation, which can reduce and shift the absorption, such that the dye no longer absorbs the wavelength provided by the laser and leads to insufficient heating at the site of action. In some embodiments, the material responsive to the exogenous source is present in an amount ranging from about 0.01 wt. % to about 25.0 wt. % by the total weight of the heat delivery medium, or particle heater. In some embodiments, the material responsive to the exogenous source is present in an amount ranging from about 1.0 wt. % to about 20.0 wt. % by the total weight of the heat delivery medium, or particle heater. In some embodiments, the material responsive to the exogenous source is present in an amount ranging from about 5.0 wt. % to about 20.0 wt. % by the total weight of the heat delivery medium or particle heater. In some embodiments, the material responsive to the exogenous source is present in an amount ranging from about 5.0 wt. % to about 15.0 wt. % by the total weight of the heat delivery medium or particle heater. In some embodiments, the material responsive to the exogenous source is present in an amount ranging from about 10.0 wt. % to about 15.0 wt. % by the total weight of the heat delivery medium or particle heater. In some embodiments, the material is present in an amount selected from the group consisting of about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, about 20.0 wt. %, about 20.5 wt. %, about 21.0 wt. %, about 21.5 wt. %, about 22.0 wt. %, about 22.5 wt. %, about 23.0 wt. %, about 23.5 wt. %, about 24.0 wt. %, about 24.5 wt. %, and about 25.0 wt. %. In some embodiments, the material is present in an amount selected from the group consisting of about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, and about 15.0 wt. % by the total weight of the heat delivery medium or particle heater. In some embodiments, the material is present in an amount selected from the group consisting of about 1.0 wt. %, about 5.0 wt. %, about 10.0 wt. %, and about 15.0 wt. % by the total weight of the heat delivery medium or particle heater.

In some embodiments, the heat delivery medium has a weight ratio of the carrier to the material of about 10:1 to about 1:10. In some embodiment, the weight ratio of the carrier to the material is selected from the group consisting of about 10:1, about 9:1, about 8:1, about 7:1, about 6:1, about 5:1, about 4:1, about 3:1, about 2:1, about 1:1, about 1; 2, about 1:3, about 1:4, about 1:5, about 1:6, about 1:7, about 1:8, about 1:9, and about 1:10. In some embodiments, the weight ratio of the carrier to the material is 1:1.

In some embodiments, the heat delivery medium has a weight ratio of the carrier to the material of about 10:1 to about 1:10. In some embodiment, the particle heater has a weight ratio of the weight ratio of the carrier to the material selected from the group consisting of about 10:1, about 9:1, about 8:1, about 7:1, about 6:1, about 5:1, about 4:1, about 3:1, about 2:1, about 1:1, about 1; 2, about 1:3, about 1:4, about 1:5, about 1:6, about 1:7, about 1:8, about 1:9, and about 1:10. In some embodiments, the particle heater has a weight ratio of the weight ratio of the carrier to the material of 1:1.

(iii) Structural Element

In some embodiments, the heat delivery composition comprises a structural element selected from the group consisting of a fiber, a film, a sheet, an implant scaffold, a stent, a hydrogel, a shape-memory hydrogel, a patch, a tape, an adhesive, a woven fabric, a nonwoven fabric, a biocompatible cross-linked polymer, and combinations thereof.

In some embodiments, the structural element comprises a biocompatible cross-linked polymer. In some embodiments, the biocompatible cross-linked polymer comprises a thermoresponsive hydrogel.

In some embodiments, the composition further comprises an inorganic filler agent. In some embodiments, the inorganic filler agent is selected from the group consisting of silicates including talc, kaolin, silica, laponite, apatite, hydroxyapatite, hydroxycarbonate apatite, calcium carbonate, calcium phosphate including monocalcium phosphate, dicalcium phosphate, tricalcium phosphate, and tetracalcium phosphate, and combinations thereof.

In some embodiments, the heat delivery medium is embedded within, dispersed in or forming a coating on the structural element. In some embodiments, the heat delivery medium is a particle, wherein the particle is embedded within or dispersed in the structural element.

### (iv) Optional Additives

In some embodiments, the heat delivery medium additionally comprise adjuvants selected from the group consisting of colorants, flavorants, medicaments, stabilizers, fillers, viscosity modifiers, and combinations thereof. Such adjuvants may optionally comprise reactive functionality so that they will be copolymerized with the resin.

In some embodiments, the heat delivery medium further includes thermal stabilizers. It should be noted that often the material that interacts with the exogenous source can be stable (low rate of degradation) at room temperature but when the heat delivery medium comprising the material is inside body, at body temperature of 37.5° C., degradation of the material can be significantly accelerated. Examples of useful thermal stabilizers include phenolic antioxidants such as butylated hydroxytoluene (BHT), 2-t-butylhydroquinone, and 2-t-butylhydroxyanisole.

In some embodiments, the adjuvant is an antioxidant, or a surfactant. In some embodiments, the adjuvant is an antioxidant for stabilizing the dyes or as scavenger for reactive oxygen species (ROS).

In some embodiments, the adjuvant is an antioxidant for stabilizing the dyes at human body temperature. In some embodiments, the antioxidants for stabilizing dyes comprise sterically hindered phenols with para-propionate groups. In some embodiments, the antioxidant for stabilizing dyes comprises pentaerythritol tetrakis(3,5-di-tert-butyl-4-hydroxyhydrocinnamate). In some embodiments, the antioxidant for stabilizing dyes comprises a phosphite such as tris(2,4-di-tert-butylphenyl)phosphite. In some embodiments, the antioxidant for stabilizing dyes comprises organosulfur compounds such as thioethers. In some embodiments, the antioxidant for stabilizing dyes comprises 1,3,5-tris(4-tert-butyl-3-hydroxy-2,6-dimethylbenzyl)-1,3,5-triazine-2,4,6-(1H,3H,5H)-trione (Cyanox® 1790); wherein the Cyanox® 1790 is colorless.

In some embodiments, the heat delivery medium further comprises a ROS scavenging agent selected from the group consisting of NADPH, uric acid, vitamin A, vitamin C, vitamin E, glutathione, beta-carotene, polyphenols, sodium pyruvate, N,N′-dimethylthiourea (DMTU), mannitol, 1H-Imidazol-1-yloxy, 2-(4-carboxyphenyl)-4,5-dihydro-4,4,5,5-tetramethyl-3-oxide, potassium salt (carboxy-PTIO), 6-hydroxy-2,5,7,8-tetramethylchroman-2-carboxylic acid (Trolox), manganese(III)-tetrakis(4-benzoic acid)porphyrin (MnTBAP), 2-phenyl-1,2-benzisoselenazol-3 (2H)-one (Ebselen), 4,5-dihydroxybenzene-1,3-disulfonate (Tiron), and combinations thereof.

In some embodiments, the particles/compositions/medium may include inhibitors of enzymatic antioxidants such as superoxide dismutase (SOD), catalase (CAT), glutathione peroxidase (GPx) and thioredoxin (Trx). These inhibitors include but are not limited by: LCS-1 (4,5-dichloro-2-m-tolylpyridazin-3(2H)-one, salicylic acid, 6-amino-5-nitroso-3-methyluracil, bis-choline tetrathiomolybdate (ATN-224); 2-methoxyoestradiol (2-ME); N—N′-diethyldithiocarbamate, 3-Amino-1,2,4-Triazole, ρ-Hydroxybenzoic acid, misonidazole, d-penicillamine hydrochloride, 1-penicillamine hydantoin, dl-Buthionine-[S, R]-sulfoximine (BSO), and Au(I) thioglucose etc.

In some embodiments, the heat delivery medium, specifically hydrogel, may optionally comprise a filler. In some embodiments, the filler is an inorganic filler selected from the group consisting of quartz; nitrides; glasses derived from Ce, Sb, Sn, Zr, Sr, Ba or Al; a composite glass composed of oxides of barium, silicon, boron, and aluminum; colloidal silica; feldspar; borosilicate glass; kaolin; talc; titania; zinc glass; zirconia-silica; fluoroaluminosilicate glass; submicron silica particles (e.g., pyrogenic silica such as the “Aerosil®” Series “OX 50”, “130”, “150” and “200” silica sold by Degussa and “Cab-O-Sil® M5” silica sold by Cabot Corp.), and combinations thereof. In some embodiments, the filler may be a silicate selected from the group consisting of laponite (lithium sodium magnesium silicate), talc, silica, kaolin, and combinations thereof. In some embodiments, wherein the filler comprises sintered ceramic composite of zirconia-silica.

In some embodiments, the filler is an organic filler selected from the group consisting of filled or unfilled pulverized polycarbonates, polyepoxides, and combinations thereof.

In some embodiments, the surface of the fillers may be treated with a surface treatment, such as a silane-coupling agent, in order to enhance the bond between the filler and the polymerizable resin. The coupling agent may be functionalized with reactive curing groups, such as acrylates, methacrylates, and the like.

In some embodiments, the heat delivery medium may further comprise a thickening agent. In some embodiments, the thickening agent may include polyacrylic acid having a molecular weight of about 200,000, polyalkylene such as polybutenes and poly-C1-C3 alkyl methacrylates, crosslinked polyacrylic acid polymers (e.g., Carbopol® polymers), acrylic acid and C10-C30 alkyl acrylate crosslinked with allyl pentaerythritol (Carbopol® copolymers), acrylic acid crosslinked with allyl sucrose or allyl pentaerythritol (Carbopol® homopolymers), polyalkylene oxides, polyethylene glycol, sodium alginate, polyvinylpyrrolidone, copolymer of N-vinylpyrrolidone and vinyl acetate, carboxymethyl cellulose calcium, carboxymethylcellulose sodium, starch, starch sodium phosphate, methylcellulose, sodium polyacrylate, alginic acid, casein, sodium casein, ethylcellulose, hydroxyethylcellulose, gluten, locust bean gum, gelatin, or hydrocolloids.

In some embodiments, the thickening agent may include hydrocolloids such as alginate, κ-carrageenan, κ-carrageenan, ι-carrageenan, carboxymethylcellulose, guar, gum arabic, locust bean gum, starch, pectin, microcrystalline cellulose, methylcellulose, konjac mannan, and xanthan gum. In some embodiments, the thickening agent may include carboxymethyl cellulose and carboxymethylcellulose.

In some embodiments, the amount of thickening agent ranges from about 0.01 wt. % to about 1.0 wt. % by the total weight of the heat delivery medium. In some embodiments, the amount of thickening agent ranges from about 0.01 wt. % to about 0.4 wt. % by the total weight of the heat delivery medium. In some embodiments, the amount of thickening agent is selected from the group consisting of 0.01 wt. %, 0.05 wt. %, 0.1 wt. %, about 0.2 wt. %, about 0.3 wt. %, about 0.4. wt. %, about 0.5 wt. %, about 0.6 wt. %, about 0.7 wt. %, about 0.8 wt. %, about 0.9 wt. %, and about 1.0 wt. %,

In some embodiments, the medium further comprises a contrast agent for imaging-guided applications. In some embodiments, the contrast agent is a radiopacifier, gold nanostructure, ICG, iron oxide, and combinations thereof. In some embodiments, the radiopacifier is BaSO₄ particle, ZrO₂ particle, a nonpolar-hydrophobic heavy metal-containing organic material, capable of forming complex with PMMA including triphenyl bismuth (TBP), tantalum powder, bismuth salicylate (BS), strontium containing hyaluronic acid (Sr-HA), polymer-based iodine contrast agent, and polymer-based bromine contrast agent.

In some embodiments, the polymer-based iodine contrast agent is selected from the group consisting of iodinated copolymer of (MMA) and 2-[4-iodobenzoyl]-oxo-ethyl-methacrylate in a 1:1 weight/weight ratio (I-copolymer), iodixanol (IDX), iohexol (IHX), 2,5-diiodo-8-quinolyl methacrylate (IHQM), (4-iodophenol methacrylate, 72,73 2-[20,30,50-triiodobenzoyl] ethyl methacrylate (TIBMA), 3,5-diiodine salicylic methacrylate (DISMA), iohexol acetate, and combinations thereof. In some embodiments, the polymer-based bromine contrast agent is selected from the group consisting of 2-(2-bromoisobutyryloxy) ethyl methacrylate, copolymer of MMA and 2-(2-bromopropionyloxy) ethyl methacrylate, and combinations thereof.

In some embodiments, the radiopacifying agent comprises a polycrystalline ceramic metal oxide. In some embodiments, the radiopacifying agent is selected from the group consisting of HfO₂, La₂O₃, SrO, ZrO₂, and combinations thereof.

In some embodiments, the medium further includes thermal stabilizers. It should be noted that often the active agents and/or the material that interacts with the exogenous source can be stable (low rate of degradation) at room temperature, but when the particle comprising the active agent and the material is inside body, at body temperature of 37.5° C., degradation of the active agent and the material can be significantly accelerated. Examples of useful thermal stabilizers include phenolic antioxidants such as butylated hydroxytoluene (BHT), 2-tert-butyl-hydroquinone, and 2-tert-butyl-hydroxyanisole.

### (v) Heat Delivery Medium for Biomedical Applications

In some embodiments, the heat delivery medium comprises a polymer fiber carrier with an IR dye dispersed within, wherein the polymer fiber carrier comprises a polymer selected from the group consisting of PLGA, PEG, PLGA-PEG, PCL, poly-1-lysine (PLL), albumin, polyethylene imine (PEI) and combinations thereof.

In some embodiments, the heat delivery medium comprises a polymer fiber of which the surface is coated with an IR dye mixed with the carrier, wherein the carrier comprises a polymer selected from the group consisting of PLGA, PEG, PLGA-PEG, PCL, poly-1-lysine (PLL), albumin, polyethylene imine (PEI) and combination thereof.

In some embodiments, the heat delivery medium comprises a gelatin fiber having an IR dye dispersed within the fiber.

In some embodiments, the heat delivery medium comprises a collagen fiber having an IR dye dispersed within the fiber.

In some embodiments, the heat delivery medium comprises a PLGA fiber having an IR dye dispersed within the fiber.

In some embodiments, the heat delivery medium comprises a gelatin fiber having a tetrakis aminium dye dispersed within the gelatin fiber.

In some embodiments, the heat delivery medium comprises a collagen fiber having a tetrakis aminium dye dispersed within the fiber.

In some embodiments, the heat delivery medium comprises a PLGA fiber having a tetrakis aminium dye dispersed within the fiber.

In some embodiments, the heat delivery medium comprises a PLGA fiber having an indocyanine green dye dispersed within the fiber.

In some embodiments, the heat delivery medium comprises a gelatin fiber having a indocyanine green dye dispersed within the gelatin fiber the fiber.

In some embodiments, the heat delivery medium comprises a collagen fiber having a indocyanine green dye dispersed within the fiber.

In some embodiments, the heat delivery medium comprises a gelatin fiber with Epolight™ 1117 IR dye dispersed within the fiber.

In some embodiments, the heat delivery medium comprises a collagen fiber with Epolight™ 1117 IR dye dispersed within the fiber.

In some embodiments, the heat delivery medium comprises a PLGA fiber with Epolight™ 1117 IR dye dispersed within the fiber.

In some embodiments, the heat delivery medium comprises an implant scaffold formed from a gelatin fiber containing Epolight™ 1117 IR dye, a collagen fiber containing Epolight™ 1117 IR dye, or a PLGA fiber containing Epolight™ 1117 IR dye.

In some embodiments, the heat delivery medium comprises an implant scaffold formed from a gelatin fiber containing indocyanine green dye, a collagen fiber containing indocyanine green dye, or a PLGA fiber containing indocyanine green dye.

In some embodiments, the heat delivery medium comprises the nonwoven fabric formed from a gelatin fiber containing Epolight™ 1117 IR dye, a collagen fiber containing Epolight™ 1117 IR dye, or a PLGA fiber containing Epolight™ 1117 IR dye.

In some embodiments, the heat delivery medium comprises the nonwoven fabric formed from a gelatin fiber containing indocyanine green dye, a collagen fiber containing indocyanine green dye, or a PLGA fiber containing indocyanine green dye.

In some embodiments, the heat delivery medium comprises the woven fabric formed from a gelatin fiber containing Epolight™ 1117 IR dye, a collagen fiber containing Epolight™ 1117 IR dye, or a PLGA fiber containing Epolight™ 1117 IR dye.

In some embodiments, the heat delivery medium comprises the woven fabric formed from a gelatin fiber containing indocyanine green dye, a collagen fiber containing indocyanine green dye, or a PLGA fiber containing indocyanine green dye.

In some embodiments, the heat delivery medium comprises a hydrogel with an IR dye dispersed within the hydrogel. In some embodiments, the heat delivery medium comprises the hydrogel having a tetrakis aminium dye dispersed within. In some embodiments, the heat delivery medium comprises the gelatin hydrogel having a tetrakis aminium dye dispersed within. In some embodiments, the heat delivery medium comprises the gelatin hydrogel having Epolight™ 1117 IR dye dispersed within. In some embodiments, the heat delivery medium comprises the collagen hydrogel having a tetrakis aminium dye dispersed within. In some embodiments, the heat delivery medium comprises the collagen hydrogel having Epolight™ 1117 IR dye dispersed within the gel. In some embodiments, the heat delivery medium comprises the gelatin hydrogel having indocyanine green dye dispersed within. In some embodiments, the heat delivery medium comprises the collagen hydrogel having indocyanine green dye dispersed within the gel.

In some embodiments, the heat delivery medium is an injectable thermoresponsive gel liquid composition containing glycol-chitin and particles comprising PPMA-BMA carrier and Epolight™ 1117 IR dye. In some embodiments, the heat delivery medium is an injectable liquid composition containing about 5.0 wt. % to 30 wt. % of glycol-chitin and about 0.5 wt. % to about 20.0 wt. % particles having Epolight™ 1117 dye and PMMA-BMA carrier.

In some embodiments, the heat delivery medium is an injectable thermoresponsive gel liquid composition containing glycol-chitin and particles comprising PPMA-BMA carrier and indocyanine green dye. In some embodiments, the heat delivery medium is an injectable liquid composition containing about 5.0 wt. % to 30 wt. % of glycol-chitin and about 0.5 wt. % to about 20.0 wt. % particles having indocyanine green dye and PMMA-BMA carrier.

In some embodiments, the heat delivery medium is an injectable thermoresponsive gel liquid composition containing PEG-PLGA-PEG and particles having Epolight™ 1117 dye admixed with PMMA-BMA carrier. In some embodiments, the heat delivery medium is an injectable liquid composition containing about 5.0 wt. % to 30 wt. % of PEG-PLGA-PEG and about 0.5 wt. % to about 20.0 wt. % of particles having Epolight™ 1117 dye and PMMA-BMA carrier. In some embodiments, the heat delivery medium is an injectable thermoresponsive gel liquid composition containing PEG-PLGA-PEG and particles having indocyanine green dye admixed with PMMA-BMA carrier. In some embodiments, the heat delivery medium is an injectable liquid composition containing about 5.0 wt. % to 30 wt. % of PEG-PLGA-PEG and about 0.5 wt. % to about 20.0 wt. % of particles having indocyanine green dye and PMMA-BMA carrier.

In some embodiments, the heat delivery medium is an injectable thermoresponsive gel liquid composition containing PDLLA-PEG-PDLLA and Epolight™ 1117-B805 particle. In some embodiments, the heat delivery medium is an injectable liquid composition containing about 5.0 wt. % to 30 wt. % of PDLLA-PEG-PDLLA and about 0.5 wt. % to about 20.0 wt. % of particles having Epolight™ 1117 dye and PMMA-BMA carrier. In some embodiments, the heat delivery medium is an injectable thermoresponsive gel liquid composition containing PDLLA-PEG-PDLLA and particles containing indocyanine green dye and PMMA-BMA carrier. In some embodiments, the heat delivery medium is an injectable liquid composition containing about 5.0 wt. % to 30 wt. % of PDLLA-PEG-PDLLA and about 0.5 wt. % to about 20.0 wt. % of particles having indocyanine green dye and PMMA-BMA carrier.

In some embodiments, the heat delivery medium is a sprayable thermoresponsive gel liquid composition containing glycol-chitin and particles having Epolight™ 1117 dye and PMMA-BMA carrier. In some embodiments, the heat delivery medium is an injectable liquid composition containing about 5.0 wt. % to 30 wt. % of glycol-chitin and about 0.5 wt. % to about 20.0 wt. % of particles having Epolight™ 1117 dye and PMMA-BMA carrier. In some embodiments, the heat delivery medium is a sprayable thermoresponsive gel liquid composition containing glycol-chitin and particles having indocyanine green dye and PMMA-BMA carrier. In some embodiments, the heat delivery medium is an injectable liquid composition containing about 5.0 wt. % to 30 wt. % of glycol-chitin and about 0.5 wt. % to about 20.0 wt. % of particles having indocyanine green dye and PMMA-BMA carrier.

In some embodiments, the heat delivery medium is a sprayable thermoresponsive gel liquid composition containing PEG-PLGA-PEG and particles having Epolight™ 1117 dye and PMMA-BMA carrier. In some embodiments, the heat delivery medium is an injectable liquid composition containing about 5.0 wt. % to 30 wt. % of PEG-PLGA-PEG and about 0.5 wt. % to about 20.0 wt. % of particles having Epolight™ 1117 dye and PMMA-BMA carrier. In some embodiments, the heat delivery medium is a sprayable thermoresponsive gel liquid composition containing PEG-PLGA-PEG and particles having indocyanine green dye and PMMA-BMA carrier. In some embodiments, the heat delivery medium is an injectable liquid composition containing about 5.0 wt. % to 30 wt. % of PEG-PLGA-PEG and about 0.5 wt. % to about 20.0 wt. % of particles having indocyanine green dye and PMMA-BMA carrier.

In some embodiments, the heat delivery medium is sprayable thermoresponsive gel liquid composition containing PDLLA-PEG-PDLLA and particles having Epolight™ 1117 dye and PMMA-BMA carrier. In some embodiments, the heat delivery medium is an injectable liquid composition containing about 5.0 wt. % to 30 wt. % of PDLLA-PEG-PDLLA and about 0.5 wt. % to about 20.0 wt. % of particles having Epolight™ 1117 dye and PMMA-BMA carrier. In some embodiments, the heat delivery medium is sprayable thermoresponsive gel liquid composition containing PDLLA-PEG-PDLLA and particles having indocyanine green dye and PMMA-BMA carrier. In some embodiments, the heat delivery medium is an injectable liquid composition containing about 5.0 wt. % to 30 wt. % of PDLLA-PEG-PDLLA and about 0.5 wt. % to about 20.0 wt. % of particles having indocyanine green dye and PMMA-BMA carrier.

In some embodiments, the heat delivery medium comprises an adhesive having a tetrakis aminium dye dispersed within. In some embodiments, the heat delivery medium comprises the adhesive having Epolight™ 1117 IR dye dispersed within. In some embodiments, the heat delivery medium comprises the pressure sensitive adhesive having a tetrakis aminium dye dispersed within. In some embodiments, the heat delivery medium comprises the pressure sensitive adhesive having Epolight™ 1117 IR dye dispersed within. In some embodiments, the heat delivery medium comprises the silicone pressure sensitive adhesive having a tetrakis aminium dye dispersed within. In some embodiments, the heat delivery medium comprises the silicone pressure sensitive adhesive having Epolight™ 1117 IR dye dispersed within. In some embodiments, the heat delivery medium comprises the polyacrylate pressure sensitive adhesive having a tetrakis aminium dye dispersed within. In some embodiments, the heat delivery medium comprises the polyacrylate pressure sensitive adhesive having Epolight™ 1117 IR dye dispersed within. In some embodiments, the heat delivery medium comprises the adhesive having indocyanine green dye dispersed within. In some embodiments, the heat delivery medium comprises the pressure sensitive adhesive having indocyanine green dye dispersed within. In some embodiments, the heat delivery medium comprises the silicone pressure sensitive adhesive having indocyanine green dye dispersed within. In some embodiments, the heat delivery medium comprises the polyacrylate pressure sensitive adhesive having indocyanine green dye dispersed within.

In some embodiments, the heat delivery composition comprises the heat delivery medium and the structural elements selected from the group consisting of a fiber, a coating, an implant scaffold, a hydrogel, an adhesive, a patch, a woven fabric, a nonwoven fabric, a film, a sheet, a biocompatible cross-linked polymer, and combinations thereof.

In some embodiments, the heat delivery medium comprises an electrospun nanofiber, having a tetrakis aminium dye uniformly distributed across the cross-section of each constituent nanofiber. In some embodiments, the heat delivery composition comprises tetrakis aminium dye loaded electrospun nanofibers forming a coating layer on the structural element.

In some embodiments, the heat delivery medium comprises an electrospun nanofiber, having Epolight™ 1117 IR dye uniformly distributed across the cross-section of each constituent nanofiber. In some embodiments, the heat delivery composition comprises Epolight™ 1117 IR dye loaded electrospun nanofibers forming a coating layer on the structural element. In some embodiments, the heat delivery medium comprises an electrospun nanofiber, having indocyanine green dye uniformly distributed across the cross-section of each constituent nanofiber. In some embodiments, the heat delivery composition comprises indocyanine green dye loaded electrospun nanofibers forming a coating layer on the structural element.

In some embodiments, the heat delivery composition comprises an electrospun PLGA, gelatin, or collagen nanofiber having a tetrakis aminium dye particle heaters uniformly distributed across the cross-section of each constituent nanofiber. In some embodiments, the heat delivery composition comprises tetrakis aminium dye loaded electrospun PLGA, gelatin, or collagen nanofibers forming a coating layer on the structural element. In some embodiments, the heat delivery composition comprises an electrospun PLGA, gelatin, or collagen nanofiber having indocyanine green dye particle heaters uniformly distributed across the cross-section of each constituent nanofiber. In some embodiments, the heat delivery composition comprises indocyanine green dye loaded electrospun PLGA, gelatin, or collagen nanofibers forming a coating layer on the structural element.

In some embodiments, the heat delivery composition comprises a tetrakis aminium dye loaded electrospun polydioxanone nanofiber, wherein the tetrakis aminium dye uniformly is distributed across the cross-section of each constituent nanofiber, and the tetrakis aminium dye loaded electrospun polydioxanone nanofibers forming a coating layer on the structural element. In some embodiments, the heat delivery composition comprises Epolight™ 1117 IR dye loaded electrospun polydioxanone nanofiber, wherein the Epolight™ 1117 IR dye uniformly distributed across the cross-section of each constituent nanofiber, and the Epolight™ 1117 IR dye loaded electrospun polydioxanone nanofibers forming a coating layer on the structural element.

In some embodiments, the heat delivery composition comprises a tetrakis aminium dye loaded electrospun poliglecaprone nanofiber wherein the tetrakis aminium dye is uniformly distributed across the cross-section of each constituent nanofiber.

In some embodiments, the heat delivery composition comprises Epolight™ 1117 IR dye loaded electrospun poliglecaprone nanofiber wherein the Epolight™ 1117 IR dye is uniformly distributed across the cross-section of each constituent nanofiber.

In some embodiments, the heat delivery composition comprises an indocyanine green dye loaded electrospun poliglecaprone nanofiber wherein the indocyanine green dye is uniformly distributed across the cross-section of each constituent nanofiber. In some embodiments, the heat delivery composition comprises indocyanine green dye loaded electrospun poliglecaprone nanofiber wherein the indocyanine green dye is uniformly distributed across the cross-section of each constituent nanofiber.

In some embodiments, the heat delivery composition comprises an Epolight™ 1117 IR dye loaded electrospun polyglactin 910 nanofiber (copolymer of 90% glycolide and 10% L-lactide), wherein the Epolight™ 1117 IR dye is uniformly distributed across the cross-section of each constituent nanofiber.

In some embodiments, the heat delivery medium comprises indocyanine green dye loaded electrospun polyglactin 910 nanofiber (copolymer of 90% glycolide and 10% L-lactide), wherein the indocyanine green dye is uniformly distributed across the cross-section of each constituent nanofiber. In some embodiments, the heat delivery composition comprises indocyanine green dye loaded electrospun polyglactin 910 nanofiber wherein the indocyanine green dye is uniformly distributed across the cross-section of each constituent nanofiber.

### 2. Particle Heaters

In some embodiments, this disclosure provides a composition comprising a particle having a carrier admixed with a material that interacts with an exogenous source; wherein the material absorbs and converts the energy from the exogenous source to heat, and the heat then initiates or accelerates a physical, chemical or biological activity, and further wherein the particle structure is constructed such that it passes the Extractable Cytotoxicity Test. In some embodiments, the material exhibits stability such that at least 20% energy-to-heat conversion efficiency is achieved.

In some embodiments, at least a portion of the exterior surface of the particle has a modification that is polar, non-polar, charged, ionic, basic, acidic, reactive, hydrophobic, or hydrophilic.

Using conventional, linear or modestly branched polymers as the carrier, it has been found that the free volume or porosity of the carrier can allow an unacceptable amount of leakage, as determined by the Extractable Cytotoxicity Test. As a result, it has been found that coating the initially formed particle with a cross-linked inorganic polymer shell improves the resistance of the particle to incursion by biological media. The high degree of cross-linking of the shell would consequentially reduce the porosity of the shell and improve particle performance in the ECT to achieve IC₃₀ or less.

In some embodiments, the particle may further comprise a shell to form a core-shell particle. In some embodiments, the shell comprises an agent selected from the group consisting of Au, Ag, Cu, iron oxide, and combinations thereof. In some embodiments, the shell comprises a plasmonic absorber. In some embodiments, the plasmonic absorber comprises plasmonic nanomaterials of noble metal gold (Au), silver (Ag) and copper (Cu) nanoparticles doped with sulfur (S), selenium (Se) or tellurium (Te) having a plasmonic resonance at NIR wavelength.

The shell may comprise inorganic polymers such as silicates, organosilicate, organo-modified silicone polymer, or may be cross-linked organic polymers such as polyureas or polyurethanes. The process to apply the cross-linked shell must be designed so as to maximize the stability of the particle components to the chemistry required in shell construction, at least until the growing shell protects the components encapsulated in the particle.

Therefore, in some embodiments, the present disclosure provides particles having a core-shell structure to reduce particle porosity and to protect the material from the degradation by the body chemicals. Therefore, the stability of the material inside the particles is improved due to the reduced incursion of the body chemicals. In some embodiments, the shell comprises a cross-linked organo-silicate polymer derived from trialkoxysilane, or trihalosilane, for example, to protect the IR absorbing dye Epolight™ 1117 encapsulated in a NeoCryl® 805 particle when introduced into human skin, a sol-gel organo-modified silicate polymer shell derived from alkyltrimethoxysilane is formed on the surface of the polymeric particle to block the free exchange of nucleophiles and free radical species between the particles and the surrounding environment.

In some embodiments, the trialkoxysilane used for making the shell is selected from the group consisting of C2-C7 alkyl-trialkoxysilane, C2-C7 alkenyl-trialkoxysilane, C2-C7 alkynyl-trialkoxysilane, aryl-trialkoxysilane, and combinations thereof. In some embodiments, the trihalosilane used for making the shell is selected from the group consisting of trichlorosilane, tribromosilane, triiodosilane, and combinations thereof. In some embodiments, the cross-linked organosilicate polymer is derived from vinyltrimethoxysilane.

### (i) Carrier

In an embodiment, the carrier forming the particle may include a lipid selected from the group consisting of lipid, polymer-lipid conjugate, carbohydrate-lipid conjugate, peptide-lipid conjugate, protein-lipid conjugate, and combinations thereof. In some embodiments, the lipid may include one or more of the following: phospholipids such as phosphatidylcholines, phosphatidylserines, phosphatidylinositides, phosphatidylethanolamines, phosphatidylglycerols, phosphatidic acids; sphingolipids such as sphingomyelins, ceramides, phytoceramides, cerebrosides; sterols such as cholesterol, desmosterol, lanthosterol, stigmasterol, zymosterol, or diosgenin. In some embodiments, the carrier comprises a polymer-lipid conjugate, wherein the polymers conjugated to polar head groups of the lipid may include polyethylene glycol, polyoxazolines, polyglutamines, polyasparagines, polyaspartamides, polyacrylamides, polyacrylates, polyvinylpyrrolidone, or polyvinylmethyether. In some embodiments, the carrier comprises a carbohydrate-lipid conjugate, wherein the carbohydrates conjugated to the lipid may include monosaccharides (glucose, fructose), disaccharides, oligosaccharides or polysaccharides such as glycosaminoglycan (hyaluronic acid, keratan sulfates, heparin sulfate or chondroitin sulfate), carrageenan, microbial exopolysaccharides, alginate, chitosan, pectin, chitin, cellulose, or starch. In one embodiment, the phospholipid is selected from the group consisting of dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC), 1,2-dimyristoyl-sn-glycero-3-phosphocholine (DMPC), 1,2-dimyristoyl-sn-glycero-3-phosphorylglycerol (DMPG), 1,2-distearoyl-sn-glycero-3-phosphoethanolamine (DSPE), 1,2-dioleoyl-sn-glycero-3-phosphocholine (DOPC), 1,2-dioleoyl-sn-glycero-3-phosphoethanolamine (DOPE), 1,2-dipalmitoyl-sn-glycero-3-phospho-(1′-rac-glycerol) (DPPG), 1,2-distearoyl-sn-glycero-3-phosphocholine (DSPC), distearoylphosphoethanolamine conjugated with polyethylene glycol (DSPE-PEG), phosphatidylserine (PS), phosphatidylethanolamine (PE), phosphatidylglycerol (PG), phosphatidylcholine (PC), and combinations thereof. In an embodiment, the particle comprise the lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, PG, and combinations thereof.

In some embodiments, the carrier forming the particle comprises a hydrophobic polymer or copolymer of polymethacrylates, polycarbonate, or combinations thereof. In some embodiments, the carrier comprises poly(methyl methacrylate) (PMMA, NeoCryl® 728 sold by DSM, T_{g}=111° C., acid value of 6.5).

In some embodiments, the carrier forming the particle comprises a copolymer of two different methacrylate monomers. In some embodiments, the carrier comprises a copolymer of methyl methacrylate monomer and C2-C6 alkyl methacrylate monomer. In some embodiments, the carrier comprises a copolymer of methyl methacrylate monomer and C2-C4 alkyl methacrylate monomer. In some embodiments, the carrier comprises a copolymer of methyl methacrylate monomer and C3-C4 alkyl methacrylate monomer. In some embodiments, the polymethacrylate copolymer is made from methyl methacrylate monomer and C4 alkyl methacrylate monomer. In some embodiments, the polymethacrylate copolymer is made from methyl methacrylate (MMA) monomer in an amount ranging from about 80.0 wt. % to about 99.0 wt. % and butyl methacrylate (BMA) monomer in an amount ranging from about 1.0 wt. % to about 20.0 wt. % by the total weight of the polymethacrylate copolymer. In some embodiments, the polymethacrylate copolymer is made from MMA monomer in an amount ranging from about 85.0 wt. % to about 96.0 wt. % and BMA monomer in an amount ranging from about 4.0 wt. % to about 15.0 wt. % by the total weight of the polymethacrylate copolymer. In some embodiments, the polymethacrylate copolymer is made from MMA monomer in an amount ranging from about 90.0 wt. % to about 96.0 wt. % and BMA monomer in an amount ranging from about 4.0 wt. % to about 10.0 wt. % by the total weight of the polymethacrylate copolymer. In some embodiments, the polymethacrylate copolymer is made from MMA monomer in an amount ranging from about 95.0 wt. % to about 96.0 wt. % and BMA monomer in an amount ranging from about 4.0 wt. % to about 5.0 wt. % by the total weight of the polymethacrylate copolymer. In some embodiments, the polymethacrylate copolymer is made from about 99.0 wt. % MMA monomer and about 1.0 wt. % BMA monomer by the total weight of the polymethacrylate copolymer. In some embodiments, the polymethacrylate copolymer is made from about 98.0 wt. % MMA monomer and about 2.0 wt. % BMA monomer by the total weight of the polymethacrylate copolymer. In some embodiments, the polymethacrylate copolymer is made from about 97.0 wt. % MMA monomer and about 3.0 wt. % BMA monomer by the total weight of the polymethacrylate copolymer. In some embodiments, the polymethacrylate copolymer is made from about 96.0 wt. % MMA monomer and about 4.0 wt. % BMA monomer by the total weight of the polymethacrylate copolymer. In some embodiments, the polymethacrylate copolymer is made from about 95.0 wt. % MMA monomer and about 5.0 wt. % BMA monomer by the total weight of the polymethacrylate copolymer. In some embodiments, the polymethacrylate copolymer is made from about 94.0 wt. % MMA monomer and about 6.0 wt. % BMA monomer by the total weight of the polymethacrylate copolymer.

In some embodiments, the weight ratio of the MMA repeating units to the BMA repeating units in the MMA/BMA copolymer is 80:20 to 99:1. In some embodiments, the weight ratio of the MMA repeating units to the BMA repeating units in the MMA/BMA copolymer is 85:15 to 96:4. In some embodiments, the weight ratio of the MMA repeating units to the BMA repeating units in the MMA/BMA copolymer is 90:10 to 96:4. In some embodiments, the weight ratio of the MMA repeating units to the BMA repeating units in the MMA/BMA copolymer is 95:5 to 96:4. In some embodiments, the weight ratio of the MMA repeating units to the BMA repeating units in the MMA/BMA copolymer is 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, or 99:1. In some embodiments, the polymethacrylate copolymer is MMA/BMA copolymer and the weight ratio of MMA to BMA is 96:4 (e.g. NeoCryl® 805 by DSM, acid value less than 1).

In some embodiments, the carrier is PMMA. In some embodiments, the carrier is a polyacrylate blend comprising 96% PMMA and 4% PBMA. In some embodiments, the carrier is a methyl methacrylate/butyl methacrylate copolymer comprising 96% methyl methacrylate repeating units and 4% butyl methacrylate repeating units. In some embodiments, the poly(methyl methacrylate) is a copolymer of methyl methacrylate/butyl methacrylate (NeoCryl® B-805, T_{g} 99° C., average molecular weight 85,000 Da).

In some embodiments, the hydrophobic polymethacrylate has an acid value less than 10. In some embodiments, the hydrophobic polymethacrylate has an acid value less than 5. In some embodiments, the hydrophobic polymethacrylate has an acid value less than 2. In some embodiments, the hydrophobic polymethacrylate has an acid value less than 1.

In some embodiments, the carrier comprises cross-linkable reactive groups selected from the group consisting of vinyl group (—CH═CH₂), ethynyl group (—C≡C—), vinyl dimethyl sulfone group, hydroxyl group (—OH), thiol group (—SH), amine group (—NH₂), aldehyde group (—CHO), carboxylic acid group (—COOH), and combinations thereof. In some embodiments, the carrier comprises the cross-linkable polysaccharides.

In some embodiments, the carrier forming the particle is present in the particle at a weight percentage by the total weight of the particle selected from the group consisting of about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, or about 20.0 wt. %, about 25.0 wt. %, about 30.0 wt. %, about 35.0 wt. %, about 40.0 wt. %, about 45.0 wt. %, about 50.0 wt. %, about 55.0 wt. %, about 60.0 wt. %, about 65.0 wt. %, about 70.0 wt. %, about 75.0 wt. %, about 80.0 wt. %, about 85.0 wt. %, about 90.0 wt. %, about 95.0 wt. %, and about 99.0 wt. %. In some embodiments, the carrier is present in the particle at a weight percentage by the total weight of the particle ranges from about 1 wt. % to about 99 wt. %. In some embodiments, the carrier is present in the particle at a weight percentage by the total weight of the particle ranges from about 10.0 wt. % to about 90.0 wt. %. In some embodiments, the carrier is present in the particle at a weight percentage by the total weight of the particle ranges from about 50.0 wt. % to about 90.0 wt. %. In some embodiments, the carrier is present in the particle at a weight percentage by the total weight of the particle ranges from about 25.0 wt. % to about 50.0 wt. %. In some embodiments, the carrier is present in the particle at a weight percentage by the total weight of the particle ranges from about 75.0 wt. % to about 90.0 wt. %.

In some embodiments, the particle comprises NeoCryl® B-805 (copolymer of 96.0 wt. % methyl methacrylate/4.0 wt. % butyl methacrylate) in an amount ranging from about 60.0 wt. % to about 80 wt. % by the total weight of the particle. In some embodiments, the particle comprises NeoCryl® B-805 in an amount selected from the group consisting of 62.0 wt. %, 70.0 wt. %, 75.0 wt. %, and 78.3 wt. % by the total weight of the particle. In some embodiments, the particle comprises NeoCryl® B-805 in an amount selected from the group consisting of about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, and about 80 wt. % by the total weight of the particle.

(ii) Material Interacting with Exogenous Sources

In some embodiments, the material interacting with the exogenous source is an IR absorbing material having significant absorption of photonic energy in the near infrared spectrum region. In some embodiments, the IR absorbing material absorbs light at a wavelength ranging from 750 nm to 1400 nm. In some embodiments, the IR absorbing material absorbs light at a wavelength ranging from 750 nm to 1200 nm. In some embodiments, the IR absorbing material absorbs light at a wavelength ranging from 900 nm to 1100 nm. In some embodiments, the IR absorbing material absorbs light at a wavelength selected from the group consisting of 700 nm, 766 nm, 777 nm, 780 nm, 783 nm, 785 nm, 800 nm, 805 nm, 808 nm, 810 nm, 820 nm, 825 nm, 900 nm, 948 nm, 950 nm, 960 nm, 980 nm, 1000 nm, 1064 nm, 1065 nm, 1070 nm, 1071 nm, 1073 nm, 1098 nm, and 1100 nm. In some embodiments, the IR absorbing material has significant absorption at 1064 nm wavelength.

In some embodiments, the material interacting with the exogenous source is an absorbing material having significant absorption of photonic energy. In some embodiments, the absorbing material absorbs light at a wavelength ranging from 400 nm to 750 nm. In some embodiments, the absorbing material absorbs light at a wavelength selected from the group consisting of 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, and 750 nm.

In some embodiments, the IR absorbing material comprises a tetrakis aminium dye or an inorganic IR absorbing material.

In some embodiments, the IR absorbing material is a tetrakis aminium dye. In some embodiments, the tetrakis aminium dye is a narrow band absorber including commercially available dyes sold under the trademark names Epolight™ 1117 (peak absorption, 1071 nm), Epolight™ 1151 (peak absorption, 1070 nm), or Epolight™ 1178 (peak absorption, 1073 nm). In some embodiments, the tetrakis aminium dyes is a broad band absorber including commercially available dyes sold under the trademark names Epolight™ 1175 (peak absorption, 948 nm), Epolight™ 1125 (peak absorption, 950 nm), and Epolight™ 1130 (peak absorption, 960 nm). In some embodiments, the tetrakis aminium dye is Epolight™ 1178.

In some embodiments, the tetrakis aminium dye is Epolight™ 1178. In some embodiments, the IR absorbing material is a tetrakis aminium dye has minimal visible color. In some embodiments, the tetrakis aminium dye is Epolight™ 1117 ((hexafluorophosphate as counterion, molecular weight, 1211 Da, peak absorption 1098 nm).

In some embodiments, the IR absorbing material is indocyanine green (ICG). After the ICG particles are irradiated with pulsed laser light, the excited ICG dye produces singlet oxygen species in the presence of cellular water. In some embodiments, the ICG nanoparticles may also co-encapsulate with reactive oxygen species scavenger (e.g. antioxidant) to augment the therapeutic efficacy of the ICG dye.

In some embodiments, the IR absorbing material comprises inorganic IR absorbing materials. In some embodiments, the inorganic IR absorbing materials comprise one or more transition metal elements in the form of an ion such as a titanium(III), a vanadium(IV), a chromium(V), an iron(II), a nickel(II), a cobalt(II) or a copper(II) ion (corresponding to the chemical formulas Ti³⁺, VO²⁺, Cr⁵⁺, Fe²⁺, Ni²⁺, Co²⁺, and Cu²⁺). In some embodiments, the materials are inorganic IR absorbing materials with near-infrared absorbing properties selected from the group consisting of zinc copper phosphate pigment ((Zn,Cu)₂P₂O₇), zinc iron phosphate pigment ((Zn,Fe)₃(PO₄)₂), magnesium copper silicate ((Mg,Cu)₂Si₂O₆ solid solutions), and combinations thereof. In some embodiments, the inorganic IR absorbing material is a zinc iron phosphate pigment.

In some embodiments, the material interacting with the exogenous source comprises plasmonic absorbers. In some embodiments, the plasmonic absorbers comprise plasmonic nanomaterials of noble metal gold (Au), silver (Ag) and copper (Cu) nanoparticles doped with sulfur (S), selenium (Se) or tellurium (Te) having a plasmonic resonance at a NIR wavelength. In some embodiments, the plasmonic absorbers comprise gold nanostructures such as nanoporous gold thin films, or gold nanospheres, gold nanorods, gold nanoshells, gold nanocages, silver nanoparticles, Cu₉S₅ nanoparticle, and iron oxide nanoparticles. In some embodiments, the plasmonic absorbers comprise gold nanostructures.

Compared to non-metal nanoparticles, plasmonic nanomaterials exhibit a unique photophysical phenomenon, called localized surface plasmon resonance (LSPR) as a result of the absorption of light at a resonant frequency. The plasmonic nanomaterials (e.g. noble metal nanostructures) show superior light absorption efficiency over conventional dye molecules. Upon exposure to electromagnetic radiation, strong surface fields are induced due to the coherent excitation of the electrons in the metallic nanoparticles. By changing the structure (e.g. size) and shape, the LSPR frequency of the noble metal nanostructures can be tuned to shift the resulting plasmonic resonance wavelength in the NIR therapeutic window (750-1300 nm), where light penetration in the tissue is optimal. The endogenous absorption coefficient of the tissue in the NIR band is nearly two orders of magnitude lower than that in the visible band of EM spectrum. In some embodiments, the plasmonic absorbers may have an LSPR ranging from about 900 nm to about 1064 nm.

In some embodiments, the particle heaters comprise core particles of 100-200 nm in size formed of the carrier and the material as described above, and a thin layer of noble metal film (5-20 nm) as particle surface coatings, wherein the noble metal is selected from the group consisting of noble metal including gold (Au) nanostructure, silver (Ag) nanoparticle, copper (Cu) nanoparticle having a plasmonic resonance at a NIR wavelength, and combinations thereof, wherein the heat delivery composition exhibits additive or synergistic photothermal therapy (PTT) resulting from LSPR of film coated nanoparticle and the conventional PTT from organic dye in the core. The LSPR wavelength is tunable by decreasing the shell thickness-to-core radius ratio.

In some embodiments, the particle heaters comprise core particles of 1000-2000 nm in size formed of the carrier and the material as described above, and a thin layer of noble metal film (5-50 nm) as particle surface coatings, wherein the noble metal is selected from the group including gold (Au) nanostructure, silver (Ag) nanoparticle, copper (Cu) nanoparticle having a plasmonic resonance at a NIR wavelength, and combinations thereof, wherein the particle heaters exhibit additive or synergistic PTT. The LSPR wavelength is tunable by decreasing the shell thickness-to-core radius ratio.

In some embodiments, the particle heaters further comprise a shell to form core-shell particles, wherein the material interacting with the exogenous source is a plasmonic absorber disposed in the shell, wherein the plasmonic absorbers are either embedded within, ionically associated with, or covalently bound to the shell. In some embodiments, the plasmonic absorbers are particles having a thin and porous gold wall with hollow interior, wherein the LSPR wavelength can be tuned by changing the wall thickness, pore size and porosity. In some embodiments, the plasmonic absorbers are core-shell particles having a gold nanoparticle core having the shape of sphere, shell, or rod, and a shell of hydrophilic polymer (e.g. chitosan, PEG) to enclose the gold nanoparticle core.

In some embodiments, the material in the particle is present in an amount ranging from about 5.0 wt. % to about 15.0 wt. % by the total weight of the particles. In some embodiments, the material of the particle is present in an amount selected from the group consisting of about 5.0 wt. %, about 5.56 wt. %, about 10.4 wt. %, about 12.0 wt. %, about 12.1 wt. %, about 13.64 wt. %, about 14.0 wt. %, and about 15.0 wt. % by the total weight of the particles. In some embodiments, the particles comprise material in an amount of about 5.0 wt. %, about 5.25 wt. %, about 5.5 wt. %, about 5.75 wt. %, about 6.0 wt. %, 6.25 wt. %, about 6.5 wt. %, about 6.75 wt. %, about 7.0 wt. %, 7.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. %, about 10.25 wt. %, about 10.5 wt. %, about 10.75 wt. %, about 11.0 wt. %, about 11.25 wt. %, about 11.5 wt. %, about 11.75 wt. %, about 12.0 wt. %, about 12.25 wt. %, about 12.5 wt. %, about 12.75 wt. %, about 13.0 wt. %, about 13.25 wt. %, about 13.5 wt. %, about 13.75 wt. %, about 14.0 wt. %, about 14.25 wt. %, about 14.5 wt. %, about 14.75 wt. %, or about 15.0 wt. %.

In some embodiments, the particle has a weight ratio of the carrier to the material ranging from 1:1 to 7:1. In some embodiments, the particle has a weight ratio of the carrier to the material selected from the group consisting of 1.0:1, 1.1:1, 1.2:1, 1.3:1, 1.4:1, 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 41.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, and 7.0:1.

(iii) Optional Additives

In some embodiments, the particle further includes thermal stabilizers. It should be noted that often the material that interacts with the exogenous source can be stable (low rate of degradation) at room temperature but when the particle comprising the material is inside body, at a body temperature of 37.5° C., degradation of the material can be significantly accelerated. Examples of useful thermal stabilizers include phenolic antioxidants such as butylated hydroxytoluene (BHT), 2-t-butylhydroquinone, and 2-t-butylhydroxyanisole.

In some embodiments, the core of the particle may optionally comprise an additive. In some embodiments, the additive is an antioxidant, or a surfactant. In some embodiments, the additive is an antioxidant for stabilizing the dyes or as a scavenger for reactive oxygen species (ROS). In some embodiments, the additive is an antioxidant for stabilizing the dyes at human body temperature. In some embodiments, the antioxidants for stabilizing dyes comprise sterically hindered phenols with para-propionate groups. In some embodiments, the antioxidant for stabilizing dyes comprises pentaerythritol tetrakis(3,5-di-tert-butyl-4-hydroxyhydrocinnamate). In some embodiments, the antioxidant for stabilizing dyes comprises a phosphite such as tris(2,4-di-tert-butylphenyl)phosphite. In some embodiments, the antioxidant for stabilizing dyes comprises organosulfur compounds such as thioethers. In some embodiments, the antioxidant for stabilizing dyes comprises 1,3,5-tris(4-tert-butyl-3-hydroxy-2,6-dimethylbenzyl)-1,3,5-triazine-2,4,6-(1H,3H,5H)-trione (Cyanox® 1790); wherein the Cyanox® 1790 is colorless.

In some embodiments, the ROS scavenging agent selected from the group consisting of NADPH, uric acid, vitamin A, vitamin C, vitamin E, glutathione, beta-carotene, polyphenols, sodium pyruvate, N,N′-dimethylthiourea (DMTU), mannitol, 1H-Imidazol-1-yloxy, 2-(4-carboxyphenyl)-4,5-dihydro-4,4,5,5-tetramethyl-3-oxide, potassium salt (carboxy-PTIO), 6-hydroxy-2,5,7,8-tetramethylchroman-2-carboxylic acid (Trolox), manganese(III)-tetrakis(4-benzoic acid)porphyrin (MnTBAP), 2-phenyl-1,2-benzisoselenazol-3(2H)-one (Ebselen), 4,5-dihydroxybenzene-1,3-disulfonate (Tiron), and combinations thereof.

In some embodiments, the medium further comprises a contrast agent for imaging guided applications. In some embodiments, the contrast agent is a radiopacifier, gold nanostructure, ICG, iron oxide, and combinations thereof. In some embodiments, the radiopacifier is BaSO₄ particle, ZrO₂ particle, a nonpolar-hydrophobic heavy metal-containing organic material, capable of forming complex with PMMA including triphenyl bismuth (TBP), tantalum powder, bismuth salicylate (BS), strontium-containing hyaluronic acid (Sr-HA), polymer-based iodine contrast agent, and polymer-based bromine contrast agent.

In some embodiments, the polymer-based iodine contrast agent is selected from the group consisting of iodinated copolymer of (MMA) and 2-[4-iodobenzoyl]-oxo-ethyl-methacrylate in a 1:1 weight/weight ratio (I-copolymer), iodixanol (IDX), iohexol (IHX), 2,5-diiodo-8-quinolyl methacrylate (IHQM), (4-iodophenol methacrylate, 72,73 2-[20,30,50-triiodobenzoyl] ethyl methacrylate (TIBMA), 3,5-diiodine salicylic methacrylate (DISMA), iohexol acetate, and combinations thereof. In some embodiments, the polymer-based bromine contrast agent is selected from the group consisting of 2-(2-bromoisobutyryloxy) ethyl methacrylate, or copolymer of MMA, 2-(2-bromopropionyloxy) ethyl methacrylate, and combinations thereof.

In some embodiments, the radiopacifying agent comprises a polycrystalline ceramic metal oxide. In some embodiments, the radiopacifying agent is selected from the group consisting of HfO₂, La₂O₃, SrO, ZrO₂, and combinations thereof.

In some embodiments, the additive is a surfactant. In some embodiments, the surfactant may include cationic, amphoteric, and non-ionic surfactants. In some embodiments, the surfactants comprise anionic surfactants selected from the group consisting of fatty acid salts, bile salts, phospholipids, carnitines, ether carboxylates, succinylated monoglycerides, mono/diacetylated tartaric acid esters of mono- and diglycerides, citric acid esters of mono- and diglycerides, sodium oleate, sodium lauryl sulfate, sodium lauryl sarcosinate, sodium dioctyl sulfosuccinate (SDS), sodium cholate, sodium taurocholate, lauroyl carnitine, palmitoyl carnitine, myristoyl carnitine, lactylic esters of fatty acids, and combinations thereof. In some embodiments, anionic surfactants include di-(2-ethylhexyl) sodium sulfosuccinate. In some embodiments, the surfactants are non-ionic surfactants selected from the group consisting of propylene glycol fatty acid esters, mixtures of propylene glycol fatty acid esters and glycerol fatty acid esters, triglycerides, sterol and sterol derivatives, sorbitan fatty acid esters and polyethylene glycol sorbitan fatty acid esters, sugar esters, polyethylene glycol alkyl ethers and polyethylene glycol alkyl phenol ethers, polyoxyethylene-polyoxypropylene block copolymers, lower alcohol fatty acid esters, and combinations thereof. In some embodiments, the surfactant may comprise fatty acids. Examples of fatty acids include caprylic acid, undecylic acid, lauric acid, tridecylic acid, myristic acid, palmitic acid, stearic acid, or oleic acid. In some embodiments, the surfactants comprise amphoteric surfactants including (1) substances classified as simple, conjugated and derived proteins such as the albumins, gelatins, and glycoproteins, and (2) substances contained within the phospholipid classification, for example lecithin. The amine salts and the quaternary ammonium salts within the cationic group also comprise useful surfactants.

In some embodiments, the surfactant comprises a hydrophilic amphiphilic surfactant polyoxyethylene (20) sorbitan monolaurate (TWEEN® 20) or polyvinyl alcohol that improves the distribution of IR absorbing material in the polymeric carrier. In some embodiments, the surfactant comprises an amphiphilic surfactant if the IR absorbing material is hydrophilic and the polymeric carrier is hydrophobic. In some embodiments, the surfactant is an anionic surfactant sodium bis(tridecyl) sulfosuccinate (Aerosol® TR-70). In some embodiments, the surfactant is sodium bis(tridecyl) sulfosuccinate, or sodium dodecyl sulfate (SDS).

### (iv) Particle Size and Morphology

In some embodiments, the particles may be nanoparticles or microparticles. In some embodiments, the particles may have a spherical shape. In some embodiments, the particles may have cylindrical shape.

In some embodiments, the particles may have a wide variety of non-spherical shapes. The non-spherical shaped particles can be used to alter uptake by phagocytic cells and thereby clearance by the reticuloendothelial system. In some embodiments, the non-spherical particles may be in the shape of rectangular disks, high aspect ratio rectangular disks, rods, high aspect ratio rods, worms, oblate ellipses, prolate ellipses, elliptical disks, UFOs, circular disks, barrels, bullets, pills, pulleys, bi-convex lenses, ribbons, ravioli, flat pill, bicones, diamond disks, emarginated disks, elongated hexagonal disks, tacos, wrinkled prolate ellipsoids, wrinkled oblate ellipsoids, or porous elliptical disks. Additional shapes beyond those are also within the scope of the definition for “non-spherical” shapes.

The term “Polydispersity Index (PdI)” is defined as the square of the ratio of standard deviation (α) of the particle diameter distribution divided by the mean particle diameter (2a), as illustrated by the formula: PdI=(σ/2a)². PdI is used to estimate the degree of non-uniformity of a size distribution of particles, and larger PdI values correspond to a larger size distribution in the particle sample. PdI can also indicate particle aggregation along with the consistency and efficiency of particle surface modifications. A sample is considered monodisperse when the PdI value is less than 0.1.

In some embodiments, the particles have a PdI from about 0.05 to about 0.15, about 0.06 to about 0.14, about 0.07 to about 0.13, about 0.08 to about 0.12, or about 0.09 to about 0.11. In some embodiments, the particles have a PdI of about 0.05, about 0.06, about 0.07, about 0.08, about 0.09, about 0.10, about 0.11, about 0.12, about 0.13, about 0.14, or about 0.15.

In some embodiments, the particle has a median particle size less than 1000 nm. In some embodiments, the median particle size ranges from about 1 nm to about 1000 nm. In some embodiments, the median particle size ranges from about 1 nm to about 500 nm. In some embodiments, the median particle size ranges from about 1 nm to about 250 nm. In some embodiments, the median particle size ranges from about 1 nm to about 150 nm. In some embodiments, the median particle size ranges from about 1 nm to about 100 nm. In some embodiments, the median particle size ranges from about 1 nm to about 50 nm. In some embodiments, the median particle size ranges from about 1 nm to about 25 nm. In some embodiments, the median particle size ranges from about 1 nm to about 10 nm. In some embodiments, the particle has a median particle size selected from the group consisting of about 1 nm, about 5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 280 nm, about 285 nm, about 290 nm, about 295 nm, about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, and about 1000 nm. In some embodiments, the particle has a median particle size of 500 nm. In some embodiments, the particle has a median particle size of 250 nm. In some embodiments, the particle has a median particle size of 750 nm.

In some embodiments, the particles are microparticles having a median particle size equal or greater than 1000 nm (1 micron). In some embodiments, the particles have a median particle size selected from the group consisting of about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 105 μm, about 110 μm, about 115 μm, about 120 μm, about 125 μm, about 130 μm, about 140 μm, about 145 μm, about 150 μm, about 155 μm, about 160 μm, about 165 μm, about 170 μm, about 175 μm, about 180 μm, about 185 μm, about 190 μm, about 195 μm, about 200 μm, about 205 μm, about 210 μm, about 215 μm, about 220 μm, about 225 μm, about 230 μm, about 235 μm, about 240 μm, about 245 μm, about 250 μm, about 255 μm, about 260 μm, about 265 μm, about 270 μm, about 275 μm, about 280 μm, about 285 μm, about 290 μm, about 295 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, and about 500 μm. In some embodiments, the particle has a median particle size in a range from about 1 μm to about 500 μm. In some embodiments, the particle has a median particle size in a range from about 1 μm to about 250 μm. In some embodiments, the particle has a median particle size in a range from about 1 μm to about 100 μm. In some embodiments, the particle has a median particle size in the range from about 1 μm to about 50 μm. In some embodiments, the particle has a median particle size in a range from about 1 μm to about 25 μm. In some embodiments, the particle has a median particle size in a range from about 1 μm to about 10 μm. In some embodiments, the particle has a median particle size in a range from about 1 μm to about 6 μm. In some embodiments, the particle has a median particle size from about 1 μm, about 2 μm, about 3 μm, about 4 μm, about 5 μm, or about 6 μm. In some embodiments, the particle has a median particle size in the range from about 1 μm to about 4 μm.

### 3. Controlled Heat Generation

Heat generation by nanoparticles under optical illumination (particle-heater) for biomedical applications have attracted much interest. An important physical property of the particle-heater for causing an actuation of a biological process or a chemical process is the increased temperature generated within a biological system and the scope and spatial span over which the temperature change occurs. In a typical biomedical application of particle-heaters, the nanoparticles are injected into a small cavity inside a tissue and are optically stimulated. When the exogenous light source is applied, the material encapsulated in the particle will interact with the light source, absorb the energy thereof, and convert the energy to heat that travels outside the particle. Tissues typically have the heat conductivity of water and heat from the particle-heater is likely to flow isotropically inside the tissue.

In many biomedical applications, it is desirable to target tissues for localized heating to provide tunable temperature raise. Techniques which effect precise localized heating would allow for producing medical benefits while minimizing the collateral damage to nearby cells and tissues.

In one embodiment, the disclosure provides a method of generating heat by triggering a particle heater, a delivery medium, or a heat delivery composition described herein (“heat delivery composition/medium/particle”) with an exogenous source.

In an embodiment, this disclosure provides a method of remotely-triggered controlled heat generation comprises the steps of: (1) providing the heat delivery composition/medium/particle as disclosed herein, (2) exposing the heat delivery composition/medium/particle to an exogenous source for sufficient period of time, wherein the material absorbs the energy from the exogenous source and converts the energy to heat, wherein the heat transfer outside of the heat delivery medium or the particle.

In some embodiments, the material exhibits sufficient stability as provided by the Material Process Stability Test, e.g., the material preserves about 50% or greater of absorbance of energy from the exogenous source after being subjected to the process conditions (e.g., exposure to laser irradiation under specific operating parameters).

In some embodiments, the exogenous source is selected from the group consisting of an electromagnetic radiation, an electrical field, a microwave, a radio wave, an ultrasonic radiation, a magnetic field, and combinations thereof. In some embodiments, the exogenous source comprises microwave.

In some embodiments, the exogenous source comprises an ultrasonic source. In some embodiments, the material comprises ICG dye.

In some embodiments the exogenous source is an ultrasonic wave produced by an ultrasound (US) producing machine. In some embodiments the therapeutic ultrasound is either pulsed or continuous.

The frequency of ultrasound dictates the depth of penetration and impacts the efficiency of particle heaters. To reach deeper tissues (up to 5 cm or more), a frequency of 1 MHz should be selected. When the target tissue is within 2.5 cm from the surface of the skin, a frequency of 3 MHz should be selected. It is important to note that 3 MHz will produce heat from particle heaters approximately 3-times faster than 1 MHz, creating a higher efficiency in heating when compared to 1 MHz ultrasound for the same particle heater. For continuous US, frequencies within the range of 1-3 MHz at intensities of 0.5-10 W/cm² for a duration of 1-15 minutes at 100% duty cycle should be useful for in vivo applications. In some embodiments the US frequencies of 1-2 MHz at intensity ranges from 0.5-5 W/cm² are applied for 1-5 minutes at 100% duty cycle. 3 MHz ultrasound is absorbed more rapidly in the tissues, and therefore is considered to be most appropriate for superficial lesions, whilst the 1 MHz energy is absorbed less rapidly with deeper progression through the tissues, and can therefore be more effective at greater depth. The boundary between superficial and deep tissues is in some ways arbitrary, but somewhere around the 2 cm depth is often taken as a useful boundary. Hence, if the target tissue is within 2 cm (or just under an inch) of the skin surface, 3 MHz treatments will be effective whilst treatments to deeper tissues will be more effectively achieved with 1 MHz ultrasound. One important factor is that some of the US energy delivered to the tissue surface will/may be lost before the target tissue (i.e. in the normal or uninjured tissues which lie between the skin surface and the target). In order to account for this, it may be necessary to deliver more US energy at the surface than is required, therefore allowing for some absorption before the target tissue, and allowing sufficient remaining US energy to achieve the desired effect. To identify the appropriate dose to set on the machine, one has to determine (a) the estimated depth of the lesion to be treated and (b) the intensity of US energy required at that depth to achieve the desired effect. For example, to achieve a 0.5 W/cm² intensity at 1 cm tissue depth, one would select 3 MHz treatment option and set machine to 0.7 W/cm² which will result in 0.5 W/cm² intensity at a 1 cm tissue depth. The rate at which US energy is absorbed in the tissues can be approximately determined by the half-value depth—this is the tissue depth at which 50% of the US energy delivered at the surface has been absorbed. The average half-value depth of 3 MHz ultrasound is taken at 2.5 cm and that of 1 MHz ultrasound as 4.0 cm though there are numerous debates that continue with regards the most appropriate half value depth for different frequencies.

In some embodiments pulsed ultrasound is used. The pulse ratio determines the concentration of the sound energy on a time basis. The pulse ratio determines the proportion of time that the ultrasound machine is “ON” compared with the “OFF” time. A pulse ratio of 1:1 for example means that the machine delivers one ‘unit’ of US energy followed by an equal duration during which no energy is delivered. The machine duty cycle is therefore 50%. A machine pulsed at a ratio of 1:4 will deliver one unit of US energy followed by 4 units of rest, therefore the machine is on for 20% of the time (some machines use ratios, and some use percentages). The selection of the most appropriate pulse ratio essentially depends on the state of the target tissue(s). The less dense the target tissue state, the more energy sensitive it is, and appears to respond more favorably to energy delivered with a larger pulse ratio (lower duty cycle). As the tissue becomes denser, it appears to respond preferentially to a more ‘concentrated’ energy delivery, thus reducing the pulse ratio (or increasing the duty cycle). It is suggested that pulse ratios of 1:4 would be best suited to the treatment of low density tissues, reducing this as the tissue increases in density, moving through 1:3 and 1:2 to end up with 1:1 or continuous modes As a general rule, pulse ratio of 1:4 or 1:3 will be for the less dense tissues, 1:2 and 1:1 for the medium density tissues and 1:1 or continuous for the dense tissues. The final compilation of the treatment dose which is most likely to be effective is based on the principle that about 1-minute worth of US energy (at an appropriate frequency and intensity) should be delivered for every treatment head that needs to be covered. The size of the treatment area will influence the treatment time, as will the pulse ratio being used. The larger the treatment area, the longer the treatment will take. The more pulsed the energy output from the machine, the longer it will take to deliver about a 1-minute worth of US energy. Desired ultrasonic dose will also depend on the particle heater concentration at the target tissue.

In some embodiments, the exogenous source comprises an electromagnetic radiation.

Exposing the heat delivery composition/medium/particle to the electromagnetic radiation includes directing electromagnetic radiation onto the heat delivery composition/medium/particle. The electromagnetic radiation may come from any source, including an LED, laser, or lamp. Any source that can provide the appropriate radiation, including wavelength and intensity, is compatible with the disclosed methods. In some embodiments, the source is a narrow-band EMR source, with a particular bandwidth tuned to wavelengths compatible with human tissue. In some embodiments, the source is a broadband EMR source. In some embodiments, the electromagnetic radiation source comprises a LED light or a laser light. In some embodiments, the source is a laser. In some embodiments, the source is a pulsed laser.

In some embodiments, the electromagnetic radiation source comprises a LED light. LEDs are solid state p-n junction devices which emit light when forward biased. An LED is a light emitting diode, a generic term. An IRED is an infrared emitting diode, a term specifically applied to IR emitters. Unlike incandescent lamps which emit light over a very broad range of wavelengths, LEDs emit light over such a narrow bandwidth that they appear to be emitting a single “color”.

In some embodiments, the material absorbs optical energy at a wavelength ranging from 400 nm to 1050 nm. In some embodiments, the material absorbs optical energy at a wavelength from 400 nm to 700 nm. In some embodiments, the material absorbs optical energy at a wavelength from 400 nm to 750 nm (e.g. a LED device). In some embodiments, the material absorbs optical energy at a wavelength from 750 nm to 950 nm (e.g. IRED by Excelitas™). In some embodiments, the material is selected from the group consisting of squaraine dye, IR 193 dye, ICG dye, IR 820 dye (new ICG dye), and combinations thereof.

In some embodiments, the electromagnetic radiation source is a laser light. In some embodiments, pulsed lasers are utilized in order to provide localized thermal heating. In some embodiments, the laser irradiation is delivered in a pulse duration longer than the thermal relaxation time (TRT) of the particle heaters containing the exogenous source interacting material such that the heat energy generated by the particle begins to travel outside the particle. In some embodiments, the flow of the heat delivery to the outside of the particles can be achieved by manipulating the fluence of the laser irradiation, particle size and the concentration of the particles. Pulses are at least microseconds or milliseconds in duration.

In some embodiments, the laser pulse duration is in a range from milliseconds to nanoseconds, and the laser has an oscillation wavelength at 805 nm, 808 nm, or 1064 nm. In some embodiments, the heat delivery composition/medium/particle heater absorbs the laser light having a wavelength from 750 nm to 1400 nm. In some embodiments, heat delivery composition/medium/particle absorbs light having a wavelength ranging from 400 nm to 750 nm. In some embodiments, the material is selected from the group consisting of indocyanine green dye (ICG), new ICG dye (IR820), IR 193 dye, iron oxide, a tetrakis aminium dye, and combinations thereof.

In some embodiments, the method further comprises heating an area in the proximity of the heat delivery composition/medium/particle by transferring heat from the heat delivery composition/medium/particle to the surrounding area. As used herein, the term “in proximity to” is defined as an area containing the heat delivery composition/medium/particle or sufficiently near the heat delivery composition/medium/particle to receive heat transferred from the composition/particle after heated by irradiation. By this step, heating the heat delivery composition/medium/particle is used to heat an area around the heat delivery composition/medium/particle so as to provide targeted heat, activated by illumination. The area can be liquid, solid, gas, or any combinations thereof. The area to be heated by the heat delivery composition/medium/particle can be liquid, solid, gas, or any combinations thereof.

In one embodiment, the area is heated to a temperature of 25° C. to 120° C. In one embodiment, the area is heated to a temperature greater than 42° C. In one embodiment, the area is heated to a temperature of 37.5° C. to 50° C. In one embodiment, the area is heated to a temperature of about 37.5° C., about 38° C., about 38.5° C., about 39° C., about 39.5° C., about 40° C., about 40.5° C., about 41° C., about 41.5° C., about 42° C., about 42.5° C., about 43° C., about 43.5° C., about 44° C., about 44.5° C., about 45° C., about 45.5° C., about 46° C., about 46.5° C., about 47° C., about 47.5° C., about 48° C., about 48.5° C., about 49° C., about 49.5° C., or about 50° C.

In one embodiment, the method further includes heating a plurality of the particle heaters. While a single particle heater may be effective in a nano- or micron-scale environment, greater area can be heated by irradiating a plurality of the particle heaters.

In one embodiment, this disclosure provides a method of accelerating a physical, chemical or biological activity at an area in proximity to a heat delivery composition/medium/particle by an exogenous source comprising the following steps: (a) administering to a tissue site the heat delivery composition/medium/particle comprising a carrier and a material that interacts with an exogenous source; (b) irradiating the composition with the exogenous source, wherein the composition absorbs the energy from the exogenous source and converts the energy into heat; wherein the heat causes the temperature at the area in proximity to the heat delivery composition/medium/particle to rise such that the physical, chemical or biological activity is accelerated.

In one embodiment, this disclosure provides a method of accelerating a physical, chemical or biological activity at a tissue site by an exogenous source comprising the following steps: (a) administering to a tissue site a composition comprising a particle that comprises a carrier encapsulating a material that interacts with an exogenous source; (b) exposing the particle to an exogenous source, wherein the particle absorbs the energy from the exogenous source and converts the energy into heat; wherein the heat dissipates from the particle to an area in the proximity of the particle, wherein the heat causes a rise of temperature at the area in the proximity to be above the body temperature such that the physical, chemical or biological activity is accelerated.

In some embodiments, it is desirable to keep the temperature in the surrounding area of the heat delivery composition/medium/particle heater to be sufficiently low to avoid collateral damage to the healthy tissues and also control the temperature rise to accelerate a chemical or biological activity.

In some embodiments, the electromagnetic radiation source is a laser light. In some embodiments, pulsed lasers are utilized in order to provide localized thermal heating. In some embodiments, the laser irradiation is delivered in a pulse duration longer than the thermal relaxation time (TRT) of the particle heaters such that the heat generated by the particle begins to travel outside the particle. In some embodiments, the flow of the heat delivery to the outside of the particles can be achieved by manipulating the fluence of the laser irradiation, particle size and the concentration of the particles. Pulses are at least nanoseconds in duration.

The advantages of the efficient localized heating achieved by the heat delivery composition/medium/particle in this disclosure are immediately evident because the temperature change is primary limited to the area surrounding the heat delivery composition/medium/particle, that is, selective placement of the heat delivery composition/medium/particle allows heating of targeted regions without significantly affecting the remainder of the tissue. In addition, the energy-to-heat conversion effect enables heat to be generated by the heat delivery composition/medium/particle as opposed to the conventional laser-based photothermal tissue treatments that deliver energy to the endogenous natural pigments and dyes in the tissue (e.g. melanin). Thus, the process of the energy delivery by the exogenous source to the heat delivery composition/medium/particle in this disclosure can include selectively applying the exogenous source only to a predefined region of the tissue that is to be treated by the selective placement of the particles.

To avoid tissue damage, it is important to ensure the energy of laser irradiation is preferentially absorbed by the heat delivery composition/medium/particle containing the material interacting with the exogenous source and not absorbed by the tissue to be treated. When the delivery time exceeds the TRT of the heat delivery composition/medium/particle, then the heat energy generated begins to travel outside the particle. In addition, the duration of the pulse can be controlled to ensure that the heat absorbed by the heat delivery composition/medium/particle will diffuse out into the surrounding environment.

Even though the specificity of the particle heaters may allow for the induction of localized hyperthermia, if the absorption coefficient differential between the target and surrounding tissue is not large enough, collateral damage at the surface of the tissue may occur, resulting in damage to the healthy tissues. To avoid healthy tissue damage, it is important to ensure the energy of laser irradiation is preferentially absorbed by the particles containing the IR absorbing dye and not absorbed by the tissue to be treated. When the pulse duration exceeds the TRT of the particle heaters, then the heat energy generated begins to travel outside the particles. In addition, the duration of the pulse can be controlled to ensure that the heat produced by the particles will diffuse out into the surrounding environment.

In some embodiments, laser wavelength has a dual impact attributable to the absorption coefficient of the photo-responsive material as well as the depth of penetration to the tissue site, which roughly increases as the wavelength increases in the visible and near infrared spectrum. After carefully choosing a proper laser wavelength and pulse duration for a particular photo-responsive material, delivering the maximum number of photons to the heat delivery composition/particle having the same photo-responsive material can be achieved.

In some embodiments, the particle heater offers tunable photon absorption by varying the particle size, particle concentration, and selection of IR absorbing material with a defined chemical structure to allow facile matching of particle absorption to the output of various commercial lasers. Additionally, the method in this disclosure affords a path to minimize tissue damage by using the least harmful wavelengths of laser light sources.

In some embodiments, radiation is applied until the temperature of the surrounding area is about 40°−60° C. The exposure time is dependent upon many factors, including but not limited to, area of radiation coverage, wavelength and intensity of the radiation, type and mass of the composition and particle heater concentration.

In some embodiments, the induced hyperthermia is a mild hyperthermia at a temperature ranging from about 38.0° C. to about 41.0° C. In some embodiments, the induced hyperthermia is a moderate hyperthermia at a temperature ranging from about 41.1° C. to about 45.0° C., wherein the hyperthermia does not cause collateral damage to healthy cells. In some embodiments, the induced hyperthermia is a profound hyperthermia at a temperature ranging from about 45.1° C. to about 52.0° C.

Due to the efficient absorption of the particles, photothermal heating to significant temperatures can be achieved without harming the tissue of a treatment subject. In one embodiment, irradiating the particle heater comprises an irradiation wavelength of 650 nm to 1350 nm. In one embodiment, irradiating the silicon nanoparticle comprises an irradiation wavelength of 785 nm to 900 nm. In one embodiment, irradiating the silicon nanoparticle comprises an irradiation wavelength of 650 nm to 1000 nm.

In some embodiments, the laser has a peak oscillation wavelength selected from the group consisting of 700 nm, 766 nm, 777 nm, 780 nm, 783 nm, 785 nm, 800 nm, 808 nm, 810 nm, 820 nm, 825 nm, 900 nm, 948 nm, 950 nm, 960 nm, 980 nm, 1000 nm, 1060 nm, 1064 nm, 1070 nm, 1071 nm, 1073 nm, 1098 nm, and 1100 nm. In some embodiments, the laser has an oscillation wavelength at 1071 nm. In some embodiments, the laser has an oscillation wavelength at 1064 nm. In some embodiments, the laser has an oscillation wavelength at 808 nm.

In some embodiments, the pulse duration of the laser is longer than the TRT of the particle. In some embodiments, the laser pulse duration is in a range from milliseconds to nanoseconds, and the laser has an oscillation wavelength at 1064 nm. In some embodiments, the laser pulse duration is in a range from milliseconds to nanoseconds, and the laser has an oscillation wavelength at 805 nm. In some embodiments, the laser pulse duration is in a range from milliseconds to nanoseconds, and the laser has an oscillation wavelength at 808 nm.

In some embodiments, the exogenous source comprises light sources such as a laser (ion laser, semiconductor laser, Q-switched laser, free-running laser, or fiber laser). Typically, the energy source is capable of emitting radiation at a wavelength from about 700 nm, 1000 nm, 2000 nm, 5000 nm, about 10,000 nm or more. In some embodiments, the photonic energy is radiation at an intensity from about 0.00005 mW/cm² to about 1000 TW/cm². The optimum intensity is chosen to induce high thermal gradients from particle heaters in a range from submicron to about 10 microns in the surrounding tissue but has minimal residual effect on heating tissue in which particles do not reside within a radius of about 100 microns or more from the nanoparticle. In certain embodiments, a differential heat gradient between the target tissue region and other tissue regions (e.g., the skin) is greater than 2-fold, 3-fold, 5-fold, 10-fold, 15-fold, 20-fold, 50-fold, 100-fold, or greater than 100-fold.

Laser sources include a pulsed laser source, which may be a single wavelength polarized (or, alternatively, unpolarized) laser source capable of emitting radiation at a frequency from about 750 nm to about 1400 nm. Alternatively, the optical source is a multiple wavelength laser source capable of emitting radiation at a wavelength from about 1000 nm to about 1200 nm. The pulsed laser source is generally capable of emitting pulsed radiation at a frequency from about 1 Hz to about 1 THz.

In some embodiments, various types of lasers may be suitable for excitation of the particle heaters of this disclosure such as Q-switched (QS) laser such as QS alexandrite lasers (operating at 755 nm), QS Nd:YAG lasers (operating at 1060 nm, 1440 nm, laser that penetrate deeper into the dermis). The selection of laser parameters used to cause a controlled heat generation may include wavelength, average power, instantaneous power, pulse duration and/or total exposure duration. The pulse duration (t_{d}), of the exposure can influence the specificity or confinement of collateral thermal damage and may be determined from the thermal relaxation time (tᵣ, also known as TRT) of the target material. The transition from specific to non-specific thermal damage can occur when the ratio is as follows: (t_{d}/tᵣ)≥1. For spheres of radius, R, and thermal diffusivity, κ, the thermal relaxation time can be provided by tᵣ=(R²/6.75κ).

To confine localization of heat inside particle selectively, the pulse duration of the laser exposure is shorter than the thermal relaxation time of the particle. The power density is sufficient to induce a localized mild hyperthermia (e.g. a temperature increase of at least 5° C. about room temperature) to the surrounding environment to the particles.

For example, a spherical IR dye particle having about 21 μm in diameter, the thermal relaxation time for the particle is estimated to be about 20 μs according to Eqn. (I) TRT=R²/6.75 k, Pulsed laser systems having shorter pulse durations (about 10 μs) can thus provide for the targeting of the 21 μm IR dye particle.

In some embodiments, the speed of the induction of localized hyperthermia is tunable by tuning the laser wavelength. To induce rapid induction of localized hyperthermia, pulsed laser irradiation at 1064 nm is employed (nanosecond). To induce induction of localized hyperthermia at a slower pace (e.g., 1 minute to several minutes), pulsed laser irradiation at 805 nm is employed. In some embodiments, one or more repeats of the laser irradiation may be employed to drive the heat diffusion and conduction to the surrounding area of the heat delivery composition/medium/particles.

In some embodiments, the laser pulse duration is longer than the particle TRT. In some embodiments, the laser pulse duration is less than a millisecond, microsecond in duration. In some embodiments, a source emitting radiation at a wavelength of 755 nm is operated in pulse mode such that the emitted radiation is pulsed at a duration of 0.25-300 milliseconds (ms) per pulse, with a pulse frequency of 1-10 Hz. In some embodiments, a source emitting radiation at a wavelength of 810 nm is pulsed at 5-100 ms with a frequency of 1-10 Hz. In some embodiments, a source emitting radiation at a wavelength of 1064 nm is pulsed at 0.25-300 ms at a frequency of 1-10 Hz. In some embodiments, a source emitting pulsed light at a wavelength of 530-1200 nm is pulsed at 0.5-300 ms at a frequency of 1-10 Hz.

In some embodiments, the particles have a TRT ranging from about 250 ns, about 250 ns, about 275 ns, about 300 ns, about 325 ns, about 350 ns, about 375 ns, about 400 ns, about 425 ns, about 450 ns, about 475 ns, about 500 ns, about 525 ns, about 550 ns, about 575 ns, about 600 ns, about 625 ns, about 650 ns, about 675 ns, about 700 ns, about 725 ns, about 750 ns, about 775 ns, about 800 ns, about 825 ns, about 900 ns, about 925 ns, about 950 ns, about 975 ns, about 1000 ns, about 1100 ns, about 1200 ns, about 1300 ns, about 1400 ns, about 1500 ns, about 1600 ns, about 1700 ns, about 1800 ns, about 1900 ns, about 2.0 ms, about 3 ms, about 4 ms, about 5 ms, about 6 ms, about 7 ms, about 8 ms, about 9 ms, about 10 ms, about 20 ms, about 30 ms, about 40 ms, about 50 ms, about 60 ms, about 70 ms, about 80 ms, about 90 ms, or about 100 ms.

In some embodiments, short pulses (100 ns to 1000 ms) are used to drive very high transient heat gradients in and around the target composition from embedded particles to localize chemistry in close proximity to particle location. In other embodiments, longer pulse lengths (1 ms to 10 ms, or 1 ms to 500 ms) are used to drive heat gradients further from the target structure to localize thermal energy to components greater than 100 μm away from the localized particles. In some such embodiments, pulses of varying durations are provided to localize thermal heating regions to be within 0.05, 0.1, 0.5, 1, 2, 5, 10, 20, 30, 50, 75, 100, 200, 300, 500, 1000 microns of the particles.

The usage of high laser irradiance (1-48 W/cm²) is practically not applicable as they exceed the skin tolerance threshold values (maximum permissible exposure (MPE) for power density at 808 nm is 350 mW/cm², with an exposure time of 10-1000 s) set by the American National Standards Institute (ANSI). The skin tolerance threshold values for power density at 968 nm is about 360 mW/cm². The skin tolerance threshold values for power density at 1064 nm is about 420 mW/cm². In some embodiments, the laser is operated at an energy density of about 0.1 W/cm² to about 4.0 W/cm². In some embodiments, the laser is operated at an energy density of about 0.1 W/cm² to about 0.75 W/cm². In some embodiments, the laser is operated at an energy density of about 0.1 W/cm², 0.11 W/cm², 0.12 W/cm², 0.13 W/cm², 0.14 W/cm², 0.15 W/cm², 0.16 W/cm², 0.17 W/cm², 0.18 W/cm², 0.19 W/cm², 0.20 W/cm², 0.21 W/cm², 0.22 W/cm², 0.23 W/cm², 0.24 W/cm², 0.25 W/cm², 0.26 W/cm², 0.27 W/cm², 0.28 W/cm², 0.29 W/cm², 0.30 W/cm², 0.31 W/cm², 0.32 W/cm², 0.33 W/cm², 0.34 W/cm², 0.35 W/cm², 0.36 W/cm², 0.37 W/cm², 0.38 W/cm², 0.39 W/cm², 0.40 W/cm², 0.41 W/cm², 0.42 W/cm², 0.43 W/cm², 0.44 W/cm², 0.45 W/cm², 0.46 W/cm², 0.47 W/cm², 0.48 W/cm², 0.49 W/cm², 0.50 W/cm², 0.51 W/cm², 0.52 W/cm², 0.53 W/cm², 0.54 W/cm², 0.55 W/cm², 0.56 W/cm², 0.57 W/cm², 0.58 W/cm², 0.59 W/cm², 0.60 W/cm², 0.61 W/cm², 0.62 W/cm², 0.63 W/cm², 0.64 W/cm², 0.65 W/cm², 0.66 W/cm², 0.67 W/cm², 0.68 W/cm², 0.69 W/cm², 0.70 W/cm², 0.71 W/cm², 0.72 W/cm², 0.73 W/cm², 0.74 W/cm², 0.75 W/cm², 0.76 W/cm², 0.77 W/cm², 0.78 W/cm², 0.79 W/cm², 0.80 W/cm², 0.81 W/cm², 0.82 W/cm², 0.83 W/cm², 0.84 W/cm², 0.85 W/cm², 0.86 W/cm², 0.87 W/cm², 0.88 W/cm², 0.89 W/cm², 0.90 W/cm², 0.91 W/cm², 0.92 W/cm², 0.93 W/cm², 0.94 W/cm², 0.95 W/cm², 0.96 W/cm², 0.97 W/cm², 0.98 W/cm², 0.99 W/cm², 1.0 W/cm², 1.1 W/cm², 1.2 W/cm², 1.3 W/cm², 1.4 W/cm², 1.5 W/cm², 1.6 W/cm², 1.7 W/cm², 1.8 W/cm², 1.9 W/cm², 2.0 W/cm², 2.1 W/cm², 2.2 W/cm², 2.3 W/cm², 2.4 W/cm², 2.5 W/cm², 2.6 W/cm², 2.7 W/cm², 2.8 W/cm², 2.9 W/cm², 3.0 W/cm², 3.1 W/cm², 3.2 W/cm², 3.3 W/cm², 3.4 W/cm², 3.5 W/cm², 3.6 W/cm², 3.7 W/cm², 3.8 W/cm², 3.9 W/cm², and 4.0 W/cm².

In some embodiments, the power density of the laser irradiation ranges from about 0.5 W/cm² to 1.0 W/cm². In some embodiments, the laser is operated at a wavelength of 750 nm, 805 nm, 808 nm, 810 nm, 1064 nm and the power density of the laser irradiation is selected from the group consisting of about 0.1 W/cm², about 0.2 W/cm², about 0.3 W/cm², about 0.4 W/cm², about 0.5 W/cm², about 0.6 W/cm², about 0.7 W/cm², about 0.8 W/cm², about 0.9 W/cm², about 1.0 W/cm², about 1.1 W/cm², about 1.2 W/cm², about 1.3 W/cm², about 1.4 W/cm², and about 1.5 W/cm². In some embodiments, the laser is operated at 750 nm, 805 nm, 808 nm, 810 nm, 1064 nm with a power density of about 40 mW/cm² to about 450 mW/cm². In some embodiments, the laser is operated at 750 nm, 805 nm, 808 nm, 810 nm, 1064 nm with a power density of about 40 mW/cm² to about 360 mW/cm². In some embodiments, the laser is operated at 750 nm, 805 nm, 808 nm, 810 nm, 1064 nm with a power density of about 100 mW/cm² to about 350 mW/cm².

In some embodiments, the 808 nm NIR laser is operated at ultra-low laser power (10 mW) to induce the generation of ROS, (dominantly photodynamic process, PD effects). Various repetition rates are used from continuous to pulsed, e.g., at less than 1 Hz, or 1-5 Hz. In some embodiments, the tissue is irradiated at a fluence of 1-60 J/cm² with laser wavelengths of about, e.g., 750 nm, 810 nm, 1064 nm, or other wavelengths, particularly in the range of infrared light. Various repetition rates are used from continuous to pulsed, e.g., at 1-10 Hz, 10-100 Hz, 100-1000 Hz. While some energy is reflected, it is an advantage of the subject matter described herein is that a substantial amount of energy is absorbed by particles, with a lesser amount absorbed by skin. Particles are delivered to the tissue site at concentration sufficient to absorb, e.g., 1.1-100× more energy than other components of the tissue of similar volume. This is achieved in some embodiments by having a concentration of particles in the tissue site with absorbance at the laser peak of 1.1-100× relative to other tissue components of similar volume.

To enable tunable localized heat delivery, light-absorbing particles are utilized in conjunction with a laser or other excitation source of the appropriate wavelength. The laser light may be applied in pulses with a single pulse or with multiple pulses of light. The intensity of heating and distance over which the photothermal effect will occur are controlled by the intensity and duration of light exposure, and the concentration of the laser excitable particles.

In some embodiments, the method employs a composition applied to the tissue site containing a low concentration of particles and a high intensity laser irradiation such that the local temperature maxima caused by photothermal conversion by the particles are within a nanometer scale distance from the excited particles. In some embodiments, the method employs a composition applied to the tissue site containing a higher concentration of laser excitable particles and a low intensity laser irradiation such that the local temperature maxima caused by photothermal conversion from the particles are at a millimeter scale distance from the excited particles (also known as collective photo-heating).

In some embodiments, the material in the heat delivery/medium/particles is at a concentration ranging from about 0.1 mg/mL to about 10.0 mg/mL. In some embodiments, the material in the heat delivery/medium/particles is at a concentration ranging from 1.0 mg/mL to 5.0 mg/mL. In some embodiments, the concentration of the material in the heat delivery/medium/particles is selected from the group consisting of about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, about 3.0 mg/mL, about 3.1 mg/mL, about 3.2 mg/mL, about 3.3 mg/mL, about 3.4 mg/mL, about 3.5 mg/mL, about 3.6 mg/mL, about 3.7 mg/mL, about 3.8 mg/mL, about 3.9 mg/mL, about 4.0 mg/mL, about 4.1 mg/mL, about 4.2 mg/mL, about 4.3 mg/mL, about 4.4 mg/mL, about 4.5 mg/mL, about 4.6 mg/mL, about 4.7 mg/mL, about 4.8 mg/mL, about 4.9 mg/mL, about 5.0 mg/mL, about 5.1 mg/mL, about 5.2 mg/mL, about 5.3 mg/mL, about 5.4 mg/mL, about 5.5 mg/mL, about 5.6 mg/mL, about 5.7 mg/mL, about 5.8 mg/mL, about 5.9 mg/mL, about 6.0 mg/mL, about 6.1 mg/mL, about 6.2 mg/mL, about 6.3 mg/mL, about 6.4 mg/mL, about 6.5 mg/mL, about 6.6 mg/mL, about 6.7 mg/mL, about 6.8 mg/mL, about 6.9 mg/mL, about 7.0 mg/mL, about 7.1 mg/mL, about 7.2 mg/mL, about 7.3 mg/mL, about 7.4 mg/mL, about 7.5 mg/mL, about 7.6 mg/mL, about 7.7 mg/mL, about 7.8 mg/mL, about 7.9 mg/mL, about 8.0 mg/mL, about 8.1 mg/mL, about 8.2 mg/mL, about 8.3 mg/mL, about 8.4 mg/mL, about 8.5 mg/mL, about 8.6 mg/mL, about 8.7 mg/mL, about 8.8 mg/mL, about 8.9 mg/mL, about 9.0 mg/mL, about 9.1 mg/mL, about 9.2 mg/mL, about 9.3 mg/mL, about 9.4 mg/mL, about 9.5 mg/mL, about 9.6 mg/mL, about 9.7 mg/mL, about 9.8 mg/mL, about 9.9 mg/mL, and about 10.0 mg/mL.

In some embodiments, the particle heaters are present in the composition in an amount ranging from about 0.5 wt. % to about 25 wt. % by the total weight of the composition. In some embodiments, the particle is present in an amount ranging from about 1.0 wt. % to about 20.0 wt. % by the total of the composition. In some embodiments, the particle heater is present in an amount ranging from about 5.0 wt. % to about 20.0 wt. % by the total of the composition. In some embodiments, the particle is present in an amount ranging from about 5.0 wt. % to about 15.0 wt. % by the total of the composition. In some embodiments, the particle is present in an amount ranging from about 10.0 wt. % to about 15.0 wt. % by the total of the composition. In some embodiments, the material responsive to the particle is present in an amount selected from the group consisting of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, about 20.0 wt. %, about 20.5 wt. %, about 21.0 wt. %, about 21.5 wt. %, about 22.0 wt. %, about 22.5 wt. %, about 23.0 wt. %, about 23.5 wt. %, about 24.0 wt. %, about 24.5 wt. %, and about 25.0 wt. % by the total weight of the composition. In some embodiments, the particle is present in an amount selected from the group consisting of about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, and about 15.0 wt. % by the total weight of the composition. In some embodiments, the particle is present in an amount selected from the group consisting of about 1.0 wt. %, 2.0 wt. %, 3.0 wt. %, 4.0 wt. %, about 5.0 wt. %, about 10.0 wt. %, and about 15.0 wt. %.

Temperatures greater than 50° C. can induce tissue fusion. (“tissue welding”). This is believed to be induced by the denaturation of the proteins and the subsequent entanglement of adjacent protein chains. In some embodiments, the induced hyperthermia in the curable bioadhesive is mild hyperthermia at a temperature ranging from about 38.0° C. to about 41.0° C. In some embodiments, the induced hyperthermia is moderate hyperthermia at a temperature ranging from about 41.1° C. to about 45.0° C. In some embodiments, the induced hyperthermia is profound hyperthermia at a temperature ranging from about 45.1° C. to about 52.0° C. In some embodiments, the temperature realized at the tissue site by particles is higher than 50° C. In some embodiments, the temperature realized at the tissue site is in a range from about 40° C. to about 50° C. In some embodiments, the temperature realized at the tissue site is in a range from about 50° C. to about 75° C.

### 4. In Situ Curable Biomedical Adhesive Formulations

### (i) In Situ Curable Tissue Adhesive

In some embodiments, this disclosure provides an in situ curable tissue adhesive containing a polymerizable and/or crosslinkable precursor, a thermal initiator, and a heat delivery medium/composition/particle described herein. The heat delivery medium/composition/particle may include a carrier and a material interacting with an exogenous source. The carrier and the material are those described herein. In some embodiments, the exogenous source is selected from the group consisting of an electromagnetic radiation, an electrical field, a microwave, a radio wave, ultrasonic radiation, a magnetic field, and combinations thereof. In some embodiments, the exogenous source is a laser light. In some embodiments, the exogenous source is a light emitting device including LED. In some embodiments, the exogenous source is a LED light. In some embodiments, wherein the material is a photothermal conversion agent (NIR light absorbing agent) as described above.

The NIR light absorbing agent absorbs the photonic energy from the laser irradiation and converts the absorbed photonic energy to heat, wherein the heat induces localized hyperthermia in the curable tissue adhesive composition, wherein the localized hyperthermia causes the degradation of the thermal initiator to generate radicals that promotes the polymerization of the polymerizable precursor. The in situ curing of the curable tissue adhesive composition is via free-radical polymerization of a polymerizable precursor initiated by radicals generated by the thermal initiator.

In some embodiments, the carrier and the material form a particle, wherein the particle is a nanoparticle, a microparticle, or mixtures thereof.

In some embodiments, this disclosure provides a method for wound repair comprising the steps: (1) applying to the wound site an in situ curable tissue adhesive composition as described above, and (2) exposing the in situ curable tissue adhesive to a laser light as described above.

The exogenous source may be applied as described generally previously in this disclosure. In some embodiments, the temperature in the in situ curable adhesive is increased to a value ranging from about 40° C. to about 90° C. In some embodiments, the temperature in the in situ curable adhesive and/or tissue site is increased to a value selected from the group consisting of about 40° C., about 45° C., about 50° C., about 55° C., about 60° C., about 65° C., about 70° C., about 75° C., about 80° C., and about 90° C.

In some embodiments, the in situ curable tissue adhesive has a curing time of less than 300 seconds, 250 seconds, 200 seconds, 150 seconds, 120 seconds, 60 second, 30 seconds, 20 seconds, 10 seconds, 5 seconds, or 1 second.

In some embodiments, the material is a light absorbing material as describe previously in the disclosure. The material may be an organic dye or an inorganic substance. In some embodiments, the material is a plasmonic absorber.

In some embodiments, the IR absorbing material is admixed within the carrier to form a homogeneous dispersion or a solid solution.

In some embodiments, the in situ curable tissue adhesive is a liquid formulation. In some embodiments, the NIR light absorbing agent in a liquid formulation of the in situ curable tissue adhesive is at a concentration ranging from about 1 mg/mL to about 5 mg/mL. In some embodiments, the NIR light absorbing agent in a liquid formulation of the in situ curable tissue adhesive is at a concentration selected from the group consisting of about 1.0 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, about 3.0 mg/mL, about 3.1 mg/mL, about 3.2 mg/mL, about 3.3 mg/mL, about 3.4 mg/mL, about 3.5 mg/mL, about 3.6 mg/mL, about 3.7 mg/mL, about 3.8 mg/mL, about 3.9 mg/mL, about 4.0 mg/mL, about 4.1 mg/mL, about 4.2 mg/mL, about 4.3 mg/mL, about 4.4 mg/mL, about 4.5 mg/mL, about 4.6 mg/mL, about 4.7 mg/mL, about 4.8 mg/mL, about 4.9 mg/mL, and about 5.0 mg/mL.

In some embodiments, the polymerizable precursors are monomers such as are commonly used in tissue adhesive compositions. As such, the polymerizable precursorsn may be selected from the group consisting of butyl cyanoacrylate, octylcyanoacrylate, dopamine methacrylamide (DMA), catechol acetonide glycidyl ether, a mixture of DMA and oligomeric ethylene glycol, mixture of DMA and monoacryloxyethyl phosphate, a mixture of DMA and methoxyethyl acrylate, and combinations thereof. In some embodiments, the in situ curable tissue adhesive comprises octyl cyanoacrylate (OCA) and polyethylene glycol (PEG). In some embodiments, the in situ curable tissue adhesive comprises a combination of PEG, OCA, and methyl cyanoacrylate (MCA), wherein the volume ratio of OCA to MCA is of 50:50, wherein the volume ratio of OCA to PEG is of 85:15.

In some embodiments, the polymerizable precursor is a prepolymer selected from the group consisting of polyethylene glycol diacrylate, gelatin modified with acryloyl groups, collagen modified with acryloyl groups, alginate modified with acryloyl groups, dextran modified with acryloyl groups, hyaluronic acid modified with acryloyl groups, and combinations thereof.

In some embodiments, the polymerizable monomer composition comprises monomer selected from the group consisting of C4-C10 alkyl methacrylate, C4-C10 alkyl acrylate, alkyl cyanoacrylate, methyl methacrylate, ethyl methacrylate, styrene methacrylate, 2-vinyl pyrrolidinone, propyl methacrylate, hexyl methacrylate, acrylic acid, vinyl acetate, vinyl acetic acid, mono-2-(methacryloyloxy)ethyl succinate, methacrylic acid, (polyethylene glycol) methacrylate, ethylene glycol dimethacrylate (EGDMA), 1,3-butylene glycol dimethacrylate (BGDMA), 1,4-butane diol diacrylate (BDDA), 1,6-hexane diol diacrylate (HDDA), isooctyl acrylate (2-EHA), tri(propylene glycol) diacrylate, hexanediol dimethacrylate (HDDMA), l-carboxy-N-methyl-N-di(2-methacryloyloxy-ethyl) methanaminium inner salt (CBMX), bis(meth)acrylamides, neopentylglycol diacrylate (NPGDA), trimethylolpropane ethoxylate triacrylate (TMPTA), acrylic acid 2-(2-acryloyloxy-ethoxymethyl)-2-acryloyloxymethyl-butyl ester (EO-TMPTA), acrylonitrile, methacrylonitrile, vinylidene cyanide, vinyl acetate, vinyl propionate, styrene, alpha-methylstyrene, maleic anhydride, and combinations thereof.

In some embodiments, the polymerizable monomer composition comprises cyanoacrylate monomer as a component for cyanoacrylate tissue adhesives. Cyanoacrylate tissue adhesives are liquid monomers and can polymerize quickly on contact with tissue surface creating a thin, flexible film. This polymer film creates a mechanical barrier, which maintains a natural healing environment. In some embodiments, the alkyl cyanoacrylate is selected from the group consisting of methoxyisopropylcyanoacrylate (MCA), octylcyanoacrylate (OCA), chloroethyl cyanoacrylate, n-propyl cyanoacrylate, i-propyl cyanoacrylate, allyl cyanoacrylate, propargyl cyanoacrylate, n-butyl cyanoacrylate, i-butyl cyanoacrylate, n-pentyl cyanoacrylate, n-hexyl cyanoacrylate, cyclohexyl cyanoacrylate, phenyl cyanoacrylate, tetrahydrofurfuryl cyanoacrylate, heptyl cyanoacrylate, 2-ethylhexyl cyanoacrylate, n-octyl cyanoacrylate, n-nonyl cyanoacrylate, oxononyl cyanoacrylate, n-decyl cyanoacrylate, n-dodecyl cyanoacrylate, 2-ethoxyethyl cyanoacrylate, 3-methoxybutyl cyanoacrylate, 2-ethoxy-2-ethoxyethyl cyanoacrylate, butoxy-ethoxy-ethyl cyanoacrylate, 2,2,2-trifluoroethyl cyanoacrylate, hexafluoroisopropyl cyanoacrylate, and combinations thereof.

In some embodiments, the polymerizable monomer composition comprises a C1-C16 alkyl methacrylate, C1-C16 alkyl acrylate, C1-C16 acrylamide, and combinations thereof. In some embodiments, the polymerizable monomer composition comprises hydrophilic monomer selected from the group consisting of hydroxymethacrylate (HEMA), hydroxyethyl methacrylate, 2-hydroxypropyl methacrylate, 2-hydroxyethyl acrylate, 2-hydroxypropyl acrylate, 3-hydroxypropyl acrylate, diethylene glycol monomethacrylate, hydroxyacrylate, glycerol dimethacrylate, glycol monomethacrylate, polyethylene glycol monomethacrylate, propylene glycol monomethacrylate, oligopropylene glycol monomethacrylate, hydroxypropyl methacrylate, polypropylene glycol monomethacrylate, hydroxyethyl-methacrylate, glycerol diacrylate, 2-tert-butylaminoethyl methacrylate, the reaction product of methacrylic acid and propylene oxide, 2-tert-butylaminoethyl methacrylate, polyethylene glycol 400 dimethacrylate, polyethylene glycol 600 dimethacrylate, polyethylene glycol 400 diacrylate, PEG 1,000 dimethacrylate, polypropylene glycol dimethacrylate, triethylene glycol di(meth)acrylate, dimethacrylates, diacrylates, monomethacrylates, monoacrylates, dipropylene glycol monomethacrylate, dipropylene glycol monoacrylate, acrylamide, methacrylamide, methylolacrylamide, methylolmethacrylamide, diacetone acrylamide, N-methylacrylamide, N-ethylacrylamide, N-hydroxyethyl acrylamide, N,N-disubstituted acrylamides, N,N-dimethylacrylamide, N,N-diethylacrylamide, N-ethylmethylacrylamide, N,N-dimethylolacrylamide, N-pyrrolidone, N-vinyl piperidone, N-acryloylpyrriolidone, N-acryloylpiperidine, N-acryloylmorpholene, N-vinyl pyrrolidinone, N-vinyl caprolactam, N-vinyl acetate, and combinations thereof.

In some embodiments, the polymerizable and/or cross-linkable precursor comprises one or more polymerizable prepolymer selected from the group consisting of polyethylene glycol 400 dimethacrylate, polyethylene glycol 600 dimethacrylate, polyethylene glycol 400 diacrylate; PEG 1,000 dimethacrylate, polypropylene glycol dimethacrylate, polyethylene glycol diacrylate, acrylated gelatin, collagen acrylate, acrylated alginate, and combinations thereof.

In some embodiments, the polymerizable monomer composition containing radical polymerization monomers may further include a radical polymerization initiator, and may optionally contain water, a water-miscible solvent, one or more reactive diluents, and/or an inert solvent. In some embodiments, the radical polymerization initiator is selected from the group consisting of 2,2′-azobis [2-(2-imidazolin-2-yl) propane] dihydrochloride, 2,2′-azobis [2-(2-imidazolin-2-yl) propane] disulfate dihydrate, 2,2′-azobis (2-methyl propionic amidine) dihydrochloride, 4,4′-azobis (4-cyano valeric acid), camphorquinone-10-sulfonic acid and its salts, camphorquinone 3-oximes, anti-(1R)-(+)-camphorquinone 3-oxime, anti-(1S)-(−)-camphorquinone 3-oxime, the addition reaction product of anti-(1R)-(+)-camphorquinone 3-oxime, anti-(1S)-(−)-camphorquinone 3-oxime with an organic anhydride, a dianhydride, a camphorquinone, a peroxide, and combinations thereof.

In some embodiments, the polymerizable and/or cross-linkable precursor composition comprises polymerizable prepolymer selected from the group consisting of polyethylene glycol diacrylate, acrylated gelatin, collagen acrylate, acrylated alginate, and combinations thereof.

In some embodiments, the polymerizable and/or cross-linkable precursor composition comprises cross-linkable prepolymer. In some embodiments, the cross-linkable prepolymer composition comprises in situ curable hydrogel adhesive precursors. In some embodiments, the cross-linkable prepolymer for hydrogel adhesive having reactive groups selected from vinyl group (—CH═CH₂), ethynyl group (—C≡C—), vinyl dimethyl sulfone group, hydroxyl group (—OH), thiol group (—SH), amine group (—NH₂), aldehyde group (—CHO), carboxylic acid group (—COOH), and combinations thereof. In some embodiments, the cross-linkable prepolymer may include natural proteins such as collagen, gelatin, fibrin, natural polysaccharides such as hyaluronic acid, alginate, pullulan, polyalkylene glycols, polypropylene oxide, poly(vinylamine), poly(ethyleneimine), poly(allylamine), poly(ethylene glycol-co-aspartic acid), poly(lysine-co-lactide), poly(cysteine-co-lactide), poly(2-aminoethylmethacrylate), polyhistidine, poly(guanidine), polylysine, polyornithine, polyarginine, poly(histidine)-co-poly(glutamic acid), copolymer of polyalanine-polylysine, poly(phenylalanine-co-glutamic acid)-polyalanine-polylysine, and starburst dendrimers, and combinations thereof.

In some embodiments, the cross-linkable prepolymer composition comprises lipid selected from the group consisting of phosphatidylethanolamine, phosphatidylserine, phosphatidylinositol, phosphatidyl glycerol, dilaurylphosphatidic acid, dipalmitoyl phosphatidyl glycerol, and combinations thereof.

In some embodiments, the cross-linkable prepolymer is selected from the group consisting of polysaccharides, polylactates, polyglycolates, polyols and proteins, and derivatives thereof.

In some embodiments, the cross-linkable prepolymer is in a partially crosslinked form in which individual molecules of the cross-linkable material are linked together through intermolecular covalent bonds. Such crosslinking can be achieved by standard techniques known in the art, for example by heat treatment and/or crosslinking agents. Depending on the nature of the cross-linkable material and/or the conditions employed to effect crosslinking, the degree of crosslinking between individual molecules can vary considerably.

In some embodiments, the in situ curable hydrogel adhesive precursors comprise of two silicone hydrogel adhesive precursors and a platinum catalyst, wherein one of the silicone hydrogel adhesive precursor has Si—H groups and the other silicone hydrogel adhesive precursor has complementary reactive Si-vinyl groups (Si—CH═CH₂).

In some embodiments, the cross-linkable prepolymer comprises cross-linkable polysaccharides. In some embodiments, the cross-linkable polysaccharides may include hyaluronic acid, chitosan, alginic acid, sodium alginate, or carrageenan.

In some embodiments, the bioadhesive comprises cross-linked polymer networks resulting from the reaction of the reactive groups attached to the cross-linkable prepolymer with a cross-linking reagent. In some embodiments, the degree of cross-linking can be tuned by controlling the weight ratio of the cross-linking reagent to the polymerizable prepolymer having cross-linkable reactive groups in the cross-linking reaction. In some embodiments, the crosslinking reagent is a biocompatible compound selected from the group consisting of genepin, tannin, catechol derivatives, 3,4-dihydroxyphenylalanine (DOPA), dopamine and its derivatives, and combinations thereof.

In some embodiments, the base polymer for the polymerizable prepolymers is selected from the group consisting of polyacrylic acids, polyethylene glycols, modified polyethylene glycols, thrombin, collagen, gelatin, fibrin, fibrin glue compositions, gelatin-resorcinol-formaldehyde-glutaraldehyde (GRFG), albumin, glycosaminoglycans, poly(N-isopropylacrylamide), alginates, chitosan, gelatin, polylactide, polyglycolide, polycaprolactone, poly(lactide-co-glycolide) acid (PLGA), poly(lactide-co-ε-caprolactone) (PLCL), and combinations thereof.

In some embodiments, the polymerizable prepolymers comprise the precursors for hydrogel adhesives selected from the group consisting of polyethylene glycol diacrylate, acrylated gelatin, collagen acrylate, acrylated alginate, and combinations thereof.

In some embodiments, the in situ curable hydrogel adhesive precursors comprise of two silicone hydrogel adhesive precursors and a platinum catalyst, wherein one of the silicone hydrogel adhesive precursor has Si—H groups and the other silicone hydrogel adhesive precursor has complementary reactive Si-vinyl groups (Si—CH═CH₂).

In some embodiments, the polymerizable precursor in the liquid formulation is at a concentration ranges from about 0.15 g/mL to about 0.3 g/mL. In some embodiments, the polymerizable precursor is at a concentration selected from the group consisting of about 0.15 g/mL, about 0.16 g/mL, about 0.17 g/mL, about 0.18 g/mL, about 0.19 g/mL, about 0.20 g/mL, about 0.21 g/mL, about 0.22 g/mL, about 0.23 g/mL, about 0.24 g/mL, about 0.25 g/mL, about 0.26 g/mL, about 0.27 g/mL, about 0.28 g/mL, about 0.29 g/mL, and about 0.30 g/mL.

In some embodiments, the thermal initiator is a free radical or cationic inititor such as commonly used in the art. The thermal initiator is selected from the group consisting of 2,2′-azobis [2-(2-imidazolin-2-yl) propane] dihydrochloride, 2,2′-azobis [2-(2-imidazolin-2-yl) propane] disulfate dihydrate, 2,2′-azobis (2-methyl propionic amidine) dihydrochloride, 4,4′-azobis (4-cyano valeric acid), and combinations thereof.

In some embodiments, the polymerization initiator that helps to start the free radical polymerization of the polymerizable monomer via a free radical polymerization reaction between the monomers. The in situ curing of the curable bioadhesive take places after mixing the solid and liquid phases if the formulation exits as a two parts formulation. The kinetics of the free-radical polymerization reaction is regulated by the concentrations and mobility of the initiator and the accelerator in the composition.

In some embodiments, the polymerization initiator is selected from the group consisting of benzoyl oxide (BPO), tri-n-butyl borane, 2-5-dimethylhexane-2-5-dihydroperoxide, the remotely-triggered particle, 2,2′-azobis [2-(2-imidazolin-2-yl) propane] dihydrochloride, 2,2′-azobis [2-(2-imidazolin-2-yl) propane] disulfate dihydrate, 2,2′-azobis (2-methyl propionic amidine) dihydrochloride, 4,4′-azobis (4-cyano valeric acid), camphorquinone-10-sulfonic acid and its salts, camphorquinone 3-oximes, anti-(1R)-(+)-camphorquinone 3-oxime, anti-(1S)-(−)-camphorquinone 3-oxime, the addition reaction product of anti-(1R)-(+)-camphorquinone 3-oxime, anti-(1S)-(−)-camphorquinone 3-oxime with an organic anhydride, a dianhydride, a camphorquinone, a peroxide, a mixture of horseradish peroxidase and hydrogen peroxide, and combinations thereof.

In some embodiments, the polymerization initiator is selected from the group consisting of 2,2′-azobis [2-(2-imidazolin-2-yl) propane] dihydrochloride, 2,2′-azobis [2-(2-imidazolin-2-yl) propane] disulfate dihydrate, 2,2′-azobis (2-methyl propionic amidine) dihydrochloride, 4,4′-azobis (4-cyano valeric acid), and combinations thereof.

In some embodiments, the polymerization initiator is BPO. In some embodiments, the polymerization initiator comprises BPO and the remotely triggered particles. In some embodiments, the polymerization initiator comprises remotely triggered particle and hydrogen peroxide.

The term “Fenton chemistry” as used herein, generally refers to the nonenzymatic reaction of Fe²⁺ with H₂O₂. Fe²⁺ is oxidized by hydrogen peroxide to Fe³⁺, forming OH. and OH⁻ in the reaction. Fe³⁺ is then reduced back to Fe²⁺ by another molecule of H₂O₂, forming a hydroperoxyl radical .OOH and a proton H⁺. The net effect is a disproportionation of hydrogen peroxide to create two different oxygen-radical species, with water as a byproduct. Iron and hydrogen peroxide are capable of oxidizing a wide range of substrates and causing biological damage. The Fenton reaction is a reaction of importance in the oxidative stress in blood cells and various tissues.

In some embodiments, the polymerization initiator is in an amount ranging from about 0.1 wt. % to about 3.0 wt. % by the total weight of the in situ curable bioadhesive. In some embodiments, the polymerization initiator is in an amount ranging from about 0.75 wt. % to about 2.6 wt. % by the total weight of the in situ curable bioadhesive. In some embodiments, the polymerization initiator is in an amount ranging from about 0.8 wt. % to about 1.4 wt. % by the total weight of the in situ curable bioadhesive. In some embodiments, the polymerization initiator is in a weight percent by the total weight of the in situ curable bioadhesive selected from the group consisting of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, and about 3.0 wt. %.

In some embodiments, the thermal initiator in the liquid formulation is at a concentration ranging from about 1.0 mg/mL to about 20.0 mg/mL. In some embodiments, the thermal initiator is at a concentration selected from the group consisting of about 1.0 mg/mL, about 2.0 mg/mL, about 3.0 mg/mL, about 4.0 mg/mL, about 5.0 mg/mL, about 6.0 mg/mL, about 7.0 mg/mL, about 8.0 mg/mL, about 9.0 mg/mL, about 10.0 mg/mL, about 11.0 mg/mL, about 12.0 mg/mL, about 13.0 mg/mL, about 14.0 mg/mL, about 15.0 mg/mL, about 16.0 mg/mL, about 17.0 mg/mL, about 18.0 mg/mL, about 19.0 mg/mL, and about 20.0 mg/mL.

In some embodiments, the radicals for initiating the polymerization curing process is generated by sono/photodynamic process, for example, reactive oxygen species include hydrogen oxide radical species can be generated by irradiating the ICG particles with laser or exposure to ultrasonic radiation.

In some embodiments, the in situ curable bioadhesive further comprises a crosslinking agent.

In some embodiments, the cross-linking reagent for cross-linking hydroxyl groups (—OH), thiol groups (—SH), or amine groups (—NH₂) may include dithiobis(succinimidyl) propionate (Lomant's reagent), cystamine bisacrylamide, bisacryloyloxyethyl disulfide, N,N′-(ethane-1,2-diyl)diacrylamide, N,N′-(2-hydroxypropane-1,3-diyl)diacrylamide, polyisocyanate, polyisothiocyanate, dimethyl adipimidate, dimethyl pimelimidate, dimethyl suberimidate, dimethyl 3,3′-dithiobispropionimidate, glutaraldehyde, glyoxal, glyoxal-trimer dihydrate, dimethyl suberimidate, dimethyl 3,3′-dithiobispropionimidate glutaraldehyde, epoxides, bis-oxiranes, p-azidobenzoyl hydrazide, N-α-maleimidoacetoxy succinimide ester, p-azidophenyl glyoxal monohydrate, bis-((beta)-(4-azidosalicylamido)ethyl)disulfide, succinimidyl iodoacetate, succinimidyl 3-(bromoacetamido)propionate, 4-(iodoacetyl)aminobenzoate, N-α-maleimidoacet oxysuccinimide ester, N-β-maleimidopropyl oxysuccinimide ester, N-γ-maleimidobutyryl oxysuccinimide ester, m-maleimidobenzoyl-N-hydroxysuccinimide ester, succinimidyl 4-(N-maleimidomethyl)cyclohexane-1-carboxylate, N-ε-malemidocaproyl oxysuccinimide ester, succinimidyl 4-(p-maleimidophenyl)butyrate, succinimidyl 6-β-maleimidopropionamido)hexanoate, succinimidyl 3-(2-pyridyldithio)propionate (SPDP), PEG4-SPDP, PEG12-SPDP, disuccinimidyl tartrate, 4-succinimidyloxycarbonyl-α-methyl-α-(2-pyridyldithio)toluene, disuccinimidyl glutarate, ethylene glycol bis(succinimidylsuccinate), bis-(sulfosuccinimidyl) (ethylene glycol) bis(succinimidylsuccinate), bis-sulfosuccinimidyl suberate, disuccinimidyl-suberate, tris-succinimidyl aminotriacetate, diacylchlorides, or polyphenolic compounds (e.g. tannic acid or tannin, dopamine and its derivatives) as cross-linker for cross-linking protein such as collagen, gelatin etc.

In some embodiments, the cross-linking reagent for cross-linking hydroxyl groups (—OH), thiol groups (—SH), or amine groups (—NH₂) may include carboxyl group terminated polyethylene glycol having 2-8 branching arms (used with carboxylic acid activation agent N-hydroxysuccinimide esters (NHS) and/or (1-Ethyl-3-(3-dimethylaminopropyl)-carbodiimide (EDC)). For example, 4-arm PEG carboxyl (pentaerythritol core), 6-arm PEG carboxyl (hexaglycerin core), 8-arm PEG carboxyl (tripentaerythritol core). In some embodiments, the cross-linking agent for cross-linking hydroxyl groups (—OH), thiol groups (—SH), or amine groups (—NH₂) may include bis-succinimide ester terminated polyethylene glycol or star shaped succinimide ester terminated polyethylene glycol having 3-8 branching arms, for example, 4-arm PEG succinimidyl (pentaerythritol core) or 6-arm PEG succinimidyl (hexaglycerin core). In some embodiments, the succinimide ester, or carboxyl group terminated polyethylene glycol type cross-linking agent may have a number average molecular weight ranging from about 150 Daltons (Da) to about 10 KDa. In some embodiments, the succinimide ester, or carboxyl group terminated polyethylene glycol type cross-linking agent may have a number average molecular weight ranging from about 1 KDa to about 10 KDa. In some embodiments, the succinimide ester, or carboxyl group terminated polyethylene glycol type cross-linking agent may have a number average molecular weight ranging from about 1 KDa to about 5 KDa. In some embodiments, the succinimide ester, or carboxyl group terminated polyethylene glycol type cross-linking agent may have a number average molecular weight ranging from about 150 Da to about 1 KDa. In some embodiments, the succinimide ester, or carboxyl group terminated polyethylene glycol type cross-linking agent may have a number average molecular weight ranging from about 150 Da to about 750 Da.

In some embodiments, the cross-linking agent for cross-linking reactive aldehyde groups, vinyl methyl sulfone groups, or carboxylic acid groups (activation with N-hydroxysuccinimide esters (NHS) or (1-Ethyl-3-(3-dimethylaminopropyl)-carbodiimide (EDC)) may include polyamine compounds such as spermine, polyspermine, low molecular weight polyethylenimine (PEI), dilysine, liner or branched trilysine, tetralysine, pentalysine, hexylysine, heptalysine, octalysine, nonalysine, decalysine, undecalysine, dodecalysine, tridecalysine, tetradecalysine, pentadecalysine, or hyperbranched polylysines, polyols such as pentaerythritol, ethylene glycol, polyethylene glycol, glycerol, polyglycerol, sucrose, sorbitol etc.

In some embodiments, the cross-linking agent for cross-linking aldehyde groups, vinyl methyl sulfone groups, or carboxylic acid groups (activation with N-hydroxysuccinimide esters (NHS) or (1-Ethyl-3-(3-dimethylaminopropyl)-carbodiimide (EDC)) may include amine terminated polyethylene glycols having 2-8 branching arms, for example, 4-arm PEG amine (pentaerythritol core), 6-arm PEG amine (hexaglycerin core), 8-arm PEG amine (tripentaerythritol core). In some embodiments, the amine terminated polyethylene glycol type cross-linking agents may have a number average molecular weight ranging from 150 Da to 10 KDa. In some embodiments, the amine terminated polyethylene glycol type cross-linking agents may have a number average molecular weight ranging from 1 KDa to 10 KDa. In some embodiments, the amine terminated polyethylene glycol type cross-linking agents may have a number average molecular weight ranging from 1 KDa to 5 KDa. In some embodiments, the amine terminated polyethylene glycol type cross-linking agents may have a number average molecular weight ranging from 150 Da to 1 KDa. In some embodiments, the amine terminated polyethylene glycol type cross-linking agent may have a number average molecular weight ranging from 150 Da to 750 Da.

In some embodiments, the in situ curable hydrogel adhesive further comprising a crosslinker selected from the group consisting of polyethylene glycol-2500 diacrylate, 8-arm PEG-2500 acrylate, 4-arm PEG-5000 acrylate, 6-arm PEG-2500-(NH₂)₆, genipin and FeCl3, thiolated pluronic F-127, dopamine or DOPA/H₂O₂, Dextran aldehyde, NHS/EDC, NHS/DCC, EDC, disuccinimidyl tartrate (DST), disuccinimidyl malate (DSM) and trisuccinimidyl citrate (TSC), 4-arm PEG-thiol, trilysine, collagen, glutaraldehyde, PEG-diacrylate, ethylene glycole dimethacrylate (EGDMA), triethylene glycol dimethacrylate (TEGDMA), poly(ethylene glycol) dimethacrylate (PEGDMA), poly(MMA-co-AA-co-allylmethacrylate), 1,3-butylene glycol dimethacrylate (BGDMA), 1,4-butane diol diacrylate (BDDA), 1,6-hexane diol diacrylate (HDDA), hexanediol dimethacrylate (HDDMA), 1-carboxy-N-methyl-N-di(2-methacryloyloxy-ethyl) methanaminium inner salt (CBMX), bis(meth)acrylamides, neopentylglycol diacrylate (NPGDA), trimethylolpropane triacrylate (TMPTA), and combinations therof.

In some embodiments, the crosslinking agent has a weight percent ranging from about 1.0 wt. % to about 10 wt. % by the total weight of the in situ curable bioadhesive. In some embodiments, the crosslinking agent has a weight percent of about 10 wt. % by the total weight of the in situ curable bioadhesive. In some embodiments, the crosslinking agent has a weight percent by the total weight of the in situ curable bioadhesive selected from the group consisting of about 1.0 wt. %, about 1.1 wt. %, about 1.2 wt. %, about 1.3 wt. %, about 1.4. wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4. wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4. wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4. wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4. wt. %, about 5.5 wt. %, about 5.6 wt. %, about 5.7 wt. %, about 5.8 wt. %, about 5.9 wt. %, about 6.0 wt. %, about 6.1 wt. %, about 6.2 wt. %, about 6.3 wt. %, about 6.4. wt. %, about 6.5 wt. %, about 6.6 wt. %, about 6.7 wt. %, about 6.8 wt. %, about 6.9 wt. %, about 7.0 wt. %, about 7.1 wt. %, about 7.2 wt. %, about 7.3 wt. %, about 7.4. wt. %, about 7.5 wt. %, about 7.6 wt. %, about 7.7 wt. %, about 7.8 wt. %, about 7.9 wt. %, about 8.0 wt. %, about 8.1 wt. %, about 8.2 wt. %, about 8.3 wt. %, about 8.4. wt. %, about 8.5 wt. %, about 8.6 wt. %, about 8.7 wt. %, about 8.8 wt. %, about 8.9 wt. %, about 9.0 wt. %, about 9.1 wt. %, about 9.2 wt. %, about 9.3 wt. %, about 9.4. wt. %, about 9.5 wt. %, about 9.6 wt. %, about 9.7 wt. %, about 9.8 wt. %, about 9.9 wt. %, and about 10.0 wt. %.

In some embodiments, the weight ratio of the polymerizable prepolymer to the crosslinker reagent ranges from 20:1 to 1:1. In some embodiments, the weight ratio of the polymerizable prepolymer to the crosslinker reagent ranges from 10:1 to 1:1. In some embodiments, the weight ratio of the polymerizable prepolymer to the crosslinker reagent ranges from 5:1 to 1:1. In some embodiments, the weight ratio of the polymerizable prepolymer to the crosslinker reagent is 10:1. In some embodiments, the weight ratio of the polymerizable prepolymer to the crosslinker reagent is 9:1. In some embodiments, the weight ratio of the polymerizable prepolymer to the crosslinker reagent is 5:1. In some embodiments, the weight ratio of the polymerizable prepolymer to the crosslinker reagent is 4:1.

In some embodiments, the in situ curable tissue adhesive further comprises a reinforcement filler such as commonly used in the art. The reinforcement filler may be selected from the group consisting of powders of high density polyethylene having a median particle size of about 50 μm or less, powders of PMMA having a median particle size of 50-60 μm, polyethylene (PE) fiber, ultra-high-strength PE, UHMWPE grafted with MMA, ultra-high-strength PE grafted with MMA, beads of rubber-toughened PMMA powder having a PMMA outer shell and an inner shell made of crosslinked butyl methacrylate-styrene copolymer, beads of poly(isobutylene), beads of acrynitrile-butadiene-styrene, beads of poly(ε-caprolactone), particles of polybutylmethacrylate (PBMA), PCL-toughened PMMA beads, polyethylene terephtahalate fiber, silanated HA particle, sintered HA particle, silane-treated fluorohydroxyapatite particle, Ca-hydroxyapatite, particles of PMAA, particles of PMETA-PMMA, particle of PEMA, particle of PEMA-n-BMA, ultra-high molecular wright polyethylene (UHMWPE), chitosan nanoparticles and combinations thereof. In some embodiments, the reinforcement filler is 50-60 μm PMMA particles.

In some embodiments, the curable tissue adhesive may further comprise one or more additives to improve the performance of the hydrogel adhesive including adhesion, tackiness, and to change the water content, water uptake, and moisture vapor transmission. The various additives include but are not limited to glycerol, polyethylene glycol, polypropyl glycol, polybutylene glycol, polyacrylic acid, celluloses, calcium alginate, sucrose, lactose, and fructose, sorbitol, mannitol, zylitol, dextrans, hyaluronic acid, polyacrylamidopropyltrimethyl ammonium chloride, calcium chloride, APOSS (Octaammonium-POSS (polyhedral oligomeric silsesquioxane)), and poly(2-acrylamido-2-methylpropane sulfonic acid).

In some embodiments, the in situ curable tissue adhesive may further include a bioactive agent that improves wound healing. In some embodiments, the active agent is selected from antimicrobial agents, wound healing factors, and combinations thereof.

In some embodiments, the antimicrobial agent is selected from the group consisting of silver nanoparticles, silver chloride, chitosan, chlorhexidene acetate, chlorhexidene gluconate, chlorhexidine hydrochloride, chlorhexidine sulfate, polymyxin, tetracycline, tobramycin, gentamicin, rifampician, bacitracin, neomycin, chloramphenical, oxolinic acid, norfloxacin, nalidix acid, pefloxacin, enoxacin, ciprofloxacin, ampicillin, amoxicillin, piracil, cephalosporins, vancomycin, bismuth tribromophenate, and combinations thereof.

In some embodiments, the wound healing factor is selected from the group consisting of metalloprotease inhibitors, curcumin, proteins and peptides such as growth factor-β (TGF-β), epidermal growth factor (EGF), insulin-like growth factor-1, platelet derived growth factors, and combinations thereof.

### (ii) In Situ Curable Hydrogel Adhesive

Tissue adhesives can simplify surgical procedures and minimize trauma. However, commercially available adhesives are limited by their slow degradation rate, toxic contents and poor adhesive strength. This disclosure provides an in situ curable hydrogel adhesive.

Marine mussels secrete protein-based adhesives, e.g. mussel foot proteins, containing a large abundance of a unique catecholic amino acid in their protein sequences. The catecholic amino acid, 3,4-dihydroxyphenylalanine (DOPA) is modified from tyrosine through post-translational hydroxylation. The catechol side chain of DOPA has the ability to form various types of chemical interactions and crosslinking, which imparts mussel foot proteins with the ability to solidify in situ. Catechol offers robust and durable adhesion to various substrate surfaces. The mussel foot proteins are known to cure rapidly to form an adhesive with high interfacial binding strength, durability and toughness.

Catechol is a unique and versatile adhesive molecule capable of binding to both inorganic and organic surfaces through either reversible or covalent bonds. Catechol forms strong, reversible bonds with metal oxides with bond strengths reaching 40% that of a covalent bond.

When catechol is oxidized to form the highly reactive quinone, it participates in intermolecular covalent cross-linking, leading to the rapid curing of catechol-containing adhesives and reacts with nucleophile (i.e., —NH₂, —SH) found on biological substrates, resulting in strong interfacial binding.

In an embodiment, this disclosure provides a curable hydrogel adhesive comprising two or more crosslinkable prepolymers, a crosslinking agent and the particle heaters as disclosed herein.

In an embodiment, this disclosure provides a curable hydrogel adhesive comprising a pair of crosslinkable prepolymers having reactive functional groups with complementary reactivity (e.g., —COOH, —NH₂), optionally a crosslinking agent (NHS/DEC), and the particle heaters as disclosed herein.

In some embodiments, this disclosure provides catechol-functionalized monomers for preparing in situ curable hydrogel adhesive. In some embodiments, catechol-functionalized monomer comprises dopamine methacrylamide (DMA), catechol acetonide glycidyl ether, a mixture of DMA and oligomeric ethylene glycol, a mixture of DMA and monoacryloxyethyl phosphate, and a mixture of DMA and methoxyethyl acrylate. DMA can be polymerized through heat-activated free radical polymerization.

In some embodiments, this disclosure provides an in situ curable hydrogel adhesive comprising catechol-functionalized polymers as precursor. Catechol and dopamine can be directly conjugated to polymers with functional groups such as —NH₂, —COOH, and —OH, through the formation of amide, urethane, and ester linkages. In some embodiments, the polymers suitable for catechol functionalization are selected from the group consisting of linear or branched polyethylene glycol (PEG), polycaprolactone (PCL), polypropylene oxide (PPO), block copolymer such as PEG-PCL, PPO-PCL, PPO-PEG, PEG-poly(methyl methacrylate), PEG-polymethacrylate, PEG-polyurethane, dextran, chitosan, hyaluronic acid, gelatin, alginate, and combinations thereof.

In some embodiments, the catechol-functionalized polymers are selected from the group consisting of dopamine-dextran, dopamine-chitosan, dopamine-hyaluronic acid, dopamine-gelatin, dopamine-alginate, poly(DOPA-lysine), poly(DOPA)-co-polypeptide, polystyrene catechol copolymers, catechol-functionalized polystyrene, and combinations thereof. Catechol-functionalized polystyrene is a product of eugenol acrylate or eugenol methacrylate with poly(styrene-co-(4-ethynyl styrene) via thiol-yne reaction. Polystyrene catechol copolymers are a product of copolymerization of 3,4-dihydroxystyrene, 4-vinylcatechol acetonide, 3-vinylcatechol acetonide, and styrene.

In some embodiments, the in situ curable hydrogel adhesives further comprises hyaluronic acid (HA) grafted with DOPA or silk fibroin protein grafted with DOPA.

In some embodiments, this disclosure provides an in situ hydrogel adhesive comprising DOPA/DOPA quinone derivative grafted hyaluronic acid (HA), carboxymethyl cellulose, alginate polymerized through a water-soluble linker such as PEG, or mixtures thereof. In some embodiments, the curable hydrogel adhesive comprises a thermal initiator and at least one catechol functionalize PEG block selected from the group consisting of PEG-glutaramide-D4 (D=DOPA, four arm PEG terminally functionalized by DOPA), hyaluronic acid-PEG-tyramine/dopamine (HA-PEG-TA/DA) (TA=tyramine, DA=dopamine), carboxymethyl cellulose-PEG-tyramine/dopamine (CMC-PEG-TA/DA) (CMC=carboxymethyl cellulose), alginate-PEG-tyramine/dopamine (ALG-PEG-TA/DA) (ALG=alginate), and combinations mixtures thereof. In the presence of horseradish peroxidase (HRP) and H₂O₂, these modified polymers may be converted into an in situ curable hydrogel adhesive having excellent tissue adhesion.

In some embodiments, the in situ curable hydrogel adhesive further comprises a reinforcement filler selected from the group consisting of powders of high density polyethylene having a median particle size of about 50 μm or less, powders of PMMA having a median particle size of 50-60 polyethylene (PE) fiber, ultra-high-strength PE, UHMWPE grafted with MMA, ultra-high-strength PE grafted with MMA, beads of rubber-toughened PMMA powder having a PMMA outer shell and an inner shell made of crosslinked butyl methacrylate-styrene copolymer, beads of poly(isobutylene), beads of acrynitrile-butadiene-styrene, beads of poly(ε-caprolactone), particles of polybutylmethacrylate (PBMA), PCL-toughened PMMA beads, polyethylene terephtahalate fiber, silanated HA particle, sintered HA particle, silane-treated fluorohydroxyapatite particle, Ca-hydroxyapatite, particles of PMAA, particles of PMETA-PMMA, particle of PEMA, particle of PEMA-n-BMA, ultra-high molecular wright polyethylene (UHMWPE), chitosan nanoparticles, and combinations thereof. In some embodiments, the reinforcement filler is 50-60 μm PMMA particles.

In some embodiments, the curable hydrogel adhesive may further comprise one or more additives to improve the performance of the hydrogel adhesive including adhesion, tackiness, and to change the water content, water uptake, and moisture vapor transmission. The various additives include but are not limited to glycerol, polyethylene glycol, polypropyl glycol, polybutylene glycol, polyacrylic acid, celluloses, calcium alginate, sucrose, lactose, and fructose, sorbitol, mannitol, zylitol, dextrans, hyaluronic acid, polyacrylamidopropyltrimethyl ammonium chloride, calcium chloride, APOSS (Octaammonium-POSS (polyhedral oligomeric silsesquioxane)), and poly(2-acrylamido-2-methylpropane sulfonic acid).

In some embodiments, the in situ curable hydrogel adhesive may further include bioactive agent that improve wound healing. In some embodiments, the active agent is selected from antimicrobial agents, wound healing factors, and combinations thereof.

In some embodiments, the antimicrobial agent is selected from the group consisting of silver nanoparticles, silve chloride, chitosan, chlorhexidene acetate, chlorhexidene gluconate, chlorhexidine hydrochloride, chlorhexidine sulfate, polymyxin, tetracycline, tobramycin, gentamicin, rifampician, bacitracin, neomycin, chloramphenical, oxolinic acid, norfloxacin, nalidix acid, pefloxacin, enoxacin, ciprofloxacin, ampicillin, amoxicillin, piracil, cephalosporins, vancomycin, bismuth tribromophenate, and combinations thereof.

In some embodiments, the wound healing factor is selected from the group consisting of metalloprotease inhibitors, curcumin, proteins and peptides such as growth factor-β (TGF-β), epidermal growth factor (EGF), insulin-like growth factor-1, platelet derived growth factors, and combinations thereof.

(iii) Additional Embodiments

Some additional bioadhesives are described in the Table 1 below.

TABLE 1
In Situ Curable Bioadhesives

| | Curable | | | | | | |
|---|---|---|---|---|---|---|---|
| | precursor | | | IR dye | | Crosslinking | Intended |
| Entry | composition | crosslinker | Material | physical form | Additive | mechanism | use |
| 1 | Butylcyano- | polyethylene | Epolight ™ | PMMA/BMA, | thermal labile | Free radical | Superficial |
| | acrylate | glycol-2500 | 1117, | PLGA/IR dye | polymerization | polymerization | tissue |
| | | diacrylate | Epolight ™ | microparticle, | inhibitor, | | |
| | | | 1175, ICG, IR | nanoparticle | thermal radical | | |
| | | | 193, IR 780, | | initiator | | |
| | | | or IR 820 | | | | |
| 2 | Octylcyano- | polyethylene | Epolight ™ | PMMA/BMA, | thermal labile | Free radical | Superficial |
| | acrylate | glycol-2500 | 1117, | PLGA/IR dye | polymerization | polymerization | tissue |
| | | diacrylate | Epolight ™ | microparticle, | inhibitor, | | |
| | | | 1175, ICG, IR | nanoparticle | thermal radical | | |
| | | | 193, IR 780, | | initiator | | |
| | | | or IR 820 | | | | |
| 3 | Butylcyano- | 8-arm PEG- | Epolight ™ | PMMA/BMA, | thermal labile | Free radical | Superficial |
| | acrylate | 2500 | 1117, Epolight ™ | PLGA/IR dye | polymerization | polymerization | tissue |
| | | acrylate | 1175, ICG, | microparticle, | inhibitor, | | |
| | | | IR 193, IR | nanoparticle | thermal radical | | |
| | | | 780, or IR 820 | | initiator | | |
| 4 | Octylcyano- | 4-arm PEG- | Epolight ™ | PMMA/BMA, | thermal labile | Free radical | Superficial |
| | acrylate | 5000 | 1117, Epolight ™ | PLGA/IR dye | polymerization | polymerization | tissue |
| | | acrylate | 1175, ICG, | microparticle, | inhibitor, | | |
| | | | IR 193, IR | nanoparticle | thermal radical | | |
| | | | 780, or IR 820 | | initiator | | |
| 5 | Aldehyde | | Epolight ™ | PMMA/BMA, | thermal radical | Free radical | Superficial |
| | modified | | 1117, Epolight ™ | PLGA/IR dye | initiator | polymerization | tissue |
| | gelatin | | 1175, ICG, | microparticle, | | | |
| | | | IR 193, IR | nanoparticle | | | |
| | | | 780, or IR 820 | | | | |
| 6 | chondroitin | 6-arm PEG- | Epolight ™ | PMMA/BMA, | — | Chemical | Deep |
| | sulfate | 2500-(NH₂)₆ | 1117, Epolight ™ | PLGA/IR dye | | conjugation | Tissue |
| | | | 1175, ICG, | microparticle, | | | |
| | | | IR 193, IR | nanoparticle | | | |
| | | | 780, or IR 820 | | | | |
| 7 | gelatin | genipin and | Epolight ™ | PMMA/BMA, | — | Chemical | Deep |
| | | FeCl₃ | 1117, Epolight ™ | PLGA/IR dye | | conjugation | Tissue |
| | | | 1175, ICG, | microparticle, | | | |
| | | | IR 193, IR | nanoparticle | | | |
| | | | 780, or IR 820 | | | | |
| 8 | chitosan | thiolated | Epolight ™ | PMMA/BMA, | — | Chemical | Deep |
| | | pluronic F- | 1117, Epolight ™ | PLGA/IR dye | | conjugation | Tissue |
| | | 127 | 1175, ICG, | microparticle, | | | |
| | | | IR 193, IR | nanoparticle | | | |
| | | | 780, or IR 820 | | | | |
| 9 | hyaluronic | thiolated | Epolight ™ | PMMA/BMA, | — | Chemical | Deep |
| | acid | pluronic F- | 1117, Epolight ™ | PLGA/IR dye | | conjugation | Tissue |
| | | 127 | 1175, ICG, | microparticle, | | | |
| | | | IR 193, IR | nanoparticle | | | |
| | | | 780, or IR 820 | | | | |
| 10 | polyethylene | dopamine or | Epolight ™ | PMMA/BMA, | — | Chemical | Deep |
| | glycol | DOPA/H₂O₂ | 1117, Epolight ™ | PLGA/IR dye | | conjugation | Tissue |
| | | | 1175, ICG, | microparticle, | | | |
| | | | IR 193, IR | nanoparticle | | | |
| | | | 780, or IR 820 | | | | |
| 11 | Dextran/ε- | Dextran | Epolight ™ | PMMA/BMA, | — | Chemical | Deep |
| | polylysine | aldehyde | 1117, Epolight ™ | PLGA/IR dye | | conjugation | Tissue |
| | | | 1175, ICG, | microparticle, | | | |
| | | | IR 193, IR | nanoparticle | | | |
| | | | 780, or IR 820 | | | | |
| 12 | Sodium | NHS/EDC | Epolight ™ | PMMA/BMA, | — | Chemical | Deep |
| | alginate/gelatin/ | | 1117, Epolight ™ | PLGA/IR dye | | conjugation | Tissue |
| | amino gelatin | | 1175, ICG, | microparticle, | | | |
| | | | IR 193, IR | nanoparticle | | | |
| | | | 780, or IR 820 | | | | |
| 13 | allyl 2- | poly(lactic | Epolight ™ | PMMA/BMA, | — | Chemical | Superficial |
| | cyanoacrylate | acid) | 1117, Epolight ™ | PLGA/IR dye | | conjugation | Tissue |
| | | | 1175, ICG, | microparticle, | | | |
| | | | IR 193, IR | nanoparticle | | | |
| | | | 780, or IR 820 | | | | |
| 14 | collagen-COO⁻/ | NHS/DCC | Epolight ™ | PMMA/BMA, | — | Chemical | Deep |
| | citric acid or | or EDC | 1117, Epolight ™ | PLGA/IR dye | | conjugation | Tissue |
| | tartaric acid, | | 1175, ICG, | microparticle, | | | |
| | malic acid | | IR 193, IR | nanoparticle | | | |
| | | | 780, or IR 820 | | | | |
| 15 | collagen-COO⁻/ | disuccinimidyl | Epolight ™ | PMMA/BMA, | — | Chemical | Deep |
| | | tartrate | 1117, Epolight ™ | PLGA/IR dye | | conjugation | Tissue |
| | | (DST), | 1175, ICG, | microparticle, | | | |
| | | disuccinimidyl | IR 193, IR | nanoparticle | | | |
| | | malate | 780, or IR 820 | | | | |
| | | (DSM) and | | | | | |
| | | trisuccinimidyl | | | | | |
| | | citrate | | | | | |
| | | (TSC) | | | | | |
| 16 | chitosan | | ICG | Free ICG dye | — | Chemical | Deep |
| | | | | | | conjugation | Tissue |
| 17 | 4-arm PEG- | 4-arm PEG- | Epolight ™ | Epolight ™ | — | Chemical | Deep |
| | glutaryl- | thiol | 1117 | 1117 | | conjugation | Tissue |
| | succinimidyl | | | microparticles | | | |
| | ester | | | | | | |
| 18 | 8-arm PEG- | trilysine | Epolight ™ | Epolight ™ | — | Chemical | Deep |
| | PEG-1200- | | 1117 | 1117 | | conjugation | Tissue |
| | glutaryl- | | | microparticles | | | |
| | succinimidyl | | | | | | |
| | ester | | | | | | |
| 19 | 4-arm PEG | (collagen | Epolight ™ | Epolight ™ | | Chemical | Deep |
| | 2500-aldehyde | from tissue) | 1117 | 1117 | | conjugation | Tissue |
| | | | | microparticles | | | |
| 20 | albumin | PEG- | Epolight ™ | Epolight ™ | | Chemical | Deep |
| | | diacrylate | 1117 | 1117 | | conjugation | Tissue |
| | | | | microparticles | | | |
| 21 | 8-arm PEG- | glutaraldehyde | Epolight ™ | Epolight ™ | | Chemical | Deep |
| | PEG-2500- | | 1117 | 1117 | | conjugation | Tissue |
| | NH₂ | | | microparticles | | | |

### (iv) Remotely-Triggered Curing of Curable Biomedical Adhesive

In an embodiment, this disclosure provides a method of remotely-triggered thermal-curing of a biomedical adhesives comprises the steps of: (1) mixing a polymerizable precursor with the particle heaters disclosed herein, (2) exposing mixture to an exogenous source for sufficient period of time, wherein the material absorbs the energy from the exogenous source and converts the energy to heat, wherein the heat induces localized hyperthermia in the curable bioadhesive, wherein the localized hyperthermia causes the polymerization of the curable bioadhesive to form cured adhesive.

In an embodiment, this disclosure provides a method of remotely-triggered thermal-curing of a biomedical adhesives at a wound site to form a wound dressing conforming to the shape of the wound comprising the steps of: (1) administering a liquid formulation of in situ curable bioadhesive as disclosed herein evenly over the wound site, (2) exposing the in situ curable bioadhesive to an exogenous source, wherein the material absorbs the energy from the exogenous source and converts the energy to heat, wherein the heat induces localized hyperthermia in the curable bioadhesive, wherein the localized hyperthermia causes the polymerization of the curable bioadhesive to form a wound dressing. In some embodiments, the in situ curable bioadhesive comprises crosslinkable hydrophilic prepolymers for hydrogel formation.

In an embodiment, this disclosure provides a method of remotely-triggered thermal-curing of a biomedical adhesives at a wound or body scissions of a patient for joining the tissue ends, wherein the method comprises the step of (1) providing the curable bioadhesive as disclosed herein, (2) applying the curable bioadhesive to at least two tissue ends at the wound or body scission, (3) pressing the tissue ends having the curable bioadhesives applications for a determined period of time, (4) exposing the tissue ends to the exogenous source, wherein the material absorbs the energy from the exogenous source and converts the energy to heat, wherein the heat induces localized hyperthermia in the curable bioadhesive, wherein the localized hyperthermia causes the polymerization of the curable bioadhesive to form a cured bioadhesive joining the tissue ends together.

In some embodiments, the speed of the curing of curable adhesive is tunable by tuning the laser wavelength. To induce rapid curing (e.g., curing within one minute), pulsed laser irradiation at 1064 nm is employed. To induce curing at a slower pace (e.g., 1 minute to several minutes), pulsed laser irradiation at 805 nm is employed. In some embodiments, one or more repeats of the laser irradiation may be employed to drive the curing at the desired level of completeness of the consumption of monomers, for example, minimize the residual toxic monomers in the cured adhesive.

In some embodiments, the induced hyperthermia in the curable bioadhesive is mild hyperthermia at a temperature ranging from about 38.0° C. to about 41.0° C. In some embodiments, the induced hyperthermia is moderate hyperthermia at a temperature ranging from about 41.1° C. to about 45.0° C. In some embodiments, the induced hyperthermia is profound hyperthermia at a temperature ranging from about 45.1° C. to about 52.0° C. In some embodiments, the hyperthermia induced the remotely triggered energy-thermal conversion is of a temperature ranging from about 38.0° C. to about 90.0° C.

In some embodiments, the temperature in the in situ curable bioadhesive is increased to a value ranging from about 50° C. to about 90° C. In some embodiments, the temperature in the in situ curable bioadhesive is increased to a value selected from the group consisting of about 50° C., about 55° C., about 60° C., about 65° C., about 70° C., about 75° C., about 80° C., and about 90° C.

In some embodiments, the disclosure provides a method for accelerating an in situ polymerization reaction of a curable bioadhesive at a tissue site. The method may include applying an in situ curable bioadhesive described herein to the tissue site, and exposing the in situ curable bioadhesive to an exogenous source. The exogenous source may be any exogenous source disclosed herein.

### 5. Hemostatic Composition

Current hemostatic agents effective in stopping the bleeding include cyanoacrylates, glutaraldehyde crosslinked albumin, zeolite-based QuickClot®, fibrin-based bandages, or gelatin based hemostatic agents. These hemostatic agents are typically used in first aid such as bandages, hemostatic sponges, and hemostatic powders. However, these hemostatic products and materials are insufficient for applications in control hemorrhaging associated with traumatic injuries. An ideal hemostatic agent should not only quickly control massive hemorrhage from large arteries, veins, and visceral organs, but also should be biocompatible and easy to use.

Death may occur in minutes after a traumatic injury due to blood loss. The body has natural mechanisms to control hemorrhaging, yet these processes may be insufficient in cases of excessive hemorrhaging, impairment due to medical conditions such as hemophilia, or compromise due to adverse effects of medications that includes blood-thinners like Coumadin. Administration of biologically derived blood products to augment the native hemostatic response and to maintain adequate oxygen delivery to the brain and vital organs carries significant risks including disease transmission, infection, pulmonary dysfunction, and immune response. Furthermore, many people have deficiencies within their hemostatic response i.e. hemophilia, which prevents them from adequately stopping blood loss. Millions of people around the world suffer from bleeding disorders and are unable to form a blood clot effectively. Current treatments are typically limited to clotting factor (Factor VIII, Factor II) replacement therapies, which are typically painful and expensive.

There exists a need for hemostatic compositions and products that possess instantaneous and high blood absorption capacity, fast shape recovery, and capability to induce rapid blood coagulation.

The blood coagulation cascade may be activated via two distinct routes: the tissue factor pathway and the intrinsic pathway, also known as the contact activation pathway. Both pathways eventually result in the activation of a common pathway, which leads to the formation of a fibrin-based hemostatic clot. One pathway relates to the use of a positively charged polymer network with adequate mechanical rigidity to induce the activation of FVII, which in turn leads to the activation of the common pathway and subsequent fibrin formation. The other pathway relates to the use of clotting material to induce the activation of FVII irrespective of calcium or platelets, which are typically vital cofactors of the process.

Accelerating the formation of a clot that blocks the blood flow from a hemorrhaging site using remote triggers to reduce the clotting time to under 120 seconds in the standard in vitro test can dramatically reduce blood loss.

In an embodiment, this disclosure provides a hemostatic composition useful for the enhancement of the clotting of blood in a subject. In an embodiment, the hemostatic composition comprises a heat delivery medium comprising a carrier and a material that interacts with an exogenous source and a physiologically acceptable medium, wherein the heat travels outside the hemostatic composition to an area surrounding the hemostatic composition, wherein the heat causes a controlled temperature rise to initiate or accelerate the formation of a blood clot, and wherein the hemostatic composition passes the Extractable Cytotoxicity Test. In an embodiment, the carrier and material comprising the heat delivery medium of the hemostatic composition may be at least one particle. In an embodiment, the heat delivery medium may be a nonwoven fabric, a woven fabric, a sheet, or a mesh.

As disclosed herein, the carrier will not only help absorb the blood but also serve to entrap the material to prevent it from causing toxicity to the body as well as protect the material from degrading chemicals in the blood. Upon exposure to the energy of the exogenous source, the material will absorb energy from the exogenous source to produce localized heating which will help accelerate clot formation.

In some embodiments, the hemostatic composition includes a physiologically acceptable medium. In some embodiments, the physiologically acceptable medium comprises a matrix, nonwoven fabric, or woven fabric, or nonwoven sheet, wherein the matrix, non-woven fabric, woven fabric, or nonwoven sheet comprises a polymer selected from PLGA, PCL, protein, gelatin, collagen, cellulose, oxidized regenerated cellulose, and combinations thereof.

In some embodiments, the physiologically acceptable medium comprises a polymer selected from the group consisting of PLGA, polycaprolactone, polyethylene glycol, block co-polymers comprising polyethylene glycol, block co-polymers comprising polyoxyalkylene, chitosan, hyaluronic acid, oxidized regenerated cellulose, polymethacrylate, copolymer of methacrylate and butyl methacrylate, block copolymer thereof, crosslinked polymer network thereof, hydrogel thereof, and combinations thereof.

In some embodiments, the physiologically acceptable medium comprises hydrogel having dendritic polymer. In some embodiments, the dendritic polymer comprises polyglycerol and dendritic polylysine.

In some embodiments, the physiologically acceptable medium comprises a biocompatible crosslinked polymer. In some embodiments, the biocompatible crosslinked polymer comprises a hydrogel. In some embodiments, the hydrogel is a water-responsive shape memory hydrogel. In some embodiments, the water-responsive shape memory hydrogel is formed from hydrogel precursors.

In some embodiments, the physiologically acceptable medium is made of polymer or co-polymers; examples include but may not limited to polycarbonate polyacrylates, polymethacrylates and copolymers thereof, polyurethanes, polyureas, cellulosic materials, polymaleic acid and its derivatives, and polyvinyl acetate. In some embodiments, the carrier comprises polymethacrylates and copolymers thereof.

In some embodiments, the physiologically acceptable medium comprises water-responsive shape memory polymers. As used herein, water-responsive shape memory polymers (SMPs) are a class of stimuli-responsive materials that can be elastically deformed and subsequently fixed into a temporary shape by network chain immobilization, and later recover to their original (permanent) shape when exposed to external water stimuli that re-mobilize the network chains. The water responsive memory polymer forms crosslinked hydrogel networks with crosslinking reagent to provide shape memory hydrogels having interconnected macroporous structure which allows water to freely flow in and out of the hydrogel network, by which the hydrogel shape can be fixed by squeezing out the free water and fast recovery to its original shape is achieved by re-absorbing water.

Compared with thermoreponsive SMPs, water-responsive SMPs are capable of regaining their original shapes simply by immersing the samples back in water.

In some embodiments, the water-induced shape-memory polymers comprise a hydrophilic or water swellable component into the structure such that the shape recovery can be greatly accelerated. In some embodiments, the water-induced shape-memory polymers comprises a poly(ethylene oxide) (PEO) block as soft-segment and a polyurethanes block modified with the hydrophobic polyhedral oligosilsesquioxane (POSS) moiety as the hard-segment. Exposure of the shape memory block copolymer to water results in the water-swelling of the PEG segment and recovery of the permanent shape.

In some embodiments, the water-induced shape-memory polymers comprise a chitosan block, a polyethylene glycol block having epoxide crosslinked networks. The equilibrium shape can be chemically fixed by crosslinking with epoxide. The polymers (chitosan and polyethylene glycol) used are relatively hydrophilic, and a subsequent immersion in water leads to rapid hydration and recovery of the permanent shape in a short period of 150 seconds.

In some embodiments, the water-responsive shape memory polymers are crosslinked hydrogels formed from poly(ε-caprolactone) (PCL) and poly(ethylene glycol) (PEG) multiblock hybrid thermoplastic polyurethanes, wherein the weight percent ratio of PCL to PEG ranges from about 30:70 to 70:30, wherein the urethane linkers are formed through the condensation reaction between isocyanate groups of the lysine methyl-ester diisocyanate (LDI) and the hydroxyl groups of either (PEG) or PCL diol. In some embodiments, the water-responsive shape memory polymers are crosslinked hydrogels formed from poly(ε-caprolactone) (PCL) and poly(ethylene glycol) (PEG) multiblock hybrid thermoplastic polyurethanes, wherein the weight percent ratio of PCL to PEG are selected from the group consisting of about 30:70, about 40:60, about 50:50, about 60:40, and about 70:30, wherein the urethane linkers are formed through the condensation reaction between isocyanate groups of the lysine methyl-ester diisocyanate (LDI) and the hydroxyl groups of either (PEG) or PCL diol.

In some embodiments, the water-responsive shape memory polymers comprise hydroxyethyl cellulose/soybean protein composite sponge agents having different microstructures formed from crosslinking hydroxyethyl cellulose and soybean protein with epichlorohydrin, or ethylene glycol diglycidyl ether at a weight percent ranging from about 10 wt. % to about 50 wt. % by the total weight of the hydroxyethyl cellulose and soybean protein.

In some embodiments, the water-responsive shape memory polymers comprise glycidyl methacrylate crosslinked quaternized chitosan hydrogel.

In some embodiments, the medium comprises self-expanding hemostatic polymer. In some embodiments, the self-expanding hemostatic polymer is a reaction product of polyvinyl alcohol, and a crosslinking agent including formaldehyde or glutaraldehyde, and multiarm PEG based crosslinking agent as disclosed herein.

In some embodiments, the self-expanding hemostatic polymer comprises the reaction product of a polyhydric alcohol, and a bi-functional substance containing at least one of a halogen atom or an epoxy group, wherein the bi-functional substance being reactive with the polyhydric alcohol, wherein the polyhydric alcohol is selected from the group consisting of saccharose, sorbitol, dextran, polyvinyl alcohol, and combinations thereof, and wherein the bi-functional substance is selected from the group consisting of diepoxybutane, diepoxypropyl ether or ethylene-glyco-bis-epoxypropyl ether, and combinations thereof.

In some embodiments, the physiologically acceptable medium comprises a hydrogel membrane, wherein the hydrogel membrane comprises a polymer selected from the group consisting of protein (e.g. silk), gelatin, collagen, hydroxyalkylmethylcellulose, PEG-PLGA block copolymer, PCL-PEG block copolymer, and combinations thereof.

In some embodiments, the physiologically acceptable medium is a porous matrix, and the particle heater is impregnated in the porous matrix, wherein the porous matrix may be a foam, a nonwoven fabric, a woven fabric, a hydrogel, or a sponge. In some embodiments, the physiologically acceptable medium may be solutions, ribbons, hemostatic gauzes, compression gauzes, pads, band-aids, occlusive dressings, liquid vehicles, granules, powder, microspheres, flakes, films, gel ointment, sponge, pastes, semisolid, hydrogel, water responsive shape memory hydrogel, crosslinkable polymers having reactive groups, crosslinked polymer networks, ribbons, hemostatic gauzes, compression gauzes, pads, band-aids, occlusive dressings, and combinations thereof. In some embodiments, the hemostatic composition and products thereof may be prepared by mixing, molding, extrusion, lyophilization, electrospinning, spray drying, crosslinking, in situ crosslinking, and any method that is known in the art.

In some embodiments, the physiologically acceptable medium comprises a hydrogel membrane, wherein the hydrogel membrane comprises a polymer selected from the group consisting of protein (e.g. silk), gelatin, collagen, hydroxyalkylmethylcellulose, PEG-PLGA block copolymer, PCL-PEG block copolymer, and combinations thereof.

In some embodiments, the physiologically acceptable medium is a porous matrix, and the particle heater is impregnated in the porous matrix, wherein the porous matrix may be a foam, a nonwoven fabric, a woven fabric, a hydrogel, or a sponge. In some embodiments, the physiologically acceptable medium may be solutions, ribbons, hemostatic gauzes, compression gauzes, pads, band-aids, occlusive dressings, liquid vehicles, granules, powder, microspheres, flakes, films, gel ointment, sponge, pastes, semisolid, hydrogel, water responsive shape memory hydrogel, crosslinkable polymers having reactive groups, crosslinked polymer networks, ribbons, hemostatic gauzes, compression gauzes, pads, band-aids, occlusive dressings, and combinations thereof. In some embodiments, the hemostatic composition and products thereof may be prepared by mixing, molding, extrusion, lyophilization, electrospinning, spray drying, crosslinking, in situ crosslinking, and any method that is known in the art.

In some embodiments, the physiologically acceptable medium takes a physical form selected from the group consisting of granules, powder, microspheres, flakes, films, gel ointment, sponge, foam, pastes, adhesives, semisolid, hydrogel, water responsive shape memory hydrogel, and combinations thereof.

In some embodiments, the medium optionally comprises water as structure constituent, e.g. water in the hydrogel. In some embodiments, the physiologically acceptable medium comprises a liquid vehicle. The liquid vehicle is selected form water, PBS buffer, saline, oil carrier such as fatty ester oil, squalene, squalene, hydrocarbon oils such as light mineral oil, and emulsions. In some embodiments, the particle heaters and the liquid vehicle in combination forms a liquid dispersion or suspension, and the particle heater dispersions and suspensions may optionally comprise a surfactant stabilizer. The surfactant suitable for incorporation in the hemostatic composition include polyethylene glycol, polyalkylene oxide, and block copolymer of polyalkyloxide (e.g., poloxamer).

In some embodiments, the physiologically acceptable medium comprises a sponge formed from hydrophilic polymers including polysaccharides (e.g. carrageenan, chitosan), proteins such as gelatin, collagen.

In some embodiments, the physiologically acceptable medium is present in the hemostatic composition at a weight percentage by the total weight of the hemostatic composition selected from the group consisting of about 20.0 wt. %, about 25.0 wt. %, about 30.0 wt. %, about 35.0 wt. %, about 40.0 wt. %, about 45.0 wt. %, about 50.0 wt. %, about 55.0 wt. %, about 60.0 wt. %, about 65.0 wt. %, about 70.0 wt. %, about 75.0 wt. %, about 80.0 wt. %, about 85.0 wt. %, about 90.0 wt. %, about 95.0 wt. %, and about 99.0 wt. %. In some embodiments, the physiologically acceptable medium is present in the particle heater at a weight percentage by the total weight of the hemostatic composition ranges from about 20.0 wt. % to about 99 wt. %. In some embodiments, the physiologically acceptable medium is present in the particle heater at a weight percentage by the total weight of the hemostatic composition ranges from about 25.0 wt. % to about 90.0 wt. %. In some embodiments, the physiologically acceptable medium is present in the hemostatic composition at a weight percentage by the total weight of the hemostatic composition ranges from about 50.0 wt. % to about 90.0 wt. %. In some embodiments, the physiologically acceptable medium is present in the hemostatic composition at a weight percentage by the total weight of the hemostatic composition ranges from about 25.0 wt. % to about 50.0 wt. %. In some embodiments, the physiologically acceptable medium is present in the hemostatic composition at a weight percentage by the total weight of the hemostatic composition ranges from about 75.0 wt. % to about 90.0 wt. %.

### (i) Optional Additional Hemostatic Agent

In some embodiments, the hemostatic composition optionally comprises additional hemostatic or coagulative agent selected from the group consisting of chitosan, calcium-loaded zeolite, silicate including kaolin, microfibrillar collagen, oxidized regenerated cellulose, anhydrous aluminum sulfate, silver nitrate, potassium alum, titanium oxide, fibrinogen, epinephrine, calcium alginate, poly-N-acetyl glucosamine, thrombin, coagulation factor(s) including Factor VII, Factor IX, Factor X, FVIIa, Von Willebrand factor, procoagulants including propyl gallate, antifibrinolytics including epsilon aminocaproic acid, coagulation proteins that generate Factor VII or FVIIa including Factor XII, Factor XIIa, Factor X, Factor Xa, protein C, protein S, and prothrombin, and combinations thereof.

Natural hemostatic agents contain, incorporate, or are derived from biological substrates, i.e. proteins, or cells. They can further be subdivided into the type of biological substrates incorporated into the system including collagen, thrombin, fibrin, albumin, and/or platelets.

Thrombin is the central activating enzyme of the common coagulation pathway. Thrombin circulates within the blood in its precursor or zymogen form, prothrombin. Prothrombin is specifically cleaved to produce the enzyme thrombin. The main role of thrombin in the coagulation pathway is to convert fibrinogen into fibrin, which in turn is covalently crosslinked to produce a hemostatic plug. Thrombin-based hemostatic agents take advantage of the natural physiologic coagulation response by augmenting, amplifying, and assisting the process. In some embodiments, the heat delivery hemostatic composition optionally comprises liquid or powder compositions of thrombin-based hemostatic agents selected from the group consisting of Thrombostat® (ParkeDavis), Thrombin-JMI® (King Pharmaceuticals, Briston, Tenn.), Quixil® (Omrix Biopharmaceuticals Ltd.), a combination of thrombin and fibrin (Evicel® by Johnson & Johnson; or FloSeal® by Baxter Healthcare Corporation), hybrids composed of bovine or porcine gelatin and thrombin (SurgiFlow®), and combinations thereof.

Fibrin is a fibrillar protein that is polymerized and crosslinked to form a mesh network, typically at the site of an injury after the induction of the coagulation cascade. The mesh network, incorporating other various proteins and platelets, forms a hemostatic plug to prevent continuous or further blood loss. Fibrin is activated from its inert zymogen, fibrinogen, by thrombin. Fibrin is in turn polymerized and covalently crosslinked by another coagulation factor, known as Factor XIIIa. In some embodiments, the heat delivery hemostatic composition optionally comprises fibrin selected from the group consisting of Tiseel® fibrin glue (Baxter HealthCare Corporation), FibRx® fibrin glue (CryoLife Inc.), Crosseel® fibrin glue (Johnson & Johnson), Hemaseel® fibrin glue (Haemacure Corporation, Montreal, Quebec), Beriplast P® fibrin glue (Aventis Behring), Bolheal® fibrin glue (Kaketsuken), and combinations thereof.

In some embodiments, the hemostatic composition comprises a gelatin fiber and a particle dispersed therein, wherein the particle comprises a carrier admixed with an IR dye, wherein the IR dye is Epolight™ 1117, or indocyanine green, wherein the carrier is selected from the group consisting of PLGA, PEG, PLGA-PEG, PCL, poly-1-lysine (PLL), albumin, polyethylene imine (PEI), and combinations thereof. In some embodiments, the hemostatic composition comprises a gelatin fiber of which the surface is coated with a particle, wherein the particle comprises a carrier admixed with an IR dye, wherein the IR dye is Epolight™ 1117, or indocyanine green, wherein the carrier is selected from the group consisting of PLGA, PEG, PLGA-PEG, PCL, poly-1-lysine (PLL), albumin, polyethylene imine (PEI), and combinations thereof. In some embodiments, the hemostatic composition comprises a gelatin fiber with a particle dispersed within, wherein the particle comprises a carrier admixed with an IR dye, wherein the IR dye is indocyanine green, and the carrier is PLGA. In some embodiments, the hemostatic composition comprises a gelatin fiber of which the surface is coated with a particle, wherein the particle comprises a carrier admixed with an IR dye, wherein the IR dye is indocyanine green, and the carrier is PLGA.

In some embodiments, the hemostatic composition comprises a collagen fiber with a particle dispersed within, wherein the particle comprises a carrier admixed with an IR dye. In some embodiments, the hemostatic composition comprises a collagen fiber of which the surface is coated with a particle, wherein the particle comprises a carrier admixed with an IR dye.

In some embodiments, the hemostatic composition comprises a PLGA fiber with an IR dye dispersed within. In some embodiments, the hemostatic composition comprises a PLGA fiber of which the surface is coated with an IR dye.

In some embodiments, the hemostatic composition comprises a compression gauze impregnated with particle heaters containing stimuli responsive agent. In some embodiments, the hemostatic composition comprises a compression gauze impregnated with particle heaters having a copolymer of methyl methacrylate/butyl methacrylate (MMA/BMA) and an IR dye. In some embodiments, the hemostatic composition comprises a compression gauze impregnated with particle heaters having a copolymer of methyl methacrylate/butyl methacrylate (MMA/BMA) and a tetrakis aminium dye. In some embodiments, the hemostatic composition comprises a compression gauze impregnated with particle heaters having a copolymer of methyl methacrylate and butyl methacrylate (MMA/BMA) and Epolight™ 1117 aminium dye. In some embodiments, the hemostatic composition comprises a compression gauze impregnated with particle heaters having a copolymer of methyl methacrylate and butyl methacrylate (MMA/BMA) and indocyanine dye.

In some embodiments, the hemostatic composition comprises a shape memory hydrogel with particle heaters having a MMA/BMA copolymer and IR dye dispersed within. In some embodiments, the hemostatic composition comprises a shape memory hydrogel with particle heaters having a MMA/BMA copolymer and tetrakis aminium dye dispersed within. In some embodiments, the hemostatic composition comprises glycidyl methacrylate, or genipin crosslinked gelatin hydrogel with particle heaters having a MMA/BMA copolymer and tetrakis aminium dye particles dispersed within. In some embodiments, the hemostatic composition comprises the glycidyl methacrylate, or genipin crosslinked gelatin hydrogel with particle heaters having a MMA/BMA copolymer and Epolight™ 1117 dispersed within. In some embodiments, the hemostatic composition comprises the glycidyl methacrylate, or genipin crosslinked gelatin hydrogel with particle heaters having a MMA/BMA copolymer and ICG dye dispersed within. In some embodiments, the hemostatic composition comprises the glycidyl methacrylate, or genipin crosslinked collagen hydrogel with a MMA/BMA copolymer encapsulated tetrakis aminium dye particle heaters dispersed within the gel. In some embodiments, the hemostatic composition comprises the glycidyl methacrylate, or genipin crosslinked collagen hydrogel with a MMA/BMA copolymer encapsulated Epolight™ 1117 particle heaters dispersed within the gel. In some embodiments, the hemostatic composition comprises the glycidyl methacrylate, or genipin crosslinked collagen hydrogel with a MMA/BMA copolymer encapsulated ICG dye particle heaters dispersed within the gel.

In some embodiments, the hemostatic composition comprises the glycidyl methacrylate crosslinked quaternized chitosan hydrogel having a MMA/BMA copolymer encapsulated tetrakis aminium dye particle heaters dispersed within the gel. In some embodiments, the hemostatic composition comprises the glycidyl methacrylate crosslinked quaternized chitosan hydrogel having a MMA/BMA copolymer encapsulated Epolight™ 1117 particle heaters dispersed within the gel. In some embodiments, the hemostatic composition comprises the glycidyl methacrylate crosslinked quaternized chitosan hydrogel with a MMA/BMA copolymer encapsulated ICG dye particle heaters dispersed within the gel.

In some embodiments, the hemostatic composition is an injectable water-responsive gel composition containing glycidyl methacrylate, or genipin crosslinked collagen hydrogel with a MMA/BMA copolymer encapsulated Epolight™ 1117 particle heaters dispersed within the gel.

In some embodiments, the heat delivery hemostatic composition is an injectable water-responsive gel composition containing glycidyl methacrylate crosslinked quaternized chitosan hydrogel and a particle heater comprising a MMA/BMA copolymer and Epolight™ 1117. In some embodiments, the hemostatic composition is an injectable liquid composition containing about 5.0 wt. % to 30 wt. % of glycidyl methacrylate crosslinked quaternized chitosan hydrogel and about 0.5 wt. % to about 20.0 wt. % Epolight™ 1117-MMA/BMA copolymer particle heaters.

In some embodiments, the heat delivery hemostatic composition is an injectable water-responsive gel composition containing glycidyl methacrylate crosslinked quaternized chitosan hydrogel and a particle heater comprising a MMA/BMA copolymer and indocyanine dye. In some embodiments, the hemostatic composition is an injectable liquid composition containing about 5.0 wt. % to 30 wt. % of glycidyl methacrylate crosslinked quaternized chitosan hydrogel and about 0.5 wt. % to about 20.0 wt. % indocyanine-MMA/BMA copolymer particle heaters.

Additional natural sourced hemostatic products suitable for incorporating into the heat delivery hemostatic composition of this disclosure may include covalently crosslinked protein networks in BioGlue® (Cryolife, Kennewsaw, Ga.), platelets containing products Costasis® (Orthovita), products containing serum albumin, and various other proteins, crosslinked with glutaraldehyde to form a rigid, insoluble matrix (BioGlue®).

### (ii) Hemostatic Product Forms

In some embodiments, this disclosure provides a hemostatic composition in a form selected from the group consisting of liquid dispersion, liquid suspension, powder, granules, particle, fiber, microgel, bulk hydrogel, emulsion, paste, semisolid, film, foam, sponges, gel, and combinations thereof. In some embodiments, the hemostatic composition is in a form selected from the group consisting of granule, sponge, bulk hydrogel, and combinations thereof.

In some embodiments, this disclosure provides hemostatic products for surgical and other medical purpose selected from the group consisting of dispersion, suspension, ribbons, hemostatic gauzes, compression gauzes, pads, band-aids, occlusive dressings, liquid bandage, water-responsive shape memory hydrogel comprising hemostatic compositions, multilayered hemostatic wound dressing, injectable in situ forming water-responsive shape memory hydrogel wound dressing, injectable hemostatic hydrogel wound dressing, injectable in situ forming thermal responsive hemostatic hydrogel dressing, sprayable hemostatic hydrogel wound dressing, sprayable in situ forming thermal responsive hemostatic hydrogel dressing, and combinations thereof.

In some embodiments, the novel hemostatic compositions in this disclosure are capable of thermally inducing or accelerating rapid coagulation via remotely controlled exogenous triggers. The hemostatic compositions in this disclosure provide rapid hemostasis that allows the emergency medical technician (EMT) or the clinician to induce rapid blood coagulation at a wound or bleeding site.

In some embodiments, the hemostatic compositions induce thermal coagulation without the use of exogenous thrombin such that it reduces the risk of blood-borne diseases and immunogenic reactions.

In an embodiment, this disclosure provides a method for treating a wound or a bleeding site in a subject comprising the administration to the wound or the bleeding site a product containing a therapeutically effective amount of a hemostatic composition as described above and irradiating the hemostatic product with a light source to induce thermal coagulation in the subject.

a. Compression Gauze Impregnated with Particle Heaters

In an embodiment, this disclosure provides a hemostatic product for rapid coagulation against extensive bleeding or hemorrhage comprising a hemostatic composition (e.g. MMA/BMA copolymer-Epolight™ 1117 particles) as described above and a compression gauze holding the hemostatic composition, wherein the compression gauze provides structure for clot formation.

In some embodiments, this disclosure provides a method of thermal coagulation for extensive bleeding or hemorrhage comprising the following steps: administering the compression gauze containing the MMA/BMA copolymer-Epolight™ 1117 particles to a bleeding site, applying slight pressure on the compression gauze on the bleeding site over a period of 3 minutes or less to provide physical restriction of the blood flow and absorption of the blood into the gauze matrix to induce aggregation of platelets, irradiating the gauze with a pulsed laser before, during, or after the application of pressure on the gauze, wherein the hemostatic composition absorbs energy of laser light and converts the energy to heat, wherein the heat travels outside the hemostatic composition to induce localized hyperthermia by causing a temperature rise at an area of tissue in proximity to the hemostatic composition. The temperature rise induces or accelerates the coagulation cascade, causes the denaturation of the proteins in the blood and the tissue structure of the blood vessels with a consequent coagulation in the blood vessel. The effects of rapid coagulation provided by the gauze holding hemostatic composition are the concurrent thermally induced coagulation by the laser light and the physical coagulation function of the gauze by compression and blood absorption.

b. Liquid Bandage

In an embodiment, the hemostatic product comprises a liquid bandage having a liquid vehicle admixed with the particle heaters as described above (e.g. MMA/BMA copolymer carrier-Epolight™ 1117 dye particles), wherein the liquid vehicle comprises at least one film forming agent, at least one non-aqueous solvent selected from the group consisting of hydrocarbons (e.g. heptane), low alcohols having 2-4 carbons (e.g. ethanol), vegetable oil (e.g. clove oil, eugenol), and combinations thereof.

In some embodiments, the disclosure provides a thermally induced coagulation of blood at a minor cutaneous bleeding site in a subject in need thereof comprising the steps of: administering the hemostatic liquid bandage over the bleeding site, irradiating the liquid bandage with a pulsed laser, wherein the particle heaters absorb the photonic energy of the laser light and convert the photonic energy to heat, the heat travels outside the particle heaters to induce a localized hyperthermia by causing a temperature rise at an area of tissue in proximity to the particle heaters, the temperature rise causes the denaturation of the proteins in the blood and the tissue structure of the blood vessels with a consequent coagulation in the blood vessel.

c. Water-Responsive Shape Memory Hydrogel Wound Dressing

In an embodiment, the hemostatic product comprises a water-responsive shape memory hydrogel admixed with the particle heaters as described above (e.g. MMA/BMA copolymer-Epolight™ 1117 particles).

In some embodiments, the disclosure provides a thermally induced coagulation for irregular and severe bleeding in a subject in need thereof comprising the steps of: administering the compressed hemostatic water-responsive shape memory hydrogel incorporating particle heaters over a bleeding site, irradiating the water-responsive shape memory hydrogel with a pulsed laser, wherein the compressed water-responsive shape memory hydrogel rapidly absorbing (e.g. less than 30 seconds) large amount of blood to cause the expansion of the hydrogel matrix that causes the physical coagulation by blocking the blood vessels, concurrently the particle heaters absorb the photonic energy of the laser light and convert the photonic energy to heat, the heat travels outside the particle to induce a localized hyperthermia by causing a temperature rise at an area of tissue in proximity to the particle heaters, the temperature rise causes the denaturation of the proteins in the blood and the tissue structure of the blood vessels with a consequent coagulation in the blood vessel.

d. Particle Heater as Hemostatic Agent for Minor Cutaneous Bleeding Site

In an embodiment, this disclosure provides the heat hemostatic product for coagulation at minor cutaneous bleeding site and comprises a powder, a dispersion or a suspension of the particle heaters as described above and a physiologically acceptable liquid carrier, wherein the liquid carrier is selected from the group consisting of water, PBS buffer, saline, oil carrier such as fatty ester oil, squalene, squalene, hydrocarbon oils such as light mineral oil, emulsions, and combinations thereof.

In some embodiments, the disclosure provides a thermally induced coagulation at a minor cutaneous bleeding site in a subject in need thereof comprising the steps of: administering a hemostatic product in the form of a power, a dispersion, or a suspension of the particle heaters to a bleeding site, irradiating the hemostatic product with a pulsed laser, wherein the particle heaters absorbing the photonic energy of the laser light and converts the photonic energy to heat, the heat transferring outside the particle to induce a localized hyperthermia by causing a temperature rise at an area of tissue in proximity to the particle heaters, the temperature rise causes the denaturation of the proteins in the blood and the tissue structure of the blood vessels with a consequent coagulation in the blood vessel.

e. Hemostatic Wound Dressing

In an embodiment, this disclosure provides a dry, removable, sterile multilayered hemostatic wound dressing that provides a dry hemostatic zone, wherein the dressing comprises a matrix holding the hemostatic compositions as described above, wherein the matrix is selected from the group consisting of film, hydrogel membrane, non-woven fabric, woven fabric, and combinations thereof; wherein the matrix is made of a biocompatible material selected from the group consisting of gelatin sponge, calcium alginate, collagen, oxidized regenerated cellulose, and combinations thereof; wherein the hemostatic composition comprising particles having a material interacting with an exogenous source encapsulated within a carrier, wherein the hemostatic composition is dispersed within, embedded within or forms a coating on the matrix. In some embodiments, the hemostatic wound dressing includes a compressed matrix, wherein the compressed matrix expands upon contacting with the blood.

In some embodiments, the hemostatic wound dressing acts as a hemostatic zone for topical applications constructed as a band-aid form, where the hemostatic zone is adhered to an adhesive backing layer, wherein the adhesive used to secure the hemostatic zone is porous in the areas contacting the skin. In some embodiments, the hemostatic wound dressing comprises one or more additional layers of wound dressing materials. In some embodiments, the hemostatic wound dressing comprises a layer containing super absorbents to wick blood at the bleeding site.

In some embodiments, the disclosure provides a method for treating a wound or a bleeding site in a subject comprising the administration of a hemostatic wound dressing containing therapeutically effective amount of a hemostatic composition as described above to the wound or bleeding site, and irradiating the hemostatic wound dressing with a pulsed laser to induce thermal coagulation in the subject.

f. Hemostatic Patch

In some embodiments, this disclosure provides a hemostatic patch comprising a matrix and the hemostatic compositions described above. In some embodiments, this disclosure provides a hemostatic patch suitable for rapidly arresting blood loss and inducing rapid clot formation at a bleeding site, wherein the patch comprises a dry sterilized flexible matrix containing the hemostatic compositions described above to provide a dry hemostatic zone, and an adhesive layer configured for facing the tissue. The patch may be used like a band-aid, or a dressing to the bleeding site to arrest blood loss and accelerate clot formation at the bleeding site. An effective way of plugging or arresting the bleeding site would be to apply the patch to the bleeding surface, holding the same with light pressure for a period adequate (e.g. 3 minutes) to induce hemostasis. During that time, in addition to hemostasis, a hermetic seal forms. Irradiation of the patch with a pulsed laser before, during, or after the application of pressure on the patch leads to the acceleration of clot formation, wherein the hemostatic composition absorbs energy of laser light and converts the energy to heat, wherein the heat travels outside the hemostatic composition to induce localized hyperthermia by causing a temperature rise at an area of tissue in proximity to the hemostatic composition. The temperature rise induces or accelerates the coagulation cascade and causes the denaturation of the proteins in the blood and the tissue structure of the blood vessels with a consequent coagulation in the blood vessel. The effects of rapid coagulation provided by the patch holding the hemostatic composition are the concurrent thermally induced coagulation by the laser light and the physical coagulation function of the patch by compression and blood absorption.

(iii) Laser Induced Thermal Blood Coagulation

The current hemostasis of blood vessels of small caliber, e.g. superficial cutaneous capillaries, is mainly achieved in three ways: (1) by local mechanical compression that interrupts the blood flow in the vessel, enabling coagulation to occur through platelet aggregation, (2) by pharmacological treatments, e.g. using natural hemostasis proteins including thrombin, fibrinogen; or (3) by a thermal photocoagulation inducing processes. These photothermal coagulation procedures generally do not have a selective action on the hematic components but induces coagulation of all the tissue. The photothermal coagulation procedure often has an excessive thermal effect and consequently causes collateral damage to the surrounding tissues.

There exists a need for selective thermal heating of blood components to minimize collateral damage to the surrounding tissues. In the laser induced thermal coagulation applications, it is desirable to target hematic components in the blood for localized heating to provide tunable temperature rise. Techniques that cause precise localized heating would allow for rapid hemostasis in the damaged blood vessels while minimizing collateral damage to the nearby cells and tissues.

This disclosure provides hemostatic compositions and hemostatic products comprising particle heaters and methods of achieving thermal coagulation via a remotely-triggered activation of the particle heaters by laser irradiation at a wavelength of 1064 nm instead of the non-selective, laser-induced heating of the water content of the tissues.

An important physical property of the particle heater for causing an actuation of a biological process or a chemical process is the increased temperature that is generated within a biological system and the scope and spatio-temporal span over which the temperature change occurs. In a typical biomedical application, the particle heaters are injected into a small cavity inside a tissue and are optically stimulated. When the exogenous light source is applied, the material encapsulated in the particle heaters will interact with the light source, absorb the energy thereof, and convert the energy to heat that travels outside the particle heaters to induce a localized temperature rise in the surrounding area of the particle heaters. Tissues typically have the heat conductivity of water and heat from the particle heater is likely to flow isotropically inside the tissue. The subsequent thermally induced coagulation results from an increase in temperature induced in the surrounding tissues near the particle heaters, in which the temperature rise causes the thermal denaturation of the proteins contained in the blood and in the structural component of the vessel.

In one embodiment, the disclosure provides a method of thermal coagulation induced by localized hyperthermia by irradiating a hemostatic composition or heat delivery products described herein. Irradiating the particle heaters incorporated within the hemostatic composition and hemostatic products includes directing electromagnetic radiation onto the particle heaters. The electromagnetic radiation may come from any source, including a LED, a laser, or a lamp. Any source that can provide the appropriate radiation, including wavelength and intensity, is compatible with the disclosed methods. In one embodiment, the source is a narrow-band EMR source, with a particular bandwidth tuned to wavelengths compatible with human tissue. In another embodiment, the source is a broadband EMR source. In some embodiments, the source is a laser. In some embodiments, the source is a pulsed laser.

In some embodiments, the method further comprises heating an area in the proximity of the hemostatic composition/hemostatic product/particle heaters by transferring heat from the hemostatic composition/hemostatic product/particle heaters to the surrounding area. As used herein, the term “in proximity to” is defined as an area containing the hemostatic composition/hemostatic product/particle heaters or sufficiently near the hemostatic composition/hemostatic product/particle heaters to receive heat transferred from the hemostatic composition/hemostatic product/particle heaters after heated by optical irradiation. By this step, heating the hemostatic composition/hemostatic product/particle heaters is used to heat an area around the hemostatic composition/hemostatic product/particle heaters to provide targeted heat, inducing localized hyperthermia, activated by light illumination. The area to be heated by the hemostatic composition/particle can be liquid, solid, gas, or any combinations thereof. In one embodiment, the area is heated to a temperature of 35° C. to 120° C. In one embodiment, the area is heated to a temperature greater than 42° C. In one embodiment, the area is heated to a temperature of 37.5° C. to 50° C. In one embodiment, the area is heated to a temperature of about 37.5° C., about 38° C., about 38.5° C., about 39° C., about 39.5° C., about 40° C., about 40.5° C., about 41° C., about 41.5° C., about 42° C., about 42.5° C., about 43° C., about 43.5° C., about 44° C., about 44.5° C., about 45° C., about 45.5° C., about 46° C., about 46.5° C., about 47° C., about 47.5° C., about 48° C., about 48.5° C., about 49° C., about 49.5° C., or about 50° C.

In some embodiments, this disclosure additionally provides a method of disinfecting a wound or surgical incisions comprising: (1) administering to the wound or the surgical incisions an amount of the particle-based hemostatic composition as disclosed herein, (2) exposing the hemostatic composition to an exogenous source for a sufficient period of time to induce localized hyperthermia having a temperature of about 41° C. to about 52° C., wherein the hyperthermia cause bacterial death due to thermal effects induced apoptosis and/or necrosis whereby provide the effects of disinfecton at the wound or the surgical incisions. In some embodiments, the hemostatic composition further comprises particles having an energy-absorbing agent suitable for photodynamic processes for generating reactive oxygen species (ROS) that is a potent agent for killing microbes.

In some embodiments, the exogenous source is selected from the group consisting of an electromagnetic radiation, an electrical field, a microwave, a radio wave, ultrasonic radiation, a magnetic field, and combinations thereof. In some embodiments, the exogenous source comprises microwave.

In some embodiments, the exogenous source may have a cold tip to cool the target tissue area before, during and after application of the exogenous energy. In some embodiments the cold tip may be at a temperature from about 2-8° C.

In some embodiments, the exogenous source comprises an ultrasonic source. In some embodiments, the material comprises ICG dye.

In some embodiments the exogenous source is an ultrasonic wave produced by an ultrasound (US) producing machine. In some embodiments the therapeutic ultrasound is either pulsed or continuous.

The frequency of US energy dictates the depth of penetration and impacts the efficiency of particle heaters. To reach deeper tissues (up to 5 cm or more), a frequency of 1 MHz should be selected. When the target tissue is within 2.5 cm from the surface of the skin, a frequency of 3 MHz should be selected. It is important to note that 3 MHz will produce heat from particle heaters approximately 3-times faster than 1 MHz, creating a higher efficiency in heating when compared to 1 MHz ultrasound for the same particle heater. For continuous US, frequencies within the range of 1-3 MHz at intensities of 0.5-10 W/cm² for a duration of 1-15 minutes at 100% duty cycle should be useful for in vivo applications. In some embodiments the US frequencies of 1-2 MHz at intensity ranges from 0.5-5 W/cm² are applied for 1-5 minutes at 100% duty cycle. 3 MHz ultrasound is absorbed more rapidly in the tissues, and therefore is considered to be most appropriate for superficial lesions, whilst the 1 MHz energy is absorbed less rapidly with deeper progression through the tissues, and can therefore be more effective at greater depth. The boundary between superficial and deep tissues is in some ways arbitrary, but somewhere around the 2 cm depth is often taken as a useful boundary. Hence, if the target tissue is within 2 cm (or just under an inch) of the skin surface, 3 MHz treatments will be effective whilst treatments to deeper tissues will be more effectively achieved with 1 MHz ultrasound. One important factor is that some of the US energy delivered to the tissue surface will/may be lost before the target tissue (i.e. in the normal or uninjured tissues which lie between the skin surface and the target). In order to account for this, it may be necessary to deliver more US energy at the surface than is required, therefore allowing for some absorption before the target tissue, and allowing sufficient remaining US energy to achieve the desired effect. To identify the appropriate dose to set on the machine, one has to determine (a) the estimated depth of the lesion to be treated and (b) the intensity of US energy required at that depth to achieve the desired effect. For example, to achieve a 0.5 W/cm² intensity at 1 cm tissue depth, one would select 3 MHz treatment option and set machine to 0.7 W/cm² which will result in 0.5 W/cm² intensity at a 1 cm tissue depth. The rate at which US energy is absorbed in the tissues can be approximately determined by the half value depth, the tissue depth at which 50% of the US energy delivered at the surface has been absorbed. The average half-value depth of 3 MHz ultrasound is taken at 2.5 cm and that of 1 MHz ultrasound as 4.0 cm though there are numerous debates that continue with regards the most appropriate half value depth for different frequencies.

In some embodiments pulsed ultrasound is used. The pulse ratio determines the concentration of the sound energy on a time basis. The pulse ratio determines the proportion of time that the ultrasound machine is “ON” compared with the “OFF” time. A pulse ratio of 1:1 for example means that the machine delivers one ‘unit’ of US energy followed by an equal duration during which no energy is delivered. The machine duty cycle is therefore 50%. A machine pulsed at a ratio of 1:4 will deliver one unit of US energy followed by 4 units of rest, therefore the machine is on for 20% of the time (some machines use ratios, and some use percentages). The selection of the most appropriate pulse ratio essentially depends on the state of the target tissue(s). The less dense the target tissue state, the more energy sensitive it is, and appears to respond more favorably to energy delivered with a larger pulse ratio (lower duty cycle). As the tissue becomes denser, it appears to respond preferentially to a more ‘concentrated’ energy delivery, thus reducing the pulse ratio (or increasing the duty cycle). It is suggested that pulse ratios of 1:4 would be best suited to the treatment of low density tissues, reducing this as the tissue increases in density, moving through 1:3 and 1:2 to end up with 1:1 or continuous modes. As a general rule, a pulse ratio of 1:4 or 1:3 will be preferred for the less dense tissues, 1:2 and 1:1 for the medium density tissues, and 1:1 or Continuous for the denser tissues. The final compilation of the treatment dose which is most likely to be effective is based on the principle that about 1-minute worth of US energy (at an appropriate frequency and intensity) should be delivered for every treatment head that needs to be covered. The size of the treatment area will influence the treatment time, as will the pulse ratio being used. The larger the treatment area, the longer the treatment will take. The lower the duty cycle of the pulsed energy output from the machine, the longer it will take to deliver about 1-minute worth of US energy. The desired ultrasonic dose will also depend on the particle heater concentration at the target tissue.

In one embodiment, this disclosure provides a method of thermal coagulation induced by localized hyperthermia caused by an exogenous source comprising the following steps: (a) administering to a bleeding site the hemostatic composition/products/particle heaters comprising a carrier and a material that interacts with an exogenous source; (b) irradiating the hemostatic composition/products/particle heaters with the exogenous source, wherein the particle heaters or the material absorbs the energy from the exogenous source and convert the energy into heat; wherein the heat causes a temperature rise in the area close to the hemostatic compositions to denature the proteins in the blood and the structural components of the blood vessels.

In some embodiments, the exogenous source comprises an electromagnetic radiation. In some embodiments, the electromagnetic radiation source comprises a LED light or a laser light.

In some embodiments, the electromagnetic radiation source comprises a LED light. LEDs are solid state p-n junction devices which emit light when forward biased. An LED is a Light Emitting Diode, a generic term. An IRED is an Infrared Emitting Diode, a term specifically applied to IR emitters. Unlike incandescent lamps which emit light over a very broad range of wavelengths, LEDs emit light over such a narrow bandwidth that they appear to be emitting a single “color”.

In some embodiments, the material absorbs optical energy at a wavelength from 750 nm-950 nm (e.g. Infrared Light Emitting Diodes (IRED) by Excelitas). In some embodiments, the material absorbing optical energy at a wavelength from 400 nm to 750 nm (e.g. a LED device). In some embodiments, the material is selected from the group consisting of a tetrakis aminium dye, a cyanine dye squaraine dye, IR 193 dye, ICG dye, IR 820 dye (new ICG dye), and combinations thereof.

In some embodiments, the exogenous source is a laser. This comprises a hollow sheath, which covers the distal end of the fiber optic conduit, defines a pocket, and a fiber optic lens in the pocket, and is modified to receive and direct the laser energy emitted from the fiber optic conduit through the lens onto the occlusion and to form a channel therethrough. The fiber optic conduit can be adapted for the specific application. Optical fibers are hair thin strands of glass or plastic that guide light. The optical fiber has an inner core surrounded by an outer cladding. In order to guide the light, the core refractive index is higher than the cladding index. A fiber grating is formed inside the core of a fiber. This is widely used in the field of fiber-optic communication for wavelength management. The optical grating reflects or transmits a certain portion, wavelength (bandwidth) or intensity, of the light along the optical fibers. A fiber Bragg grating is based on the interference of multiple reflections of a light beam in a fiber segment whose index of refraction varies periodically along the length of the fiber. Variations of the refractive index constitute discontinuities that emulate a Bragg structure. If the spacing of the index periods is equal to one half of the wavelength of the light, then the waves will interfere constructively (the round trip of each reflected wave is one wavelength) and a large reflection will occur from the periodic array. Optical signals whose wavelengths are not equal to one half the spacing will travel through the periodic array unaffected. In one embodiment, the optical grating is a Bragg grating or a long period grating. In another embodiment, the optical grating is coated with a composition having a thermal coefficient that is greater than the thermal coefficient of the fiber. In a further embodiment, at least one optical fiber further comprises an optical diffraction means for simultaneously measuring multiple peaks of the reflected light beam. In a further embodiment, the optical grating has a length between 0.2 and 40 mm.

Endoscopes are well-known medical instruments used to visualize the interior of a body cavity or organ. Endoscopes are used in a variety of operative procedures, including laparoscopic surgery where endoscopes are used to visually examine the peritoneal cavity. Typical endoscopes are configured in the form of a probe having a distal end for insertion through a small incision in the body. The probe includes components for delivery of illumination light and collection of an image from inside the body. Optical fibers or optically transmissive composition in a tubular formation typically provides illumination light delivery to a distal end of the probe. Imaging is typically carried out by an objective lens and relay optics that receive and deliver an image to the proximal end of the probe, which may be equipped with an eyepiece or an electronic image capture device such as a CCD (charge coupled device) sensor array. Endoscope probes may be rigid or flexible, with the light delivery and image retrieval components configured accordingly. Flexible bundles of optical fibers are used to produce a flexible probe, while rigid probes may have fused optical fiber assemblies, rigid light pipes and/or imaging rods and lenses. The intended use of the endoscope dictates the length of the probe, the need for flexibility and the necessary image quality.

In some embodiments, the method for delivery of therapeutic light for particle-based therapy comprises: (a) providing an endoscope with a light delivery optical pathway transmissive of said therapeutic light, said endoscope also including an image retrieval optical pathway and imaging system for generating an image of a target area; (b) providing a light generator that selectively produces said therapeutic light and also generates visible light; (c) inserting said endoscope into a cavity of a living organism to identify and illuminate a target area, said inserting including employing the image to direct said insertion and identify said target area; (d) activating said light generator to produce said therapeutic light to achieve a therapeutic objective at said target area; and (e) removing said endoscope from said cavity. In some embodiments, the therapeutic light has a wavelength from 750 nm to 1100 nm.

In some embodiments the therapeutic light delivery endoscope comprises: 1. a broad spectrum light source that generates pulses of light having wavelengths between about 700 nm and about 1100 nm; 2. a control circuit operatively connected to said broad spectrum light source, said control circuit providing adjustable control over the frequency, power and wavelength of said light pulses; 3. a light delivery optical pathway constructed of components selected to transmit light including UV light having a wavelength between 200 nm and 300 nm, said light delivery optical pathway arranged to receive and transmit light generated by said broad spectrum light source to a target area; 4. an image retrieval optical pathway arranged to receive light reflected from said target area; 5. an image generating system which employs light from said image retrieval optical pathway to generate an image of said target area; and 6. an interface allowing a user to adjust the frequency, power and wavelength of said pulses of light, thereby controlling the quantity of the light delivered to the target tissue area.

In some embodiments, it is desirable to keep the temperature in the surrounding area of the heat delivery composition/medium/particle to be sufficiently low to avoid collateral damage to the healthy tissues and also control the temperature rise to be sufficiently high to accelerate a physical, chemical or biological activity.

In some embodiments, the electromagnetic radiation source is a laser light. In some embodiments, pulsed lasers are utilized in order to provide localized thermal heating. In some embodiments, the laser irradiation is delivered in a pulse duration longer than the thermal relaxation time (TRT) of the particle heaters such that the heat generated by the particle begins to travel outside the particle. In some embodiments, the flow of the heat delivery to the outside of the particles can be achieved by manipulating the fluence of the laser irradiation, particle size and the concentration of the particles. Pulses are at least nanoseconds in duration.

In some embodiments, the laser pulse duration is in a range from milliseconds to nanoseconds, and the laser has an oscillation wavelength at 805 nm, 808 nm, or 1064 nm. In some embodiments, the particle heater absorbs the laser light having a wavelength from 750 nm to 1100 nm. In some embodiments, the material is selected from the group consisting of a tetrakis aminium dye, a cyanine dye, indocyanine green dye (ICG), new ICG dye (IR820), IR 193 dye, iron oxide, a tetrakis aminium dye, and combinations thereof.

In some embodiments, laser wavelength has a dual impact attributable to the absorption coefficient of the material as well as the depth of penetration to the tissue site, which roughly increases as the wavelength increases in the visible and near infrared spectrum. After carefully choosing a proper laser wavelength and pulse duration for a particular material, delivering the optimum number of photons to the hemostatic composition/particle having the same material can be achieved.

In some embodiments, the particle heater offers tunable photon absorption by varying the particle size, particle concentration, and selection of IR absorbing material with a defined chemical structure to allow facile matching of particle absorption to the output of various commercial lasers. Additionally, the method in this disclosure affords a path to minimize tissue damage by using the least harmful wavelengths of laser light sources.

The selection of laser parameters used to cause a controlled heat generation may include wavelength, average power, instantaneous power, pulse duration and/or total exposure duration. The pulse duration (t_{d}), of the exposure can influence the specificity or confinement of collateral thermal damage, and may be determined from the thermal relaxation time (tᵣ, also known as TRT) of the target material. The transition from specific to non-specific thermal damage can occur when the ratio is as follows: (t_{d}/tᵣ)≥1. For spheres of radius, R, and thermal diffusivity, κ, the thermal relaxation time can be provided by tᵣ=(R²/6.75κ).

To transfer the heat outside the particle preferably, the pulse duration of the laser exposure is selected to be greater than the thermal relaxation time of the particle. The power density is selected so as to be sufficient to induce localized mild hyperthermia (e.g. a temperature increase of at least 5° C. about room temperature) in the surrounding environment of the particles.

In some embodiments, the laser is operated at 750 nm, 805 nm, 808 nm, 810 nm, 1064 nm with a power density of about 40 mW/cm² to about 450 mW/cm². In some embodiments, the laser is operated at 750 nm, 805 nm, 808 nm, 810 nm, 1064 nm with a power density of about 40 mW/cm² to about 360 mW/cm². In some embodiments, the laser is operated at 750 nm, 805 nm, 808 nm, 810 nm, 1064 nm with a power density of about 100 mW/cm² to about 350 mW/cm². In some embodiments, the 808 nm NIR laser is operated at ultra-low laser power (10 mW) to generate more ROS. Various repetition rates are used from continuous to pulsed, e.g., at less than 1 Hz, or 1-5 Hz.

In some embodiments, the laser pulse duration is longer than the particle TRT. In some embodiments, the laser pulse duration is less than a millisecond or a microsecond in duration. In some embodiments, a source emitting radiation at a wavelength of 755 nm is pulsed at a duration of 0.25-400 milliseconds (ms) per pulse, with a pulse frequency of 1-10 Hz. In some embodiments, a source emitting radiation at a wavelength of 810 nm is pulsed at 5-400 ms with a frequency of 1-10 Hz. In some embodiments, a source emitting radiation at a wavelength of 1064 nm is pulsed at 0.25-400 ms at a frequency of 1-10 Hz. In some embodiments, a source emitting pulsed light at a wavelength of 530-1200 nm is pulsed at 0.5-400 ms at a frequency of 1-10 Hz.

In some embodiments, the particle heaters have a TRT of about 250 ns, about 275 ns, about 300 ns, about 325 ns, about 350 ns, about 375 ns, about 400 ns, about 425 ns, about 450 ns, about 475 ns, about 500 ns, about 525 ns, about 550 ns, about 575 ns, about 600 ns, about 625 ns, about 650 ns, about 675 ns, about 700 ns, about 725 ns, about 750 ns, about 775 ns, about 800 ns, about 825 ns, about 900 ns, about 925 ns, about 950 ns, about 975 ns, about 1000 ns, about 1100 ns, about 1200 ns, about 1300 ns, about 1400 ns, about 1500 ns, about 1600 ns, about 1700 ns, about 1800 ns, about 1900 ns, about 2.0 ms, about 3 ms, about 4 ms, about 5 ms, about 6 ms, about 7 ms, about 8 ms, about 9 ms, about 10 ms, about 20 ms, about 30 ms, about 40 ms, about 50 ms, about 60 ms, about 70 ms, about 80 ms, about 90 ms, or about 100 ms.

In some embodiments, short pulses (100 ns to 1000 ms) are used to drive very high transient heat gradients in and around the target tissue structure from embedded particles to localize damage in close proximity to particle location. In other embodiments, longer pulse lengths (1 ms to 10 ms, or 1 ms to 500 ms) are used to drive heat gradients further from the target structure to localize thermal energy to components greater than 100 μm away from the localized particles. In some of such embodiments, pulses of varying durations are provided to localize thermal heating regions to be within 0.05, 0.1, 0.5, 1, 2, 5, 10, 20, 30, 50, 75, 100, 200, 300, 500, 1000 microns of the particles.

In some embodiments, the induced hyperthermia is mild hyperthermia at a temperature ranging from about 38.0° C. to about 41.0° C. In some embodiments, the induced hyperthermia is moderate hyperthermia at a temperature ranging from about 41.1° C. to about 45.0° C. In some embodiments, the induced hyperthermia is profound hyperthermia at a temperature ranging from about 45.1° C. to about 52.0° C.

To avoid healthy tissue damage, it is important to ensure the energy of laser irradiation is preferentially absorbed by the particles containing the IR absorbing dye and not absorbed by the tissue to be treated. When the pulse duration exceeds the TRT of the particle heaters, then the heat energy generated begins to travel outside the particles. In addition, the duration of the pulse can be controlled to ensure that the heat produced by the particles will diffuse out into the surrounding environment. The cooling procedure allows maintaining viability of endothelium while safely applying cell-specific laser irradiation into the deep vascular tissues. The coolant can be delivered using the same catheter that delivers the laser optical fibers to the tissue site.

In some embodiments, the particle heaters are present in the hemostatic composition in an amount ranging from about 0.5 wt. % to about 25 wt. % by the total weight of the hemostatic composition. In some embodiments, the particle heater is present in an amount ranging from about 1.0 wt. % to about 20.0 wt. % by the total of the hemostatic composition. In some embodiments, the particle heater is present in an amount ranging from about 5.0 wt. % to about 20.0 wt. % by the total of the hemostatic composition. In some embodiments, the particle heater is present in an amount ranging from about 5.0 wt. % to about 15.0 wt. % by the total of the hemostatic composition. In some embodiments, the particle heater is present in an amount ranging from about 10.0 wt. % to about 15.0 wt. % by the total of the hemostatic composition. In some embodiments, the particle heater is present in an amount selected from the group consisting of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, about 20.0 wt. %, about 20.5 wt. %, about 21.0 wt. %, about 21.5 wt. %, about 22.0 wt. %, about 22.5 wt. %, about 23.0 wt. %, about 23.5 wt. %, about 24.0 wt. %, about 24.5 wt. %, and about 25.0 wt. % by the total weight of the hemostatic composition. In some embodiments, the particle heater is present in an amount selected from the group consisting of about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, and about 15.0 wt. % by the total weight of the hemostatic composition. In some embodiments, the particle heater is present in an amount selected from the group consisting of about 1.0 wt. %, 2.0 wt. %, 3.0 wt. %, 4.0 wt. %, about 5.0 wt. %, about 10.0 wt. % and about 15.0 wt. % by the total weight of the hemostatic composition.

The hemostatic compositions and hemostatic products thereof provided herein have the advantages that the blood coagulation is induced exogenously, wherein the composition rapidly absorbs the water in blood and tissue fluid to form a gel, sealing the blood capillary ends to perform the function of physical coagulation due to mechanical compression and blood vessel blockage. Further, the composition following interaction with the exogenous source exhibits heat induced biocidal activity.

TABLE 2
Hemostatic Composition

| | | | | Product physical |
|---|---|---|---|---|
| Entry | Clotting agent | Heating agent | Carrier | forms |
| 1 | zeolite granules | 2 μm Epolight ™ 1117 | PMMA, or PLGA, | composite powder |
| | | PMMA/BMA particles | starch, carrageenan, | |
| | | | alginate, or chitosan | |
| 2 | Smectite mineral clay | 2 μm Epolight ™ 1117 | Crosslinked | composite gel or |
| | | PMMA/BMA particles | polyacrylic acid, | granular preparation |
| | | | PMMA, or PLGA, | |
| | | | starch, carrageenan, | |
| | | | alginate, or chitosan | |
| 3 | kaolin | 2 μm Epolight ™ 1117 | Crosslinked | composite gel or |
| | | PMMA/BMA particles | polyacrylic acid, | granular preparation |
| | | | PMMA, or PLGA, | |
| | | | starch, carrageenan, | |
| | | | alginate, or chitosan | |
| 4 | chitosan | 2 μm Epolight ™ 1117 | — | granules, bandage |
| | | PMMA/BMA particles | | |
| 5 | chitosan and | 2 μm Epolight ™ 1117 | PMMA, or PLGA, | composite granules or |
| | polyacrylic acid | PMMA/BMA particles | carrageenan, | composite gel |
| | embedded within | | alginate, or chitosan | |
| | mesoporous silica | | | |
| 6 | microporous starch | 2 μm Epolight ™ 1117 | PMMA, or PLGA, | composite granules or |
| | particles | PMMA/BMA particles | carrageenan, | composite gel |
| | | | alginate, or chitosan | |
| 7 | porous polyethylene | 2 μm Epolight ™ 1117 | carrageenan, starch, | nonwoven |
| | nonwoven fabric fibers | PMMA/BMA particles | alginate, or chitosan | |
| | fdled with silica and | | | |
| | coated with chitosan, | | | |
| | the fibers having 20 to | | | |
| | 100 μm in diameter | | | |
| 8 | polyethylene glycol | 2 μm Epolight ™ 1117 | carrageenan, starch, | Sponge |
| | sponge filled with | PMMA/BMA particles | alginate, or chitosan | |
| | silica coated with | | | |
| | chitosan | | | |
| 9 | Self-expanding | 2 μm Epolight ™ 1117 | | physical mixture, |
| | hemostatic polymer | PMMA/BMA particles | | composite gel |
| 10 | kaolin bonded to | 2 μm Epolight ™ 1117 | carrageenan, starch, | gauze |
| | polyester/rayon gauze | PMMA/BMA particles | alginate, or chitosan | |
| 11 | Fibrinogen, dirombin | 2 μm Epolight ™ 1117 | polyglactin 910 | mesh |
| | and calcium chloride | PMMA/BMA particles | mesha (copolymer | |
| | | | made from 90% | |
| | | | glycolide and 10% | |
| | | | L-lactide) | |
| 12 | polyethylene glycol | Epolight ™ 1117, ICG, | polyethylene | sponge |
| | sponge filled with | IR 820, or IR 193 dye | glycol, carrageenan, | |
| | nanosize zeolite | | starch, alginate, or | |
| | molecular sieve | | chitosan | |
| 13 | micron-size zeolite | Epolight ™ 1117, ICG, | polyethylene | microgranule, gel, |
| | molecular sieve | IR 820, or IR 193 dye | glycol, carrageenan, | sponge |
| | | | starch, alginate, or | |
| | | | chitosan | |
| 14 | zeolite granules | Epolight ™ 1117, ICG, | PMMA, or PLGA, | composite powder |
| | | IR 820, or IR 193 dye | starch, carrageenan, | |
| | | | alginate, or chitosan | |
| 15 | Smectite mineral clay | Epolight ™ 1117, ICG, | Crosslinked | composite gel or |
| | | IR 820, or IR 193 dye | polyacrylic acid, | granular preparation |
| | | | PMMA, or PLGA, | |
| | | | starch, carrageenan, | |
| | | | alginate, or chitosan | |
| 16 | porous polyethylene | Epolight ™ 1117, ICG, | Crosslinked | nonwoven |
| | nonwoven fabric fibers | IR 820, or IR 193 dye | polvacrylic acid, | |
| | filled with silica coated | | PMMA, or PLGA, | |
| | with chitosan | | starch, carrageenan, | |
| | | | alginate | |

### 6. Remotely-Triggered In Situ Curable Dental Composition

Dental compositions generally have unique requirements as compared to the broad spectrum of composite materials. For health reasons, dental compositions should be suitable for use in the oral environment. In certain instances, durability of a dental composition is important to ensure satisfactory performance. For example, high strength and durability is desirable for dental work that is performed at dentition locations where mastication forces are generally great. In other instances, aesthetic character or quality is highly desired. This is often the case where dental work is performed at locations where a tooth repair or restoration can be seen from a relatively short distance.

It is also generally desired that the dental restorative material blend well with the surrounding dentition and that the dental restorative material looks life-like. Aesthetic quality in dental compositions is typically achieved by creating material that has tooth-like colors/shades. Many fills, however, generally have less mechanical strength than is desired.

In an embodiment, this disclosure provides an in situ curable dental composition composing a filler dispersed in a polymerizable resin composition. In some embodiments, the curable dental composition is formulated as a dental adhesive, an artificial crown, anterior or posterior fillings, casting materials, cavity liners, cements, coating compositions, mill blanks, orthodontic devices, restoratives, prostheses, or sealants.

### (i) Monomer for the Curable Resin

Many different monomers have been used for the resin, including alkanediol acrylates or methacrylates, polyalkyleneglycol acrylates or methacrylates, bisphenol an acrylate or methacrylate esters, alkoxylated bisphenol an acrylate or methacrylate, methacrylate-terminated polyurethanes, and combinations thereof.

In some embodiments, the polymerizable resin composition contains methacrylate, acrylate, vinyl, or other groups capable of free-radical polymerization.

In some embodiments, the polymerizable resin composition comprises monomer selected from the group consisting of C4-C10 alkyl methacrylate, C4-C10 alkyl acrylate, methyl methacrylate, ethyl methacrylate, styrene methacrylate, 2-vinyl pyrrolidinone, propyl methacrylate, hexyl methacrylate, acrylic acid (AA), vinyl acetate, vinyl acetic acid, mono-2-(methacryloyloxy)ethyl succinate, methacrylic acid (MAA), (polyethylene glycol) methacrylate, ethylene glycol dimethacrylate (EGDMA), 1,3-butylene glycol dimethacrylate (BGDMA), 1,4-butane diol diacrylate (BDDA), 1,6-hexane diol diacrylate (HDDA), isooctyl acrylate (2-EHA), tri(propylene glycol) diacrylate, hexanediol dimethacrylate (HDDMA), 1-carboxy-N-methyl-N-di(2-methacryloyloxy-ethyl) methanaminium inner salt (CBMX), (meth)acrylamides, neopentylglycol diacrylate (NPGDA), trimethylolpropane ethoxylate triacrylate (TMPTA), Acrylic acid 2-(2-acryloyloxy-ethoxymethyl)-2-acryloyloxymethyl-butyl ester (EO-TMPTA), acrylonitrile, methacrylonitrile, vinylidene cyanide, vinyl acetate, vinyl propionate, styrene, alpha-methylstyrene, maleic anhydride, ethoxylated bisphenol A acrylate or methacrylate ester, bisphenol A diglycidyl dimethacrylate (BisGMA), urethane dimethacrylate (UDMA), triethylene glycol dimethacrylate (TEGDMA), and combinations thereof. Ethoxylated bisphenol A acrylate or methacrylate ester is a compound having formula

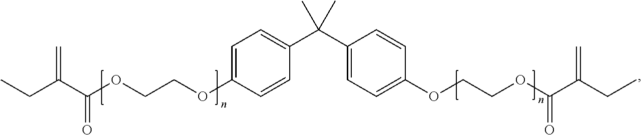

when n is 6 (BisEMA6).

In some embodiments, the monomers suitable for bone cement applications are selected from the group consisting of MAA, a mixture of MMA and acrylic acid (AA) (MMA+AA), 2-hydroxyethyl methacrylate (HEMA), a mixture of bisGMA, EGDMA and MMA, and a methacrylated amino acid containing anhydride oligomer as a reaction product of maleic acid, alanine and 6-aminocaproic acid and TEGMDA, and combinations thereof.

In some embodiments, the monomer composition suitable for cure dental composition application includes a mixture of BisEMA6, BisGMA, UDMA and TEGMDA.

In some embodiments, the polymerizable resin composition comprises a C1-C16 alkyl methacrylate, C1-C16 alkyl acrylate, C1-C16 acrylamide, and combinations thereof. In some embodiments, the polymerizable resin composition comprises hydrophilic monomer selected from the group consisting of hydroxymethacrylate (HEMA), hydroxyethyl methacrylate, 2-hydroxypropyl methacrylate, 2-hydroxyethyl acrylate, 2-hydroxypropyl acrylate, 3-hydroxypropyl acrylate, diethylene glycol monomethacrylate, hydroxyacrylate, glycerol dimethacrylate, glycol monomethacrylate, polyethylene glycol monomethacrylate, propylene glycol monomethacrylate, oligopropylene glycol monomethacrylate, hydroxypropyl methacrylate, polypropylene glycol monomethacrylate, hydroxyethyl-methacrylate, glycerol diacrylate, 2-tert-butylaminoethyl methacrylate, the reaction product of methacrylic acid and propylene oxide, 2-tert-butylaminoethyl methacrylate, polyethylene glycol 400 dimethacrylate, polyethylene glycol 600 dimethacrylate, polyethylene glycol 400 diacrylate, PEG 1,000 dimethacrylate, polypropylene glycol dimethacrylate, triethylene glycol di(meth)acrylate, dimethacrylates, diacrylates, monomethacrylates, monoacrylates, dipropylene glycol monomethacrylate, dipropylene glycol monoacrylate, acrylamide, methacrylamide, methylolacrylamide, methylolmethacrylamide, diacetone acrylamide, N-methylacrylamide, N-ethylacrylamide, N-hydroxyethyl acrylamide, N,N-substituted acrylamides, N,N-dimethylacrylamide, N,N-diethylacrylamide, N-ethylmethylacrylamide, N,N-dimethylolacrylamide, N-pyrrolidone, N-vinyl piperidone, N-acryloylpyrriolidone, N-acryloylpiperidine, N-acryloylmorpholene, N-vinyl pyrrolidinone, N-vinyl caprolactam, N-vinyl acetate, and combinations thereof.

In some embodiments, the monomer comprises one or more polymerizable prepolymer selected from the group consisting of polyethylene glycol 400 dimethacrylate, polyethylene glycol 600 dimethacrylate, polyethylene glycol 400 diacrylate; PEG 1,000 dimethacrylate, polypropylene glycol dimethacrylate, polyethylene glycol diacrylate, acrylated gelatin, collagen acrylate, acrylated alginate, and combinations thereof.

In an embodiment, this disclosure provides remotely-triggered curable dental compositions comprising 70.0-90.0 wt. % of a filer, 10.0-30.0 wt. % of a curable resin, 1 wt. % to 10 wt. % of a particle heater, an polymerization initiator, and a contrast agent, wherein the curable resin comprises 15.0 wt. % to 45.0 wt. % of BisEMA6, 15.0 wt. % to 45.0 wt. % of UDMA, 10.0 wt. % to 40.0 wt. % of BisGMA, and 0 wt. % to 10.0 wt. % of TEGDMA.

The curable resin suitable for the dental composition disclosed herein is described in U.S. Pat. No. 6,030,606, the content incorporated hereby by reference in its entirety. In some embodiments, the curable resin comprises 30.0 wt. % to 40.0 wt. % of BisEMA6, 30.0 wt. % to 40.0 wt. % of UDMA, 20.0 wt. % to 30.0 wt. % of BisGMA, and 0 wt. % to 10.0 wt. % of TEGDMA.

In some embodiments, the curable resin comprises 33.0 wt. % to 37.0 wt. % of BisEMA6, 33.0 wt. % to 37.0 wt. % of UDMA, 23.0 wt. % to 27.0 wt. % of BisGMA, and 0 wt. % to 5.0 wt. % of TEGDMA.

(ii) Filler for in situ Curable Dental Composition

A desirable property for dental composites is durability. Durability sometimes can be improved by increasing the percentage of filler particles in the composite.

In some embodiments, the filler is an inorganic filler selected from the group consisting of quartz; nitrides; glasses derived from Ce, Sb, Sn, Zr, Sr, Ba or Al; a composite glass composed of oxides of barium, silicon, boron, and aluminum; colloidal silica; feldspar; borosilicate glass; kaolin; talc; titania; zinc glass; zirconia-silica; fluoroaluminosilicate glass; submicron silica particles (e.g., pyrogenic silica such as the “Aerosil®” Series “OX 50”, “130”, “150” and “200” silica sold by Degussa and “Cab-O-Sil® M5” silica sold by Cabot Corp.), and combinations thereof.

An example of the composite glass has 67% SiO₂, 16.4% BaO, 10% B₂O₃ and 6.6% Al₂O₃, wherein the % is mole percent.

In some embodiments, the filler is an organic filler selected from the group consisting of filled or unfilled pulverized polycarbonates, and polyepoxides.

In some embodiments, the surface of the fillers may be treated with a surface treatment, such as a silane-coupling agent, in order to enhance the bond between the filler and the polymerizable resin. The coupling agent may be functionalized with reactive curing groups, such as acrylates, methacrylates, and the like.

In some embodiments, the filler comprises sintered ceramic composite of zirconia-silica. In some embodiments, the sintered ceramic composite of zirconia-silica comprises submicron particles having median particle size of 600 nm to 900 nm. In some embodiments, the sintered ceramic composites of zirconia-silica are amorphous, substantially crystalline or a mixture of amorphous, and crystalline oxide. In some embodiments, the sintered ceramic composites have a crystallinity index of less than about 0.1. In some embodiments, the dental fillers have a crystallinity index of less than about 0.05. In some embodiments, the sintered ceramic composites have a refractive index less than 1.60.

(iii) Contrast Agent

In some embodiments, the filler can be radiopaque, radiolucent or non-radiopaque. Radiopacity is a very desirable property for dental composites. Radiopaque composites can be examined using standard dental X-ray equipment, thereby facilitating long-term detection of marginal leakage or caries in tooth tissue adjacent to the cured composite. However, a dental composite should also have low visual opacity, that is, it should be substantially transparent or translucent to visible light. Low visual opacity is desired so that the cured dental composite will have a lifelike luster.

In some embodiments, the in situ curable dental composition further comprises a radiopacifying agent. In some embodiments, the radiopacifying agent comprises a polycrystalline ceramic metal oxide. In some embodiments, the radiopacifying agent is selected from the group consisting of HfO₂, La₂O₃, SrO, ZrO₂, and combinations thereof.

### (iv) Heat Dissipating Agent

The increase in temperature of the composition due to exothermic polymerization of the monomeric component may be as low as 5° C. and as high as 70° C., depending on the monomer and initiator utilized. A temperature increase of as little as 40° C. of the curable dental composition placed on the surface of living tissue will generally cause necrosis or thermal damage. Temperature increases of lesser amounts will generally cause discomfort and irritation of the tissue. In order to minimize these problems, heat-dissipating agents are introduced into the curable dental composition. The heat dissipating agents include liquids or solids that may be soluble or insoluble in the monomer.

In some embodiments, the curable dental composition additionally comprises a heat-dissipating agent to reduce temperature increase during the exothermic polymerization of the curable dental composition. In some embodiments, the heat dissipating agent is selected from the group consisting of a volatile liquid, a solid having a melting point of from about 20° C. to about 150° C., and a solid having a sublimation point of from about 20° C. to about 150° C.

In some embodiments, solids that act as a heat sink or that readily adsorb heat may be utilized. Suitable heat-adsorbing substances include alkaline metal oxide such as aluminum oxide, barium oxide, titanium oxide, manganese oxide and calcium oxide; metal nanoparticles such as copper, lead, nickel, aluminum, and zinc, carbon black and carbides; organic compounds such as urea, paraffin wax and polyvinyl fluoride; and salts such as ammonium nitrate, potassium nitrate, sodium acetate trihydrate, sodium sulfate decahydrate (Glauber's salt), barium hydroxide octahydrate, calcium oxalate dihydrate, magnesium oxalate dihydrate, aluminum hydroxide, ammonium sulfate, zinc sulfate, and ammonium phosphate.

In some embodiments, the heat dissipating agent is selected from the group consisting of potassium nitrate, sodium acetate trihydrate, sodium sulfate decahydrate, barium hydroxide octahydrate, calcium oxalate dihydrate, magnesium oxalate dihydrate, aluminum hydroxide, zinc sulfate, aluminum oxide, barium oxide, titanium oxide, manganese oxide, calcium oxide, metal nanoparticles such as copper, lead, nickel, aluminum, and zinc, carbon black and carbides, graphene nanoparticle, graphene oxide nanoparticle, urea, paraffin wax and polyvinyl fluoride, poly(N-isopropylacrylamide) (PNIPAAm) composite incorporating glycidyl methacrylate functionalized graphene oxide (GO-GMA), 2-hydroxy-2-trimethylsilanyl-propionitrile, 1-fluoropentacycloundecane, 6,7-diazabicyclo[3.2.1]oct-6-ene, 5,5,6,6-tetramethylbicyclo[2.2.1]heptan-2-ol, N-benzyl-2,2,3,3,4,4,4-heptafluoro-butyramide, 3-isopropyl-5,8a-dimethyl-decahydronaphthalen-2-ol, 2-hydroxymethyl-1,7,7-trimethyl-bicyclo[2.2.1]heptan-2-ol, 3,5-dichloro-3-methyl-cyclopentane-1,2-dione, (5-methyl-2-oxo-bicyclo[3.3.1]non-3-en-1-yl)-acetic acid, 4b,6a,11,12-tetrahydro-indeno[2,1-a]fluorene-5,5,6,6-tetracarbonitrile, tetracosafluoro-tetradecahydro-anthracene, 4,5-dichlorobenzene-1,2-dicarbaldehyde, bicyclo[4,3.1]dec-3-en-8-one, 3-tert-butyl-1,2-bis-(3,5-dimethylphenyl)-3-hydroxyguanidine, 1-[2,6-dihydroxy-4-methoxy-3-methylphenyl]butan-1-one, 2,3,6,7-tetrachloronaphthalene, 2,3,6-trimethylnaphthalene, dodecafluoro-cyclohexane, 2,2,6,6-tetramethyl-4-hepten-3-one, 1,1,1-trichloro-2,2,2-trifluoro-ethane, [5-(9H-beta-carbolin-1-yl)-furan-2-yl]methanol, 5-nitro-benzo[1,2,3]thiadiazole, 4,5-dichloro-thiophene-2-carboxylic acid, 2,6-dimethyl-isonicotinonitrile, nonafluoro-2,6-bis-trifluoromethyl-piperidine, (dimethylamino)difluoroborane, dinitrogen pentoxide, chromyl fluoride, chromium hexacarbonyl, 1-methylcyclohexanol, phenyl ether, nonadecane, 1-tetradecanol, 4-ethylphenol, benzophenone, maleic anhydride, octacosane, dimethyl isophthalate, butylated hydroxytoluene, glycolic acid, vanillin, magnesium nitrate hexahydrate, cyclohexanone oxime, glutaric acid, D-sorbitol, phenanthrene, fluorene, trans-stilbene, neopentyl glycol, pyrogallol, and diglycolic acid, and combinations thereof.

In some embodiments, the temperature reduction caused by incorporating the dissipating agent is of about 1° C. to 70° C. In some embodiments, the temperature reduction as a result of incorporating the dissipating agent is selected from the group consisting of about 1° C., about 5° C., about 10° C., about 15° C., about 20° C., about 25° C., about 30° C., about 35° C., about 40° C., about 45° C., about 50° C., about 55° C., about 60° C., about 65° C., and about 70° C.,

### 7. Remotely-Triggered In Situ Curing of Bone Cement

Several of the drawbacks of the PMMA based bone cements are reported in many publications. One well-known drawback of the acrylic bone is its inflammatory or genotoxic effects, with the toxic effects on the cells being directly proportional to the initial amount of polymerization initiator BPO. This toxicity may be mediated by free radicals, whose release from the cement is long-lived event.

The polymerization of acrylic bone cement is exothermic, with the maximum polymerization temperature (typically, 70-110° C.) being high enough that thermal necrosis of the periprosthetic tissue may occur. Thermochemical initiators such as benzoyl peroxide (BPO) are commonly used in curable bone cement composition. The rates of decomposition follow first-order reaction kinetics and are accelerated at elevated temperature. In principle, the higher the reaction temperature is, the faster the polymerization should proceed. In practice, however, if the reaction temperature is set too high, the rapid decomposition of the initiator generates high concentration of propagating radicals in a reaction mixture, which accelerate termination more than propagation.

In some embodiments, heat-dissipating agents are introduced into the curable bone cement composition to minimize the problem of high polymerization temperature. The heat dissipating agents include liquids or solids that may be soluble or insoluble in the monomer. In some embodiments, the in situ curable bone cement additionally comprises a heat-dissipating agent to reduce temperature increase during the exothermic polymerization of the bone cement.

In some embodiments, the heat dissipating agent is selected from the group consisting of a volatile liquid, a solid having a melting point of from about 20° C. to about 150° C., and a solid having a sublimation point of from about 20° C. to about 150° C. In some embodiments, the heat-dissipating agent is at least one substance and the use amount ranges as described in the curable dental composition section above. In some embodiments, the heat-dissipating agent may be included in the solid phase or liquid phase of the curable bone cement.

Further, the radiopacifier BaSO₄ and ZrO₂ particles are known to have harmful effects on causing differentiation of macrophage into bone absorbing osteoclasts (contributing to bone resorption), evoking significant pathological response on the periprosthetic zone, releasing of inflammatory of factors, and increase of debris.

Moreover, acrylic bone cement is not bioactive. This means that formation of an excellent interface with the cancellous bone does not occur with the cement. The interface with the cancellous bone is important for enhancing mechanical fixation and biological performance. There exists a need for controlled radical polymerization for the curing of the monomer based curable composition, e.g., controlling the concentration of the propagating radical by controlling the rate of radical species generation in the monomer mixture.

Reactive oxygen species (ROS) are emerging as important elements in the biological response to lethal stress. The biological bodies contain protective proteins (catalase/peroxidases) that can detoxify ROS and counter damage. There are three naturally occurring ROS species: singlet oxygen (¹O₂), hydrogen peroxide (H₂O₂), and hydroxyl radical (.OH). Superoxide and hydrogen peroxide arise when molecular oxygen adventitiously oxidizes redox enzymes that normally transfer electrons to other substrates. Hydrogen peroxide, which can also be produced from dismutase of superoxide, serves as a substrate for .OH formation through Fenton chemistry. This oxidative process can kill cells if hydroxyl radical accumulation is not controlled, since hydroxyl radical breaks nucleic acids, carboxylates proteins, and peroxidizes lipids. The ROS pathway can be blocked with iron chelators and antioxidant treatment by inhibiting catalase/peroxidase activity.

Photothermal processes employ NIR light induced localized hyperthermia to cause cytotoxic effects on tissue cells (e.g., apoptosis or necrosis depending on the laser dosage, type and irradiation duration).

Photodynamic processes involve the use of photosensitizing agent to generate ROS under appropriate light irradiation.

This disclosure provides a bone cement having heat-generating particles as an additive to address several of the drawbacks of the conventional bone cement composition. This disclosure also provides a method of controlling the radical polymerization of the curable bone cement composition by replacing all or parts of the thermochemoinitiator BPO with the particle heaters capable of photodynamic process.

For example, the particle can be activated on demand to produce propagating radical species from the ROS resulting from a photodynamic process.

In an additional embodiment, this disclosure provides hyperthermia as an adjuvant treatment for the bone healing process via the hyperthermia induced ROS protective response. In some embodiments, the particle heater can be activated on demand to induce localized mild to moderate hyperthermia within the cured cement after implantation. Mild hyperthermia induces bone healing by promoting osteogenesis and formation of new bone (See Cao et al., Science China Life Sci., 2018, p. 1-9).

In an embodiment, this disclosure provides an in situ curable bone cement comprising: a solid phase comprising PMMA powder, a contrast agent and a polymerization initiator, and a liquid comprising methyl-methacrylate monomer (MMA), an accelerator, and a polymerization inhibitor; wherein the polymerization initiator capable of generating free radicals to catalyze the in situ polymerization of MMA monomer to provide a cured bone cement.

In some embodiments, the polymerization initiator is a particle having a carrier and a material interacting with an exogenous source, and wherein the particle is constructed such that it passes the Extractable Cytotoxicity Test. In some embodiments, the particle further passes the Efficacy Determination Protocol.

In some embodiments, the material absorbs the energy from the exogenous source and causes the production of reactive oxygen species. In some embodiments, the accelerator is a divalent iron salt, wherein the divalent iron ion catalyzes the ROS degradation to hydroxyl free radical.

The in situ curable bone cement containing the particle heaters disclosed herein is also useful for the treatment of bone metastasis in a patient having cancer.

In some embodiments, pulsed laser irradiation is employed to induce a photodynamic process. In some embodiments, pulsed laser irradiation is employed to induce a photothermal process. In some embodiments, the laser is operated at a wavelength of 805 nm or 1064 nm and at fluences of 10-100 J/cm² with pulse durations ranging from about 10 μs to about 400 ms.

(i) Solid phase

(a) Polymer for the Solid Phase

In some embodiments, the solid phase comprises a solid polyacrylate. In some embodiments, the polyacrylate includes all polymers and copolymers of acrylic acid and acrylic acid esters that are suitable for bone cements that include an acrylate monomer listed herein below. In some embodiments, the ester is derived from an aliphatic C1-C6 alcohol. More preferably, the ester is the methyl ester. A polymerizable acrylate monomer as used herein is defined to include a methacrylate or acrylate monomer having at least one unsaturated double bond. Suitable polymerizable acrylate monomer for this disclosure includes methyl methacrylate, ethyl methacrylate, isopropyl methacrylate, 2-hydroxyethyl methacrylate, n-butyl methacrylate, isobutyl methacrylate, 3-hydroxypropyl methacrylate, tetrahydrofurfuryl methacrylate, glycidyl methacrylate, 2-methoxyethyl methacrylate, 2-ethylhexyl methacrylate, benzyl methacrylate, 2,2-bis(methacryloxyphenyl)propane, 2,2-bis[4-(2-hydroxy-3-metltacryloxypropoxy)phenyl]propane, 2,2-bis(4-memacryloxypolyethoxyl-phenyl)propane, ethylene glycol dimethacrylate, diethylene glycol dimethacrylate, triethylene glycol dimethacrylate, butylene glycol dimethacrylate, neopentyl glycol dimethacrylate, 1,3-butanediol dimethacrylate, 1,4-butanediol dimethacrylate, 1,6-hexanediol dimethacrylate, trimethylolpropane trimethacrylate, trimetliylolethane trimethacrylate, pentaerythritol trimethacrylate, trimethylolmethane trimethacrylate and pentaerythritol tetramethaciylate, and methacrylates and acrylates having urethane bonds therein. Specific urethane including acrylates include di-2memacryloxyemyl-2,2,4-trimethylhexamethylene dicarbomate and its acrylate.

In some embodiments, the polymer in the solid phase is selected from the group consisting of polymethylmethacrylate (PMMA), poly(hydroxyalkenoate), poly([R]-3-hydroxybutyrate (PHB), poly(ethyl methacrylate) n-butyl methacrylate (PEM-BMA), PMMA-graft-PHB, cornstarch and cellulose acetate (SCA), SCA reinforced hyaluronic acid (HA), HA particles silanized with 3-(triethoxysilyl)propyl methacrylate, poly(MMA-co-EMA), and combinations thereof.

In some embodiments, the polymer is in the form of a powder having a median particle size of about 10 μm to about 100 μm. In some embodiments, the polymer powder has a median particle size of about 10 μm to about 60 μm. In some embodiments, the polymer powder has a median particle size of about 50 μm to about 60 μm, and a relative particle size distribution of about 10 μm to about 140 μm. In some embodiments, the polymer powder has a median particle size selected from the group consisting of about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, and about 100 μm.

In some embodiments, the polyacrylate in the solid phase has a weight percent ranging from about 80.0 wt. % to about 99.0 wt. % by the total weight of the liquid phase. In some embodiments, the polyacrylate in the solid phase has a weight percent ranging from about 83.0 wt. % to about 99.0 wt. % by the total weight of the liquid phase. In some embodiments, the monomers in the liquid phase has a weight percent by the total weight of the liquid phase selected from the group consisting of about 80.0 wt. %, about 80.1 wt. %, about 80.2 wt. %, about 80.3 wt. %, about 80.4 wt. %, about 80.5 wt. %, about 80.6 wt. %, about 80.7 wt. %, about 80.8 wt. %, about 80.9 wt. %, about 81.0 wt. %, about 81.1 wt. %, about 81.2 wt. %, about 81.3 wt. %, about 81.4 wt. %, about 81.5 wt. %, about 81.6 wt. %, about 81.7 wt. %, about 81.8 wt. %, about 81.9 wt. %, about 82.0 wt. %, about 82.1 wt. %, about 82.2 wt. %, about 82.3 wt. %, about 82.4 wt. %, about 82.5 wt. %, about 82.6 wt. %, about 82.7 wt. %, about 82.8 wt. %, about 82.9 wt. %, about 83.0 wt. %, about 83.1 wt. %, about 83.2 wt. %, about 83.3 wt. %, about 83.4 wt. %, about 83.5 wt. %, about 83.6 wt. %, about 83.7 wt. %, about 83.8 wt. %, about 83.9 wt. %, about 84.0 wt. %, about 84.1 wt. %, about 84.2 wt. %, about 84.3 wt. %, about 84.4 wt. %, about 84.5 wt. %, about 84.6 wt. %, about 84.7 wt. %, about 84.8 wt. %, about 84.9 wt. %, about 85.0 wt. %, about 85.1 wt. %, about 85.2 wt. %, about 85.3 wt. %, about 85.4 wt. %, about 85.5 wt. %, about 85.6 wt. %, about 85.7 wt. %, about 85.8 wt. %, about 85.9 wt. %, about 86.0 wt. %, about 86.1 wt. %, about 86.2 wt. %, about 86.3 wt. %, about 86.4 wt. %, about 86.5 wt. %, about 86.6 wt. %, about 86.7 wt. %, about 86.8 wt. %, about 86.9 wt. %, about 87.0 wt. %, about 87.1 wt. %, about 87.2 wt. %, about 87.3 wt. %, about 87.4 wt. %, about 87.5 wt. %, about 87.6 wt. %, about 87.7 wt. %, about 87.8 wt. %, about 87.9 wt. %, about 88.0 wt. %, about 88.1 wt. %, about 88.2 wt. %, about 88.3 wt. %, about 88.4 wt. %, about 88.5 wt. %, about 88.6 wt. %, about 88.7 wt. %, about 88.8 wt. %, about 88.9 wt. %, about 89.0 wt. %, about 89.1 wt. %, about 89.2 wt. %, about 89.3 wt. %, about 89.4 wt. %, about 89.5 wt. %, about 89.6 wt. %, about 89.7 wt. %, about 89.8 wt. %, about 89.9 wt. %, about 90.0 wt. %, about 90.1 wt. %, about 90.2 wt. %, about 90.3 wt. %, about 90.4 wt. %, about 90.5 wt. %, about 90.6 wt. %, about 90.7 wt. %, about 90.8 wt. %, about 90.9 wt. %, about 91.0 wt. %, about 91.1 wt. %, about 91.2 wt. %, about 91.3 wt. %, about 91.4 wt. %, about 91.5 wt. %, about 91.6 wt. %, about 91.7 wt. %, about 91.8 wt. %, about 91.9 wt. %, about 92.0 wt. %, about 92.1 wt. %, about 92.2 wt. %, about 92.3 wt. %, about 92.4 wt. %, about 92.5 wt. %, about 92.6 wt. %, about 92.7 wt. %, about 92.8 wt. %, about 92.9 wt. %, about 93.0 wt. %, about 93.1 wt. %, about 93.2 wt. %, about 93.3 wt. %, about 93.4 wt. %, about 93.5 wt. %, about 93.6 wt. %, about 93.7 wt. %, about 93.8 wt. %, about 93.9 wt. %, about 94.0 wt. %, about 94.1 wt. %, about 94.2 wt. %, about 94.3 wt. %, about 94.4 wt. %, about 94.5 wt. %, about 94.6 wt. %, about 94.7 wt. %, about 94.8 wt. %, about 94.9 wt. %, about 95.0 wt. %, about 95.1 wt. %, about 95.2 wt. %, about 95.3 wt. %, about 95.4 wt. %, about 95.5 wt. %, about 95.6 wt. %, about 95.7 wt. %, about 95.8 wt. %, about 95.9 wt. %, about 96.0 wt. %, about 96.1 wt. %, about 96.2 wt. %, about 96.3 wt. %, about 96.4 wt. %, about 96.5 wt. %, about 96.6 wt. %, about 96.7 wt. %, about 96.8 wt. %, about 96.9 wt. %, about 97.0 wt. %, about 97.1 wt. %, about 97.2 wt. %, about 97.3 wt. %, about 97.4 wt. %, about 97.5 wt. %, about 97.6 wt. %, about 97.7 wt. %, about 97.8 wt. %, about 97.9 wt. %, about 98.0 wt. %, about 98.1 wt. %, about 98.2 wt. %, about 98.3 wt. %, about 98.4 wt. %, about 98.5 wt. %, about 98.6 wt. %, about 98.7 wt. %, about 98.8 wt. %, about 98.9 wt. %, and about 99.0 wt. %.

(b) Polymerization Initiator

In some embodiments, the curable hard tissue composition further comprises additives such as polymerization initiators to generate radicals for initiating the polymerization reactions. The in situ curing of the curable bone cement occurs via free-radical polymerization of a polymerizable precursor initiated by radicals generated by the polymerization initiator.

In some embodiments, the polymerization initiator helps to start the free radical polymerization of the polymerizable monomer via a free radical polymerization reaction between the monomers. The in situ curing of the curable bone cement take places after mixing the solid and liquid phases. The kinetics of the free-radical polymerization reaction are regulated by the concentrations and mobility of the initiator and the accelerator.

In some embodiments, the polymerization initiator is selected from the group consisting of benzoyl oxide (BPO), tri-n-butyl borane, 2-5-dimethylhexane-2-5-dihydroperoxide, the particle heater, 2,2′-azobis [2-(2-imidazolin-2-yl) propane] dihydrochloride, 2,2′-azobis [2-(2-imidazolin-2-yl) propane] disulfate dihydrate, 2,2′-azobis (2-methyl propionic amidine) dihydrochloride, 4,4′-azobis (4-cyano valeric acid), camphorquinone-10-sulfonic acid and its salts, camphorquinone 3-oximes, anti-(1R)-(+)-camphorquinone 3-oxime, anti-(1S)-(−)-camphorquinone 3-oxime, the addition reaction product of anti-(1R)-(+)-camphorquinone 3-oxime, anti-(1S)-(−)-camphorquinone 3-oxime with an organic anhydride, a dianhydride, a camphorquinone, a peroxide, a mixture of horseradish peroxidase and hydrogen peroxide, and combinations thereof.

In some embodiments, the polymerization initiator is selected from the group consisting of 2,2′-azobis [2-(2-imidazolin-2-yl) propane] dihydrochloride, 2,2′-azobis [2-(2-imidazolin-2-yl) propane] disulfate dihydrate, 2,2′-azobis (2-methyl propionic amidine) dihydrochloride, 4,4′-azobis (4-cyano valeric acid), and combinations thereof.

In some embodiments, the polymerization initiator is BPO. In some embodiments, the polymerization initiator comprises BPO and the remotely triggered particles. In some embodiments, the polymerization initiator comprises remotely triggered particle and hydrogen peroxide.

The term “Fenton chemistry” as used herein, generally refers to the nonenzymatic reaction of Fe²⁺ with H₂O₂. Fe²⁺ is oxidized by hydrogen peroxide to Fe³⁺, forming OH. and OH— in the reaction. Fe³⁺ is then reduced back to Fe²⁺ by another molecule of H₂O₂, forming a hydroperoxyl radical .OOH and a proton H. The net effect is a disproportionation of hydrogen peroxide to create two different oxygen-radical species, with water as a byproduct. Iron and hydrogen peroxide are capable of oxidizing a wide range of substrates and causing biological damage. The Fenton reaction is a reaction of importance in the oxidative stress in blood cells and various tissues.

In some embodiments, the polymerization initiator is in an amount ranging from about 0.1 wt. to about 3.0 wt. % by the total weight of the in situ curable bone cement. In some embodiments, the polymerization initiator is in an amount ranging from about 0.75 wt. % to about 2.6 wt. % by the total weight of the in situ curable bone cement. In some embodiments, the polymerization initiator is in an amount ranging from about 0.8 wt. % to about 1.4 wt. % by the total weight of the in situ curable bone cement. In some embodiments, the polymerization initiator is in a weight percent by the total weight of the in situ curable bone cement selected from the group consisting of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, and about 3.0 wt. %.

In some embodiments, the polymerization initiator in the liquid formulation is at a concentration ranging from about 1.0 mg/mL to about 20.0 mg/mL. In some embodiments, the polymerization initiator is at a concentration selected from about 1.0 mg/mL, about 2.0 mg/mL, about 3.0 mg/mL, about 4.0 mg/mL, about 5.0 mg/mL, about 6.0 mg/mL, about 7.0 mg/mL, about 8.0 mg/mL, about 9.0 mg/mL, about 10.0 mg/mL, about 11.0 mg/mL, about 12.0 mg/mL, about 13.0 mg/mL, about 14.0 mg/mL, about 15.0 mg/mL, about 16.0 mg/mL, about 17.0 mg/mL, about 18.0 mg/mL, about 19.0 mg/mL, and about 20.0 mg/mL.

### (ii) Liquid Phase

(a) Monomer in the Liquid Phase

In some embodiments, the monomer in the liquid phase is selected from the group consisting of methyl-methacrylate monomer (MMA), lysineurethanedimethacrylate (LUDM), n-butyl methacrylate, ethyl methacrylate, isopropylmethacrylate, 3-hydroxypropyl methacrylate, tetrahydrofurfuryl methacrylate, glycidyl methacrylate, 2-methoxyethyl methacrylate, 2-ethylhexyl methacrylate, benzyl methacrylate, 2,2-bis(methacryloxyphenyl)propane, 2,2-bis[4-(2-hydroxy-3-methacryloxypropoxy)phenyl]propane, 2,2-bis(4-methacryloxypolyethoxylphenyl)propane, ethylene glycol dimethacrylate, diethylene glycol dimethacrylate, triethylene glycol dimethacrylate, butylene glycol dimethacrylate, neopentyl glycol dimethacrylate, 1,3-butanediol dimethacrylate, 1,4-butanediol dimethacrylate, 1,6-hexanediol dimethacrylate, trimethylolpropane trimethacrylate, trimethylolethane trimethacrylate, pentaerythritol trimethacrylate, trimethylolmethane trimethacrylate and pentaerythritol tetramethacrylate, mixture of MMA and acrylic acid (AA) (MMA+AA), mixture of 80% MMA and 20% ethoxytriethylene glycol monomethacrylate (TEG), 2-hydroxyethyl methacrylate (HEMA), a mixture of bisGMA, EGDMA and MMA, and a methacrylated amino acid containing anhydride oligomer as a reaction product of maleic acid, alanine and 6-aminocaproic acid and TEGMDA, and combinations thereof. In some embodiments, the monomer in the liquid phase comprises MMA only. In some embodiments, the monomer in the liquid phase comprises lysineurethanedimethacrylate (LUDM) only. In some embodiments, the monomer in the liquid phase is a mixture of 80% MMA and 20% ethoxytriethylene glycol monomethacrylate (TEG).

In some embodiments, the monomers in the liquid phase has a weight percent ranging from about 95.0 wt. % to about 99.0 wt. % by the total weight of the liquid phase. In some embodiments, the monomers in the liquid phase has a weight percent ranging from about 97.0 wt. % to about 99.0 wt. % by the total weight of the liquid phase. In some embodiments, the monomers in the liquid phase has a weight percent by the total weight of the liquid phase selected from the group consisting of about 95.0 wt. %, about 95.1 wt. %, about 95.2 wt. %, about 95.3 wt. %, about 95.4 wt. %, about 95.5 wt. %, about 95.6 wt. %, about 95.7 wt. %, about 95.8 wt. %, about 95.9 wt. %, about 96.0 wt. %, about 96.1 wt. %, about 96.2 wt. %, about 96.3 wt. %, about 96.4 wt. %, about 96.5 wt. %, about 96.6 wt. %, about 96.7 wt. %, about 96.8 wt. %, about 96.9 wt. %, about 97.0 wt. %, about 97.1 wt. %, about 97.2 wt. %, about 97.3 wt. %, about 97.4 wt. %, about 97.5 wt. %, about 97.6 wt. %, about 97.7 wt. %, about 97.8 wt. %, about 97.9 wt. %, about 98.0 wt. %, about 98.1 wt. %, about 98.2 wt. %, about 98.3 wt. %, about 98.4 wt. %, about 98.5 wt. %, about 98.6 wt. %, about 98.7 wt. %, about 98.8 wt. %, about 98.9 wt. %, and about 99.0 wt. %.

### 8. Wound Closure Device

In an embodiment, this disclosure provides a wound closure device comprising a structural element and a heat delivery composition, wherein the heat delivery composition comprises a material interacting with an exogenous source, wherein the material absorbs the energy from the exogenous source and converts the absorbed energy to heat, wherein the heat causes the tightening of the suture, and wherein the wound closure device passes the Extractable Cytotoxicity Test. In some embodiments, the heat delivery composition further comprises a carrier.

In an embodiment, any of the heat delivery compositions described herein can be used to form the wound closure device. In some embodiments, the heat delivery composition is coated on, embedded within, crosslinked with, or otherwise associated with the structural element. In some embodiments, the heat delivery composition is a particle, which can be a nanoparticle, a microparticle, or combinations thereof.

In some embodiments, the structural element is configured as a suture, staple, screw, tape, patch, adhesive, sealant, or the like. In some embodiments, the structural element is configured as a suture selected from the group consisting of monofilament suture, braided multifilament suture, and combinations thereof.

In some embodiments, the wound closure device includes an antimicrobial agent in the structural element. In some embodiments, the wound closure device can include a scar reducing agent in the structural element.

In some embodiments, the structural element for the wound closure device is configured as a suture, staple, screw, patch, tape, adhesive, biological glue, or sealant. In an embodiment, the structural element for the wound closure device is configured as a suture. In an embodiment, the structural element for the wound closure device is configured as a staple. In an embodiment, the structural element for the wound closure device is configured as a patch. In an embodiment, the structural element for the wound closure device is configured as a tape. In an embodiment, the structural element for the wound closure device is configured as an adhesive. In an embodiment, the structural element for the wound closure device is configured as a screw. In an embodiment, the structural element for the wound closure device is configured as a sealant.

In some embodiments, the structural element for the wound closure device comprises a plurality of multifilament strands braided together. In some embodiments, the structural element for the wound closure device comprises a single strand of filament.

In some embodiments, the structural element for the wound closure device is configured as a braided multifilament suture. In some embodiments, the structural element for the wound closure device is configured as a braided multifilament suture having a heat delivery coating composition as described above. In some embodiments, the structural element for the wound closure device is configured as a braided multifilament suture having a heat delivery particles described above dispersed with the multifilament.

In some embodiments, the structural element for the wound closure device is configured as a single strand filament suture having a heat delivery coating composition described above dispersed with the filaments. In some embodiments, the structural element for the wound closure device is configured as a single strand filament suture coated with heat delivery particles dispersed in a film-forming agent as described above.

In some embodiments, the structural element for the wound closure device is biodegradable and/or bioabsorbable.

In some embodiments, the structural element for the wound closure device comprises a substance selected from the group consisting of gut, chromic gut, nylon, rayon, polyethylene, pluronic F127, chitosan, collagen, laminin, fibronectin, polyacrylamide, aminoglycoside hydrogels, fibrin, poly(lactic acid), poly(glycolic acid), poly(lactic-co-glycolic acid) (PLGA), polyglyconate, polydioxanone, poly(trimethylene carbonate), silk, poly-(glycolic acid-caprolactone), cotton, gelatin, polypropylene, titanium, metal, polysulfone, poly(ethylene terephthalate) (PETE), and combinations thereof. In some embodiments, the structural element for the wound closure device comprises homopolymers or copolymers of lactide, glycolide, β-hydroxybutylcarboxylic acid, β-propiolactone, γ-butyrolactone, γ-valero-3-methylbutyrolactone, δ-valerolactone, chitin, and ξ-caprolactone. In some embodiments, the structural element for the wound closure device comprises an absorbable polymer selected from the group consisting of homopolymers of lactide and glycolide, copolymers of lactide and glycolide, and combinations thereof.

The structural element is a suture and is made of a polymer selected from the group consisting of collagens, polydioxanone, polyesters, polyester-carbonates, polyethers, polyether-ester, and copolymers of thereof. In some embodiments, the suture is made of a bioabsorbable polymer selected from the group consisting of a homopolymer or copolymer of polyglycolide (PGA), poly(lactic-co-glycolic acid) (PLGA), poly(glycolide-co-trimethylene carbonate) (PGA-co-MC), poly(glycolide-co-caprolactone-co-trimethylene carbonate) (PGA-co-PCL-co-TMC), polyglycolic acid (PGA) and copolymers thereof, a polyhydroxyalkanoate (PHA), polyhydroxybutyrate (PHB), poly-4-hydroxybutyrate (P4HB), polyhydroxyvalerate (PHV), polyhydroxyhexanoate (PHH), polyhydroxyoctanoate (PHO) and their copolymers, polycaprolactone (PCL), and combinations thereof. In some embodiments, the suture is made of a bioabsorbable polyester. In some embodiments, the suture is made of a polymer that is degradable by hydrolysis or other biodegradation mechanisms and contains the following monomeric units of trimethylene carbonate, lactide, glycolide, ε-caprolactone, and para-dioxanone. In some embodiments, the suture is made of collagen. In some embodiments, the suture is made of PLGA. In some embodiments, the suture is made of polydioxanone.

In some embodiments, the suture comprises polymers that are susceptible to thermally induced shrinkage. In some embodiments, the polymers susceptible to thermally induced shrinkage are selected from the group consisting of collagen (e.g., Chromic Gut®), copolymer of polyglycolide and poly(ε-caprolactone) (Monocryl®, poliglecaprone 25), PLGA, polydioxanone (PDS® II suture), polycaprolactone, and combinations thereof. In some embodiments, the suture is made of collagen. In some embodiments, the suture is made of polydioxanone.

In some embodiments, the material interacting with the exogenous source forms a coating layer on the surface of a suture, wherein the suture is made of biodegradable polymers as disclosed herein. In some embodiments, the filament of the suture is made from the material interacting with the exogenous source admixed with the biodegradable polymers as disclosed herein. In some embodiments, the material interacting with the exogenous source and the carrier forms a particle and are dispersed within the biodegradable polymers as disclosed herein to form the filament of the suture. In some embodiments, the suture is a monofilament. In some embodiments, the suture comprises multiple filaments braided together.

In some embodiments, the structural element for the wound closure device comprises polydioxanone. In some embodiments, the structural element for the wound closure device comprises chromic gut. In some embodiments, the structural element for the wound closure device comprises poly-lactic-co-glycolic acid (PLGA). In some embodiments, the structural element for the wound closure device comprises a copolymer made from 90% glycolide and 10% L-lactide. In some embodiments, the structural element for the wound closure device comprises polyethylene terephthalate (PETE) (sold under the tradename Dacron®).

In some embodiments, the structural element is configured as a gelatin fiber having a tetrakis aminium dye dispersed within the gelatin fiber. In some embodiments, the structural element is configured as a collagen fiber having a tetrakis aminium dye dispersed within. In some embodiments, the structural element is configured as a PLGA fiber having a tetrakis aminium dye dispersed within.

In some embodiments, the structural element is configured as a PLGA fiber having an indocyanine green dye dispersed within. In some embodiments, the structural element is configured as a gelatin fiber having an indocyanine green dye dispersed within the gelatin fiber. In some embodiments, the structural element is configured as a collagen fiber having an indocyanine green dye dispersed within.

In some embodiments, the structural element is configured as a PLGA fiber having an IR 193 dye dispersed within. In some embodiments, the structural element is configured as a gelatin fiber having an IR 193 dye dispersed within the gelatin fiber. In some embodiments, the structural element is configured as a collagen fiber having an IR 193 dye dispersed within.

In some embodiments, the structural element is configured as a gelatin fiber with Epolight™ 1117 IR dye dispersed within. In some embodiments, the structural element is configured as a collagen fiber with Epolight™ 1117 IR dye dispersed within. In some embodiments, the structural element is configured as a PLGA fiber with Epolight™ 1117 IR dye dispersed within.

In some embodiments, the structural element is configured as a gelatin fiber having the heat delivery particles as described above dispersed within. In some embodiments, the structural element is configured as a collagen fiber having the heat delivery particles as described above dispersed within. In some embodiments, the structural element is configured as a PLGA fiber having the heat delivery particles as described above dispersed within.

### Photothermal Suturing

In an embodiment, this disclosure provides remotely-triggered wound closure devices. Wound closure devices such as sutures, staples and tacks have been widely used in superficial and deep surgical procedures in humans and animals for closing wounds, repairing traumatic injuries or defects, joining tissues together (bringing severed tissues into approximation, closing an anatomical space, affixing single or multiple tissue layers together, creating an anastomosis between two hollow/luminal structures, adjoining tissues, attaching or reattaching tissues to their proper anatomical location), attaching foreign elements to tissues (affixing medical implants, devices, prostheses and other functional or supportive devices), and for repositioning tissues to new anatomical locations (repairs, tissue elevations, tissue grafting and related procedures).

In some embodiments, the remotely-triggered wound closure device is a suture comprising a material, wherein the material is a light (e.g. 700 nm to 1400 nm wavelength) absorbing material including organic dyes such as tetrakis aminium dye including Epolight™ 1117 (maximum absorbance at 1064 nm), cyanine dyes including indocyanine green (maximum absorbance at 805 nm), and gold nanorods (maximum absorbance ranging from 700 nm to 1300 nm). These light absorbing materials absorb in the optical spectrum of 700 nm to 1350 nm light wavelength and convert the laser energy into heat.

In some embodiments, the remotely-triggered wound closure devices are staples comprising a material, wherein the material is a light (e.g. 700 nm to 1400 nm wavelength) absorbing material including organic dyes such as tetrakis aminium dye including Epolight™ 1117 (maximum absorbance at 1064 nm), cyanine dyes including indocyanine green (maximum absorbance at 805 nm), and gold nanorods (maximum absorbance ranging from 700 nm to 1300 nm). These light absorbing materials absorb in the optical spectrum of 700 nm to 1350 nm light wavelength and convert the laser energy into heat.

The wound closure devices described herein have high flexibility, high tensile strength, suppleness, and controlled degradability. The use of a certain wound closure device and the closure technique depend on the kind of tissue and the wound. The exact repositioning of the tissue interfaces is important to obtain the best wound healing. One of ordinary skill in the art would know which suture to use.

In some embodiments, the method of remotely-triggered suture tightening further includes a step of cooling the tissue area before applying laser pulse. In some embodiments, the pulsed laser system is used together with a dynamic cooling device (DCD) in the method of heat assisted suture tightening as disclosed herein. In some embodiments, the method includes a step of spraying the skin with a brief application of cryogen prior to the laser irradiation to reduce potential overheating associated with pulsed laser treatment on the suture and incisions or wound area. In some embodiments the exogenous source may have a cold tip to cool the target tissue area before, during and after application of the exogenous energy. In some embodiments the cold tip may be a temperature from about 2-8° C.

In some embodiments, this disclosure provides a method for thermally activated suturing of biological tissues by applying laser energy to join two or more tissue segments via a suture having a photothermal conversion material.

In some embodiments, this disclosure provides a method for joining tissues comprising the steps of: delivering a wound closure device to fissures or incisions at an anastomotic site, the wound closure device having a structural element coated with a heat delivery composition comprising a carrier and a material that absorbs laser light at one or more wavelengths to the tissue to be joined; and exposing the heat delivery composition on the wound closure device to laser light at one or more wavelengths, wherein the material absorbs the photonic energy and converts the photonic energy into heat, wherein the heat induces localized hyperthermia in the fissures or the incisions at the anastomotic site to promote closure.

In some embodiments, this disclosure provides a method for joining tissue comprising the steps of: delivering a wound closure device to fissures or incisions at an anastomotic site, the wound closure device having a structural element coated with a heat delivery composition comprising particles having a carrier admixed with a material that absorbs laser light at one or more wavelengths to the tissue to be joined; and exposing the heat delivery composition on the wound closure device to laser light at one or more wavelengths, wherein the particle absorbs the photonic energy and converts the photonic energy into heat, wherein the heat induces localized hyperthermia in the fissures or the incisions at the anastomotic site to promote closure.

In some embodiments, the structural element of the wound closure device comprises biodegradable material.

In some embodiments, the structural element of the wound closure device is configured as a suture. In some embodiments, the suture can take various forms. In some embodiments, the suture comprises a strip or strand having the material associated with the carrier (e.g. collagen fibers) which can be sewn or draped upon a fissure or incision to provide closure. Once in place, the suture is irradiated with laser or other high intensity light energy to fuse the suture to the anastomotic site.

In some embodiments, this disclosure provides a biodegradable shape memory polymeric suture that can be formed in a compressed temporary shape and then on demand be expanded to its permanent shape to fit as required. In some embodiments, this disclosure provides a biodegradable shape memory polymeric suture that can be knotted in a confined space.

In one embodiment, this disclosure provides a method of closing a wound or body scission comprising the steps of (1) providing a suture formed of a shape memory polymer; (2) stitching the wound or body scission using the suture; and (3) irradiating the suture to close the wound or body scission.

In some embodiments, this disclosure provides a method for joining tissue at a wound site or body scission comprises the steps of (1) providing a suture formed of a shape memory polymer; (2) stitching the wound or body scission using the suture and tying a knot; (3) irradiating the suture with a pulsed laser; wherein the irradiated suture shrinks and tightens the knot by increasing the temperature higher than glass transition temperature (T_{g}). In some embodiments, the knot is tightened in 20 seconds when heated to 40° C. In some embodiments, the suture is irradiated with a pulsed laser at a wavelength of 1064 nm, at an energy efficiency of 0.5 W/cm2 for 30 second. In some embodiments, the suture is irradiated with a pulsed laser at a wavelength of 805 nm, with a fluence of 40 J/cm² and a 100 ms pulse.

Photoreactive Biological Glue enhanced Photothermal Suturing

In some embodiments, this disclosure provides a method for joining tissue comprising: delivering a wound closure device to an anastomotic site, the wound closure device comprises a structural element and a heat delivery composition having a material interacting with an exogenous energy source admixed with a carrier; exposing the wound closure device with the exogenous source, wherein the material absorbs the energy from the exogenous source and converts the energy into heat, wherein the heat induces localized hyperthermia in a fissure or an incision at the anastomotic site to promote closure.

In some embodiments, the carrier comprises a biological glue capable of forming a bond to tissue segments due to the chemical reaction between the reactive chemical groups (—CHO,—SH) carried by biological glue and the reactive group of the tissue proteins (i.e. —NH₂ from lysine, —SH from cysteine residue) and thereby tightening the wound closure. In some embodiments, the biological glue is selected from the group consisting of collagen, elastin, fibrin, albumin, and combinations thereof.

In some embodiments, this disclosure provides a method for joining tissue comprising the steps of: delivering a wound closure device to fissures or incisions at an anastomotic site, the wound closure device having a structural element coated with a heat delivery composition comprising particles having a carrier admixed with a material that absorbs laser light at one or more wavelengths to the tissue to be joined; and exposing the heat delivery composition to laser light at one or more wavelengths, wherein the particle absorbs the photonic energy and converts the photonic energy into heat, wherein the heat induces localized hyperthermia in the fissures or the incisions at the anastomotic site to promote closure, wherein the carrier comprises biological glue capable of forming a bond to fissures or incisions due to the chemical reaction between the reactive chemical groups (—CHO,—SH) carried by biological glue and the reactive group of the tissue proteins (i.e. —NH₂ from lysine, —SH from cysteine residue) and thereby tightening the wound closure. In some embodiments, the biological glue is selected from the group consisting of collagen, elastin, fibrin, albumin, and combinations thereof.

Any of the curable compositions disclosed herein, including the in situ curable bioadhesive, the remotely triggered in situ curable dental composition, the remotely triggered in situ curable bone cement, the in situ curable tissue adhesives, and the in situ curable hydrogel compositions may additionally or optionally include components such as bioagents, toughener, polymerization inhibitors, crosslinking agents, reinforcement fillers, contrast agents, accelerators, or other similar accounts.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### General Procedures

The compositions of this invention may be made by various methods known in the art. Such methods include those of the following examples, as well as the methods specifically exemplified below.

### Example 1 Particle Fabrication

Reagents source: Chemical reagents sodium dodecyl sulfate (SDS), aqueous polyvinyl alcohol (PVA), NeoCryl® B-805 polymer (MMA/BMA copolymer, weight average molecular weight=85,000 Da, glass transition temperature T_{g}=99° C.) was purchased from DSM. Epolight™ 1117 (tetrakis aminium, absorbing at 800 nm-1071 nm, melting point: 185-188° C., soluble in acetone, methylethylketone and cyclohexanone) was purchased from Epolin Inc. Antioxidant Cyanox® 1790 (1,3,5-tris(4-tert-butyl-3-hydroxy-2,6-dimethyl benzyl)-1,3,5-triazine-2,4,6-(1H,3H,5H)-trione, CAS NUMBER 040601-76-1) was purchased from Cytec Industries Inc.

### Example 1 (i) Synthesis and Characterization of Tetrakis Aminium Dye/B805 Particles (Uncoated Particles Synthesized Through Emulsion Method)

Abbreviations: n-BMA: n-butyl methacrylate; MMA: methyl methacrylate

The preparation of the aqueous phase: under the stirring with an IKA Ultra-Turrax® T 25 homogenizer at 8000 RPM, 1.2 g of sodium dodecyl sulfate (SDS) was added into 190 g of 4.9% aqueous polyvinyl alcohol (PVA) solution placed in a round bottom flask. An aqueous solution of SDS containing 4.9% PVA was formed after the dissolution of SDS (the aqueous phase).

The preparation of the organic phase: to 88 g of dichloromethane was added 8.0 g of DSM NeoCryl® B-805 polymer (MMA/BMA copolymer), 1.82 g of Epolight™ 1117 dye, and 0.65 g of Cyanox® 1790 in 88 g to allow the formation of a clear solution of B805 polymer and dyes (the polymer: dye weight ratio=4.4:1).

The organic phase (polymer and dyes dissolved in dichloromethane) was injected directly into the aqueous phase (PVA solution with SDS surfactant) at the tip of the Turrax's roto-stator (i.e. directly into the flow being sheared by the roto-stator). The shear mixing at 8000 RPM was continued for 30 minutes. The resulting mixture was decanted into an open-mouth container and stirred magnetically for 16 hours. A solid suspension of particles containing IR dye was obtained.

The solid suspension was centrifuged at 5000 RPM for 30 minutes and the particles were collected. The collected particles were washed with distilled water by resuspending the particles into distilled water and centrifuging as before to collect the particles. This washing process was repeated three times to remove residual PVA. The resulting MMA/BMA copolymer particles containing IR dye were air-dried.

### Example 1 (ii) Synthesis of 25% VTMS Coated Tetrakis Aminium Dye/B805 Particles

In a first vessel, 1.52 g (0.01 mmol) of vinyltrimethoxysilane (CH₂═CHSi(OMe)₃, VTMS, MW=148 Da) was mixed with 4.58 g of dilute aqueous hydrochloric acid at a pH of 3.5 under magnetic stirring (24.9 wt. % solution of CH₂═CHSi(OMe)₃ in diluted HCl). The resulting mixture was stirred for 2 hours to allow complete hydrolysis of VTMS to give vinylsilanetriol (CH₂═CHSi(OH)₃, MW=106 Da).

In a second vessel, under magnetic stirring, 3.0 g of pre-made uncoated IR dye particles of Example 1 (i) were dispersed in 57 grams of water to provide a 5.0 wt. % dye particle dispersion. The pH value of the resulting IR dye particle aqueous dispersion was adjusted to 10.0 with the addition of dilute aqueous ammonium hydroxide. To this particle dispersion at pH 10, an aliquot of 3.99 g of the hydrolyzed 25 wt. % VTMS solution was added at a rate of 2 drops per second to the particle suspension. The pH value of the resulting suspension was monitored after the hydrolyzed 25% VTMS solution addition and adjusted with ammonium hydroxide solution to maintain a pH of 10 for 60 minutes. After 60 minutes, the suspension was neutralized with glacial acetic acid to lower the pH from 10 to 4.6-5.7. The weight ratio of VTMS to the uncoated particle was 0.33:1.

The resulting particle suspension was centrifuged for 30 minutes at 5000 RPM to collect the vinylsilicate-coated dye particles. The particles collected after the centrifugation were redispersed in distilled water and subjected to centrifugation to collect the particles. The washing procedure was repeated 3 times to remove any unreacted chemical reagents. The resulting vinylsilicate-coated particles were suspended in distilled water.

Multiple commercially available infrared dyes were screened to find a preferred composition to provide localized heat delivery to a tissue site with sufficient temperature rise to accelerate a reaction outside of the particle. The infrared dyes screened include Lumogen IR 1050, Epolight™ 1117, Epolight™ 1125, and Epolight™ 1178.

In the emulsion method of encapsulation, a surfactant is necessary to help keep the emulsion stable. While Aerosol® TR-70 (sodium bis(tridecyl) sulfosuccinate) could be used as an emulsifier to prepare polymer particles encapsulating Epolight™ 1117 tetrakis aminium dye, TR-70 only provided limited stabilization effects on the tetrakis aminium dye. Sodium dodecyl sulfate was found to have a better stabilizing effect on the Epolight™ 1117 during the emulsion and evaporation process, shifting retention in the particles from 50% retention, to up to 85-90% retention. Reducing the amount of SDS in the aqueous phase led to lower Epolight™ 1117 retention and larger particle size (Table 3).

TABLE 3
Stabilization effects of the surfactant type and quantity on
tetrakis aminium dye in aqueous phase during emulsification

| | Surfactant in aqueous phase | | | |
|---|---|---|---|---|
| | 0.6% TR-70 | 0.6% SDS | 0.4% SDS | 0.2% SDS |
| Median Particle size | 1.20 μm | 0.47 μm | 0.68 μm | 1.08 μm |
| % Epolight ™ 1117 | 51.70% | 82.96% | 80.17% | 74.97% |
| Retention | | | | |

The polymer used for this application is preferred to have a glass transition temperature significantly greater than the temperature of the environment for the intended use.

Various commercially available acrylic polymers were screened for preferred particle performance characteristic such as particle size distribution, IR dye stability and encapsulation efficiency. NeoCryl® B-851, a butyl acrylate/styrene copolymer proved to have a hydroxyl value too high, leading to a more polar particle and poor retention of the embedded tetrakis aminium dyes. NeoCryl® B-818, an ethyl acrylate/ethyl methacrylate copolymer, had a lower hydroxyl value, but was still swellable in low molecular weight alcohols. NeoCryl® B-805, a methyl methacrylate/butyl methacrylate copolymer, had a suitably low hydroxyl value and a T_{g} (99° C.) high enough for human body applications. Use of a pure methyl methacrylate polymer, NeoCryl® B-728, led to greater degradation of the Epolight™ 1117 dye.

The loading of dyes within the particles is as high as possible without degrading the cohesion of the polymer. The additives that stabilize the dye within the particles have been studied. The antioxidant Cyanox® 1790 was found to have a positive impact on dye stability.

### Example 1(iii). Particle Characterization and Stability Testing

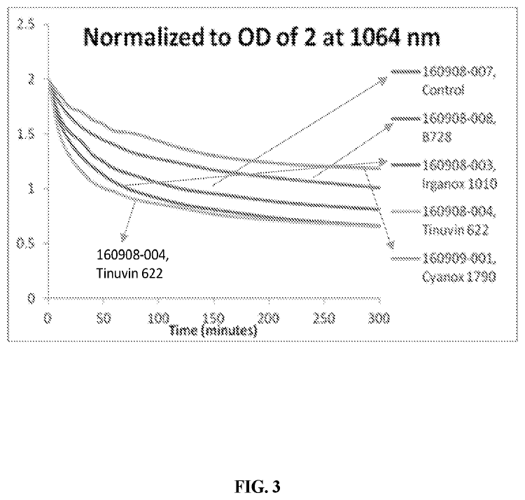

Particle Size and Distribution for the particle heaters The particle size and size distribution of the NIR dye/MMA/BMA copolymer particles were measured by a Horiba LA-950 Particle Size Analyzer in distilled water with pH 7.4 (FIG. 3). All the particle size measurements were carried out at room temperature (17-23° C.).

Various additional Epolight™ 1117 particles are prepared according to the procedures set forth in the Example 1(i) above. The physicochemical properties of the resulting particles are summarized in Table 4 below.

TABLE 4
Particle Structure

| | | | particle size | polymer/dye | |
|---|---|---|---|---|---|
| | | polymer | range | weight ratio | |
| entry | IR dye | carrier | (micron) | range | additive |
| 1 | Epolight ™ | B805^{a} | 0.47, 0.68, | 4.4:1 | Cyanox ® |
| | 1117 | | 1.08, 1.20 | | 1790^{b} |
| | | | | | SDS^{c} |

^{a}Polymer B805 ®: copolymer of 96% methyl methacrylate and 4% butyl methacrylate.
^{b}Cyanox ®1790: dye stabilizer mixed in the polymer matrix.
^{c}SDS = sodium dodecyl sulfate, surfactant for emulsion solvent evaporation particle fabrication method.

### Example 1(iii) Optical Properties of the Epolight™ 1117 IR Dye-B805 Particles

The optical properties of the Epolight™ 1117 IR dye-B805 particles dispersed in an aqueous water are determined by UV-VIS spectroscopy.

TABLE 5
Properties of Epolight ™ 1117 IR Dye

| | | Peak | | |
|---|---|---|---|---|
| | | absorption | Extinction | Non- |
| | Molecular | wavelength | coefficient | cytotoxic |
| | Weight | (nm) | (M⁻¹*cm⁻¹) | concentrations |
| Dye | (g/mol) | (in DCM^{a}) | (in DCM) | (μM) |
| Epolight ™ | 1211 | 1098 | 105,000 | 32 |
| 1117 | | | | |

DCM is the abbreviation for dichloromethane.

### Example 1(iv): Preparation of Biodegradable Particle Heaters

Poly(lactide-co-glycolide) (PLGA) (MW: 10,000-15,000 Da), Methoxy poly(ethylene glycol)-b-poly(lactide-co-glycolide) (mPEG-PLGA) (MW: 2-15 kDa) are purchased from PolySciTech® (West Lafayette, Ind., USA). Epolight® 1117 was purchased from Epolin Inc. (Newark, N.J., USA) and; ICG was purchased from AFG Biosciences (Northbrook, Ill., USA), IR-193 dye was a gift from Polaroid (Cambridge, Mass.) to Bambu Vault; All cell lines are obtained from ATCC (Manassas, Va.). The 3-(4,5-dimethylthiazol-2-yl)-5-(3-carboxymethoxyphenyl)-2-(4-sulfophenyl)-2H-tetrazolium (MTS) assay kit is purchased from Promega Corporation® (Madison, Wis., USA), Triton-X and other HPLC grade organic solvents are obtained from Fisher Scientific™ (Agawam, MA, USA).

Multiple commercially available infrared dyes are screened to find a preferred composition to provide localized heat delivery to a tissue site with sufficient temperature rise to accelerate a reaction outside of the particle. The infrared dyes screened include ICG, IR-193 dye, Lumogen® IR 1050, Epolight® 1117, Epolight® 1125, and Epolight® 1178.

Amphiphilic co-polymers of PLGA and PEG are used to prepare PLGA/PLGA-PEG NPs with a blend of 75:25 of PLGA and PLGA-PEG. Epolight™ 1117 or ICG loaded NPs are synthesized by adding Epolight™ 1117 or ICG to the polymer solution containing blend of 75:25 of PLGA and PLGA-PEG. Similarly, empty NPs (without the IR dye) are prepared.

IR Dye concentration is measured by NIR spectrophotometry by measuring absorbance and using Beer's law to estimate concentration. Particle size, polydispersity index and zeta potential are confirmed by dynamic light scattering using a Zetasizer (ZS-90 from Malvern Instruments) and scanning/transmission electron microscopy. Encapsulation efficiency is calculated for the IR Dye by estimating the final amount of IR Dye in the purified particles (using concentration measured by UV spectrophotometry) and dividing that by amount that is originally used during the synthesis of the particles.

$$\% dye encapsulation efficiency = \frac{( Amount of dye in mg from spectrophotometry ) \times 100}{( Amount of dye in mg that was added during synthesis )}$$

(a) Particle Characterization and Stability Testing

Particle Size and Distribution for the Particle Heaters

Particle size, polydispersity index and zeta potential are confirmed by dynamic light scattering using a Zetasizer® (ZS-90 from Malvern Instruments). Particle sizes are measured in deionized water and phosphate buffered saline. The zeta potential for the particles is measured using particles dispersed in buffers (10 mM) with different pHs. The particles are imaged with a Tecnai F20 transmission electron microscope (FEI, Hillsboro, Oreg.) after negative staining with 2% phosphotungstic acid (PTA). All the particle size measurements are carried out at 25° C. All the measurements are performed in triplicate.

In Vitro Stability Study

In vitro stability of the particle heaters is evaluated by storing the sample at 4° C. and 37° C. The particle size change, the PDI change and the zeta potential change is measured by Zetasizer® Dynamic Light Scattering (DLS) instrument. Particle formulations containing the material are stored in a vial covered in foil and stored at 4° C. for a week to study the stability of the particles for their storage shelf life. The particles are also resuspended in 1:1 ratio (by volume) in MEM alpha modification media containing 10% FBS and stored at 37° C. to study their stability under physiological conditions. Samples are periodically removed from these two storage conditions and particle size; polydispersity index and zeta potential are confirmed by dynamic light scattering using a Zetasizer® (ZS-90 from Malvern Instruments) for particles stored under these conditions.

(b) Particle Content Test

UV/VIS/NIR: The absorbance spectrum for the material (IR dye) is measured using Shimadzu UV-3600 UV-NIR Spectrophotometer.

The Percentage of Dye Loading Determination

The percentage of IR dye loaded into the particles can be determined according to the following procedure: Known quantities of particles in deionized water are added to a solution of 2% Triton-X solution in a 1:1 volume ration. The UV-VIS-NIR absorbance spectrum of the IR dye is measured using Shimadzu UV-3600 UV-VIS-NIR Spectrophotometer. The concentration of the IR dye in the particles is determined from application of Beer's law.

$$[ Dye ] ( μM ) = \frac{{Absorbance}_{λ}}{{ɛ}_{λ} \times l} \times {10}^{6}$$

where the path length, l, is 1 cm.

The quantity of IR dye is determined from the product of the concentration, the amount of total solution, and the molecular weight of the IR dye. The IR dye loading as a percentage of the total particle mass is determined from:

$$Dye Loading ( \% ) = \frac{Amount of dye in solution}{Amount of particle used} \times 100 \%$$

### Example 2. Efficacy Determination Protocol

An Efficacy Determination Protocol is used to evaluate the effect of biological chemicals including bodily fluid on the material that are encapsulated inside the particle. Briefly, a known quantity of the particles containing the material is incubated with 1 mL of complete cell culture media (for example macrophage or neutrophil cell growth media) containing 10% fetal bovine serum at 37° C. As a negative control, the same quantity of particles containing the material is suspended in 1 mL of distilled water and incubated at 37° C. At different time intervals (for example: 24 h, 48 h, 72 h, 120 h) following incubation, for both the test and control, a small portion of the sample is removed and diluted with distilled water. If the material absorbs UV-VIS-IR, then the UV-VIS-IR absorbance spectrum of each solution is measured using a UV-VIS-IR spectrophotometer. Degradation of the material by the cell culture medium is determined by comparing the peak absorption in the spectrum of the test sample to the absorption of the control sample at the same spectral peak, and degradation is generally reported as the percentage in the reduction in the peak absorbance. If the material does not absorb UV-VIS-IR, other analytical tools, like NMR, HPLC, LCMS etc., would be used to quantify the concentration of the material in the test and control. The particles can be designed to ensure that no more than 90% degradation is observed at 24 h following incubation with relevant cell culture media.

### Example 3. Extractable Cytotoxicity Test

100 mg of dry particles were weighed out and then suspended in 1 mL of cell culture media Dulbecco's Modified Eagle's medium (DMEM) containing 10% fetal bovine serum (FBS) and vortexed five times to ensure thorough mixing. This suspension was incubated at 37° C. for 24 hrs. After the incubation period, the suspension was centrifuged at 10,000 G for 10 minutes and the supernatant was collected. The supernatant solution was filtered through a 0.2 micron syringe filter and was used for cytotoxicity evaluation as the “neat” or 1× sample. This 1× neat extract was serially diluted with media containing 10% FBS for cytotoxicity testing. The following serial dilutions were made using the neat extract and the DMEM supplemented with 10% FBS: 2× (2-fold dilution), 4× (4-fold dilution), 8× (8-fold dilution), 16× (16-fold dilution) and 32× (32-fold dilution).

Inhibitory Concentration for 30% cell killing (IC₃₀) of the particle extract on NIH-3T3 cells (obtained from ATCC) was determined by performing an MTS assay, a standard colorimetric method to measure the cell viability following incubation with different dilutions of the 1× extract obtained above. NIH-3T3 cells were plated in a 96-well culture plate at a density of 10,000 cells per well and allowed to adhere to the surface overnight. Extract concentrations ranging from 1× to 32× were added and incubated for 24 hours at 37° C., in a 5% CO₂ incubator. Controls for the cytotoxicity experiment included “live” and “dead” (cells were killed due to osmotic pressure by adding D.I. water). “Live” cells had nothing except cell culture media containing 10% FBS added to them and were used to obtain the 100% viability data. The “dead” control was used to obtain the 0% viability data point. After 24 hours, to a final volume of 100 μL of media in the cells, 20 μL of PMS-activated MTS reagent was added and incubated for 90 minutes. The absorbance was measured at 490 nm using a plate reader (Spectramax M2e, Molecular Devices). Viability of cells was calculated using the absorbance measured at 1× dilution of the extract and the results of absorbance for serial dilutions 1× to 32× of the extract were plotted in MS Excel using linear regression curve fitting algorithm to obtain the IC₃₀. All the samples were tested in triplicate and results were averaged over the three repeats. A particle that results in a 70% cell viability in the cytotoxicity test is considered passing the Extractable Cytotoxicity Test.

In an embodiment, a particle example that results in 70% cell viability (or higher) in the Extractable Cytotoxicity Test at the original extract concentration (1×) is considered passing the ECT criteria. In an embodiment, a particle example demonstrating results in 70% cell viability (or higher) in the cytotoxicity test at 10-fold dilution (10×) is considered passing the ECT criteria. In an embodiment, a particle example showing results in 70% cell viability (or higher) in the cytotoxicity test at 100-fold dilution (100×) is considered passing the ECT criteria. In some instances, if the neat or dilution concentration of the material in the leachate is independently less than IC₁₀, IC₃₀, IC₄₀, IC₅₀, IC₆₀, IC₇₀, IC₈₀, or IC₉₀, the particle passes the Extractable Cytotoxicity Test.

### Example 4. Thermal Cytotoxicity Test

NIH-3T3 cells (obtained from ATCC) are plated in a 48-well culture plate at a density of 20,000 cells per well and allowed to adhere to the plate surface overnight. The following day, the media in each well is replaced with fresh, cell growth media containing 10% fetal bovine serum. The composition to be irradiated is placed on a sterile, empty, CellCrown™ insert which includes a transparent PET filter with a pore size of 1 μm (allowing heat to easily spread out of the filter into the surrounding media) and these are inserted into the well, such that the insert is just submerged in the media but not directly in contact with the cells. The specific composition (which includes the carrier and the material at a specific composition) to be tested is exposed to the exogenous source (e.g., irradiation with a laser at three different fluences, each at three different pulse durations) to ensure the composition (i.e. carrier and material) are functional (i.e. adequate heat is produced to cause the required physical, chemical or biological activity). Viability of the NIH-3T3 cells following the irradiation is determined by performing an MTS assay, a standard colorimetric method to measure the cell viability 24 h after the irradiation. 1 h following the irradiation, the insert is removed, and cells are incubated for an additional 23 hours at 37° C., in a 5% CO₂ incubator. Controls for the thermal cytotoxicity experiment included “live”, “dead” (cells were killed due to osmotic pressure by adding D.I. water) and the composition to be tested alone, (i.e. with no laser irradiation) and “light only”. “Live” cells will have nothing except cell culture media containing 10% FBS added to them and are used to obtain the 100% viability data. The “dead” control is used to obtain the 0% data point. “Light only” control includes exposing cells to the equivalent light dose without the composition present in the well. Light doses will be selected to ensure little to no killing of cells is observed using the light only control. At the end of the 24 hours, to a final volume of 200 μL of media in the cells, 40 μL of PMS activated MTS reagent is added and incubated for 90 minutes. The absorbance is measured at 490 nm using a plate reader (Spectramax M2e, Molecular Devices). Viability of cells is calculated using the absorbance measured and the results plotted in MS Excel. The composition and light dose(s) that do not kill more than 30% of the cells are considered passing the Thermal Cytotoxicity Test.

### Example 5. Material Process Stability Test

Particle heaters of Example 1(i) are dispersed in a 2% solution of gelatin in warm water. The suspension is vortexed and transferred to 50 mm plastic culture dishes and allowed to gel, producing a greenish gel. The optical density is measured by reflectance spectroscopy to provide a baseline absorbance.

Areas on the culture dishes are irradiated over a range of pulse widths and fluences that span the conditions expected for use. Generally, pulse widths range from about 100 μs to about 1 second, with fluences that range from about 0.1 J/cm² to about 60 J/cm². The absorbance is measured for each exposure condition and compared to the baseline absorbance. Conditions for which the loss in absorbance is less than 50% are considered to pass the Material Process Stability Test.

### Example 6. Controlled Heat Generation from Laser-Excited Particle Heaters in Gelatin

The test is to determine threshold conditions for controlled heat generation that produces a thermal increase to 50° C. Heat was generated by exposing a gelatin gel suspension of particle heaters of Example 1(ii) with a red thermochromic pigment with 50° C. thermal threshold for color loss to laser irradiations with various operating parameters.

Thermochromic MC Pigment 50° C. Red (a red thermochromic dye with a threshold temperature for color loss at 50° C., TM PD 50 3111, Lot #MC1204191) was purchased from Sandream Enterprises. Unflavored, commercial, food grade Knox® gelatin was used as received.

A 2.0 wt. % stock solution of gelatin in water was prepared by wetting one gram of gelatin with 12 g of cold water, then adding 37 g of water at 75° C. and stirring until dissolved. A 30.0 wt. % stock suspension of particle heaters in water was prepared by suspending of 3.0 g of the particles from Example 1(ii) in 7.0 mL of water.

To 65.0 mg of the particle heater suspension in a 4 dram glass vial was added 25 mg of red thermochromic pigment to form a mixture. To this mixture was added 2.0 g of the 2% gelatin solution, and the glass vial was vortexed for 5 minutes and set aside for use.

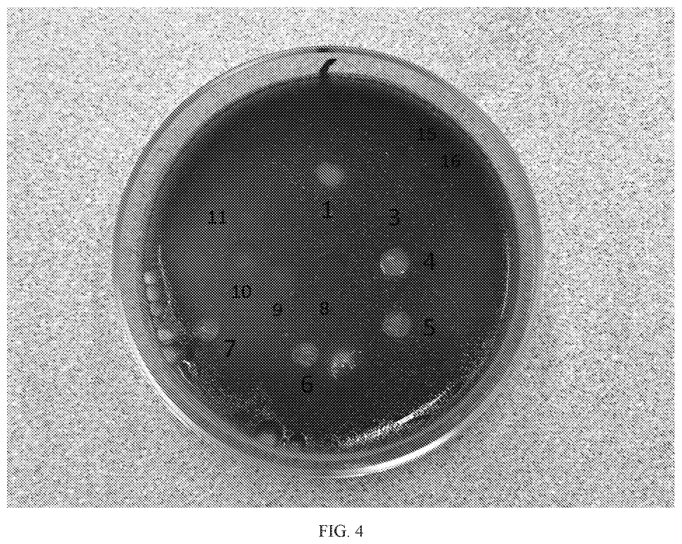

The vortexed suspension was transferred by pipette to a 50 mm plastic culture dish, spread evenly, and allowed to cool to form a gel. The particle heaters were spread uniformly within the gelatin gel matrix and gave a greenish color. The particles of the red thermochromic pigment were distributed unevenly within the gelatin matrix (see FIG. 4).

A control sample of red thermochromic pigment, but lacking the particle heaters, was also prepared using the procedure described above by suspending 25 mg of dye in 2 g of 2% gelatin solution, vortexing, spreading evenly in a 50 mm plastic culture dish and allowing to gel.

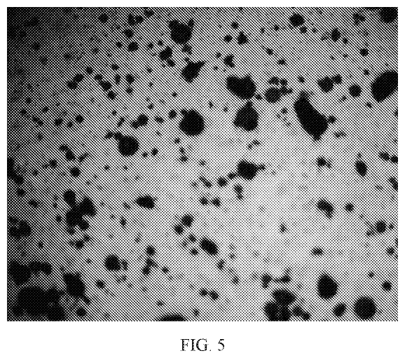

After the gel had set, it was irradiated with a laser under a variety of different operating parameters. Several regions of the gel (spots 1-3) were first irradiated at 1064 nm in spots of 5 mm diameter with a Lutronic solid state laser, with exposures of 3.51 J/cm² using a 10 ns pulse (Q-switched mode) (Spot 1) and of 2.01 J/cm² (Spot 2) and 3.51 J/cm² (Spot 3) using a 350 μs pulse (Spectra mode). A second set of regions (spots 8-16) were irradiated at 980 nm in spots of about 3 mm diameter with a 10 Watt, electrically switched, CW semiconductor laser with pulse widths ranging from 10-250 ms and delivered energies ranging from 0.5-5 J. The color change effects caused by the laser exposures were photographically recorded using a iPhone camera or microscope camera. The visual results of color changes are shown in FIGS. 4-7. These experiments are summarized in Table 6.

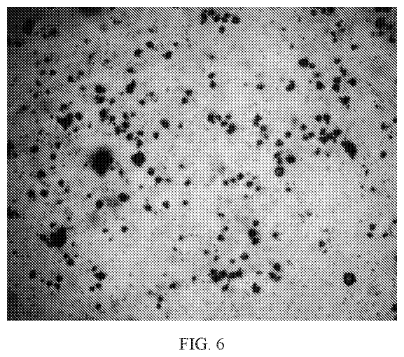

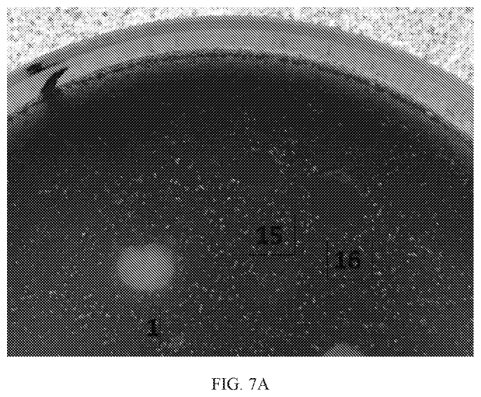

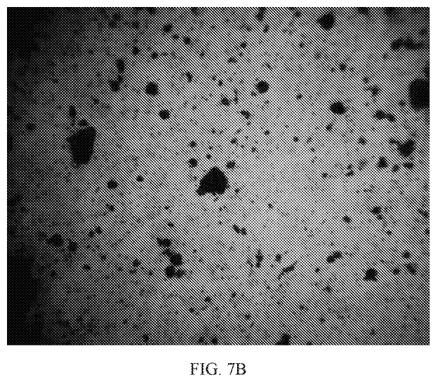

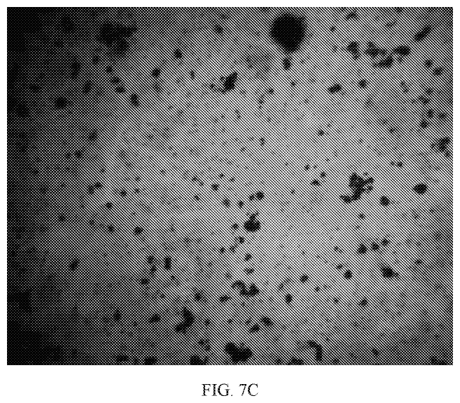

TABLE 6
Results of laser exposure of particle heaters and thermochromic pigment in gelatin

| | | | | Fluence, | | |
|---|---|---|---|---|---|---|
| Spot | Laser | Pulse width | | J/cm² | Result | Image |
| 1 | Lutronic (1064 nm) | 10 | ns | 3.51 | White spot, red pigment | |
| | | | | | decolorized, IR dye color gone | |
| 2 | Lutronic (1064 nm) | 350 | μs | 2.01 | Minimal disturbance of gelatin | |
| 3 | Lutronic (1064 nm) | 350 | μs | 3.51 | Slight depression in gelatin, IR | |
| | | | | | dye not changed. Red pigment | |
| | | | | | melted and color gone. | |
| 8 | Semiconductor | 200 | ms | 28.3 | A spot was formed in the | |
| | laser (980 nm) | | | | gelatin. IR dye was not | |
| | | | | | changed, but red pigment | |
| | | | | | appeared to be melted and | |
| | | | | | color gone. | |
| 9 | Semiconductor | 2 × 250 | ms | 70.7 | Same as spot 8 but bigger spot | FIG. 6 |
| | laser (980 nm) | | | | | |
| 10 | Semiconductor | 250 | ms | 35.4 | Same as spot 8 but slightly | |
| | laser (980 nm) | | | | bigger spot | |
| 11 | Semiconductor | 100 | ms | 14.1 | Approximately 3 mm spot, | |
| | laser (980 nm) | | | | surface particles of red pigment | |
| | | | | | mostly gone | |
| 12 | Semiconductor | 50 | ms | 7.1 | Same effect on gelatin, smaller | |
| | laser (980 nm) | | | | spot, surface particles of red | |
| | | | | | pigment evident | |
| 13 | Semiconductor | 10 | ms | 0.7 | Minimal disturbance of gelatin | |
| | laser (980 nm) | | | | observed | |
| 14 | Semiconductor | 30 | ms | 2.1 | Slight “melting” of gelatin | |
| | laser (980 nm) | | | | | |
| 15 | Semiconductor | 7 × 30 | ms | 14.9 | Similar to spots 11 and 16. | FIG. 7B |
| | laser (980 nm) | | | | Slightly smaller spot than 16 | |
| | | | | | but red pigment melted and | |
| | | | | | color gone | |
| 16 | Semiconductor | 200 | ms | 14.1 | Similar to spot 15 but larger | FIG. 7C |
| | laser (980 nm) | | | | spot. Red pigment melted, | |
| | | | | | color gone. | |

The results in Table 6 show that 1064 nm Q-switched laser irradiation of 3.51 J/cm² led to significant loss of IR dye and decolorization of red thermochromic pigment. Irradiation with a similar fluence but longer pulse width (Spectra mode) does not show IR dye degradation but does show melting and decolorization of the red thermochromic pigment. Reducing the fluence to 2.01 J/cm² led to no decolorization and little evidence of heat generation as evidenced by distortion of the gelatin.

Irradiation using the semiconductor laser at 980 nm required greater fluence to produce an equivalent decolorization of the thermochromic pigment. For example, a dose of 14 J/cm² was required to demonstrate complete loss of red color; lower fluence led to no or minimal observable effect. In all cases with this laser, no loss of IR dye was observed. The retention of the IR dye was evidenced by the ability to provide enough energy to decolorize the red pigment using several sequential with lower energy pulsed to achieve the same result as irradiation with a single pulse of equivalent total fluence.

The control sample, with red thermochromic pigment only, showed no change when exposed to the semiconductor laser using the settings described in Table 6.

In this study, the presence or absence of color clearance at each of the 16 test sample spots was examined. The presence or absence of color clearing for IR dye at the each one of the 16 test spots indicated the effectiveness of energy-to-heat conversion of the IR dye, e.g. the IR dye was able to absorb the photonic energy and converted the absorbed photonic energy into heat. The presence or absence of color clearing for the Red 50° C. Thermochromic dye at the each one of the 16 test spots indicated the effectiveness of the heat traveling from the IR dye particles to the surrounding gelatin medium near the particles (the spot being irradiated) and induction of a detectable temperature increase to a temperature at least above 50° C.

### Example 7. PMMA Strengthened Curable n-Butyl Acrylate Adhesive

In this study, in order to enhance the adhesive bond strength and flexibility of cyanoacrylate adhesives, PMMA was chosen as an additive for n-butyl cyanoacrylate (3M™ Vetbond™ Tissue Adhesive). The influence of the PMMA additive on the bond strength of the cured n-butyl cyanoacrylate with PMMA and IR dye particles was evaluated by measuring the mechanical force needed to break the bonding of the cured adhesive that joined the two pieces of pig skin. Pig skin was cut into small 1×0.5 inch pieces and left to soak in an artificial wound fluid (AWF) solution for at least 30 minutes prior to testing them with the adhesive. The AWF fluid contains 10 mM CaCl2, 200 mM NaCl, 40 mM tris(hydroxymethyl)aminomethane and 2% BSA (Bovine Serum Albumin) with the pH of the solution adjusted to 7.5.

7(i) Preparation of the Curable n-Butyl Cyanoacrylate Adhesive Composition Containing IR Dye Particles

The IR dye particles used throughout Example 7 are 2 micron particles composed of a core having Epolight™ 1117 dye and NeoCry® B-805 polymer (MMA/BMA copolymer), and a 25% VTMS/PEG shell (c=5.0 mg/mL, weight ratio of the VTMS/PEG shell to the uncoated PMMA core is 0.33:1) (referred as IR dye particles thereafter). The loading amount of Epolight™ 1117 in the PMMA particle was about 12 wt. % by the total weight of the particle. The IR dye particles were prepared according to the method described in Examples 1(i)-(ii) above.

7a(i): The preparation of a curable adhesive stock composition containing Epolight™ 1117 dye particles suspended in n-butyl cyanoacrylate monomer (concentration 1, c₁=5.0 mg/mL): to 500 μl of n-butyl cyanoacrylate (Vetbond™ curable cyanoacrylate adhesive) was added 2.5 mg of Epolight™ 1117 dye particles. The mixture was vortexed for 1 minute to allow the formation of a homogenous suspension of the of Epolight™ 1117 dye particles in the n-butyl cyanoacrylate monomer.

7a(ii): The preparation of a curable adhesive stock composition containing Epolight™ 1117 dye particles suspended in n-butyl cyanoacrylate monomer modified with NeoCryl® B-805 polymer (PMMA) additive (concentration, c=5.0 mg/mL): to 500 μl of n-butyl cyanoacrylate (Vetbond™ curable cyanoacrylate adhesive) was added 2.5 mg of 2 micron Epolight™ 1117 dye particles (concentration 2, c₂=5.0 mg) and 2.5 mg NeoCryl® B-805 polymer (concentration 3, c₃=5 mg/mL). The mixture was vortexed for 1 minute to allow the formation of a homogenous suspension of the of Epolight™ 1117 dye particles in the n-butyl cyanoacrylate monomer and the PMMA.

7(ii) Curing of Epolight™ 1117 Dye Particles Modified n-Butyl Cyanoacrylate Monomer with Laser Having Cool Tip Off

An aliquot of 25 μL of curable n-butyl cyanoacrylate adhesive of Examples 6(a)(i) or 6(a)(ii) was evenly spread over the edge of one piece of pig skin. The adhesive treated edge of the first piece of pig skin was joined with the end of a second piece of pig skin. Then the two ends were held together in place for 5 seconds. Laser treatment was applied if necessary (805 nm). The two pieces of the pig skin held together by the curable n-butyl cyanoacrylate adhesive 6(a)(i) or 6(a)(ii) were secured in the ADMET Expert Biomechanical Testing Machine. The 805 nm laser was set to a fluence of 30 J/cm² and a 100 ms (millisecond) pulse. The laser was held approximately 0.5 inches from the middle of the pig skin pieces joined by the curable adhesive 5(a)(i) or 5(a)(ii) and pulsed once. The test was repeated with the laser setting to a fluence of 15 J/cm² and 100 ms pulse and 10 J/cm² and 100 ms pulse with the cold setting off. This was repeated five times for each setting to test for reproducibility.

After the laser treatment, force was applied with the mechanical puller to pull the two pieces of pig skin apart. The amount of the force applied was expressed in unit of Newtons (N) and was measured and documented as observed on the MTESTQuattro software. The instrument records the force in Newtons required to break the bond formed by the cured adhesive. The results are summarized in Table 7 below.

The pulsed laser may also be operated at a wavelength of 1064 nm, but the bond strength of the cured Vetbond™ adhesive was inferior to the pulsed laser irradiation operated at a wavelength of 805 nm due to the higher degree of degradation with the 1064 nm laser condition (data not shown).

TABLE 7
PMMA Additive Strengthened Curable N-Butyl Cyanoacrylate

| | | Exp. 1 | Exp. 2 | Exp. 3 | Exp. 4 | Exp. 5 | Exp. 6 | Avg. | St. |
|---|---|---|---|---|---|---|---|---|---|
| Entry | | (N) | (N) | (N) | (N) | (N) | (N) | (N) | Dev. |
| Laser condition 1 | | 30 J/cm² and a 100 ms slow | | | | | | | |
| 1 | 25 μL | 8.8 | 12.7 | 10.3 | 1.1 | 4.3 | | 7.4 | 4.7 |
| | Vetbond ™ | | | | | | | | |
| | opened bottle | | | | | | | | |
| | No laser | | | | | | | | |
| 2 | 25 μL | 12.1 | 7.8 | 13.7 | | | | 11.2 | 3.0 |
| | Vetbond ™ New | | | | | | | | |
| | bottle | | | | | | | | |
| Laser condition 2 | | 15 J/cm² and a 100 ms slow | | | | | | | |
| 3 | 25 μL 5.0 | | | | | | | | |
| | mg/mL IR dye | | | | | | | | |
| | in Vetbond ™ | | | | | | | | |
| 4 | 25 μL 5.0 | 13.3 | | | 13.1 | 8.8 | 10.0 | 12.1 | 2.1 |
| | mg/mL IR dye | | | | | | | | |
| | and 5.0 mg/mL | | | | | | | | |
| | PMMA in | | | | | | | | |
| | Vetbond ™ | | | | | | | | |
| | No laser | | | | | | | | |
| 5 | 25 μL 5.0 | 7.8 | 4.7 | 8.7 | | | | 7.1 | 2.1 |
| | mg/mL IR dye | | | | | | | | |
| | and 5.0 mg/mL | | | | | | | | |
| | PMMA in | | | | | | | | |
| | Vetbond ™ | | | | | | | | |
| Laser condition 3 | | 10 J/cm² and a 100 ms slow | | | | | | | |
| 6 | 25 μL 5.0 | 4.5 | 7.5 | 8.1 | 1.3 | 4.5 | | 5.2 | 2.7 |
| | mg/mL IR dye | | | | | | | | |
| | in Vetbond ™ | | | | | | | | |
| | No laser | | | | | | | | |
| 7 | 25 μL 5.0 | 3.7 | 8.0 | 5.2 | 5.4 | 6.0 | | 5.6 | 1.6 |
| | mg/mL IR dye | | | | | | | | |
| | in Vetbond ™ | | | | | | | | |

The PMMA+cyanoacrylate+IR Dye particles+laser cured rapidly, and the bond was as strong as just the cyanoacrylate. However, addition of PLGA to the cyanoacrylate mixture with the IR Dye particles followed by laser irradiation did not improve the bond strength.

In the absence of PMMA additive, the addition of IR dye particle as catalyst for laser accelerated curing of the Vetbond™ adhesive caused weakening of the bond strength. The heat generated from energy-to-heat conversion from IR dye particles caused the degradation of the cured n-butyl cyanoacrylate adhesive (entries 1-2 vs. entries 6-7 of Table 7).

The cured Vetbond™ adhesive modified with the IR dye particles without PMMA was considerably weaker than that of the cured Vetbond™ adhesive modified with IR dye particles and PMMA (entries 4-5 vs. entries 6-7 of Table 7). The addition of 5.0 mg/mL of PMMA to Vetbond™ brand adhesive modified with IR dye particles made the bond strength much greater.

### Example 8. PMMA Strengthened Curable Dental Cyanoacrylate Adhesive

In this study, in order to enhance the adhesive bond strength and flexibility of dental adhesives, PMMA is chosen as an additive for curable dental adhesive compositions. The influence of the PMMA additive on the process stability under curing conditions with PMMA and IR dye particles was evaluated by measuring the mechanical force needed to break the bonding of the cured dental adhesive that joined the two pieces of pig skin. Pig skin was cut into small 1×0.5 inch pieces and left to soak in an artificial wound (AWF) fluid for at least 30 minutes prior to testing them with the adhesive. The AWF fluid contains 10 mM CaCl₂), 200 mM NaCl, 40 mM tris(hydroxymethyl)aminomethane and 2% BSA (Bovine Serum Albumin) with the pH of the solution adjusted to 7.5.

8(i) Preparation of the Curable n-Butyl Cyanoacrylate Adhesive Composition Containing IR Dye Particles

The IR dye particles used throughout example 6 are 2 micron particles composed of a core having Epolight™ 1117 dye and NeoCry® B-805 polymer (MMA/BMA copolymer), and a 25% VTMS/PEG shell (c=5.0 mg/mL, weight ratio of the VTMS/PEG shell to the uncoated PMMA core is 0.33:1) (referred as IR dye particles thereafter). The loading amount of Epolight™ 1117 in the PMMA particle was about 12 wt. % by the total weight of the particle. The IR dye particles were prepared according to the method described in Examples 1(i)-(ii) above.

The preparation of a curable adhesive stock composition containing Epolight™ 1117 dye particles suspended in n-butyl cyanoacrylate monomer (concentration 1, c1=5.0 mg/mL): to 500 μl of n-butyl cyanoacrylate (Vetbond™ curable cyanoacrylate adhesive) was added 2.5 mg of Epolight™ 1117 dye particles. The mixture was vortexed for 1 minute to allow the formation of a homogenous suspension of the of Epolight™ 1117 dye particles in the n-butyl cyanoacrylate monomer.

The preparation of a curable adhesive stock composition containing Epolight™ 1117 dye particles suspended in n-butyl cyanoacrylate monomer modified with NeoCryl® B-805 polymer (PMMA) additive (concentration, c=5.0 mg/mL): to 500 μl of n-butyl cyanoacrylate (Vetbond™ curable cyanoacrylate adhesive) was added 2.5 mg of 2 micron Epolight™ 1117 dye particles (concentration 2, c2=5.0 mg) and 2.5 mg NeoCryl® B-805 polymer (concentration 3, c3=5 mg/mL). The mixture was vortexed for 1 minute to allow the formation of a homogenous suspension of the of Epolight™ 1117 dye particles in the n-butyl cyanoacrylate monomer and the PMMA.

8(ii) Curing of Epolight™ 1117 Dye Particles Modified n-Butyl Cyanoacrylate Monomer with Laser Having Cool Tip Off

An aliquot of 25 μL of curable n-butyl cyanoacrylate adhesive of examples 9(a)(i) or 9(a)(ii) was evenly spread over the edge of one piece of pig skin. The adhesive treated edge of the first piece of pig skin was joined with the end of a second piece of pig skin. Then the two ends were held together in place for 5 seconds. Laser treatment was applied if necessary (805 nm). The two pieces of the pig skins held together by the curable n-butyl cyanoacrylate adhesive 9(a)(i) or 9(a)(ii) were secured in the ADMET Expert Biomechanical Testing Machine. The 805 nm laser was set to a fluence of 30 J/cm² and a 100 ms (millisecond) pulse. The laser was held approximately 0.5 inches from the middle of the pig skin pieces joined by the curable adhesive 9(a)(i) or 9(a)(ii) and pulsed once. The test was repeated with the laser setting to a fluence of 15 J/cm² and 100 ms pulse and 10 J/cm² and 100 ms pulse with the cold setting off. This was repeated five times for each setting to test for reproducibility.

After the laser treatment, force was applied with the mechanical puller to pull the two pieces of pig skin apart. The amount of the force applied was expressed in unit of Newtons (N) and was measured and documented as observed on the MTESTQuattro software. The instrument records the force in Newtons required to break the bond formed by the cured adhesive. The results are summarized in Table 8 below.

The pulsed laser may also be operated at a wavelength of 1064 nm, but the bond strength of the cured Vetbond™ adhesive was inferior to the pulsed laser irradiation operated at a wavelength of 805 nm due to the higher degree of degradation with the 1064 nm laser condition (data not shown).

TABLE 8
PMMA Additive Strengthened Curable N-Butyl Cyanoacrylate

| | | Exp. 1 | Exp. 2 | Exp. 3 | Exp. 4 | Exp. 5 | Exp. 6 | Avg. | St. |
|---|---|---|---|---|---|---|---|---|---|
| Entry | | (N) | (N) | (N) | (N) | (N) | (N) | (N) | Dev. |
| Laser condition 1 | | 30 J/cm² and a 100 ms slow | | | | | | | |
| 1 | 25 μL | 8.8 | 12.7 | 10.3 | 1.1 | 4.3 | | 7.4 | 4.7 |
| | Vetbond ™ | | | | | | | | |
| | opened bottle | | | | | | | | |
| | No laser | | | | | | | | |
| 2 | 25 μL | 12.1 | 7.8 | 13.7 | | | | 11.2 | 3.0 |
| | Vetbond ™ New | | | | | | | | |
| | bottle | | | | | | | | |
| Laser condition 2 | | 15 J/cm² and a 100 ms slow | | | | | | | |
| 3 | 25 μL 5.0 | | | | | | | | |
| | mg/mL IR dye | | | | | | | | |
| | in Vetbond ™ | | | | | | | | |
| 4 | 25 μL 5.0 | 13.3 | | | 13.1 | 8.8 | 10.0 | 12.1 | 2.1 |
| | mg/mL IR dye | | | | | | | | |
| | and 5.0 mg/mL | | | | | | | | |
| | PMMA in | | | | | | | | |
| | Vetbond ™ | | | | | | | | |
| | No laser | | | | | | | | |
| 5 | 25 μL 5.0 | 7.8 | 4.7 | 8.7 | | | | 7.1 | 2.1 |
| | mg/mL IR dye | | | | | | | | |
| | and 5.0 mg/mL | | | | | | | | |
| | PMMA in | | | | | | | | |
| | Vetbond ™ | | | | | | | | |
| Laser condition 3 | | 10 J/cm² and a 100 ms slow | | | | | | | |
| 6 | 25 μL 5.0 | 4.5 | 7.5 | 8.1 | 1.3 | 4.5 | | 5.2 | 2.7 |
| | mg/mL IR dye | | | | | | | | |
| | in Vetbond ™ | | | | | | | | |
| | No laser | | | | | | | | |
| 7 | 25 μL 5.0 | 3.7 | 8.0 | 5.2 | 5.4 | 6.0 | | 5.6 | 1.6 |
| | mg/mL IR dye | | | | | | | | |
| | in Vetbond ™ | | | | | | | | |

The PMMA+cyanoacrylate+IR Dye particles+laser cured rapidly, and the bond was as strong as just the cyanoacrylate. However, addition of PLGA to the cyanoacrylate mixture with the IR Dye particles followed by laser irradiation did not improve the bond strength.

In the absence of PMMA additive, the addition of IR dye particle as catalyst for laser accelerated curing of the Vetbond™ adhesive caused weakening of the bond strength. The heat generated from photothermal conversion from IR dye particles caused the degradation of the cured n-butyl cyanoacrylate adhesive (entries 1-2 vs. entries 6-7 of Table 8).

The cured Vetbond™ adhesive modified with the IR dye particles without PMMA was considerably weaker than that of the cured Vetbond™ adhesive modified with IR dye particles and PMMA (entries 4-5 vs. entries 6-7 of Table 8). The addition of 5.0 mg/mL of PMMA to Vetbond™ brand adhesive modified with IR dye particles made the bond strength much greater.

### Example 9. Photothermal In Situ Curing of In Situ Curable Dental Composition

(1) To an ethanol solution of butyl cyanoacrylate at concentration of about 0.20 g/mL in a series of glass test tubes under stirring, Epolight™ 1117 IR dye loaded 96/4 PMMA/BMA particles is added at a concentration of 0, 1.0 mg/mL, 2.0 mg/mL, 3.0 mg/mL, 4.0 mg/mL, 5 mg/mL to form a liquid mixture. The liquid mixture in each of the test tube is irradiated with a pulsed laser at a wavelength of 1064 nm, and a power density of 0.25 W/cm² for 5 minutes. The butyl cyanoacrylate liquid formulation rapidly gels inside the test tube.

(2) Prepare a stock solution of thermal initiator by dissolving 10 mg 2,2′-azobis [2-(2-imidazolin-2-yl) propane] dihydrochloride in 2 mL water to form a solution having 5 mg/mL concentration.

Prepare a stock solution of in situ curable hydrogel precursor solution by dissolving 1 g of polyethylene glycol diacrylate in 5 mL water to provide a solution having 0.2 g/mL concentration.

To a water solution of polyethylene glycol diacrylate at concentration of about 0.20 g/mL in a series of glass test tubes under stirring, an aliquot of 1 mL of thermal initiator solution is added, followed by the addition of Epolight™ 1117 IR dye loaded 96/4 PMMA/BMA particles at a concentration varies at 0, 1.0 mg/mL, 2.0 mg/mL, 3.0 mg/mL, 4.0 mg/mL, 5 mg/mL to form a liquid mixture. The liquid mixture in each of the test tube is irradiated with a pulsed laser at a wavelength of 1064 nm, and a power density of 0.25 W/cm² for 5 minutes. The butyl cyanoacrylate liquid formulation rapidly gels inside the test tube.

### Example 10. On-demand, Remotely Generated Radicals as Accelerators for Curable Bone Cement Compositions

In this study, in order to lower the maximum polymerization temperature (Tₘₐₓ) of the bone cement, 5 mg/mL of IR Dye (ICG) particles disclosed herein will be added to modify the liquid phase of the Surgical Simplex® P brand bone cement. The ICG in the particles is a photosensitizing agent that interacts with light to generate reactive radical species that act as polymerization accelerator for the curing of the bone cement composition. This leads to reduction in the setting time for the composition while maintaining the mechanical strength achieved by the cured Surgical Simplex® P brand bone cement.

The Surgical Simplex® P brand bone cement has the following composition: Part 1 solid phase containing: 83-99 wt. % of PMMA beads, 9-15 wt. % of a radiopacifier of BaSO₄ or ZrO₂ particles, 0.75 wt. % to 2.60 wt. % benzoyl peroxide (BPO) initiator; Part 2 liquid phase containing: 97-99 wt. % of methyl methacrylate monomer (MMA), 0.8 wt. % to 1.4 wt. % of N,N-dimethyl-p-toludine (DMPT as accelerator, reduced from 2.5 wt. % in the conventional bone cement sold on the market), 15-75 ppm of hydroquinone as an inhibitor. The computed polymerization rate at 37° C. is 97% lower than that of the liquid phase containing 2.5% DMPT. The maximum polymerization temperature Tₘₐₓ is ˜7% lower and the setting time for Surgical Simplex® P brand bone cement is 14 minutes (54% higher than that of the liquid phase contains 2.5% DMPT). The longer setting time at 14 minutes poses a problem for its preparation and handing for use in a cemented total hip joint replacements (THJR) due to the fact that the maximum cement setting time limit for THJR recommended is at about 14 minutes per ISO 5833. The advantageous property provided by the Surgical Simplex® P brand bone cement is its reduced amount of residual monomer and the long-term stability of the cured bone cement.

This study aims to reduce the setting time of the Surgical Simplex® P Brand bone cement from 14 minutes to under 5 minutes using the radicals produced by the inventive particle heaters containing the material (e.g., ICG) as disclosed herein while maintaining the low Tₘₐₓ by accelerating the polymerization reaction with the generated radical species.

The IR dye particles used throughout example 8 are 2 micron particles composed of a core having ICG dye and NeoCryl™ B-805 polymer (MMA/BMA copolymer), and a 25 VTMS/PEG shell (c=5.0 mg/mL, weight ratio of the VTMS/PEG shell to the uncoated PMMA core is 0.33:1) (referred as IR dye particles thereafter). The loading amount of ICG in the PMMA particle is about 12 wt. % by the total weight of the particle.

The solid phase of the Surgical Simplex® P bone cement is used as it is. The liquid phase of the curable bone cement containing ICG particles is prepared by suspending 2.5 mg of ICG particles (concentration 1, c₁=5.0 mg/mL): in 500 μl of the liquid phase of the Surgical Simplex® P bone cement. The mixture is vortexed for 1 minute to allow the formation of a homogenous suspension of the ICG particles in the liquid phase containing the methyl methacrylate monomer. Two other concentrations of ICG particles are tested; concentration 2, c₂=2.5 mg/ml and concentration 3, c3=10 mg/ml. For 2.5 mg/ml, 1.25 mg of ICG particles are suspended in 500 μl of the liquid phase of the Surgical Simplex® P bone cement. For the 10 mg/ml concentration, 5 mg of ICG particles are suspended in 500 μl of the liquid phase of the Surgical Simplex® P bone cement.

In a mixing bowl, the solid phase of the Surgical Simplex® P bone cement is blended with the modified liquid phase of the Surgical Simplex® P bone cement containing the ICG particles. This is done separately for all the three concentrations of the ICG particles in the liquid phase of the Surgical Simplex® P bone cement. The curable bone cement mixtures are then poured into separate plastic culture dish. The timer is started to measure setting time.

For each concentration of the ICG particles, laser treatment is applied over the curable ICG particles modified Surgical Simplex® P bone cement in the plastic culture dish. The 805 nm laser is set to a fluence of 30 J/cm² and a 100 ms (millisecond) pulse. The laser is held approximately 0.5 inches above the surface of the culture dish and pulsed once. The test is repeated with the laser setting to a fluence of 15 J/cm² and 100 ms pulse and 10 J/cm² and 100 ms pulse with the cold setting on and off. To establish reproducibility this will be repeated five times for each setting.

The setting time of the curable bone cement is recorded and compared with the 14 minutes setting time of the Surgical Simplex® P bone cement. During the setting process, the temperature of the bone cement is recorded using a thermocouple. The maximum temperature reached during the curing process (Tₘₐₓ) is recorded and compared for all the samples.

### Example 11. Controlled Heat Generation from Heat Delivery Coating Composition Containing IR Dyes

(i) Preparation of Thermoresponsive SMP Fibers having a Coating Containing IR Absorbing Material

The SMP fibers of poly(p-dioxanone) were purchased from Ethicon. The IR absorbing materials include Epolight™ 1117 dye, ICG dye and IR 193 squarylium dye. ICG dye was purchased as DMSO stock solution. The filming forming agents used for preparing the coating formulations include PMMA/BMA and poly(lactide-co-glycolide) PLGA. NeoCryl® B-805 polymer (MMA/BMA copolymer, weight average molecular weight=85,000 Da, glass transition temperature T_{g}=99° C.) was purchased from DSM. PLGA 75:25 (lactide:glycolide=75:25, MW: 10,000-15,000 Da) was purchased from PolySciTech (West Lafayette, Ind., USA). NeoCryl® B-805 polymer was used as film forming material for Epolight™ 1117 dye and IR 193 squarylium dye.

The poly(p-dioxanone) fibers as purchased were cut into ten 1 inch long pieces.

The preparation of the polymer-based coating solutions of Epolight™ 1117 dye and IR 193 squarylium dye (c₁=1.0 mg/mL): to 100 ml of dichloromethane was added 1.0 g of DSM NeoCryl® B-805 polymer (MMA/BMA copolymer) (c=10.0 mg/mL), 0.1 g of Epolight™ 1117 dye or 0.1 g of IR 193 dye to allow the formation of a clear solution of NeoCryl® B805 polymer and the dyes.

The preparation of the polymer-based coating solutions of IR 193 squarylium dye (c₂=2 mg/mL): to 100 ml of dichloromethane was added 1.0 g of DSM NeoCryl® B-805 polymer (MMA/BMA copolymer) (c=10 mg/mL), 0.2 g of IR 193 dye to allow the formation of a clear solution of NeoCryl® B805 polymer and the IR 193 dye.

The preparation of the polymer-based coating solutions of ICG dye (c₁=1 mg/mL): to 100 ml of acetone was added 1.0 g of PLGA 75:25 (c=10 mg/mL), 0.1 g of ICG dye to allow the formation of a clear solution of PLGA 75:25 and ICG.

The preparation of the polymer-based coating solutions of ICG dye (c₂=2 mg/mL): to 100 ml of acetone was added 1.0 g of PLGA 75:25 (c=10 mg/mL), 0.2 g of ICG dye to allow the formation of a clear solution of PLGA 75:25 and ICG.

The 1 inch long fiber pieces were coated with the various dye stock solutions as above by dipping the fibers into the coating solution and held in there for 10 minute. The wet coated fibers were air dried for 5 minutes followed by drying at 40° C. in an oven for 30 minutes.

(ii) Laser Induced Shrinkage of Thermoresponsive Poly(p-Dioxanone) Fibers with 1060 nm and 805 nm Laser

(a) Laser Induced Shrinkage of Epolight™ 1117 Dye Coated Poly(p-Dioxanone) Fibers in the Absence of Cold Setting

The Epolight™ 1117 dye coated poly(p-dioxanone) fibers as in Experiment 8a was secured in the ADMET Expert Biomechanical Testing Machine. The 1060 nm laser was set to a fluence of 40 J/cm² and a 30 ms (millisecond) pulse. The laser was held approximately 0.5 inches from the middle of the poly(p-dioxanone) fibers and pulsed once. After laser treatment, the amount of the force created by the contraction expressed in unit of Newtons was measured and documented as observed on the MTESTQuattro software. The test was repeated with the laser setting to a fluence of fluence of 40 J/cm² and 100 ms pulse with the cold setting on. This was repeated for reproducibility with five independently coated fibers for each setting.

(b) Laser Induced Shrinkage of Epolight™ 1117 Dye Coated Poly(p-Dioxanone) Fibers with 1060 Nm Laser at Lower Frequency (0.97 Hz) in the Presence of Cold Setting

The Epolight™ 1117 dye coated poly(p-dioxanone) fibers as in Experiment 8a was secured in the ADMET Expert Biomechanical Testing Machine. The 1060 nm laser was set to a fluence of 40 J/cm² and a 100 ms pulse and at a frequency of 0.97 Hz with the cold setting on. The laser was held approximately 0.5 inches from the middle of the poly(p-dioxanone) fibers and pulsed once. After laser treatment, the amount of the force created by the contraction expressed in unit of Newtons was measured and documented as observed on the MTESTQuattro software. The tests were repeated with 4 more Epolight™ 1117 dye coated poly(p-dioxanone) fibers.

(c) Laser Induced Shrinkage of Epolight™ 1117 Dye Coated Poly(p-Dioxanone) Fibers with 805 Nm Laser at Lower Frequency (0.97 Hz) in the Presence of Cold Setting

The Epolight™ 1117 dye coated poly(p-dioxanone) fibers as in Experiment 8a was secured in the ADMET Expert Biomechanical Testing Machine. The 805 nm laser was set to a fluence of 40 J/cm² and a 100 ms pulse and at a frequency of 0.97 Hz with the cold setting on. The laser was held approximately 0.5 inches from the middle of the poly(p-dioxanone) fibers and pulsed once. After laser treatment, the amount of the force created by the contraction expressed in unit of Newtons was measured and documented as observed on the MTESTQuattro software. The tests were repeated with 4 more Epolight™ 1117 dye coated poly(p-dioxanone) fibers.

TABLE 9
Laser induced shrinkage of Epolight ™
1117 dye coated poly(p-dioxanone) fibers ^{a}

| 1060 nm laser @ 40 J/cm²-100 | | 805 nm laser @ 40 J/cm²-100 | |
|---|---|---|---|
| ms-0.97 Hz-cold on | | ms-0.97 Hz-cold on | |
| Test | Max Force (N) | Test | Max Force (N) |
| 1 | 0.066 | 1 | 0.114 |
| 2 | 0.088 | 2 | 0.064 |
| 3 | 0.092 | 3 | 0.133 |
| 4 | 0.037 | 4 | 0.045 |
| 5 | 0.075 | 5 | 0.077 |
| Average | 0.072 | Average | 0.087 |
| Median | 0.072 | Median | 0.077 |
| STEDV | 0.022 | STEDV | 0.036 |

^{a} Tensile strength of the poly(p-dioxanone) fiber after laser treatment was measured as max force in Newtons.

(d) Laser Induced Shrinkage of ICG Dye Coated Poly(p-Dioxanone) Fibers with 805 nm Laser at Lower Frequency (0.97 Hz) in the Presence of Cold Setting

The poly(p-dioxanone) fibers coated with ICG dye as in Experiment 8a was secured in the ADMET Expert Biomechanical Testing Machine. The 805 nm laser was set to a fluence of 40 J/cm² and a 100 ms pulse and at a frequency of 0.97 Hz with the cold setting on. The laser was held approximately 0.5 inches from the middle of the poly(p-dioxanone) fibers and pulsed once. After laser treatment, the amount of the force created by the contraction expressed in unit of Newtons was measured and documented as observed on the MTESTQuattro software. The tests were repeated with 4 more ICG coated poly(p-dioxanone) fibers.

TABLE 10
Laser induced shrinkage of ICG dye coated poly(p-dioxanone)
fibers having two concentrations of ICG dye in the coating^{a}

| | |
|---|---|
| 805 nm laser @ 40 J/cm²-100 | 805 nm laser @ 40 J/cm²-100 |
| ms-0.97 Hz-cold on, ICG (c1) | ms-0.97 Hz-cold on, ICG (c2) |

| Test | Max Force (N) | Test | Max Force (N) |
|---|---|---|---|
| 1 | 0.101 | 1 | 0.041 |
| 2 | 0.108 | 2 | 0.096 |
| 3 | 0.028 | 3 | 0.096 |
| 4 | 0.057 | 4 | 0.092 |
| 5 | 0.094 | 5 | 0.070 |
| Average | 0.078 | Average | 0.079 |
| Median | 0.094 | Median | 0.092 |
| STEDV | 0.034 | STEDV | 0.024 |

^{a}Tensile strength of the ICG dye coated poly(p-dioxanone) fibers after laser treatment was measured as max force in Newtons.

(e) Laser Induced Shrinkage of IR 193 Dye Coated Polyp-Dioxanone) Fibers with 805 nm Laser at Lower Frequency (0.97 Hz) in the Presence of Cold Setting

The poly(p-dioxanone) fibers coated with IR 193 dye as in Experiment 8a was secured in the ADMET Expert Biomechanical Testing Machine. The 805 nm laser was set to a fluence of 40 J/cm² and a 100 ms pulse and at a frequency of 0.97 Hz with the cold setting on. The laser was held approximately 0.5 inches from the middle of the poly(p-dioxanone) fibers and pulsed once. After laser treatment, the amount of the force created by the contraction expressed in unit of Newtons was measured and documented as observed on the MTESTQuattro software. The tests were repeated for reproducibility with four more IR 193 dye coated poly(p-dioxanone) fibers.

TABLE 11
Laser induced shrinkage of IR 193 dye coated poly(p-dioxanone)
fibers having two IR 193 dye concentrations in the coating

| | |
|---|---|
| 805 nm laser @ 40 J/cm²-100 | 805 nm laser @ 40 J/cm²-100 |
| ms-0.97 Hz-cold on, | ms-0.97 Hz-cold on, |
| IR 193 dye (c1) | IR 193 dye (c2) |

| Test | Max Force (N) | Test | Max Force (N) |
|---|---|---|---|
| 1 | 0.041 | 1 | 0.080 |
| 2 | 0.081 | 2 | 0.064 |
| 3 | 0.086 | 3 | 0.035 |
| 4 | 0.049 | 4 | 0.044 |
| 5 | 0.077 | 5 | 0.050 |
| Average | 0.067 | Average | 0.055 |
| Median | 0.077 | Median | 0.050 |
| STEDV | 0.020 | STEDV | 0.018 |

A Tensile strength of the IR 193 dye coated poly(p-dioxanone) fibers after laser treatment was measured as max force in Newtons.

(f) Uncoated Poly(p-Dioxanone) Fibers as Negative Control with 1060 nm and 805 nm Laser

The poly(p-dioxanone) fibers without coating was secured in the ADMET Expert Biomechanical Testing Machine. The 1060 nm and 805 nm laser were set to a fluence of 40 J/cm² and a 100 ms pulse and at a frequency of 0.97 Hz with the cold setting on. The laser was held approximately 0.5 inches from the middle of the poly(p-dioxanone) fibers and pulsed once. After laser treatment, the amount of the force created by the contraction expressed in unit of Newtons was measured and documented as observed on the MTESTQuattro software. The tests were repeated for reproducibility with 4 more poly(p-dioxanone) fibers without coating.

TABLE 12
Laser induced shrinkage of poly(p-dioxanone)
fibers without coating^{a} (blank control)

| 1060 nm laser @ 40 J/cm²-100 | | 805 nm laser @ 40 J/cm²-100 | |
|---|---|---|---|
| ms-0.97 Hz-cold on | | ms-0.97 Hz-cold on | |
| Test | Max Force (N) | Test | Max Force (N) |
| 1 | −0.001 | 1 | −0.004 |
| 2 | −0.010 | 2 | −0.008 |
| 3 | 0.000 | 3 | −0.001 |
| 4 | −0.008 | 4 | −0.005 |
| 5 | −0.004 | 5 | −0.005 |
| Average | −0.005 | Average | −0.005 |
| Median | −0.004 | Median | −0.005 |
| STEDV | 0.004 | STEDV | 0.003 |

^{a}Tensile strength of the uncoated poly(p-dioxanone) fiber after laser treatment was measured as max force in Newtons.

The poly(p-dioxanone) fibers having various IR dye coating are susceptible to thermal induced shrinkage resulted in the tightening of poly(p-dioxanone) fibers s as evidenced by the tensile strength of the poly(p-dioxanone) fibers after laser treatment was measured as max force in Newtons with an ADMET Expert Biomechanical Testing Machine. The IR coatings have been tested include coatings containing the IR absorbing dye Epolight™ 1117, as well as ICG and squariynium dye IR193 dye. Treating the poly(p-dioxanone) fibers with the IR laser caused them to shrink with exhibiting an average max force that was almost identical between Epolight™ 1117 dye, ICG, and IR193 dye. The average max force for the IR dye coated poly(p-dioxanone) fibers was similar in both the 1064 nm and 805 nm laser, but it was observed that the 805 nm resulted in less melting of the poly(p-dioxanone) fibers. This is most likely due to the fact that 1064 nm is very close to the peak of 1064 nm of the IR dye which in turn generates more heat when hit with the laser pulse. It was also observed that the cool setting helped prevent melting of the poly(p-dioxanone) fibers. Thus, the preferred conditions were the 805 nm laser with the cold setting on.

The results in Tables 9-12 demonstrated that IR dye modified coatings on the surface of thermoresponsive SMP fibers (e.g., poly(p-dioxanone)) was capable of absorbing photonic energy from laser irradiation, converting the photonic energy to heat, and transmitting the heat from the IR dye modified coatings to the underlie thermoresponsive SMP fibers, and the heating causing a change to the physical property (i.e., shape) of the thermoresponsive SMP fibers. The heat delivery capability of the IR dye modified coating was evidenced by the fact that the thermoresponsive SMP fibers with the IR dye modified coating shrank (exhibited shape memory effects) after the exposure to pulsed laser irradiations (See the examples below). The shape memory effects of the thermoresponsive SMP fibers were quantified in term of the amount of the force created by the contraction expressed in unit of Newtons (See Tables 9-12).

### Example 12. Hemolysis Test

Hemolysis, which refers to the destruction of red blood cells, in vivo can lead to anemia, jaundice and other pathological conditions. Therefore, the hemolytic potential of all intravenously administered pharmaceuticals, including the compositions presented herein must be evaluated. The assay presented below is an adaptation of existing ASTM standard F-756-00, which is based on colorimetric detection of red-colored cyanmethemoglobin in solution. Single donor human blood is purchased from Innovative Research (Novi, Mich.). Blood is pooled from three single donors for the hemolysis test. Innovative Research's single donor human whole blood is drawn from healthy in FDA-licensed facilities. All lots have been tested by FDA-approved for human immunodeficiency virus RNA (HIV-1RNA), antibodies to immunodeficiency virus (Anti-HIV1/2), antibodies to hepatitis c virus (HCV), hepatitis c virus ma (hcv ma), hepatitis b virus (hbv dna), hepatitis b surface antigen (hbsag), and syphilis. Lithium heparin or sodium citrate are added as anticoagulant. In this assay, particles or compositions are incubated in blood, exposed to the exogenous source and the resulting blood is analyzed and compared to samples that receive no exogenous energy and those that have nothing added to them. The hemoglobin released by damaged cells in these samples is oxidized to methemoglobin by ferricyanide in the presence of bicarbonate, and then cyanide converts the methemoglobin to cyanmethemoglobin. The undamaged erythrocytes are removed by centrifugation, and the amount of cyanmethemoglobin in the supernatant is measured by spectrophotometry at its absorbance maximum wavelength, 540 nm. This measured absorbance is compared to a standard curve to determine the concentration of hemoglobin in the supernatant, and this hemoglobin concentration is compared to that in the supernatant of a blood sample not treated with nanoparticles to obtain the percentage particle-induced hemolysis (referred to as percent hemolysis). The standard curve is created from a linear fit of several absorbance measurements made at 540 nm on a hemoglobin standard sample (treated with ferricyanide and bicarbonate) over a range of hemoglobin concentrations from 0.025 mg/mL to 80 mg/mL (calibration standards). To compare intra-assay performance, the positive and the negative control samples are analyzed six times in one validation run.

The precision of the measured hemoglobin concentrations (determined as percent coefficient variation, % CV) and accuracy (determined as percent difference from theoretical, PDFT), with the theoretical concentration corresponding to the value of the standard curve are calculated for each sample over all assay runs. [h] is the mean measured hemoglobin concentration for a particular sample over all runs, and % CV is the percentage of the mean of the standard deviation (% CV=100×SD/[h]) and % DFT is the percent difference of the mean concentration from the theoretical concentration (PDFT=100× (_{([h]-[h]}_{_{theory)}}_{/[h]}_{_{theory}})). In addition to the calibration standards, the assay includes measurement on hemoglobin standard samples (treated with ferricyanide and bicarbonate) with known concentrations, which are referred to as “quality controls” for this assay. Results are presented as percent of hemolysis for different particles/compositions at different concentrations. Less than 30% hemolysis at the intended particle/composition dose is considered as the passing criterion for this test.

The experimental procedure described in the ASTM standard was modified by: 1) scaling it to a 96-well plate format, 2) introduction of particle-relevant controls, and 3) modification of acceptance criteria to reflect ICHS6 requirements for bioanalytical method validation. The results of this assay (expressed as percentage hemolysis with respect to negative control) are used to evaluate the acute in vitro hemolytic properties of the particles and/or the compositions.

### Example 13. Laser Triggered Photothermal Blood Clotting by the Particle Heaters

The IR dye particles used throughout the Example 11 is a 2 micron particles composed of a core having Epolight™ 1117 dye and Neocryl™ B-728 polymer (MMA copolymer), and a 25% VTMS/PEG shell (c=5.0 mg/mL, weight ratio of the VTMS/PEG shell to the uncoated PMMA core is 0.33:1) (referred as IR dye particles thereafter). The loading amount of Epolight™ 1117 in the PMMA particle of about 12 wt. % by the total weight of the particle.

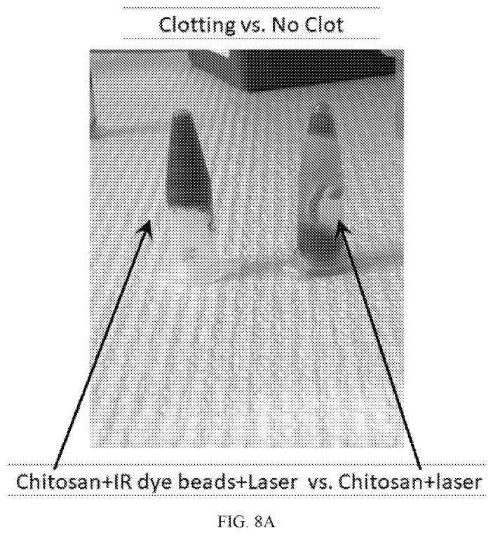

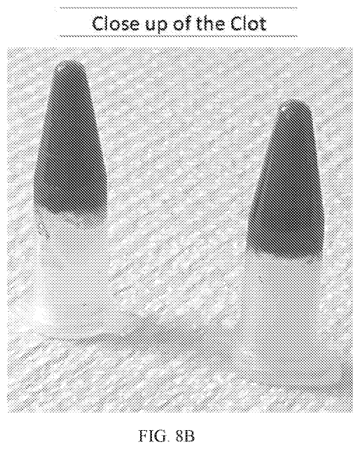

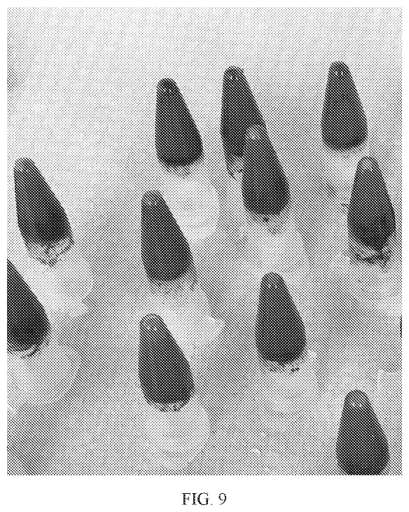

0.5 mL of citrated animal blood was mixed with 20 μL of calcium chloride in an Eppendorf plastic tube. Either chitosan alone (a commonly used hemostat) or a mixture of chitosan and IR dye beads (2 μm Epolight™ 1117 MMA/BMA copolymer particles) were deposited on the top of the blood in the tube. Both samples were irradiated with a 1064 nm pulsed laser at a fluence of 100 J/cm², 400 ms pulse. The tubes were inverted after the laser irradiation. If the top layer of blood clotted, the inversion of the tube did not lead to the blood flowing downwards under the action of gravity. This was observed in the sample wherein a mixture of chitosan and IR Dye beads were added to the top of the blood. However, for the comparable sample containing chitosan alone, it was observed that the blood rapidly flowing to the bottom of the inverted Eppendorf tube. Laser light alone and IR Dye beads mixed with chitosan alone (i.e. no laser irradiation) did not show any clot formation. However, IR dye beads added to the blood followed by laser irradiation also induced clot formation. Visibly, the clot formed by the mixture of chitosan and IR dye beads was a thicker clot than that obtained by only IR dye beads that were irradiated (FIGS. 8A-B). No controls are shown in the Figures. This experiment proved that particle heaters could induce rapid clot formation either by themselves or in conjunction with existing hemostats. A clot when formed was visible almost instantaneously after laser exposure. Under lower light fluences of 30 J/cm², the blood samples had to be exposed to the laser three times before a full clot in the top layer could be observed.

TABLE 13
Results for IR dye enhanced blood clotting
following laser irradiation^{a}

| | | Blood | | | Blood |
|---|---|---|---|---|---|
| | | sample^{b} | | | sample |
| | | temperature | | | temperature |
| | | before laser | No. of | | after laser |
| | hemostatic | irradiation | pulse | Clotting | irradiation |
| entry | composition | (° C.) | applied | status | (° C.) |
| 1 | 20 mg chitosan | 37 | 1 | no | N/A |
| 2 | 20 mg chitosan | 37 | 2 | no | N/A |
| 3 | 20 mg chitosan | 37 | 3 | no | N/A |
| 4 | 20 mg chitosan | 37 | 5 | yes | N/A |
| 6 | Laser alone | 30 | 5 | yes | |
| 7 | Laser alone | 30 | 5 | yes | 70 |
| 8 | 5 mg IR dye | 30 | 2 | yes | 41 |
| | particles | | | | |
| 9 | 10 mg IR dye | 30 | 1 | yes | 39 |
| | particles | | | | |
| 10 | 20 mg | 4 | 1 | yes | N/A |
| | chitosan + 5 mg | | | | |
| | IR Dye Beads | | | | |
| 11 | Laser Alone | 4 | 1 | no | N/A |
| 12 | Laser Alone | 4 | 2 | no | N/A |
| 13 | Laser Alone | 4 | 3 | no | N/A |
| 14 | Laser Alone | 4 | 4 | no | |
| 15 | Laser Alone | 4 | 5 | yes | The surface |
| | | | | | of the |
| | | | | | blood sample |
| | | | | | burned |

^{a}laser operating parameters for all experiments in Table 13: laser wavelength: 1064 nm; fluence: 100 J/cm²; pulse duration: 400 ms; and cool tip on.
^{b}Blood samples used: 500 μl of citrated blood admixed with 75 μL of CaCl₂ placed in a 1.5 mL eppendorf plastic tube.

The results in Table 13 demonstrated that the addition of an exogenous IR absorbing material accelerated the blood clotting process after the exposure to the laser irradiation (See entries 8-10 of Table 13). The result in entry 7 showed that the endogenous IR absorbing agent hemoglobin could serve as an agent of photothermal conversion. Without the IR absorbing material, the application of IR laser light could trigger clotting, but only after a bulk rise in temperature of about 40° C. (entry 3 vs. entry 7). Chitosan offered no improvement in clotting under the laser-induced hyperthermia, requiring exposure similar to that of samples lacking chitosan.

The results in Table 13 also demonstrated that chitosan hemostat alone did not absorb laser photonic energy such that there is no additional heat generation. The results also indicated that the absorbance of laser light by the exogenous IR dye is more efficient than that of the endogenous hemoglobin (a red protein carries oxygen) in the blood sample. Irradiation of blood alone required 5 laser pulses to trigger the clot formation, whereas samples containing the IR dye-containing particles needed only one laser pulse for clot formation (entries 1-7 vs. entries 8-10 of Table 13).

In the absence of IR dye in the blood sample, laser treatment of hypothermic blood samples at 4° C. triggered the clot formation, but only after 5 pulses of laser (entries 11-15 of Table 13), which led to charring of the surface. The addition of 20 mg of exogenous IR dye particles to the hypothermic blood samples at 4° C. accelerated the blood clotting process as compared with the sample without IR dye (entry 10 vs. entries 11-15, Table 13).

While the concepts of the present technology have been particularly shown and described above with reference to exemplary embodiments thereof, it will be understood by those of ordinary skill in the art, that various changes in form and detail can be made without departing from the spirit and scope of the concepts described herein. It is to be understood that features from any one embodiment described herein may be combined with features of any other embodiment described herein to form another embodiment of the invention.

1. A heat delivery medium comprising a carrier and a material that interacts with an exogenous source, wherein the material absorbs energy from the exogenous source and converts the absorbed energy to heat, wherein the heat travels outside the medium in a controlled temperature range to initiate or accelerate a physical, chemical or biological activity, and wherein the medium passes an Extractable Cytotoxicity Test.

2. The heat delivery medium of claim 1, wherein the heat delivery medium further passes a Thermal Cytotoxicity Test.

3. The heat delivery medium of claim 1, wherein the heat delivery medium further passes an Efficacy Determination Protocol.

4. The heat delivery medium of claim 1, wherein the material exhibits at least 20% energy-to-heat conversion efficiency.

5. The heat delivery medium of claim 1, wherein the material exhibits at least 20% efficiency of conversion of energy from the exogenous source to heat.

6. The heat delivery medium of claim 1, wherein the carrier and the material form a particle.

7. The heat delivery medium of claim 6, wherein the particle maintains integrity after interacting with the exogenous source.

8. The heat delivery medium of claim 6, wherein the particle structure is altered after interacting with the exogenous source.

9. The heat delivery medium of any one of claims 1-8, wherein the carrier is selected from the group consisting of oil carrier including fatty ester oils, squalene, hydrocarbon oil, light mineral oil, isoparaffin, paraffin oil, water, alcohol solution in water (C1-C4 alcohols), aqueous solution of polyhydric alcohol (e.g. glycerol, ethylene glycol, 1,3-propanediol, 1,4-butanediol), emulsion, saline, PBS buffer, and combinations thereof.

10. The heat delivery medium of any one of claims 1-8, wherein the carrier is selected from the group consisting of lipid, film forming polymer, thermoresponsive polymer, pressure sensitive adhesive, shape memory polymer, hydrogel, and combinations thereof.

11. The heat delivery medium of any one of claims 1-8, wherein the carrier is a coating composed of film forming polymer.

12. The heat delivery medium of claim 11, wherein the film forming polymer is selected from the group consisting of poly(methyl methacrylate), poly(lactide-co-glycolide) (PLGA), block copolymer of PLGA, polyethylene glycol (PLGA-PEG), and combinations thereof.

13. The heat delivery medium of any one of claims 1-12, wherein the material has absorption of photonic energy in the near infrared spectrum region having a wavelength range from 750 nm to 1100 nm.

14. The heat delivery medium of any one of claims 1-12, wherein the material interacting with the exogenous source has absorption of photonic energy in the visible spectrum region having a wavelength ranging from 400 nm to 750 nm.

15. The heat delivery medium of claim 14, wherein the material absorbs light at a wavelength selected from the group consisting of 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, and 750 nm.

16. The heat delivery medium of any one of claims 1-15, wherein the material is selected from the group consisting of a tetrakis aminium dye, a cyanine dye, a squaraine dye, a squarylium dye, iron oxide, a plasmonic absorber, a zinc iron phosphate pigment, and combinations thereof.

17. A heat delivery composition comprising the heat delivery medium of any one of claims 1-16 and a structural element selected from a group consisting of a fiber, a film, a sheet, an implant scaffold, a tape, a stent, a hydrogel, a patch, an adhesive, a woven fabric, a nonwoven fabric, a biocompatible cross-linked polymer, and combinations thereof.

18. The heat delivery composition of claim 17, wherein the heat delivery medium is embedded within or disposed on the surface of the structural element as a coating.

19. The heat delivery composition of any one of claims 17-18, wherein the composition comprises a biocompatible cross-linked polymer.

20. The heat delivery composition of claim 19, wherein the biocompatible cross-linked polymer comprises a thermoresponsive hydrogel.

21. The heat delivery composition of claim 17, wherein the heat delivery composition further comprises an inorganic agent.

22. The heat delivery composition of claim 21, wherein the inorganic agent is selected from the group consisting of apatite, hydroxyapatite, hydroxycarbonate apatite, calcium carbonate, calcium phosphate including monocalcium phosphate, dicalcium phosphate, tricalcium phosphate, and tetracalcium phosphate, and combinations thereof.

23. The heat delivery composition of any one of claims 17-22, wherein the composition comprises a liquid formulation, a fiber, a coating, an implant scaffold, a hydrogel, an adhesive, a tape, a patch, a woven fabric, a nonwoven fabric, a film, a sheet, a multilayered structure, or a biocompatible cross-linked polymer.

24. The heat delivery composition of claim 23, wherein the biocompatible cross-linked polymer comprises reactive functional groups selected from the group consisting of vinyl methyl sulfone group, hydroxyl group (—OH), thiol group (—SH), amine group (—NH₂), aldehyde group (—CHO), carboxylic acid group (—COOH), epoxy group, and combinations thereof.

25. A particle heater comprising a particle comprising a carrier admixed with a material that interacts with an exogenous source, wherein the material absorbs the energy from the exogenous source and converts the absorbed energy to heat, wherein the heat travels outside the particle in a controlled temperature range to initiate or accelerate a physical, chemical or biological activity, and further wherein the particle structure is constructed such that it passes the Extractable Cytotoxicity Test.

26. The particle heater of claim 25, wherein the particle heater further passes the Thermal Cytotoxicity Test.

27. The particle heater of claim 25, wherein the particle heater further passes the Efficacy Determination Protocol.

28. The particle heater of claim 25, wherein the particle is a nanoparticle or a microparticle.

29. The particle heater of claim 25, wherein the particle maintains integrity after interacting with the exogenous source.

30. The particle heater of claim 25, wherein the particle structure is altered after interacting with the exogenous source.

31. The particle heater of any one of claims 25-30, wherein the particle further comprises a shell to form a core-shell particle.

32. The particle heater of claim 31, wherein the shell comprises iron oxide.

33. The particle heater of claim 31, wherein the shell comprises a plasmonic absorber.

34. The particle heater of claim 33, wherein the plasmonic absorber comprises plasmonic nanomaterials of noble metal gold (Au), silver (Ag) and copper (Cu) nanoparticles doped with sulfur (S), selenium (Se) or tellurium (Te) having a plasmonic resonance at a NIR wavelength.

35. The particle heater of any one of claims 25-34, wherein the material has significant absorption of photonic energy in the near infrared spectrum region having a wavelength range from 750 nm to 1100 nm.

36. The particle heater of any one of claims 25-34, wherein the material interacting with the exogenous source has significant absorption of photonic energy in the visible spectrum region.

37. The particle heater of claim 36, wherein the material absorbs light at a wavelength ranging from 400 nm to 750 nm.

38. The particle heater of any one of claims 36-37, wherein the material absorbs light at a wavelength selected from the group consisting of 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, and 750 nm.

39. The particle heater of any one of claims 17-38, wherein the material is selected from the group consisting of a tetrakis aminium dye, a cyanine dye, a squaraine dye, a squarylium dye, iron oxide, a plasmonic absorber, a zinc iron phosphate pigment, and combinations thereof.

40. The particle heater of any one of claims 25-39, wherein the carrier is selected from the group consisting of a lipid, an inorganic agent, an organic polymer, and combinations thereof.

41. The particle heater of claim 40, wherein the carrier is selected from the group consisting of poly (lactic acid) (PLA); poly(glycolic acid) (PGA); poly(lactide-co-glycolide) (PLGA); block copolymer of polyethylene glycol-b-poly lactic acid-co-glycolic acid (PEG-PLGA); polycaprolactone (PCL); poly-L-lysine (PLL); random graft co-polymer with a poly(L-lysine) backbone and poly(ethylene glycol) (PLL-g-PEG); dendritic polymer including polyethyleneimine (PEI) and derivatives thereof, dendritic polyglycerol and derivatives thereof, dendritic polylysine; and combinations thereof.

42. The particle heater of claim 40, wherein the carrier comprises a polyester selected from the group consisting of poly(lactic acid) (PLA), poly(glycolic acid) (PGA), PLGA, and combinations thereof.

43. The particle heater of claim 40, wherein the carrier comprises a polymer blend containing PLGA 75:25 and PLGA-PEG 75:25 with lactide:glycolide monomer ratio of 75:25.

44. The particle heater of claim 25, wherein the carrier is a lipid.

45. The particle heater of claim 44, wherein the lipid is selected from the group consisting of 1,2-dipalmitoyl-sn-glycero-3-phospho-(1′-rac-glycerol) (DPPG), 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-distearoyl-sn-glycero-3-phosphoethanolamine (DSPE, 18:0), 1,2-diarachidyl-sn-glycero-3-phosphoethanolamine (20:0 PE), 1-stearoyl-2-linoleoyl-sn-glycero-3-phosphoethanolamine, 1,2-dipalmitoyl-sn-glycero-3-phosphocholine (DPPC, 16:0 PC), 1,2-diheptadecanoyl-sn-glycero-3-phosphocholine (17:0 PC), 1,2-distearoyl-sn-glycero-3-phosphocholine (DSPC, 18:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

46. The particle heater of claim 44, wherein the lipid comprises a thermoresponsive lipid/polymer hybrid.

47. The particle heater of claim 46, wherein the thermoresponsive lipid/polymer hybrid is selected from the group consisting of triblock copolymer of [poly(2-isopropyl-2-oxazoline)-b-poly(dimethylsiloxane)-b-poly(2-isopropyl-2-oxazoline] and 1,2-dimyristoyl-sn-glycero-3-phosphocholine (DMPC) composite, and block copolymers poly(cholesteryl acrylate)-b-poly(N-isopropylacrylamide) (PNIPAAm), lipid composite, and combinations thereof.

48. A method for controlled heat generation comprising contacting the heat delivery medium of claim 1, or the particle heater of claim 25, with an exogenous source.

49. The method of claim 48, wherein the exogenous source is selected from the group consisting of an electromagnetic radiation, an electrical field, a microwave, a radio wave, ultrasonic radiation, a magnetic field, and combinations thereof.

50. The method of any one of claims 48-49, wherein the exogenous source comprises LED light or a laser light.

51. The method of claim 50, wherein the laser light is a pulsed laser light.

52. The method of claim 50, wherein the exogenous source comprises an LED light.

53. The method of claim 51, wherein the laser pulse duration is in a range from milliseconds to nanoseconds, and the laser has an oscillation wavelength at 805 nm, 808 nm, or 1064 nm.

54. The method of any one of claims 48-53, wherein the heat delivery medium absorbs the laser light having a wavelength ranging from 750 nm to 1400 nm.

55. The method of any one of claims claim 48-53, wherein the heat delivery medium absorbs the light having a wavelength ranging from 400 nm to 750 nm.

56. The method of any one of claims 48-53, wherein the material is a tetrakis aminium dye.

57. The method of any one of claims 48-53, wherein the material is indocyanine green.

58. The method of any one of claims 48-53, wherein the material is a squaraine dye.

59. The method of any one of claims 48-53, wherein the material is a squarylium dye.

60. The method of any one of claims 48-53, wherein the material is iron oxide.

61. The method of any one of claims 48-53, wherein the material is a plasmonic absorber.

62. The method of any one of claims 48-53, wherein the material is a zinc iron phosphate pigment.

63. The method of any one of claims 48-62, wherein the method further comprises heating the surrounding area in the proximity of the heat delivery medium, the heat delivery composition, or the particle heater by transferring heat to the surrounding area to induce localized hyperthermia.

64. The method of claim 63, wherein the induced hyperthermia is a mild hyperthermia at a temperature ranging from about 38.0° C. to about 41.0° C.

65. The method of claim 63, wherein the induced hyperthermia is a moderate hyperthermia at a temperature ranging from about 41.1° C. to about 45.0° C.

66. The method of claim 63, wherein the induced hyperthermia is a profound hyperthermia at a temperature ranging from about 45.1° C. to about 52.0° C.

67. An in situ curable bioadhesive comprising: (a) a polymerizable and/or crosslinkable precursor, and (b) the heat delivery medium of claim 1 or the particle heater of claim 25, wherein the heat induces localized hyperthermia in the bioadhesive, wherein the localized hyperthermia induces or accelerates an in situ curing reaction to provide a cured bioadhesive, and wherein the curable and cured bioadhesives pass the Extractable Cytotoxicity Test.

68. The in situ curable bioadhesive of claim 67, wherein the curable bioadhesive passes the Efficacy Determination Protocol.

69. The in situ curable bioadhesive of claim 67, wherein the curable bioadhesive passes the Thermal Cytotoxicity Test.

70. The in situ curable bioadhesive of claim 67, wherein the heat delivery medium comprises a carrier admixed with the material to form a particle.

71. The in situ curable bioadhesive of claim 70, wherein the particle maintains integrity after interacting with the exogenous source.

72. The in situ curable bioadhesive of claim 70, wherein the particle structure is altered after interacting with the exogenous source.

73. The in situ curable bioadhesive of claim 70, wherein the particle further comprises a shell to form a core-shell particle.

74. The in situ curable bioadhesive of claim 73, wherein the shell comprises a crosslinked inorganic polymer.

75. The in situ curable bioadhesive of any one of claims 73-74, wherein the shell comprises a crosslinked inorganic polymer selected from the group consisting of mesoporous silica, organo-modified silicate polymer derived from condensation of organotrisilanol or halotrisilanol, and combinations thereof.

76. The in situ curable bioadhesive of claim 74, wherein the crosslinked inorganic polymer comprise organo-modified polysilicates.

77. The in situ curable bioadhesive of claims 73-75, wherein the shell comprises a plasmonic absorber selected from the group consisting of a monomolecular film of noble metals including gold (Au), silver (Ag), copper (Cu), nanoporous gold thin film, and combinations thereof.

78. The in situ curable bioadhesive of claim 67, wherein the polymerizable and/or crosslinkable precursor is selected from the group consisting of a polymerizable monomer, a polymerizable prepolymer, a cross-linkable prepolymer, and combinations thereof.

79. The in situ curable bioadhesive of claim 67, wherein the polymerizable and/or crosslinkable precursor is a polymerizable monomer for radical polymerization.

80. The in situ curable bioadhesive of claim 67, wherein the polymerizable and/or crosslinkable precursor is a polymerizable prepolymer for radical polymerization.

81. The in situ curable bioadhesive of claim 67, wherein the carrier comprises a lipid or a biocompatible organic polymer.

82. The in situ curable bioadhesive of claim 81, wherein the lipid is selected from the group consisting of dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-dimyristoyl-sn-glycero-3-phosphorylglycerol (DMPG); 1,2-distearoyl-sn-glycero-3-phosphoethanolamine (DSPE); 1,2-dioleoyl-sn-glycero-3-phosphocholine (DOPC); 1,2-dioleoyl-sn-glycero-3-phosphoethanolamine (DOPE); 1,2-dipalmitoyl-sn-glycero-3-phospho-(1′-rac-glycerol) (DPPG); 1,2-distearoyl-sn-glycero-3-phosphocholine (DSPC), distearoylphosphoethanolamine conjugated with polyethylene glycol (DSPE-PEG); phosphatidylserine (PS), phosphatidylethanolamine (PE), phosphatidylglycerol (PG), phosphatidylcholine (PC), and combinations thereof.

83. The in situ curable bioadhesive of claim 67, wherein the carrier is selected from the group consisting of a polyester, a polyurea, a polyanhydride, a polysaccharide, a polyphosphoester, a poly (ortho ester), a poly (amino acid), a protein, and combinations thereof.

84. The in situ curable bioadhesive of claim 67, wherein the carrier is selected from the group consisting of poly(lactic acid) (PLA), poly(glycolic acid) (PGA), PLGA, poly(lactic acid)-polyethylene glycol-poly(lactic acid) (PLA-PEG-PLA), poly (L-co-D, L lactic acid) 70:30 (PLDLA); poly(L-lactic acid-co-glycolic acid), poly(D,L-lactic acid-co-glycolic acid); poly-valerolactone, poly-hydroxyl butyrate and poly-hydroxyl valerate, polycaprolactone (PCL), γ-polyglutamic acid graft with poly (L-phenylalanine) (γ-PGA-g-L-PAE), poly(cyanoacrylate) (PCA), polydioxanone, polyvinylpyrrolidone (povidone, PVP), poly(butylene succinate), polyalkyleneoxalate, polyalkylene succinate, poly(maleic acid), poly(trimethylene carbonate), poly(p-dioxanone), poly(butylene terephthalate), poly(P-hydroxyalkanoate)s, poly(hydroxybutyrate), and poly(hydroxybutyrate-co-hydroxyvalerate), poly (ε-lysine), poly-L-lysine (PLL), poly(valeric acid), and poly-L-glutamic acid, poly(ester amide), poly(ester ether) diblock copolymer of poly(sebacic acid) and polyethylene glycol (PSA-PEG), trimethylene carbonate, poly(β-hydroxybutyrate), poly(g-ethyl-L-glutamate), poly(iminocarbonate), poly(bisphenol A iminocarbonate), polyphosphazene, collagen, albumin, gluten, chitosan, hyaluronate, hyaluronic acid, cellulose, alginate, starch, gelatin, pectin, crosslinked dextran as reaction product of dextran with epihalogenohydrins, dihalogenohydrins, 1:2,3:4-diepoxybutane, diepoxy-propylether, and combinations thereof.

85. The in situ curable bioadhesive of any one of claims 67-84, further comprising a reinforcement filler selected from the group consisting of powders of high density polyethylene having a median particle size of about 50 μm or less, powders of PMMA having a median particle size of 50-60 μm, polyethylene (PE) fiber, ultra-high-strength PE, UHMWPE grafted with MMA, ultra-high-strength PE grafted with MMA, beads of rubber-toughened PMMA powder having a PMMA outer shell and an inner shell made of crosslinked butyl methacrylate-styrene copolymer, beads of poly(isobutylene), beads of acrynitrile-butadiene-styrene, beads of poly(ε-caprolactone), particles of poly(butylmethacrylate) (PBMA), PCL-toughened PMMA beads, polyethylene terephtahalate fiber, silanated HA particle, sintered HA particle, silane-treated fluorohydroxyapatite particle, Ca-hydroxyapatite, particle of PMAA, particle of PMETA-PMMA, particle of PEMA, particle of PEMA-n-BMA, ultra-high molecular wright polyethylene (UHMWPE), chitosan nanoparticles and combinations thereof.

86. The in situ curable bioadhesive of claim 85, wherein the reinforcement filler is 50-60 μm PMMA particles.

87. The in situ curable bioadhesive of any one of claims 67-86, wherein the material has significant absorption of photonic energy in the spectrum region having a wavelength range from 400 nm to 1400 nm.

88. The in situ curable bioadhesive of any one of claims 67-86, wherein the material has absorption of photonic energy in the near infrared spectrum region having a wavelength range from 750 nm to 1200 nm.

89. The in situ curable bioadhesive of any one of claims 67-86, wherein the material has absorption of photonic energy in the near infrared spectrum region having a wavelength range from 900 nm to 1100 nm.

90. The in situ curable bioadhesive of any one of claims 67-86, wherein the material has absorption of photonic energy in the near infrared spectrum region having a wavelength range from 750 nm to 850 nm.

91. The in situ curable bioadhesive of any one of claims 67-86, wherein the material has absorption of photonic energy in the spectrum region having a wavelength range from 400 nm to 750 nm.

92. The in situ curable bioadhesive of any one of claims 67-86, wherein the material is selected from the group consisting of organic dyes, inorganic dyes, near-infrared absorbing dyes, tetrakis aminium dyes, squaraine dye, squarylium dye, zinc iron phosphate pigments, indocyanine green, and combinations thereof.

93. The in situ curable bioadhesive of any one of claims 67-92, wherein the heat delivery medium comprises two or more materials and each absorbs energy from a different exogenous source.

94. The in situ curable bioadhesive of any one of claims 67-92, wherein the exogenous source is selected from the group consisting of a body chemical, an electromagnetic radiation, an electrical field, a microwave, a radio wave, ultrasonic radiation, a magnetic field, and combinations thereof.

95. An in situ curable tissue adhesive for wound repair comprising the curable bioadhesive of claim 67.

96. An in situ curable bioadhesive of claim 67, wherein the crosslinkable precursor comprises at least two crosslinkable hydrogel adhesive prepolymers

97. The in situ curable bioadhesive of claim 96, wherein the cross-linkable prepolymer comprises a reactive functional group selected from vinyl group (—CH═CH₂), ethynyl group (—CCH), hydroxyl groups (—OH), thiol groups (—SH), amine groups (—NH₂), aldehyde groups (—CHO), carboxylic acid groups (—COOH), epoxy groups, isocyanate groups, thioisocyante groups, and combinations thereof.

98. The in situ curable hydrogel of claim 96, further comprising a crosslinker selected from the group consisting of polyethylene glycol-2500 diacrylate, 8-arm PEG-2500 acrylate, 4-arm PEG-5000 acrylate, 6-arm PEG-2500-(NH₂)₆, genipin and FeCl₃, thiolated pluronic F-127, dopamine or DOPA/H₂O₂, Dextran aldehyde, NHS/EDC, NHS/DCC, EDC, disuccinimidyl tartrate (DST), disuccinimidyl malate (DSM) and trisuccinimidyl citrate (TSC), 4-arm PEG-thiol, trilysine, collagen, glutaraldehyde, PEG-diacrylate, ethylene glycole dimethacrylate (EGDMA), triethylene glycol dimethacrylate (TEGDMA), poly(ethylene glycol) dimethacrylate (PEGDMA), poly(MMA-co-AA-co-allylmethacrylate), 1,3-butylene glycol dimethacrylate (BGDMA), 1,4-butane diol diacrylate (BDDA), 1,6-hexane diol diacrylate (HDDA), hexanediol dimethacrylate (HDDMA), 1-carboxy-N-methyl-N-di(2-methacryloyloxy-ethyl) methanaminium inner salt (CBMX), bis(meth)acrylamides, neopentylglycol diacrylate (NPGDA), trimethylolpropane triacrylate (TMPTA), and combinations thereof.

99. The in situ curable bioadhesive of claim 96, wherein the in situ curable hydrogel adhesive prepolymers comprise a two-component in situ curable silicone hydrogel adhesive precursor and a platinum catalyst, wherein one of the silicone hydrogel adhesive precursor has Si—H groups and the other silicone hydrogel adhesive precursor has complementary reactive Si-vinyl groups (Si—CH═CH₂).

100. The in situ curable bioadhesive of claim 96, wherein the in situ curable hydrogel adhesive prepolymers comprise cross-linkable polydopamines.

101. A method for accelerating an in situ polymerization reaction of a curable bioadhesive at a tissue site comprising the steps: (1) applying the in situ curable bioadhesive of claim 67 to the tissue site; and (2) exposing the in situ curable bioadhesive to the exogenous source.

102. The method of claim 101, wherein the exogenous source is selected from the group consisting of a body chemical, an electromagnetic radiation, an electrical field, a microwave, a radio wave, ultrasonic radiation, a magnetic field, and combinations thereof.

103. The method of claim 101, wherein the temperature in the in situ curable bioadhesive is increased to a value ranging from about 50° C. to about 90° C.

104. An in situ curable composition for hard tissue repair comprising: (1) a curable resin comprising a polymerizable precursor for radical polymerization, and (2) the medium of claim 1 or the particle heater of claim 25, wherein the material absorbs energy from the exogenous source and converts the absorbed energy to heat to generate reactive oxygen species, and wherein the curable compositions pass the Extractable Cytotoxicity Test.

105. The in situ curable composition of claim 104, wherein the curable compositions further passes the Efficacy Determination Protocol.

106. The in situ curable composition of claim 104, wherein the curable compositions further passes the Thermal Cytotoxicity Test.

107. The in situ curable composition of claim 104, wherein the curable compositions further comprises a toughener.

108. The in situ curable composition of claim 107, wherein the toughener is an elastomeric rubber selected from the group consisting of polyethylene, polypropylene, polybutene, polypentene, ethylene-propylene copolymers, isoprene-butene copolymers, ethylene-butene copolymers, polybutadiene, polyisoprene, hydrogenated polybutadiene, hydrogenated polyisoprene, ethylene-propylene-diene copolymers, ethylene-butene-diene copolymers, butyl rubber, polystyrene, styrene-butadiene copolymers, styrene-hydrogenated butadiene copolymers, and combinations thereof.

109. The in situ curable composition of claim 104, wherein the curable compositions further comprises a heat-dissipating agent to reduce temperature increase during the exothermic polymerization of the curable dental composition.

110. The in situ curable composition of claim 109, wherein the heat dissipating agent is selected from the group consisting of a volatile liquid, a solid having a melting point of from about 20° C. to about 150° C., and a solid having a sublimation point of from about 20° C. to about 150° C.

111. The in situ curable composition of any one claims 109-110, wherein the heat dissipating agent is selected from the group consisting of potassium nitrate, sodium acetate trihydrate, sodium sulfate decahydrate, barium hydroxide octahydrate, calcium oxalate dihydrate, magnesium oxalate dihydrate, aluminum hydroxide, zinc sulfate, aluminum oxide, barium oxide, titanium oxide, manganese oxide, calcium oxide, metal nanoparticles such as copper, lead, nickel, aluminum, and zinc, carbon black and carbides, graphene nanoparticle, graphene oxide nanoparticle, urea, paraffin wax and polyvinyl fluoride, poly(N-isopropylacrylamide) (PNIPAAm) composite incorporating glycidyl methacrylate functionalized graphene oxide (GO-GMA), 2-hydroxy-2-trimethylsilanyl-propionitrile, 1-fluoropentacycloundecane, 6,7-diazabicyclo[3.2.1]oct-6-ene, 5,5,6,6-tetramethylbicyclo[2.2.1]heptan-2-ol, complex of dimethyl magnesium and trimethylaluminum, N-benzyl-2,2,3,3,4,4,4-heptafluoro-butyramide, 3-isopropyl-5,8a-dimethyl-decahydronaphthalen-2-ol, 2-hydroxymethyl-1,7,7-trimethyl-bicyclo[2.2.1]heptan-2-ol, 3,5-dichloro-3-methyl-cyclopentane-1,2-dione, (5-methyl-2-oxo-bicyclo[3.3.1]non-3-en-1-yl)-acetic acid, 4b,6a,11,12-tetrahydro-indeno[2,1-a]fluorene-5,5,6,6-tetracarbonitrile, tetracosafluoro-tetradecahydro-anthracene, 4,5-dichlorobenzene-1,2-dicarbaldehyde, bicyclo[4,3.1]dec-3-en-8-one, 3-tert-butyl-1,2-bis-(3,5-dimethylphenyl)-3-hydroxyguanidine, 1-[2,6-dihydroxy-4-methoxy-3-methylphenyl]butan-1-one, 2,3,6,7-tetrachloronaphthalene, 2,3,6-trimethylnaphthalene, dodecafluoro-cyclohexane, 2,2,6,6-tetramethyl-4-hepten-3-one, 1,1,1-trichloro-2,2,2-trifluoro-ethane, [5-(9H-beta-carbolin-1-yl)-furan-2-yl]methanol, 5-nitro-benzo[1,2,3]thiadiazole, 4,5-dichloro-thiophene-2-carboxylic acid, 2,6-dimethyl-isonicotinonitrile, nonafluoro-2,6-bis-trifluoromethyl-piperidine, (dimethylamino)difluoroborane, dinitrogen pentoxide, chromyl fluoride, chromium hexacarbonyl, 1-methylcyclohexanol, phenyl ether, nonadecane, 1-tetradecanol, 4-ethylphenol, benzophenone, maleic anhydride, octacosane, dimethyl isophthalate, butylated hydroxytoluene, glycolic acid, vanillin, magnesium nitrate hexahydrate, cyclohexanone oxime, glutaric acid, D-sorbitol, phenanthrene, fluorene, trans-stilbene, neopentyl glycol, pyrogallol, and diglycolic acid, and combinations thereof.

112. The in situ curable composition of claim 104, wherein the carrier is admixed with the material to form a particle.

113. The in situ curable composition of claim 112, wherein the particle further comprises a shell to form a core-shell particle.

114. The in situ curable composition of claim 113, wherein the shell comprises a crosslinked inorganic polymer selected from the group consisting of mesoporous silica, organo-modified silicate polymer derived from condensation of organotrisilanol or halotrisilanol, and combinations thereof.

115. The in situ curable composition of claim 113, wherein the shell comprises an agent selected from the group consisting of Au, Ag, Cu, iron oxide, and combinations thereof.

116. The in situ curable composition of claim 113, wherein the shell comprises a plasmonic absorber.

117. The in situ curable composition of claim 116, wherein the plasmonic absorber comprises plasmonic nanomaterials of noble metal gold (Au), silver (Ag) and copper (Cu) doped with sulfur (S), selenium (Se) or tellurium (Te) having a plasmonic resonance at a NIR wavelength.

118. The in situ curable composition of claim 116, wherein the plasmonic absorber is a gold nanostructure selected from the group consisting of gold nanorod, gold nanocage, gold nanofilm, gold nanosphere, and combinations thereof.

119. The in situ curable composition of any one of claims 112-118, wherein the particle heater maintains integrity or alters its structure after interacting with the exogenous source.

120. The in situ curable composition of claim 104, wherein the polymerizable precursor is selected from the group consisting of a polymerizable monomer, and a polymerizable prepolymer.

121. The in situ curable composition of claim 120, wherein the polymerizable and/or crosslinkable precursor is a polymerizable monomer for radical polymerization.

122. The in situ curable composition of claim 120, wherein the polymerizable and/or crosslinkable precursor is a polymerizable prepolymer for radical polymerization.

123. The in situ curable composition of claim 104, wherein the carrier comprises a lipid or a biocompatible organic polymer.

124. The in situ curable composition of claim 123, wherein the lipid is selected from the group consisting of dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-dimyristoyl-sn-glycero-3-phosphorylglycerol (DMPG); 1,2-distearoyl-sn-glycero-3-phosphoethanolamine (DSPE); 1,2-dioleoyl-sn-glycero-3-phosphocholine (DOPC); 1,2-dioleoyl-sn-glycero-3-phosphoethanolamine (DOPE); 1,2-dipalmitoyl-sn-glycero-3-phospho-(1′-rac-glycerol) (DPPG); distearoylphosphoethanolamine conjugated with polyethylene glycol (DSPE-PEG); phosphatidylserine (PS), phosphatidylethanolamine (PE), phosphatidylglycerol (PG), phosphatidylcholine (PC), and combinations thereof.

125. The in situ curable composition of claim 123, wherein the lipid is selected from the group consisting of DPPC, MPPC, PEG, DMPC, DMPG, DSPE, DOPC, DOPE, DPPG, DSPC, DSPE-PEG, MSPC, cholesterol, PS, PC, PE, PG, 1,2-dipalmitoyl-sn-glycero-3-phospho-(1′-rac-glycerol) (DPPG), 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-diarachidyl-sn-glycero-3-phosphoethanolamine (20:0 PE), 1-stearoyl-2-linoleoyl-sn-glycero-3-phosphoethanolamine, 1,2-diheptadecanoyl-sn-glycero-3-phosphocholine (17:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), carbohydrate-lipid conjugate, polymer-lipid conjugate, peptide-lipid conjugate, protein-lipid conjugate, and combinations thereof.

126. The in situ curable composition of claim 123, wherein the biocompatible organic polymer is selected from the group consisting of poly (dimethyl siloxane) (PDMS), polydioxanone, poly (meth) acrylamides, polyetheretherketone (PEEK), poly(methyl methacrylate), polyester including poly(lactic acid-co-glycolic acid) (PLGA), polyglycolic acid (PGA), polylactic acid (PLA), polycaprolactone (PCL), poly(trimethylene carbonate), poly (alpha-esters), polyurethanes, poly(allylamine hydrochloride), poly(ester amides), poly (ortho esters), polyanyhydrides, poly (anhydride-co-imide), crosslinked polyanhydrides, pseudo poly(amino acids), poly (alkylcyanoacrylates), polyphosphoesters, polyphosphazenes, chitosan, collagen, gelatin, natural or synthetic poly(amino acids), elastin, elastin-linked polypeptides, albumin, fibrin, polysiloxanes, polycarbosiloxanes, polysilazanes, polyalkoxysiloxanes, polysaccharides, cross-linkable polymers, thermoresponsive polymers, thermo-thinning polymers, thermo-thickening polymers, block co-polymers comprising polyethylene glycol, and combinations thereof.

127. The in situ curable composition of any one of claims 104-126, wherein the material has significant absorption of photonic energy in the near infrared spectral region having a wavelength range from 750 nm to 1400 nm.

128. The in situ curable composition of any one of claims 104-126, wherein the material has absorption of photonic energy in the near infrared spectral region having a wavelength range from 750 nm to 1200 nm.

129. The in situ curable composition of any one of claims 104-126, wherein the material has absorption of photonic energy in the near infrared spectral region having a wavelength range from 900 nm to 1100 nm.

130. The in situ curable composition of any one of claims 104-126, wherein the material has absorption of photonic energy in the near infrared spectral region having a wavelength range from 750 nm to 850 nm.

131. The in situ curable composition of any one of claims 104-126, wherein the material is selected from the group consisting of organic dyes, inorganic dyes, near-infrared absorbing dyes, tetrakis aminium dyes, zinc iron phosphate pigments, iron oxide nanoparticle, and combinations thereof.

132. The in situ curable composition of any one of claims 104-123, wherein the exogenous source is selected from the group consisting of a chemical, an electromagnetic radiation, an electrical field, a microwave, a radio wave, ultrasonic radiation, a magnetic field, and combinations thereof.

133. An in situ curable dental composition comprising the curable composition of claim 104, wherein the material converts the absorbed energy from the exogenous source to heat to induce localized hyperthermia, wherein the localized hyperthermia causes the polymerization of the curable resin to form a cured resin reinforced with the filler.

134. The remotely-triggered in situ curable dental composition of claim 133, wherein the in situ curable dental composition comprises 70.0-90.0 wt. % of a filer, 10.0-30.0 wt. % of the curable resin, a 1 wt. % to 10 wt. % of the particle heater, an polymerization initiator, and a contrast agent.

135. The remotely-triggered in situ curable dental composition of claim 133, wherein the curable resin comprising a mixture of 15.0 wt. % to 45.0 wt. % of ethoxylated bisphenol A bisethylmethacrylate ester having 6 units ethoxyl repeating groups (BisEMA6), 15.0 wt. % to 45.0 wt. % of urethane dimethacrylate (UDMA), 10.0 wt. % to 40.0 wt. % of BisGMA, and 0 wt. % to 10.0 wt. % of triethylene glycol dimethacrylate (TEGDMA), and wherein the percentage weight of the monomers are by the total weight of the curable resin.

136. The remotely-triggered in situ curable dental composition of claim 133, wherein the curable resin is a mixture of 30.0 wt. % to 40.0 wt. % of BisEMA6, 30.0 wt. % to 40.0 wt. % of UDMA, 20.0 wt. % to 30.0 wt. % of BisGMA, and 0 wt. % to 10.0 wt. % of TEGDMA.

137. The remotely-triggered in situ curable dental composition of claim 133, wherein the curable resin is a mixture of 33.0 wt. % to 37.0 wt. % of BisEMA6, 33.0 wt. % to 37.0 wt. % of UDMA, 23.0 wt. % to 27.0 wt. % of BisGMA, and 0 wt. % to 5.0 wt. % of TEGDMA.

138. The remotely-triggered in situ curable dental composition of claim 133, wherein the filler is an inorganic filler selected from the group consisting of quartz; nitrides; glasses derived from Ce, Sb, Sn, Zr, Sr, Ba or Al; colloidal silica; a composite glass composed of oxides of barium, silicon, boron, and aluminum, feldspar; borosilicate glass; kaolin; talc; titania; zinc glass; zirconia-silica; fluoroaluminosilicate glass; submicron silica particles, and combinations thereof.

139. The remotely triggered in situ curable dental composition of claim 133, wherein the filler is an organic filler selected from the group consisting of filled or unfilled pulverized polycarbonates, polyepoxides, and combinations thereof.

140. The remotely-triggered in situ curable dental composition of any one of claims 138-139, wherein the surface of the fillers may be treated with a surface treatment comprising a silane coupling agent to enhance the bond between the filler and the curable resin.

141. The remotely triggered in situ curable dental composition of claim 140, wherein the coupling agent may be functionalized with reactive curing groups selected from the group consisting of acrylates, methacrylates, and combinations thereof.

142. The remotely triggered in situ curable dental composition of claim 133, wherein the filler comprises sintered ceramic composite of zirconia-silica.

143. The remotely triggered in situ curable dental composition of claim 142, wherein the sintered ceramic composite of zirconia-silica comprises submicron particles having a median particle size of 600 nm to 900 nm.

144. The remotely triggered in situ curable dental composition of claim 133, further comprising a radiopacifying agent.

145. The remotely triggered in situ curable dental composition of claim 144, wherein the radiopacifying agent is selected from the group consisting of HfO₂, La₂O₃, SrO, ZrO₂, and combinations thereof.

146. An in situ curable bone cement comprising a solid phase comprising a polymer powder, a contrast agent and a polymerization initiator, and a liquid phase comprising an acrylate monomer for radical polymerization, an accelerator, and a polymerization inhibitor; wherein the polymerization initiator is capable of generating free radicals to catalyze the in situ polymerization of the monomer to provide a cured bone cement.

147. The in situ curable bone cement of claim 146, wherein the polymer powder containing a polymer selected from the group consisting of polymethylmethacrylate (PMMA); poly(hydroxyalkenoate), poly([R]-3-hydroxybutyrate (PHB), PMMA-graft-PHB, corn starch and cellulose acetate (SCA); SCA reinforced hyaluronic acid (HA), HA particles silanized with 3-(triethoxysilyl)propyl methacrylate, poly(MMA-co-EMA), and combinations thereof.

148. The in situ curable bone cement of claim 146, wherein the monomer is selected from the group consisting of methyl-methacrylate monomer (MMA); a mixture of MMA and acrylic acid (AA) (MMA+AA); 2-hydroxyethyl methacrylate (HEMA); a mixture of bisGMA, EGDMA and MMA; and a methacrylated amino acid containing anhydride oligomer as a reaction product of maleic acid, alanine and 6-aminocaproic acid and TEGMDA, and combinations thereof.

149. The in situ curable cement of any one of the claims 146-148, wherein the polymer powder has a particle size of about 10 μm to about 100 μm.

150. The in situ curable bone cement of claim 146, wherein the polymerization initiator comprises a particle for producing reactive oxygen species (ROS) comprising a carrier and a material interacting with an exogenous source, and wherein the particle is constructed such that it passes the Extractable Cytotoxicity Test.

151. The in situ curable bone cement of claim 146, wherein the particle further passes the Efficacy Determination Protocol.

152. The in situ curable bone cement of claim 146, wherein the particle further passes the Thermal Cytotoxicity Test.

153. The in situ curable bone cement of any one of claims 146-152, wherein the exogenous source is selected from the group consisting of a chemical, an electromagnetic radiation, a microwave, an electrical field, a magnetic field, sound (ultrasonic) wave, and combinations thereof.

154. The in situ curable bone cement of any one of claims 146-152, wherein the material absorbs the energy from the exogenous source and causes the production of reactive oxygen species.

155. The in situ curable bone cement of any one of claims 146-152, wherein the accelerator is a divalent iron salt, wherein the divalent iron ion catalyzes the ROS degradation to hydroxyl free radical.

156. The in situ curable bone cement of claim 146, wherein the exogenous source comprises a LED light or a laser light.

157. The in situ curable bone cement of claim 146, wherein the exogenous source comprises a LED light.

158. The bone cement of any one of claims 146-157, wherein the particle maintains its integrity after its exposure to the exogenous source.

159. The in situ bone cement of any one of claims 146-157, wherein the particles are microparticles or nanoparticles.

160. The in situ bone cement of any one of claims 146-157, wherein the particle further comprises a shell to enclose the particle to form a core-shell particle.

161. The in situ bone cement of claim 160, wherein the shell comprises a thin layer of plasmonic absorber selected from the group consisting of Au, Ag, Cu, iron oxide, polydopamine, and combinations thereof.

162. The in situ curable bone cement of claim 146, wherein the material is a plasmonic absorber, a cyanine dye, a sqaurylynium dye, iron oxide, or a tetrakis aminium dye.

163. The in situ curable bone cement of claim 146, wherein the material is a plasmonic absorber.

164. The in situ curable bone cement of claim 163, wherein the plasmonic absorber is selected from the group consisting of gold nanostructures including gold nanorod, gold nanosphere, gold nanocage, nanoporous gold thin film, gold nanoshell, silver nanoparticle, polydopamine coated gold-silver alloy nanoparticle, iron oxide, graphene oxide, Cu₂S, Cu₃BiS₃ nanoparticle, and combinations thereof.

165. The in situ curable bone cement of claim 146, wherein the material is iron oxide nanoparticles or iron oxide coating on the particle surface.

166. The in situ curable bone cement of any one of claims 146-165, wherein the material is indocyanine green, (ICG) or new ICG dye (IR 820).

167. The in situ curable bone cement of any one of claims 146-165, wherein the material is gold nanostructures.

168. The in situ curable bone cement of claim 146, wherein the polymerization initiator is selected from the group consisting of benzoyl oxide, tri-n-butyl borane, 2-5-dimethylhexane-2-5-dihydroperoxide, the particle heater, and combinations thereof.

169. The in situ curable bone cement of claim 146, wherein the contrast agent is a radiopacifier, gold nanostructure, ICG, iron oxide, and combinations thereof.

170. The in situ curable bone cement of claim 169, wherein the radiopacifier is a BaSO₄ particle of diameter of 100 nm, a BaSO₄ particle of diameter of 1000 nm, ZrO₂ particle, a nonpolar-hydrophobic heavy metal-containing organic material, capable of forming complex with PMMA including triphenyl bismuth (TBP), tantalum powder, bismuth salicylate (BS), strontium containing hyaluronic acid (Sr-HA), polymer-based iodine contrast agent, and polymer-based bromine contrast agent.

171. The in situ curable bone cement of claim 170, wherein the polymer-based iodine contrast agent is selected from the group consisting of iodinated copolymer of (MMA) and 2-[4-iodobenzoyl]-oxo-ethyl-methacrylate in a 1:1 weight/weight ratio (I-copolymer), iodixanol (IDX), iohexol (IHX), 2,5-diiodo-8-quinolyl methacrylate (IHQM), (4-iodophenol methacrylate, 2-[2′,3′,5′-triiodobenzoyl] ethyl methacrylate (TIBMA), 3,5-diiodine salicylic methacrylate (DISMA), iohexol acetate, and combinations thereof.

172. The in situ curable bone cement of claim 170, wherein the polymer-based bromine contrast agent is selected from the group consisting of 2-(2-bromoisobutyryloxy) ethyl methacrylate, a copolymer of MMA and 2-(2-bromopropionyloxy) ethyl methacrylate, and combinations thereof.

173. The in situ curable bone cement of claim 146, wherein the accelerator is selected from the group consisting of N,N-dimethyl-p-toluidine (DMPT), 2-5-dimethylhexane-2-5-dihydroperoxide, 4,N,N-(diethylamino) phenethanol, 4,4-(dimethylamino) phenyl acetic acid, 4-dimethylamino benzyl methacrylate, 4-dimethylamino benzyl alcohol, 4,4-dimethylamino benzydrol, 4-N,N-dimethylamino-4-benzyl laurate (DMAL), 4-N,Ndimethylamino-4-benzyl oleate (DMAO), and combinations thereof.

174. The in situ curable bone cement of claim 146, wherein the polymerization inhibitor is an antioxidant.

175. The in situ curable bone cement of claim 174, wherein the antioxidant is selected from the group consisting of hydroquinone, vitamin E, butylated hydroxytoluene (BHT), 2-t-butylhydroquinone, and 2-t-butylhydroxyanisole, pentaerythritol tetrakis(3,5-di-tert-butyl-4-hydroxyhydrocinnamate), tris(2,4-di-tert-butylphenyl)phosphite, 1,3,5-tris(4-tert-butyl-3-hydroxy-2,6-dimethylbenzyl)-1,3,5-triazine-2,4,6-(1H,3H,5H)-trione, and combinations thereof.

176. The in situ curable bone cement of claim 146, wherein the bone cement further comprises a crosslinking agent.

177. The in situ curable bone cement of claim 176, wherein the crosslinking agent is selected from the group consisting of ethylene glycole dimethacrylate (EGDMA), triethylene glycol dimethacrylate (TEGDMA), poly(ethylene glycol) dimethacrylate (PEGDMA), poly(MMA-co-AA-co-allylmethacrylate), and combinations thereof.

178. The in situ curable bone cement of claim 146, wherein the bone cement further comprises a reinforcement filler.

179. The in situ curable bone cement of claim 178, wherein the reinforcement filler is selected from the group consisting of the remotely triggered particle of claim 1, graphite fiber, carbon fiber, titanium fiber, trimethyl silane plasma-, cold plasma-, or hexaethylsiloxane plasma-treated graphite or carbon fiber, polyethylene (PE) fiber, polyethylene terephtahalate fiber, stainless steel fiber, stainless steel having surface bound methacryloxypropyl-trichlorosilane, ultra-high molecular wright polyethylene (UHMWPE), ultra-high-strength PE, UHMWPE grafted with MMA, ultra-high-strength PE grafted with MMA, beads of rubber-toughened PMMA powder having a PMMA outer shell and an inner shell made of crosslinked butyl methacrylate-styrene copolymer, beads of poly(isobutylene), beads of acrynitrile-butadiene-styrene; beads of poly(ε-caprolactone), particles of poly(butyl methacrylate) (PBMA), PCL-toughened PMMA beads, α- and δ alumina powder, alumina particles treated with a silane, silanized HA particle, sintered HA particle, silane-treated fluorohydroxyapatite particle, particle of PMAA, particle of PMETA-PMMA, particle of PEMA, particle of PEMA-n-BMA, chitosan nanoparticles, and combinations thereof.

180. The in situ curable bone cement of claim 179, wherein the reinforcement filler is the particle heater of claim 1, wherein the material converts the absorbed energy to heat, wherein the heat induces localized hyperthermia, wherein the hyperthermia as adjuvant for bone healing process.

181. A wound closure device comprising a structural element and the heat delivery medium of claim 1 or the particle heater of claim 25, wherein the heat causes thermally induced shrinkage of the structural element, and wherein the wound closure device passes the Extractable Cytotoxicity Test.

182. The wound closure device of claim 181, wherein the heat delivery composition further comprises a carrier.

183. The wound closure device of claim 182, wherein the carrier and the material form a particle.

184. The wound closure device of claim 181, wherein the wound closure device further passes the Thermal Cytotoxicity Test.

185. The wound closure device of claim 181, wherein the wound closure device further passes the Efficacy Determination Protocol.

186. The wound closure device of any one of claims 181-185, wherein the structural element is biodegradable and/or bioabsorbable.

187. The wound closure device of any one of claims 181-186, wherein the structural element is derived from the group consisting of gut, chromic gut, nylon, rayon, polyethylene, pluronic F127, chitosan, collagen, laminin, fibronectin, polyacrylamide, aminoglycoside hydrogels, fibrin, poly-lactic acid, poly-glycolic acid, poly(lactic-co-glycolic acid) (PLGA), polyglyconate, polydioxanone, poly(trimethylene carbonate), silk, poly(glycolic acid-ε-caprolactone), cotton, gelatin, polypropylene, titanium, metal, polysulfone, poly(ethylene terephthalate) (PETE), and combinations thereof.

188. The wound closure device of claim 181, wherein the carrier comprises a lipid, a biological glue agent, an inorganic polymer, or an organic polymer.

189. The wound closure device of claim 181, wherein the carrier comprises a biological glue agent capable of forming a bond to tissue segments and thereby hold them together while natural healing processes occur.

190. The wound closure device of claim 189, wherein the biological glue agent is selected from the group consisting of collagen, elastin, fibrin, albumin, and combinations thereof.

191. The wound closure device of claim 188, wherein the organic polymer is selected from the group consisting of PLGA, PLGA-PEG, polycaprolactone (PCL), poly-1-lysine (PLL), albumin, silk, milk protein, chitosan, polymer or a copolymer of methyl methacrylate, and combinations thereof.

192. The wound closure device of claim 188, wherein the lipid is selected from the group consisting of 1,2-dipalmitoyl-sn-glycero-3-phospho-(1′-rac-glycerol) (DPPG), 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-distearoyl-sn-glycero-3-phosphoethanolamine (DSPE, 18:0), 1,2-diarachidyl-sn-glycero-3-phosphoethanolamine (20:0 PE), 1-stearoyl-2-linoleoyl-sn-glycero-3-phosphoethanolamine, 1,2-dipalmitoyl-sn-glycero-3-phosphocholine (DPPC, 16:0 PC), 1,2-diheptadecanoyl-sn-glycero-3-phosphocholine (17:0 PC), 1,2-distearoyl-sn-glycero-3-phosphocholine (DSPC, 18:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

193. The wound closure device of claim 188, wherein the inorganic polymer is selected from the group consisting of mesoporous silica, organo-modified silicate polymer derived from condensation of organotrisilanol or halotrisilanol, and combinations thereof.

194. The wound closure device of any one of claims 181-193, wherein the carrier comprises reactive aldehydes or epoxy groups capable of reacting with amines, hydroxyls, or carboxyl groups of tissue proteins.

195. The wound closure device of claim 181, wherein the material has significant absorption of photonic energy in the near infrared spectrum region having a wavelength range from 750 nm to 1400 nm.

196. The wound closure device of claim 181, wherein the material has absorption of photonic energy in the near infrared spectrum region having a wavelength range from 750 nm to 1200 nm.

197. The wound closure device of claim 181, wherein the material has absorption of photonic energy in the near infrared spectrum region having a wavelength range from 900 nm to 1100 nm.

198. The wound closure device of claim 181, wherein the material has absorption of photonic energy in the near infrared spectrum region having a wavelength range from 750 nm to 850 nm.

199. The wound closure device of claim 181, wherein the material has absorption of photonic energy in the spectrum region having a wavelength range from 400 nm to 750 nm.

200. The wound closure device of any one of the claims 195-199, wherein the material is selected from the group consisting of organic dyes, inorganic dyes, near-infrared absorbing dyes, tetrakis aminium dyes, a cyanine dye, a squaraine dye, a squarylium dye, zinc iron phosphate pigments, indocyanine green, and combinations thereof.

201. The wound closure device of claim 181, wherein the heat delivery composition comprises two or more materials and each absorbs energy from a different exogenous source.

202. The wound closure device of claim 181, wherein the material interacting with exogenous comprises a plasmonic absorber.

203. The wound closure device of claim 202, wherein the plasmonic absorber comprises plasmonic nanomaterials of noble metal gold (Au), silver (Ag) and copper (Cu) nanoparticles doped with sulfur (S), selenium (Se) or tellurium (Te) having a plasmonic resonance at a NIR wavelength.

204. The wound closure device of claim 181, wherein the exogenous source is selected from the group consisting of a body chemical, an electromagnetic radiation, an electrical field, a microwave, a radio wave, ultrasonic radiation, a magnetic field, and combinations thereof.

205. The wound closure device of claim 204, wherein the body chemical is blood, blood components, water, amines, hydroxyls, or carboxyl groups.

206. The wound closure device of any one of claims 181-205, wherein the heat delivery composition forms a coating on the structural element.

207. The wound closure device of claim 183, wherein the particle is dispersed in the structural element.

208. The wound closure device of claim 207, wherein the particle is a nanoparticle or a microparticle.

209. The wound closure device of any one of claims 207-208, wherein the particle maintains integrity after interacting with the exogenous source.

210. The wound closure device of any one of claims 207-208, wherein the particle structure is altered after interacting with the exogenous source.

211. The wound closure device of any one of claims 181-210, wherein the structural element is configured as a suture, staple, screw, tape, patch, adhesive, or sealant.

212. A method for joining tissue at a wound site or body scission comprising the steps of (1) delivering the wound closure device of claim 181 further comprising a shape memory polymer to the tissue at the wound site or body scission; (2) applying the wound closure device loosely in its temporary shape, (3) tying a loose knot of the wound closure device; (4) irradiating the wound closure device with a pulsed laser to convert photonic energy of the laser irradiation into heat, wherein the heat causes the wound closure device to join the tissue at the wound site or body scission; wherein the heated wound closure device shrinks and tightens the knot by applying an optimum force by increasing the temperature higher than glass transition temperature (T_{g}), wherein the suture passes the Extractable Cytotoxicity Test.

213. The method of claim 212, wherein the suture is irradiated with a pulsed laser at a wavelength of 1064 nm, at a fluence of 10 J/cm² with a 100 ms pulse.

214. The method of claim 212, wherein the suture is irradiated with a pulsed laser at a wavelength of 805 nm, at a fluence of 40 J/cm² with a 100 ms pulse.

215. A hemostatic composition useful for enhancement of clotting of blood in a subject that comprises (i) the medium of claim 1 or the particle of claim 25, and (ii) a physiologically acceptable medium, wherein the heat travels outside the hemostatic composition to an area surrounding the hemostatic composition, wherein the heat causes a controlled temperature rise to initiate or accelerate the formation of a blood clot, and wherein the hemostatic composition passes the Extractable Cytotoxicity Test.

216. The hemostatic composition of claim 215, wherein the subject is a warm-blooded animal.

217. The hemostatic composition of claim 215, wherein the subject is a human.

218. The hemostatic composition of claim 215, wherein the hemostatic composition passes the Thermal Cytotoxicity Test.

219. The hemostatic composition of claim 215, wherein the hemostatic composition passes the Efficacy Determination Protocol.

220. The hemostatic composition of claim 215, wherein the particle heater is a microparticle, or nanoparticle.

221. The hemostatic composition of claim 220, wherein the particle maintains its integrity after exposure to the exogenous source.

222. The hemostatic composition of claim 220, wherein the particle structure is altered after exposure to the exogenous source.

223. The hemostatic composition of claim 220, wherein the particle further comprises a shell to form a core-shell structure.

224. The hemostatic composition of claim 223, wherein the core comprises an agent selected from the group consisting of Au, Ag, Cu, iron oxide, and combinations thereof.

225. The hemostatic composition of claim 223, wherein the shell comprises a plasmonic absorber.

226. The hemostatic composition of claim 225, wherein the plasmonic absorber comprises plasmonic nanomaterials of noble metal gold (Au), silver (Ag) and copper (Cu) nanoparticles doped with sulfur (S), selenium (Se) or tellurium (Te) having a plasmonic resonance at a NIR wavelength.

227. The hemostatic composition of claim 215, wherein the material interacting with the exogenous source is an absorbing material having significant absorption of photonic energy.

228. The hemostatic composition of claim 215, wherein the exogenous source is a laser light.

229. The hemostatic composition of claim 215, wherein the exogenous source is a LED light.

230. The hemostatic composition of claim 228, wherein the material interacting with the exogenous source has significant absorption of photonic energy in the near infrared spectrum region having a wavelength range from 750 nm to 1400 nm.

231. The hemostatic composition of claim 229, wherein the material interacting with the exogenous source has significant absorption of photonic energy in the spectrum region having a wavelength range from 400 nm to 750 nm.

232. The hemostatic composition of claim 215, wherein the material interacting with the exogenous source has absorption of photonic energy in the near infrared spectrum region having a wavelength range from 750 nm to 850 nm, or 750 nm to 1200 nm.

233. The hemostatic composition of claim 215, wherein the material interacting with the exogenous source has absorption of photonic energy in the near infrared spectrum region having a wavelength range from 900 nm to 1100 nm.

234. The hemostatic composition of claim 215, wherein the material absorbs light at a wavelength selected from the group consisting of 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, and 750 nm.

235. The hemostatic composition of any one of the claims 215-234, wherein the material interacting with the exogenous source is a tetrakis aminium dye, a cyanine dye, a squarylium dye, squaraine dye, iron oxide, or a zinc iron phosphate pigment.

236. The hemostatic composition of claim 215, wherein the exogenous source is selected from the group consisting of an electromagnetic radiation, an electrical field, a microwave, a radio wave, ultrasonic radiation, a magnetic field, and combinations thereof.

237. The hemostatic composition of any one claims 215-236, wherein the carrier comprises a biocompatible polymer selected from the group consisting of mesoporous silica, polymethyl methacrylate, polyester including poly(lactic acid-co-glycolic acid) (PLGA), polyglycolic acid (PGA), polylactic acid (PLA), polycaprolactone (PCL), poly(trimethylene carbonate), poly (alpha-esters), polyurethanes, poly(allylamine hydrochloride), poly(ester amides), poly (ortho esters), polyanyhydrides, poly (anhydride-co-imide), crosslinked polyanhydrides, pseudo poly(amino acids), poly (alkylcyanoacrylates), polyphosphoesters, polyphosphazenes, chitosan, collagen, gelatin, natural or synthetic poly(amino acids), elastin, elastin-linked polypeptides, albumin, fibrin, polysiloxanes, polycarbosiloxanes, polysilazanes, polyalkoxysiloxanes, polysaccharides (e.g. chitosan), cross-linkable polymers, block co-polymers comprising polyethylene glycol, block co-polymers comprising polyoxyalkylene, and combinations thereof.

238. The hemostatic composition of any one of claims 215-236, wherein the carrier comprises a crosslinked biocompatible and biodegradable, polymer wherein the biocompatible and biodegradable polymer is selected from the group consisting of chitosan and derivatives thereof, hyaluronic acid, alginate, alginic acid, starch, carrageenan, and combinations thereof.

239. The hemostatic composition of any one of claims 215-236, wherein the carrier comprises a methyl methacrylate/butyl methacrylate copolymer comprising 96% methyl methacrylate repeating units and 4% butyl methacrylate repeating units.

240. The hemostatic composition of any one of claims 215-236, wherein the carrier comprises a lipid.

241. The hemostatic composition of claim 240, wherein lipid is selected from the group consisting of 1,2-dipalmitoyl-sn-glycero-3-phospho-(1′-rac-glycerol) (DPPG), 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-distearoyl-sn-glycero-3-phosphoethanolamine (DSPE, 18:0), 1,2-diarachidyl-sn-glycero-3-phosphoethanolamine (20:0 PE), 1-stearoyl-2-linoleoyl-sn-glycero-3-phosphoethanolamine, 1,2-dipalmitoyl-sn-glycero-3-phosphocholine (DPPC, 16:0 PC), 1,2-diheptadecanoyl-sn-glycero-3-phosphocholine (17:0 PC), 1,2-distearoyl-sn-glycero-3-phosphocholine (DSPC, 18:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

242. The hemostatic composition of claim 240, wherein the lipid comprises a thermoresponsive lipid/polymer hybrid.

243. The hemostatic composition of claim 242, wherein the thermoresponsive lipid/polymer hybrid is selected from the group consisting of a triblock copolymer of [poly(2-isopropyl-2-oxazoline)-b-poly(dimethylsiloxane)-b-poly(2-isopropyl-2-oxazoline] and 1,2-dimyristoyl-sn-glycero-3-phosphocholine (DMPC) composite, block copolymers poly(cholesteryl acrylate)-b-poly(N-isopropylacrylamide) (PNIPAAm), lipid composite, and combinations thereof.

244. The hemostatic composition of any one of claims 215-243, wherein the physiologically acceptable medium is selected from the group consisting of liquid vehicles, granules, powder, microspheres, flakes, films, gel ointment, sponge, pastes, semisolid, hydrogel, water responsive shape memory hydrogel, crosslinkable polymers having reactive groups, crosslinked polymer networks, ribbons, hemostatic gauzes, compression gauzes, pads, band-aids, occlusive dressings, and combinations thereof.

245. The hemostatic composition of claim 215, wherein the physiologically acceptable medium comprises chitosan and oxidized regenerated cellulose.

246. The hemostatic composition of claim 215, wherein the physiologically acceptable medium further comprises chitosan and chitosan derivatives.

247. The hemostatic composition of claim 215, wherein the physiologically acceptable medium comprises a water responsive shape memory polymer.

248. The hemostatic composition of claim 215, wherein the particle heater is embedded within, dispersed, in or forms a coating layer on a surface of the physiologically acceptable medium.

249. The hemostatic composition of any one of claims 215-248, further comprising a hemostatic or coagulative agent selected from the group consisting of chitosan, calcium-loaded zeolite, silicate including kaolin, microfibrillar collagen, oxidized regenerated cellulose, anhydrous aluminum sulfate, silver nitrate, potassium alum, titanium oxide, fibrinogen, epinephrine, calcium alginate, poly-N-acetyl glucosamine, thrombin, coagulation factor(s) including Factor VII, Factor IX, Factor X, FVIIa, Von Willebrand factor, procoagulants including propyl gallate, antifibrinolytics including epsilon aminocaproic acid, coagulation proteins that generate Factor VII or FVIIa including Factor XII, Factor XIIa, Factor X, Factor Xa, protein C, protein S, and prothrombin, and combinations thereof.

250. A method of blood clotting in a subject in need thereof comprising administering to the subject a therapeutically effective amount of a hemostatic composition comprising (i) the medium of claim 1 or the particle heater of claim 25, and (ii) a physiologically acceptable medium; and contacting the hemostatic composition with an exogenous source, and optionally applying slight pressure on the hemostatic composition to reduce or stop bleeding, wherein the heat travels outside the hemostatic composition to an area surrounding the hemostatic composition, wherein the heat causes a controlled temperature rise to initiate or accelerate the formation of a blood clot, and wherein hemostatic composition passes the Extractable Cytotoxicity Test.

251. The method of claim 250, wherein the exogenous source is a pulsed laser light having an oscillation wavelength at 1064 nm.

252. The method of claim 250, wherein the exogenous source is a pulsed laser light having an oscillation wavelength from 780 to 810 nm.
